(19) United States
(12) Patent Application Publication
(10) Pub. No.: US 2022/0398426 A1
(43) Pub. Date: Dec. 15, 2022

(54) Novel Class 2 Type II and Type V CRISPR-Cas RNA-Guided Endonucleases

(71) Applicants: IONQ, INC., College Park, MD (US); Consejo Nacional De Investigaciones Cientificas Y Tecnicas (Conicet), Buenos Aires (AR)

(72) Inventors: Carla Alejandra Gimenez, Buenos Aires (AR); Guillermo Daniel REPIZO, San Francisco, CA (US); Federico Alberto PEREYRA BONNET, Caba (AR); Lucía Ana CURTI, Buenos Aires (AR); Franco GOYTIA, Miami Beach, FL (US); Maria Eugenia FARÍAS, Yerba Buena (AR)

(21) Appl. No.: 17/607,970

(22) PCT Filed: Sep. 10, 2020
(86) PCT No.: PCT/US2020/050237

§ 371 Date: Nov. 1, 2021

Related U.S. Application Data
(60) Provisional application No. 63/058,448, filed on Jul. 29, 2020.
(60) Provisional application No. 62/898,340, filed on Sep. 10, 2019.

(51) Int. Cl. G06N 3/00; G06N 10/40

(52) U.S. Cl. CPC G06N 3/002; G06N 10/40

(57) ABSTRACT

Provided herein are novel Class 2 Type II and Type V CRISPR-Cas RNA-guided endonucleases, e.g. Cas9 and Cas12 endonucleases, and systems comprising the same. Provided also are methods of making, and methods of use thereof. Exemplary methods of use include modifying target DNAs and detecting targeting DNAs, useful for therapeutic and diagnostic applications. Some of the diagnostic applications may utilise the collateral nuclease activity of an enzyme bound to a target sequence.

### CROSS REFERENCE TO RELATED APPLICATIONS

This application claims priority to U.S. Provisional Patent Application Ser. No. 63/058,448 filed Jul. 29, 2020 and U.S. Provisional Patent Application Ser. No. 62/898,340 filed Sep. 10, 2019, each of which are herein incorporated by reference in their entirety.

### DESCRIPTION OF THE TEXT FILE SUBMITTED ELECTRONICALLY

The sequence listing associated with this application is provided in text format in lieu of a paper copy, and is hereby incorporated by reference into the specification. The name of the text file containing the sequence listing is “CABI_002_02WO_SeqList_ST25.txt”. The text file is 456 kb, was created on Sep. 10, 2020, and is being submitted electronically via EFS-Web.

### BACKGROUND

Bacterial adaptive immune systems have in place CRISPRs (clustered regularly interspaced short palindromic repeats) and CRISPR-associated (Cas) proteins for RNA-guided nucleic acid cleavage. The CRISPR-Cas systems act to confer adaptive immunity in bacteria and archaea via RNA-guided nucleic acid interference. To provide immunity against invaders, processed CRISPR array transcripts (crRNAs) assemble with Cas protein-containing surveillance complexes that recognize nucleic acids bearing sequence complementarity to the invader's derived segment of the crRNAs, known as the spacer.

Class 2 CRISPR-Cas systems are streamlined versions in which a single Cas protein (an effector endonuclease protein) bound to RNA is responsible for binding to and cleavage of a targeted sequence. The programmable nature of these minimal systems has facilitated their use as a versatile technology that continues to revolutionize the field of genome manipulation.

There however is a need for improved Class 2 Type II and Type V CRISPR-Cas RNA-guided endonuclease variants. Provided herein are such variants, methods of making, methods of testing, and methods of using the same.

### SUMMARY

Provided herein are novel Class 2 Type II and novel Type V CRISPR-Cas RNA-guided systems, methods of making, and methods of use. More specifically, provided are novel Cas9 variants, novel Cas12a variants, and novel Cas12 subtypes.

In one aspect provided herein is an engineered system comprising: (a) a Cas9.1, Cas9.2, Cas9.3 or Cas9.4 protein, or a nucleic acid encoding the a Cas9.1, Cas9.2, Cas9.3 or Cas9.4 protein; and (b) a Cas9.1, Cas9.2, Cas9.3 or Cas9.4 guide RNA (gRNA), or a nucleic acid encoding the Cas9.1, Cas9.2, Cas9.3 or Cas9.4 gRNA, wherein the gRNA and the Cas9.1, Cas9.2, Cas9.3 or Cas9.4 protein do not naturally occur together, wherein the gRNA is capable of hybridizing to a target sequence in a target DNA, and the gRNA is capable of forming a complex with the Cas9.1, Cas9.2, Cas9.3 or Cas9.4 protein.

In another aspect, provided herein is an engineered single-molecule gRNA, comprising: (a) a targeter-RNA comprising a spacer sequence that is capable of hybridizing with a target sequence in a target DNA; and (b) an activator-RNA that is capable of hybridizing with the targeter-RNA to form a double-stranded RNA duplex, the activator-RNA comprising a activator-RNA, wherein the targeter-RNA and the activator-RNA are covalently linked to one another, wherein the single-molecule gRNA is capable of forming a complex with a Cas9.1, Cas9.2, Cas9.3 or Cas9.4 protein, and wherein hybridization of the spacer sequence to the target sequence is capable of targeting the Cas9.1, Cas9.2, Cas9.3 or Cas9.4 protein to the target DNA.

In another aspect, provided herein is an engineered system comprising: a Class 2 Type V CRISPR-Cas RNA-guided endonuclease protein and a single guide RNA, wherein the gRNA and the Class 2 Type V CRISPR-Cas RNA-guided endonuclease protein do not naturally occur together, wherein the gRNA is capable of hybridizing to a target sequence in a target DNA, wherein the gRNA is capable of forming a complex with the Class 2 Type V CRISPR-Cas RNA-guided endonuclease protein, and wherein the Class 2 Type V CRISPR-Cas RNA-guided endonuclease protein possesses collateral activity and is capable of collaterally cleaving a single stranded polynucleotide comprising RNA, without the use of a tracrRNA. In some embodiments, the Class 2 Type V CRISPR-Cas RNA-guided endonuclease protein comprises the amino acid sequence of SEQ ID NO: 4, or at least 70% sequence identity thereto. In some embodiments, the Class 2 Type V CRISPR-Cas RNA-guided endonuclease protein is capable of collaterally cleaving a single stranded RNA. In some embodiments, the Class 2 Type V CRISPR-Cas RNA-guided endonuclease protein is capable of collaterally cleaving a single stranded DNA/RNA hybrid.

In another aspect, provided herein is an engineered system comprising: (a) a Cas12a.1, Cas12p, or Cas12q protein, or a nucleic acid encoding the Cas12a.1, Cas12p, or Cas12q protein; and (b) a Cas12a.1, Cas12p, or Cas12q gRNA, or a nucleic acid encoding a Cas12a.1, Cas12p, or Cas12q gRNA, wherein the gRNA and the Cas12a.1, Cas12p, or Cas12q protein do not naturally occur together, wherein the gRNA is capable of hybridizing to a target sequence in a target DNA, and the gRNA is capable of forming a complex with the Cas12a.1, Cas12p, or Cas12q protein.

In another aspect, provided herein is an engineered single-molecule gRNA, comprising the scaffold sequence of SEQ ID NO: 116 or SEQ ID NO: 117 and a spacer sequence that is capable of hybridizing with a target sequence in a target DNA. In some embodiments, the target DNA is viral DNA, plant DNA, fungal DNA, or bacterial DNA. In some embodiments, the target sequence is a sequence of a target provided in any of Tables 6a-6f. In some embodiments, the target is a coronavirus. In some embodiments, the target is a SARS-CoV-2 virus. In some embodiments, the target DNA is cDNA, and has been obtained by reverse transcription.

In another aspect, provided herein is a method of detecting a target DNA in a sample, the method comprising: (a) contacting the sample with: (i) a Cas12a.1, Cas12p, or Cas12q protein; (ii) a Cas12a.1, Cas12p, or Cas12q gRNA comprising a spacer sequence that is capable of hybridizing with a target sequence in a target DNA; and (iii) a labeled detector oligonucleotide that does not hybridize with the spacer sequence of the gRNA; and (b) measuring a detectable signal produced by cleavage of the labeled detector by the Cas12a.1, Cas12p, or Cas12q protein, thereby detecting the target DNA. This method is useful for diagnostics, e.g. detection of a viral or bacterial pathogen in a sample.

In another aspect, provided herein is a method of modifying a target DNA, the method comprising (a) contacting the target DNA with (i) a Cas9.1, Cas9.2, Cas9.3, Cas9.4, Cas12a.1, Cas12p, or Cas12q protein or a nucleotide encoding the same; and (ii) a Cas9.1, Cas9.2, Cas9.3, Cas9.4, Cas12a.1, Cas12p, or Cas12q gRNA comprising a spacer sequence that is capable of hybridizing with a target sequence in a target DNA. This method is useful for gene therapeutic applications, and generation of cells for therapeutic delivery purposes and for the preparation of cell lines.

In various embodiments, provided herein are compositions, pharmaceutical compositions, vectors, host cells, and kits comprising any of the proteins or polynucleotides of the engineered systems described herein.

### BRIEF DESCRIPTION OF THE DRAWINGS

FIGS. 1A-1B show expression vector maps for Cas9.1 and Cas9.2.

FIGS. 2A-2C show expression vector maps for Cas12a.1, Cas12p, and Cas12q.

FIG. 3A is a schematic representation of the CRISPR Cas cluster around the novel Cas9.1 gene. FIG. 3B shows the secondary structure of the direct repeat for the Cas9.1 pre-crRNA. FIG. 3C is a schematic representation of the CRISPR Cas cluster around the novel Cas9.2 gene. FIG. 3D is a schematic representation of the CRISPR Cas cluster around the novel Cas9.3 gene. FIG. 3E shows the secondary structure of the direct repeat for the Cas9.3 pre-crRNA. FIG. 3F is a schematic representation of the CRISPR Cas cluster around the novel Cas9.4 gene. FIG. 3G shows the secondary structure of the direct repeat for the Cas9.4 pre-crRNA.

FIG. 4A shows the key catalytic amino acids for Cas9 proteins (SEQ ID NOs: 137-168), and alignments of conserved motifs in selected representatives of the Cas9 protein family.

FIG. 4B shows the alignment of RuvC1, Bridge Helix, RuvCII, and RuvCIII domains for Cas9.1 (SEQ ID NO: 1) and other selected representatives of the Cas9 protein family (SEQ ID NOs: 169-176). FIG. 4C shows the alignment of RuvC1, Bridge Helix, RuvCII, and RuvCIII domains for Cas9.2 (SEQ ID NO: 2) and other selected representatives of the Cas9 protein family (SEQ ID NOs: 170-174 and 169). FIG. 4D shows the alignment of RuvC1, Bridge Helix, RuvCII, and RuvCIII domains for Cas9.3 (SEQ ID NO: 10) and other selected representatives of the Cas9 protein family (SEQ ID NOs: 169-176). FIG. 4E shows the alignment of RuvC1, Bridge Helix, RuvCII, and RuvCIII domains for Cas9.4 (SEQ ID NO: 11) and other selected representatives of the Cas9 protein family (SEQ ID NOs: 169-176).

FIG. 5A is a schematic representation of the CRISPR Cas cluster around the novel Cas12a.1 gene. FIG. 5B shows the secondary structure of the direct repeat for the Cas12a.1 pre-crRNA (SEQ ID NO: 177). FIG. 5C is a schematic representation of the CRISPR Cas cluster around the novel Cas12p gene. FIG. 5D shows the secondary structure of the direct repeat for a first Cas12p pre-crRNA (SEQ ID NO: 178) and a second Cas12p pre-crRNA (SEQ ID NO: 179). FIG. 5E is a schematic representation of the CRISPR Cas cluster around the novel Cas12q gene. FIG. 5F shows the secondary structure of the direct repeat for the Cas12q pre-crRNA (SEQ ID NOs: 180 and 181).

FIG. 6A shows the key catalytic amino acids for Cas12 proteins (SEQ ID NOs: 182-217, and alignments of conserved motifs in selected representatives of the Cas12a protein family.

FIG. 6B shows the alignment of Cas12a.1 (SEQ ID NO: 3) vs. SEQ ID NO: 81 of US20160208243 (SEQ ID NO: 218), and has a 46.8% sequence identity; and FIG. 6C shows the alignment of Cas12a.1 (SEQ ID NO: 3) vs. SEQ ID NO: 3 of U.S. Pat. No. 10,253,365 (SEQ ID NO: 219), and has a 46.5% sequence identity.

FIG. 6D shows the amino acid sequence of Cas12p (SEQ ID NO: 4) with the RuvC motifs underlined. The FnCas12a sequence referenced in Shmakov et al., 2015 was used as a reference for identification of the Ruv motifs.

FIG. 6E shows the alignment of Cas12p (SEQ ID NO: 4) with Cas12g1 (SEQ ID NO: 220). This figure shows an alignment of Cas12p with Cas12g1.

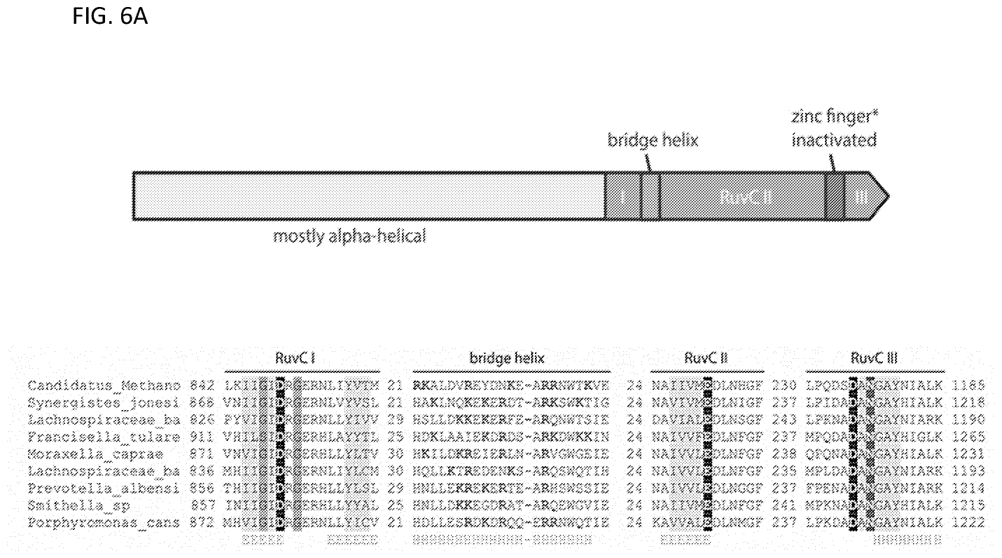

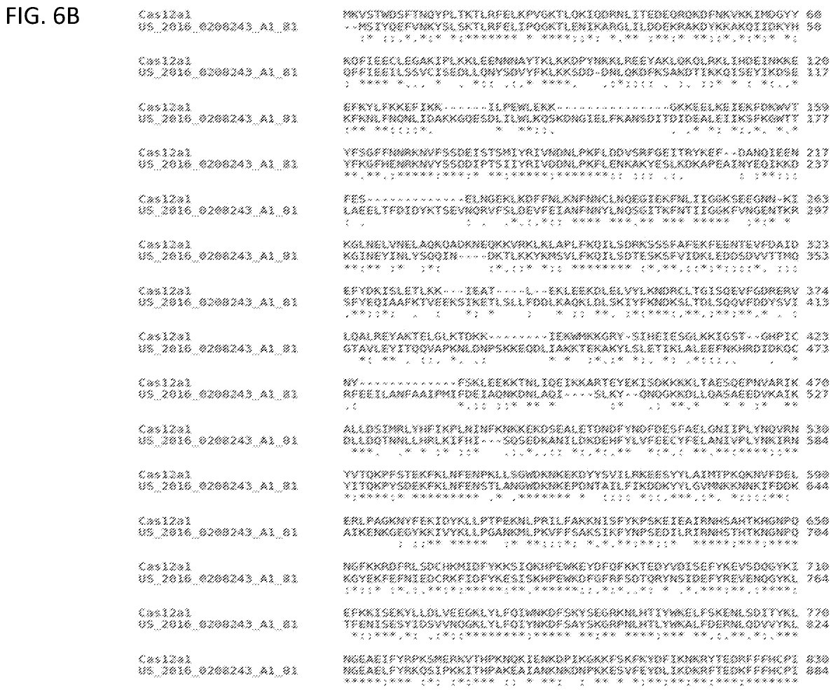

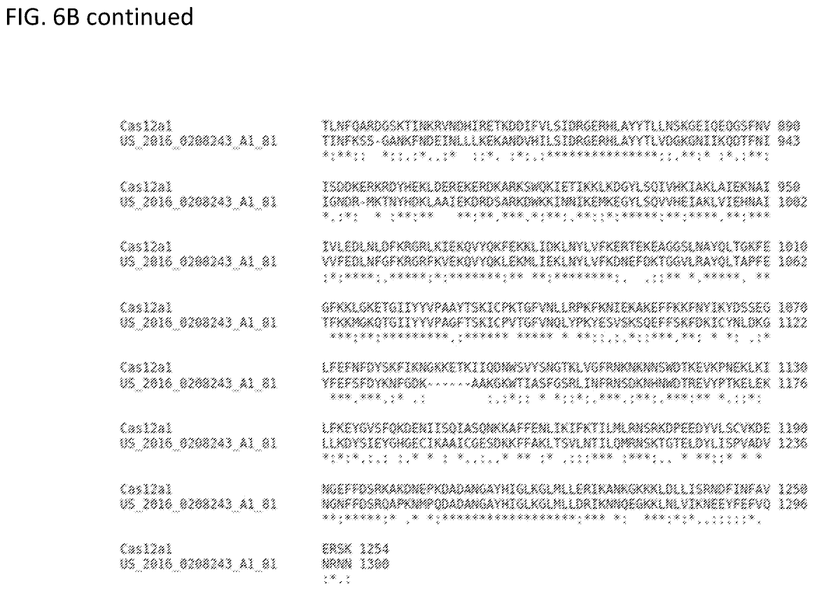

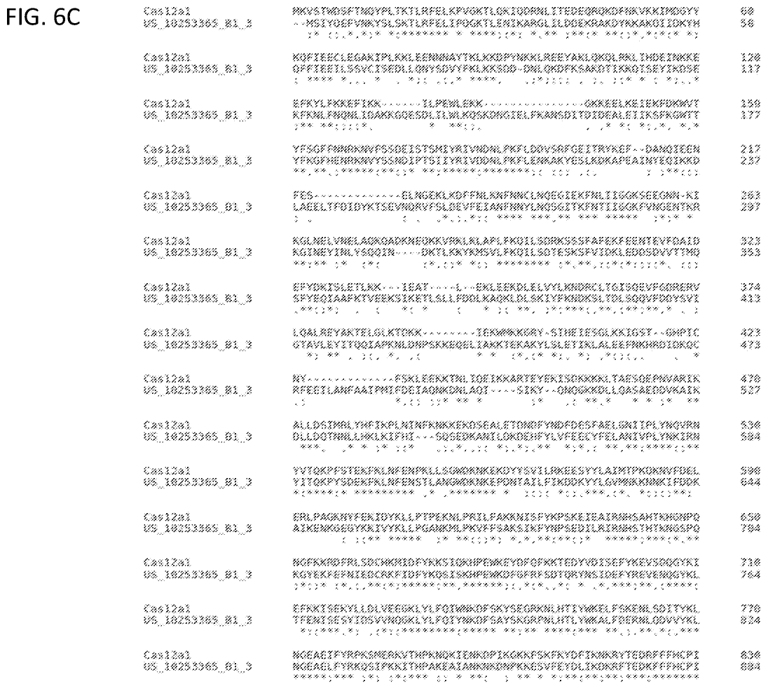

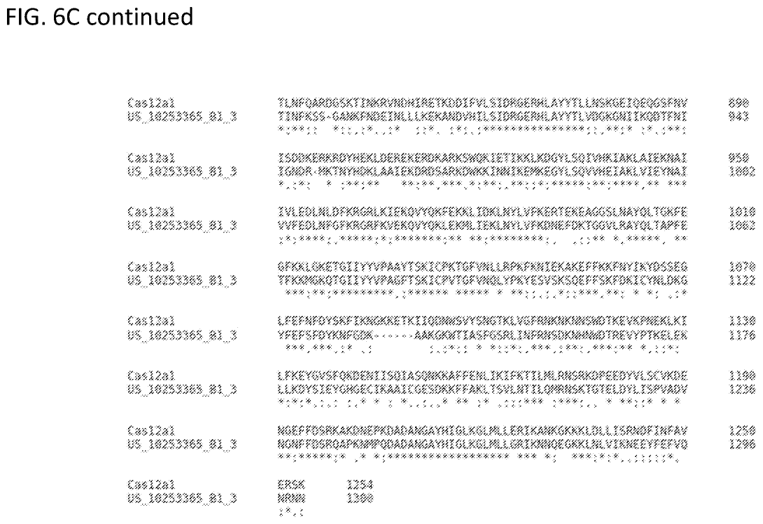

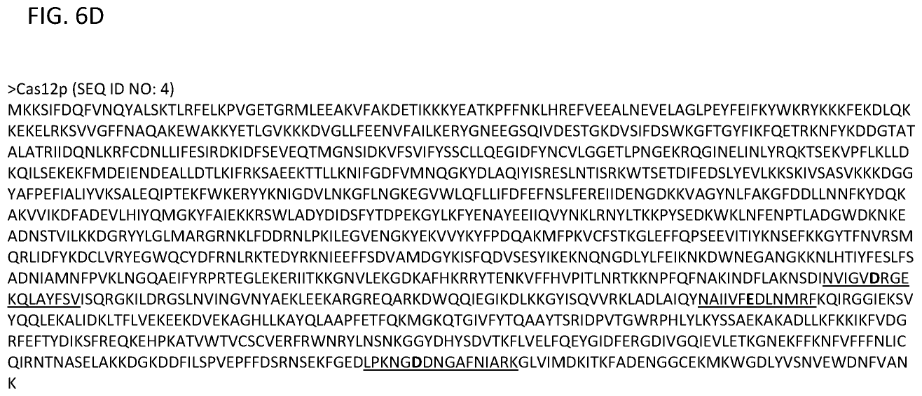

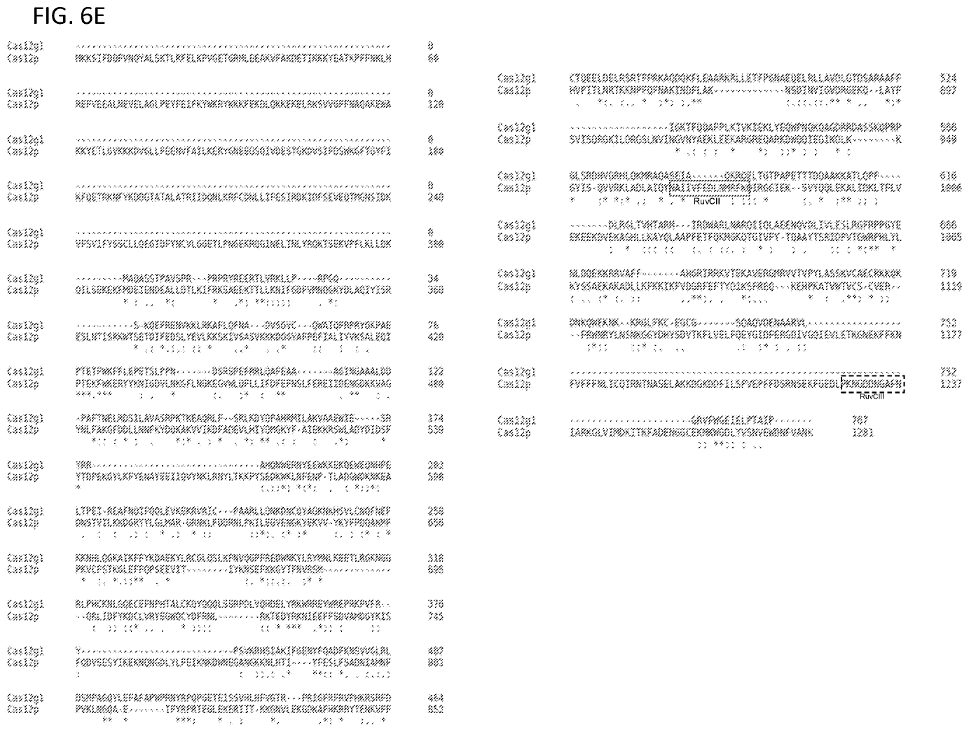

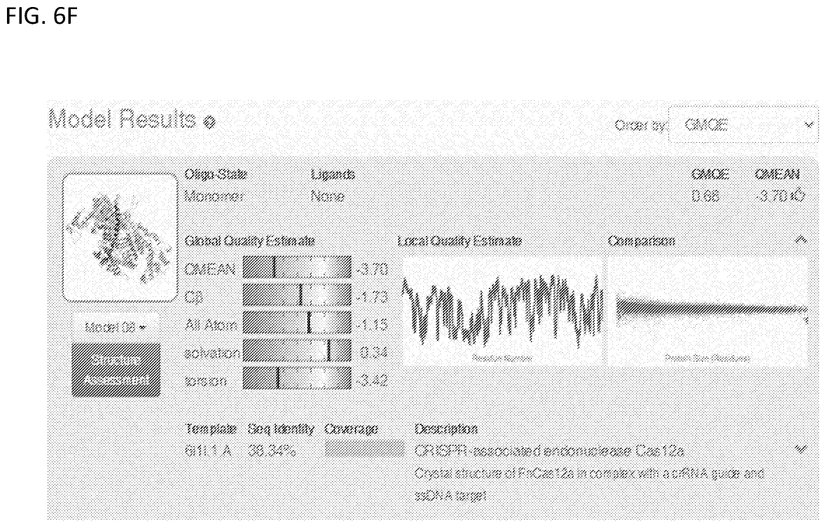

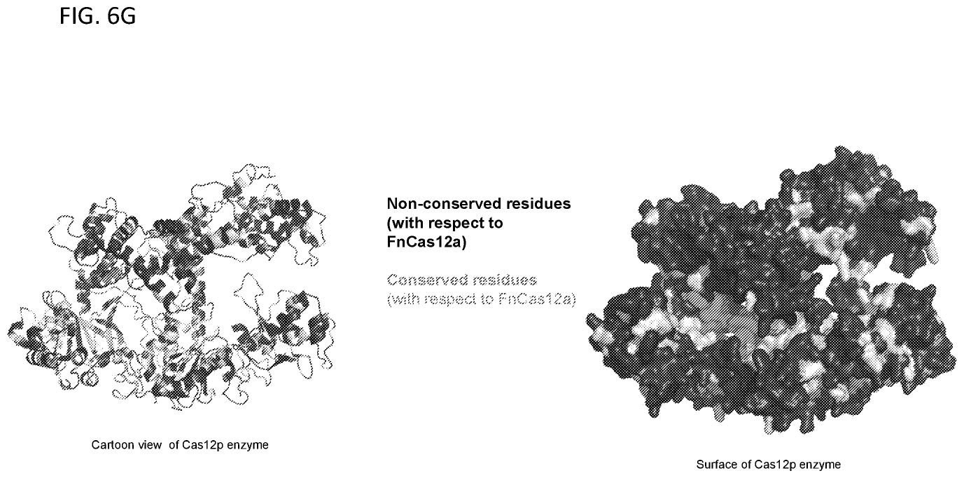

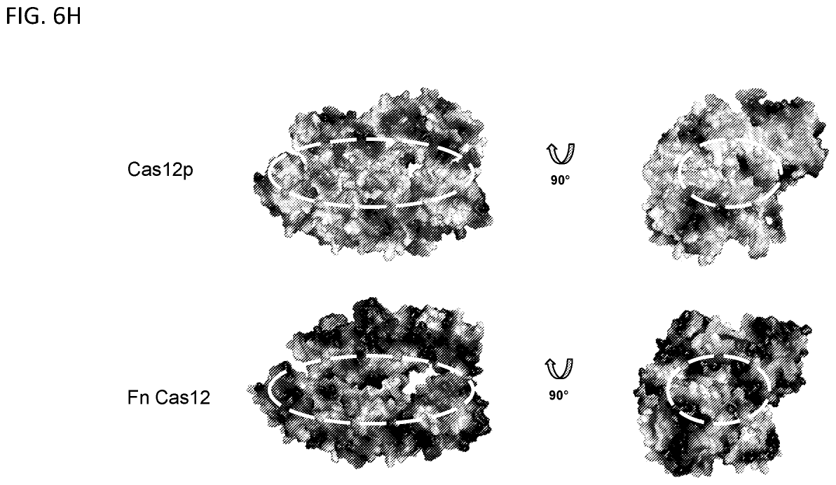

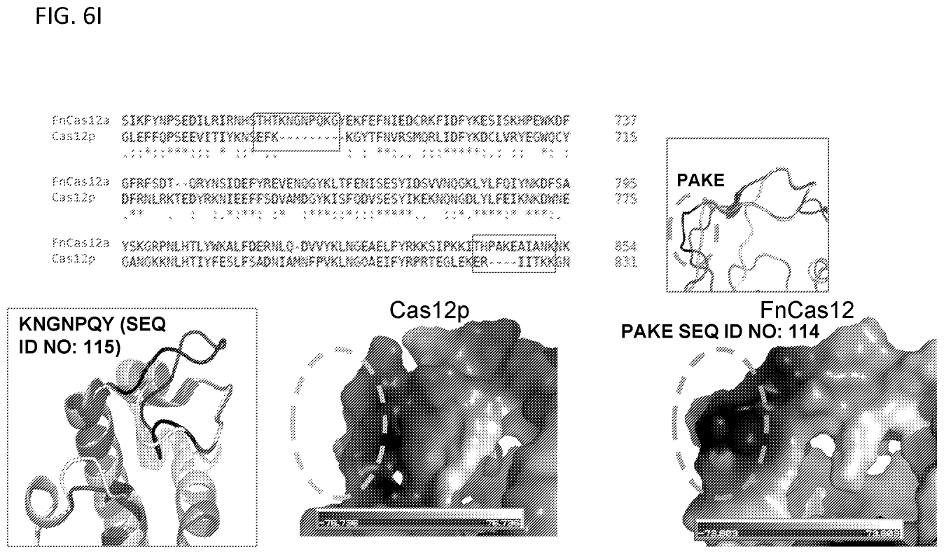

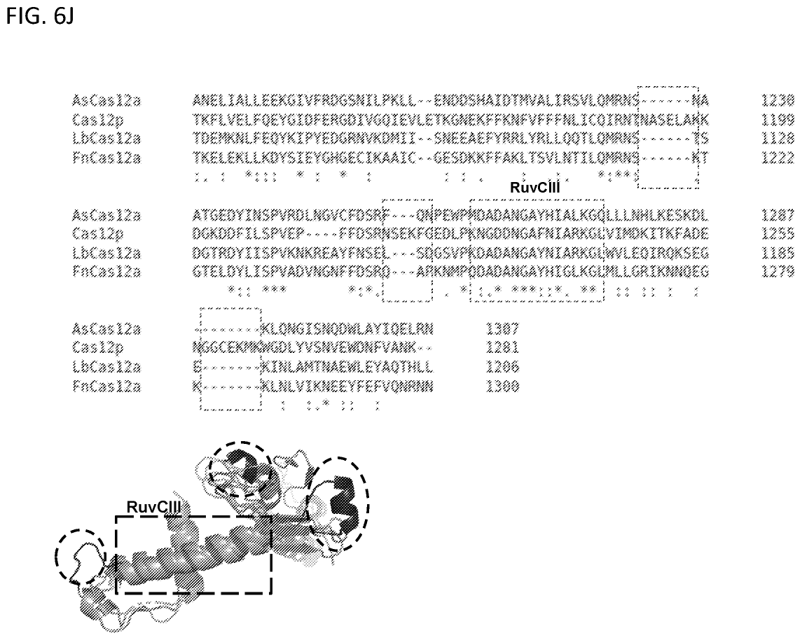

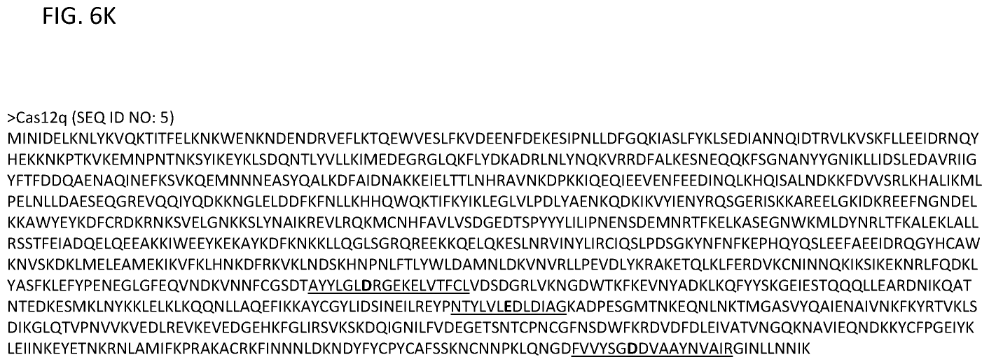

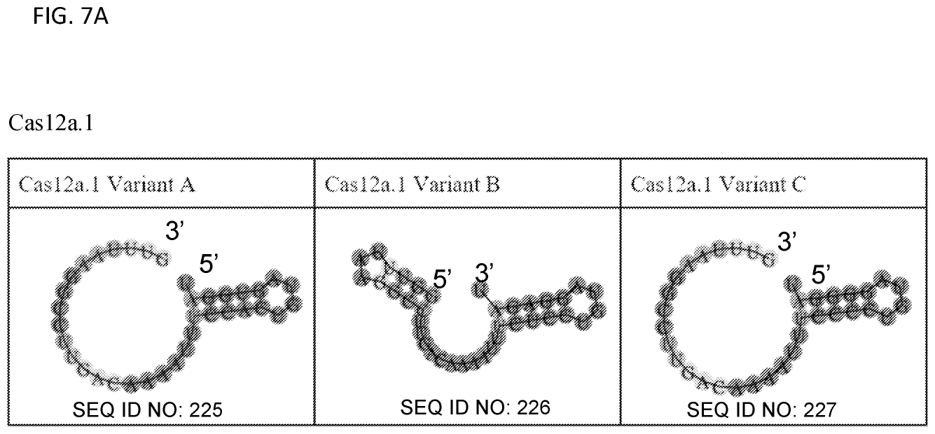

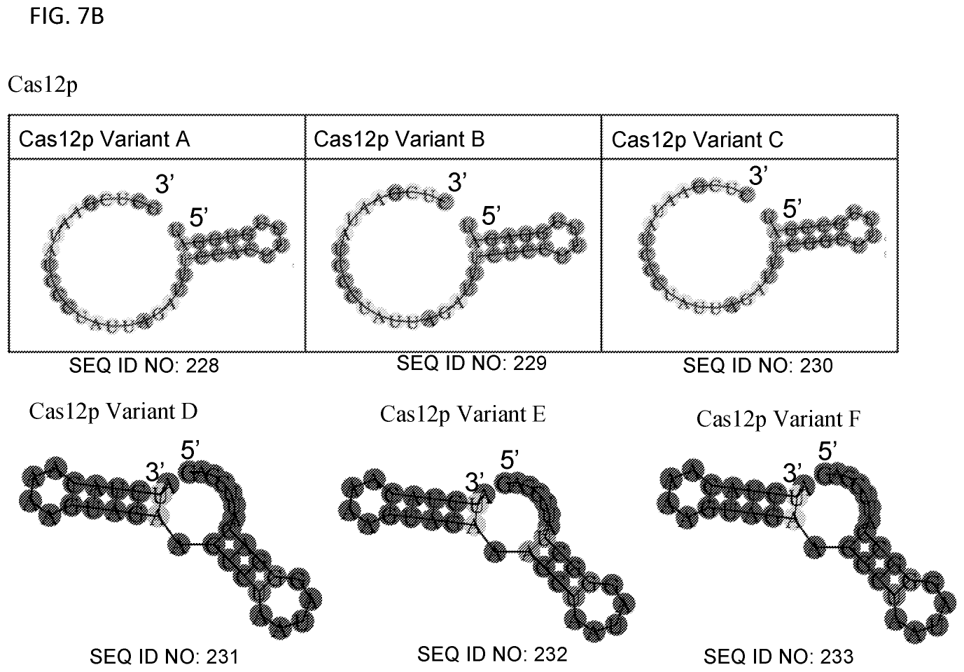

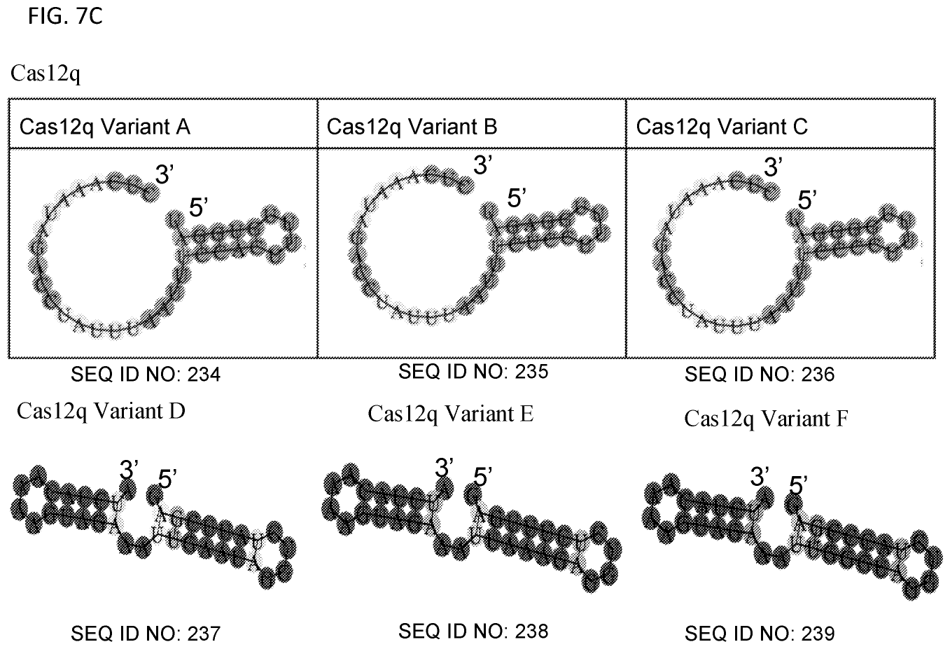

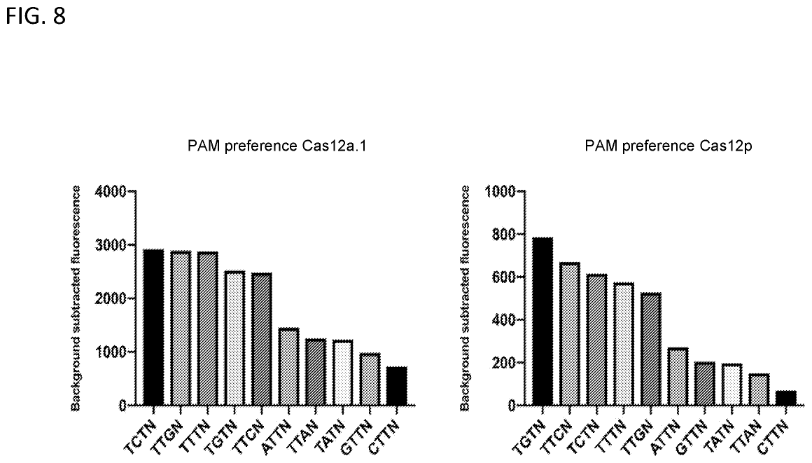

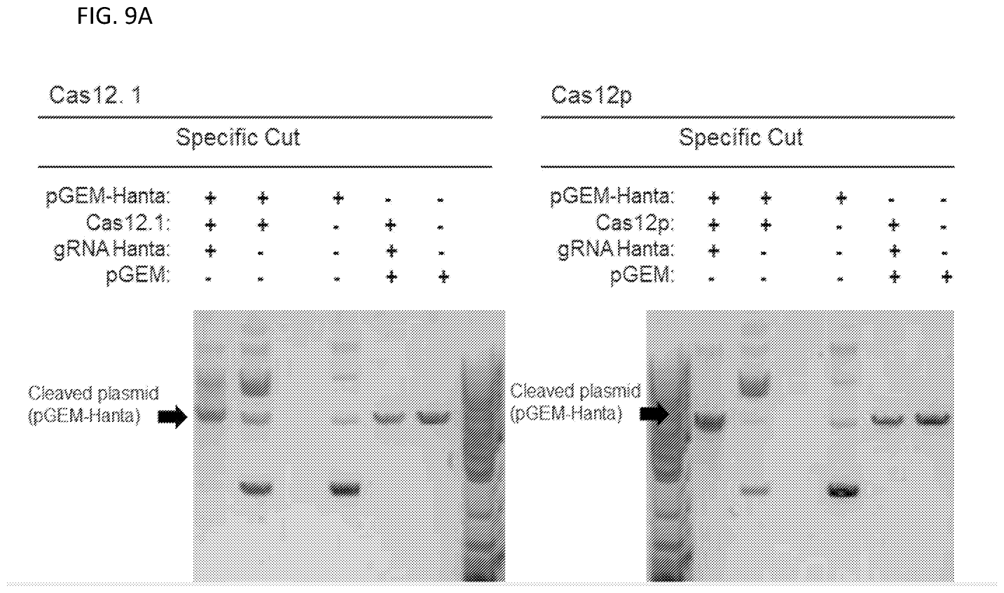

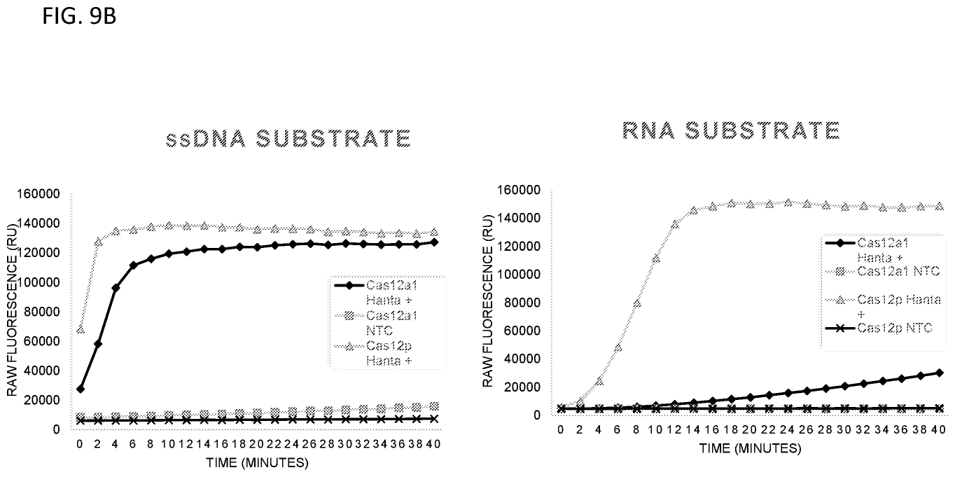

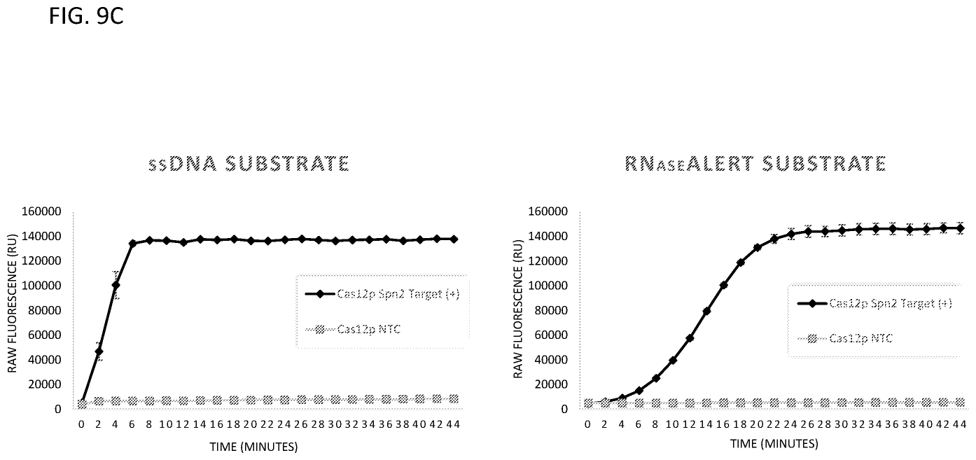

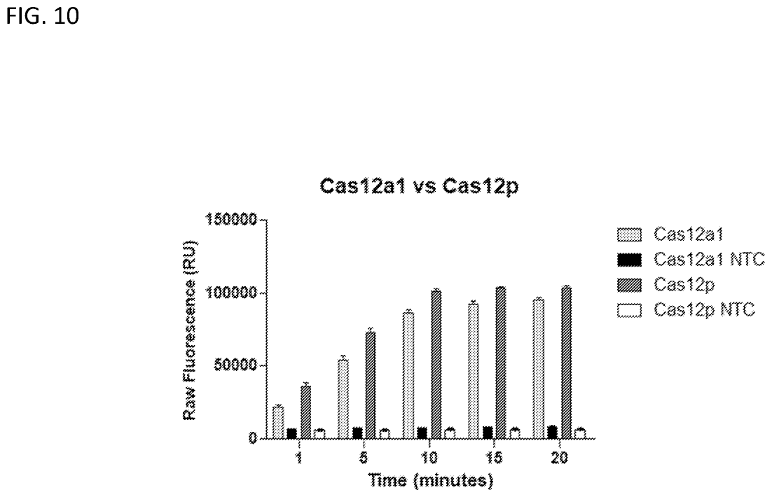

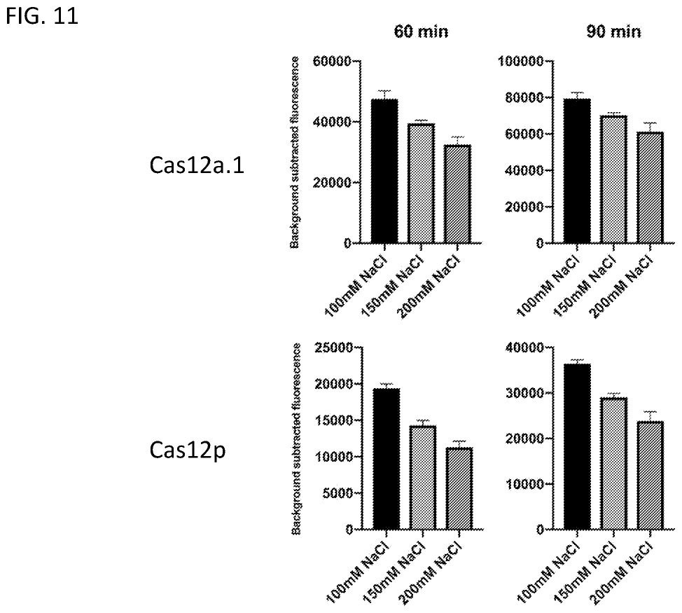

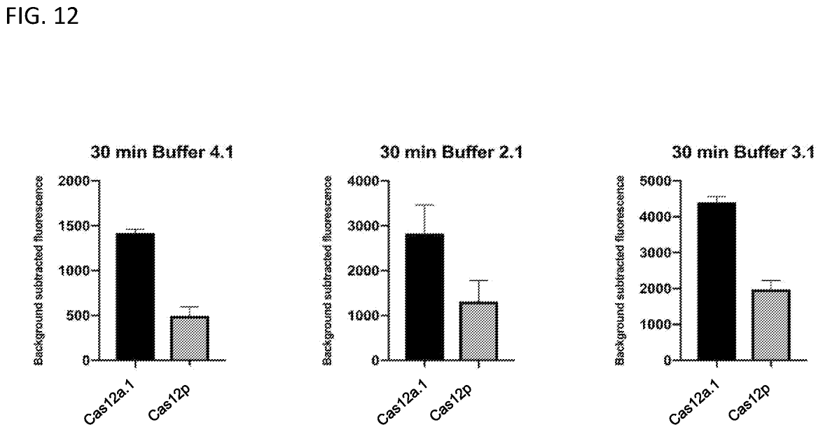

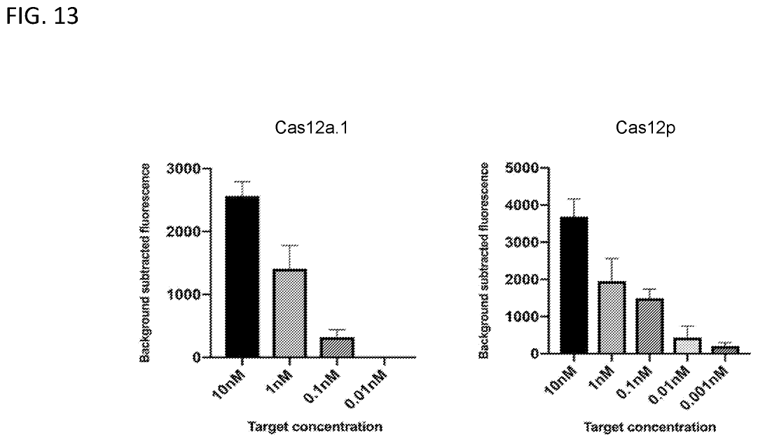

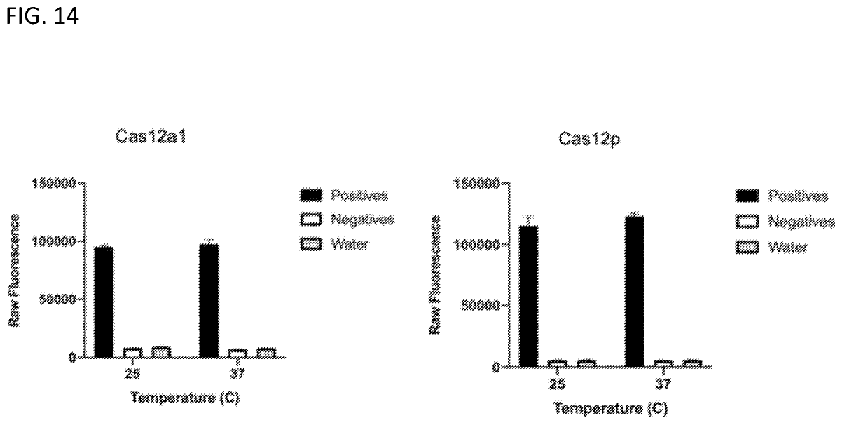

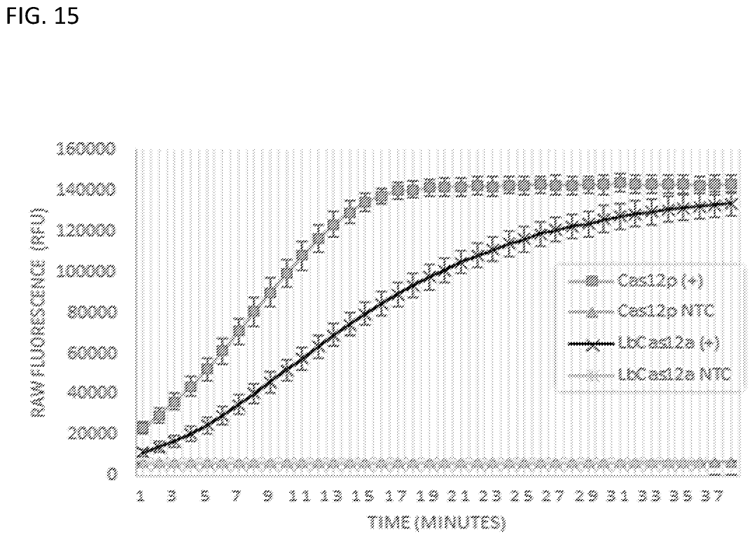

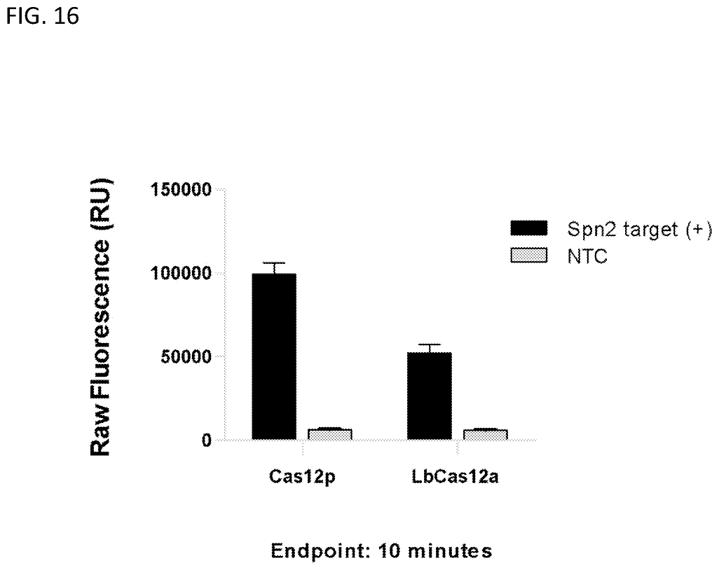

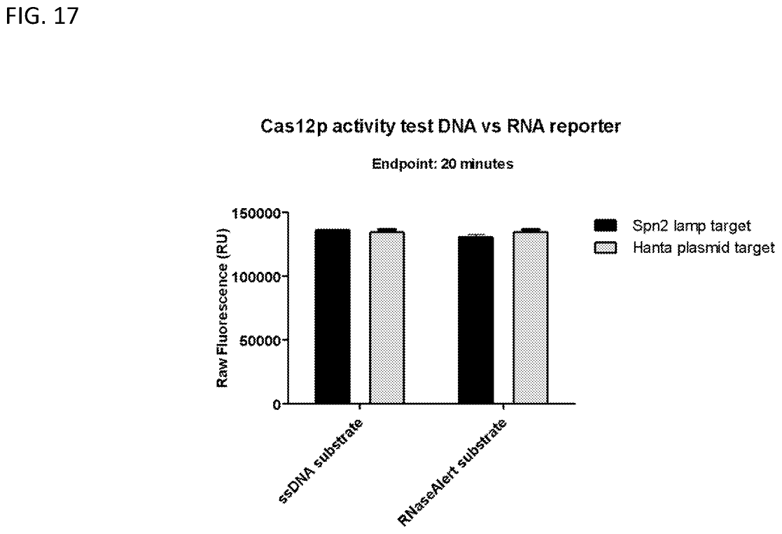

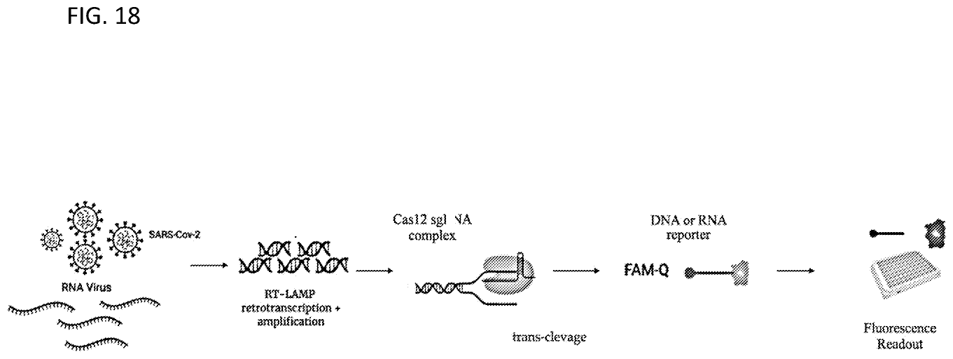

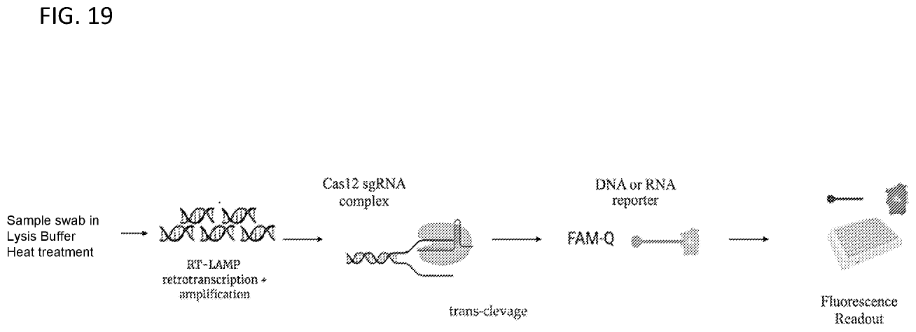

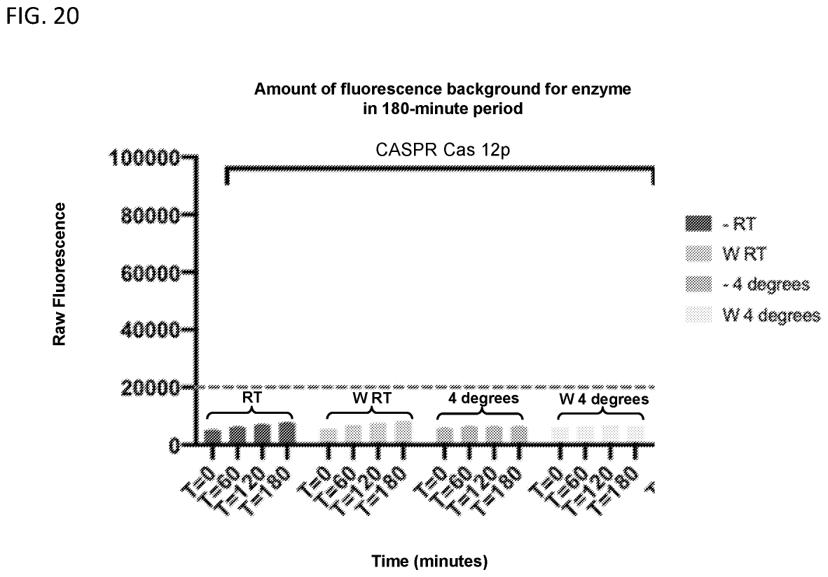

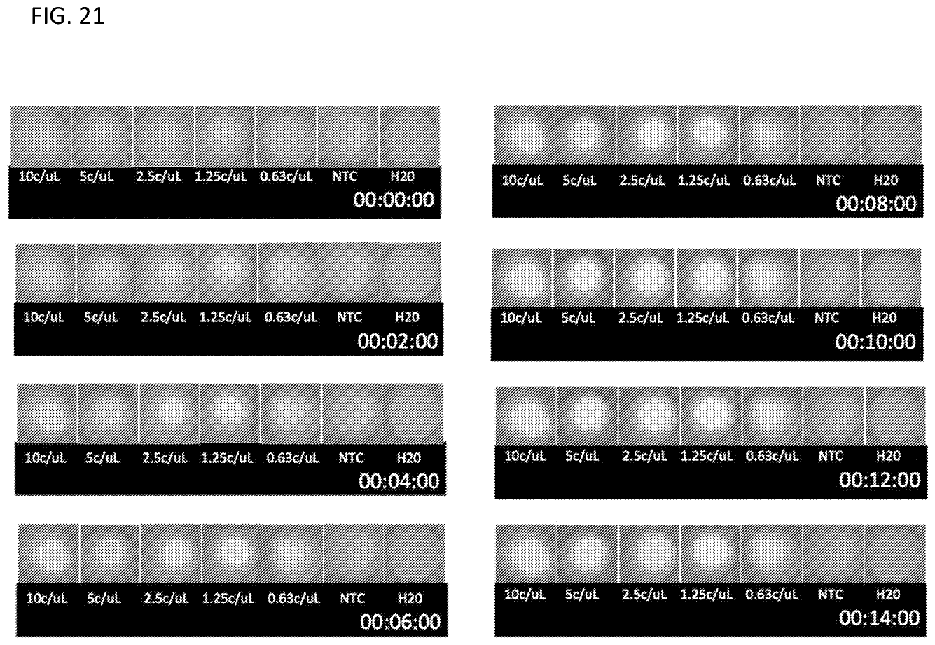

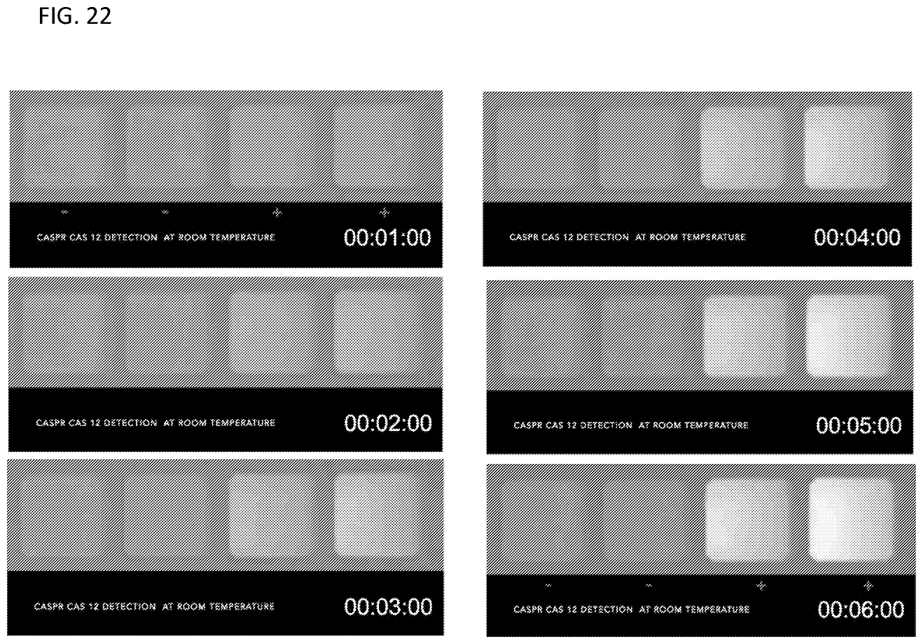

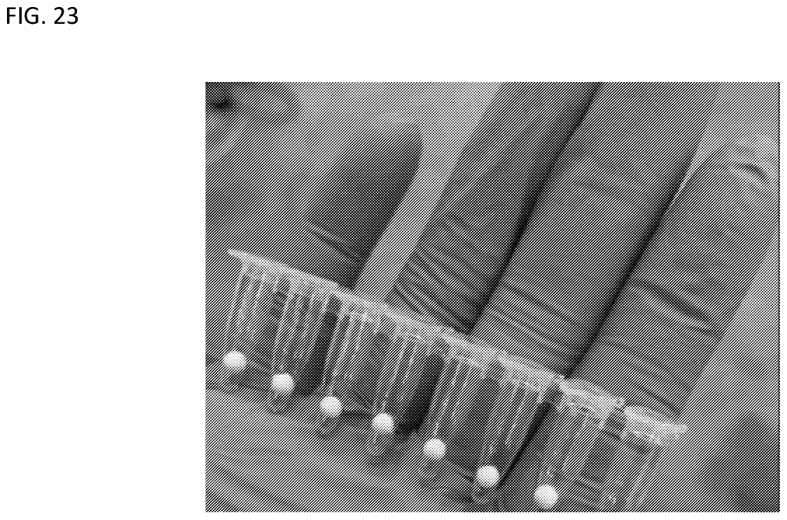

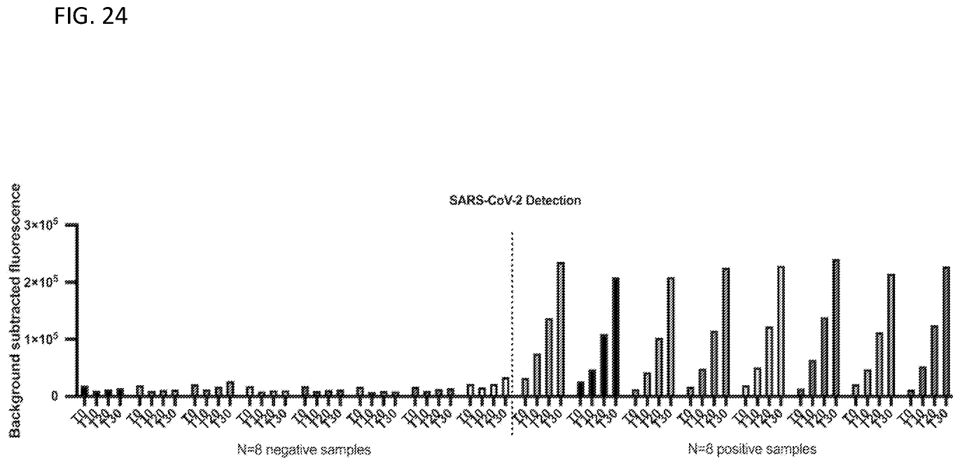

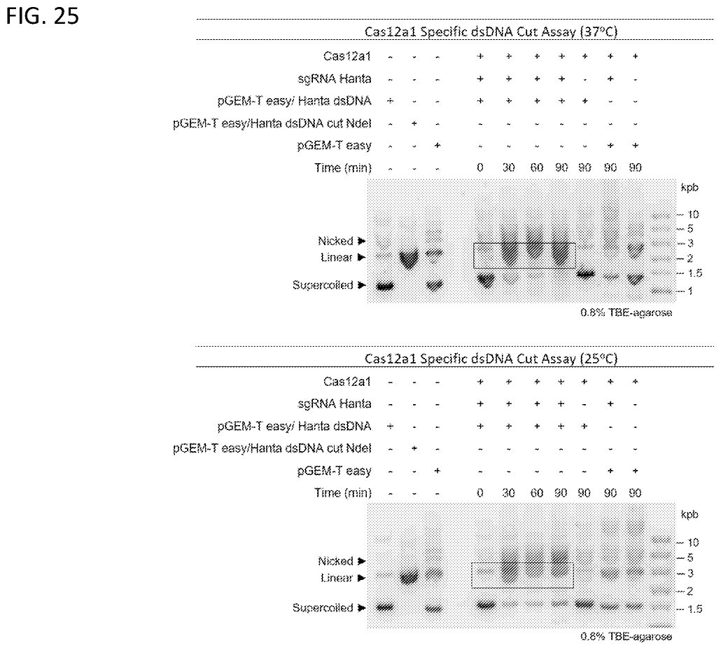

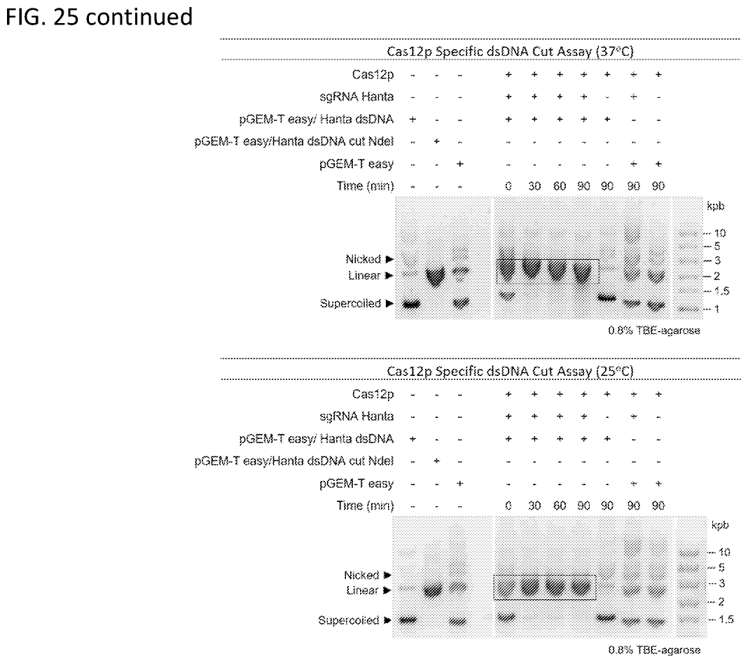

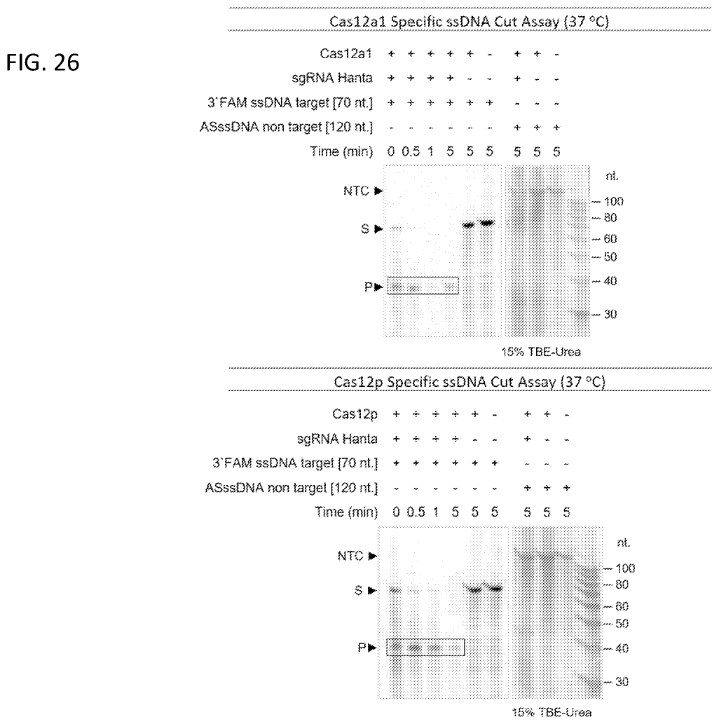

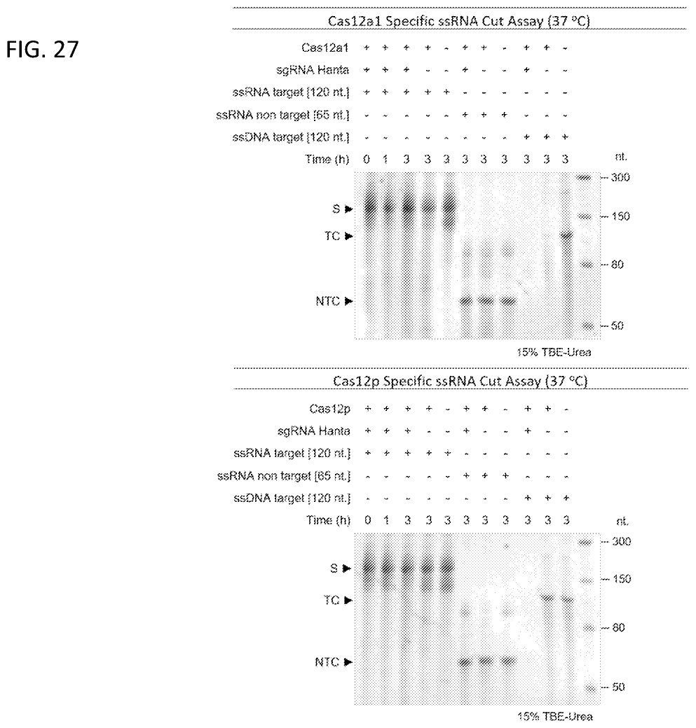

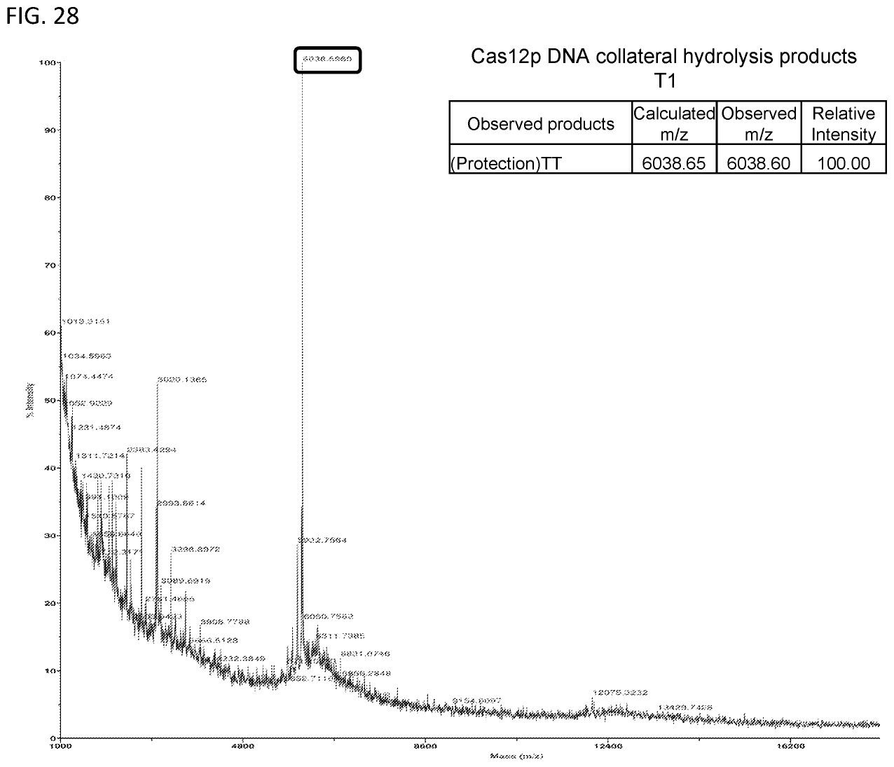

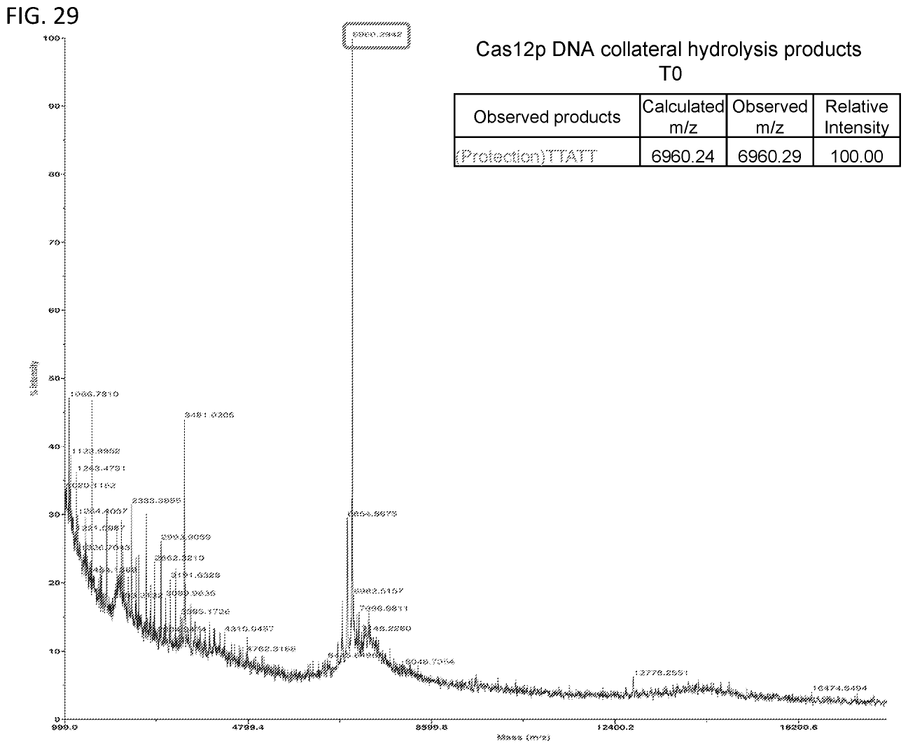

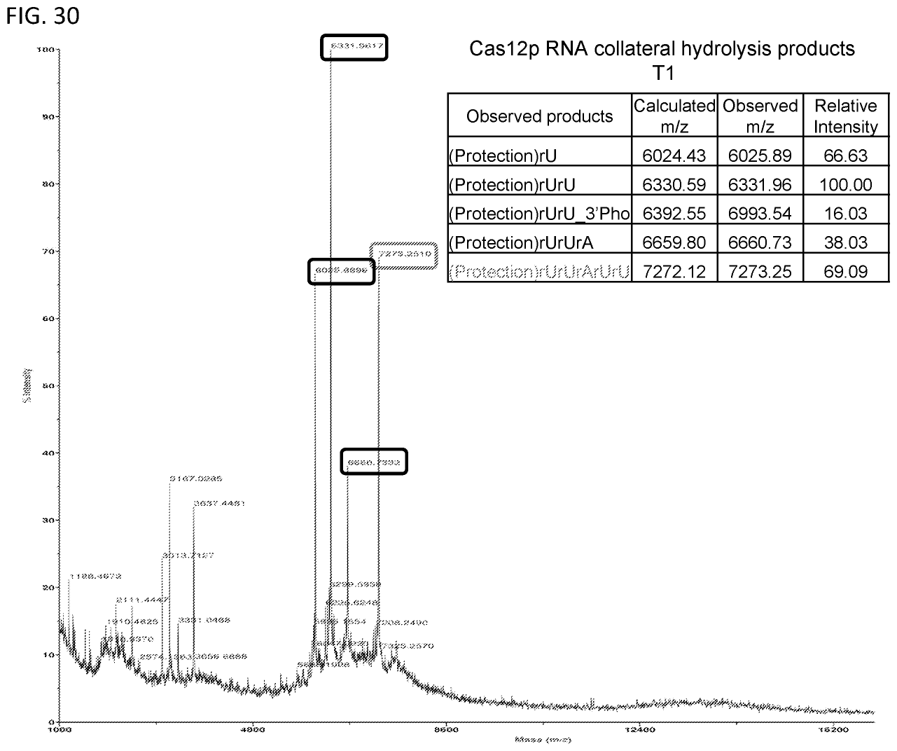

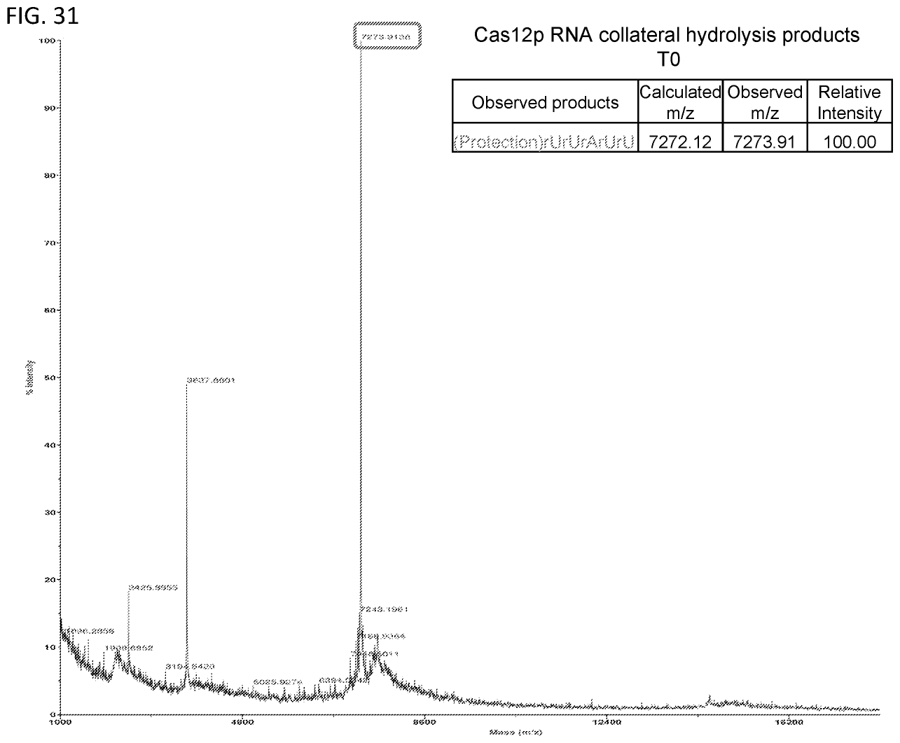

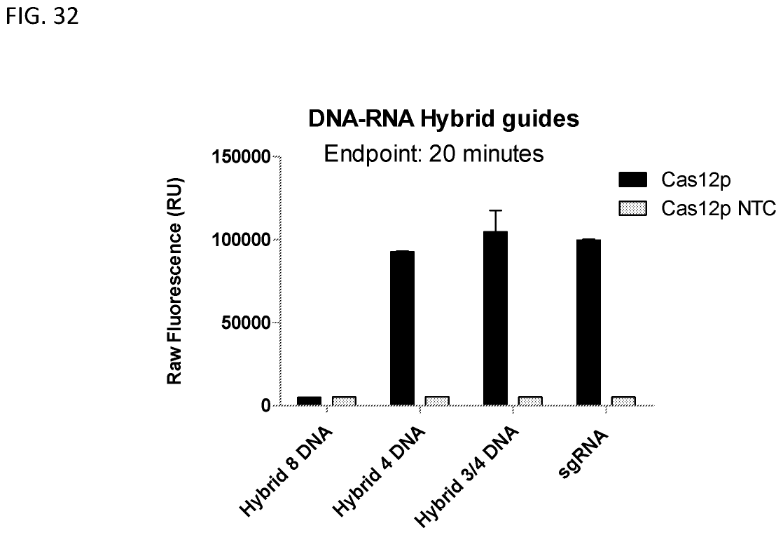

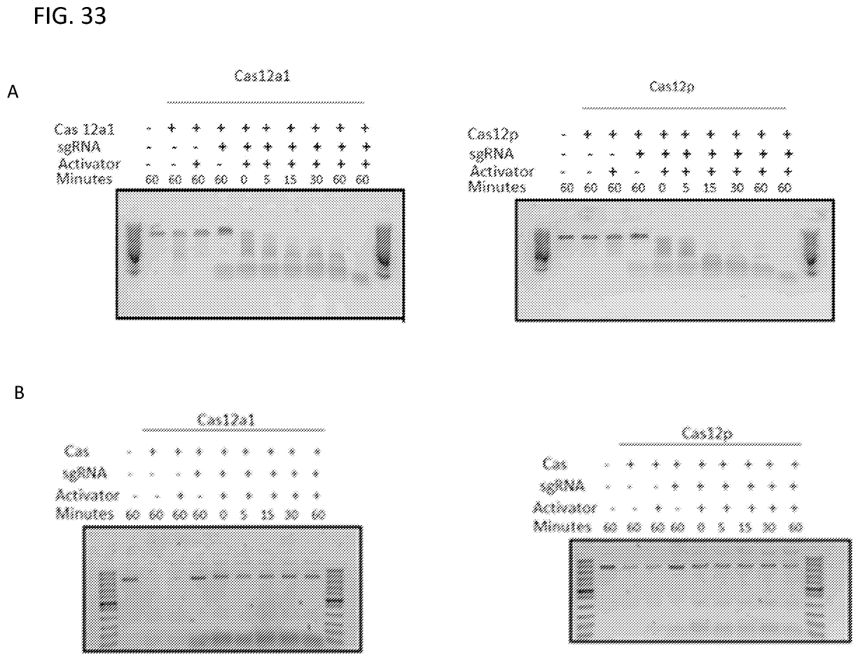

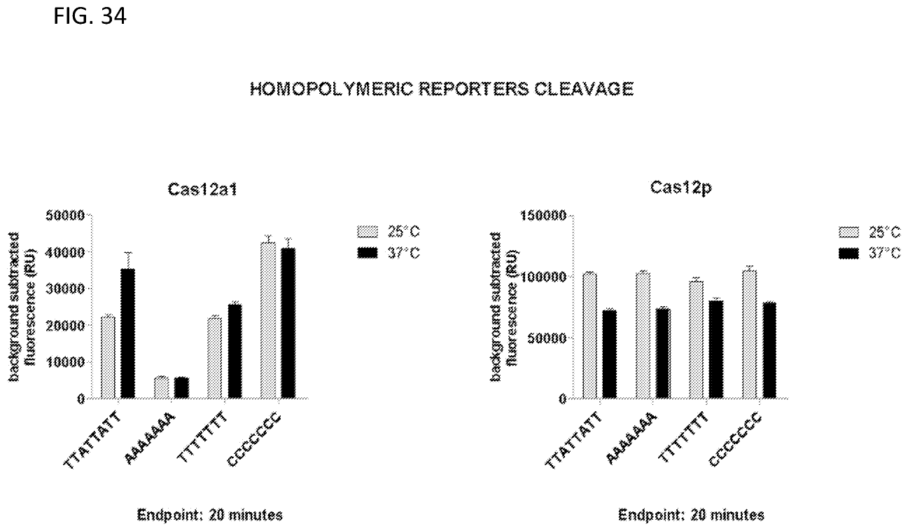

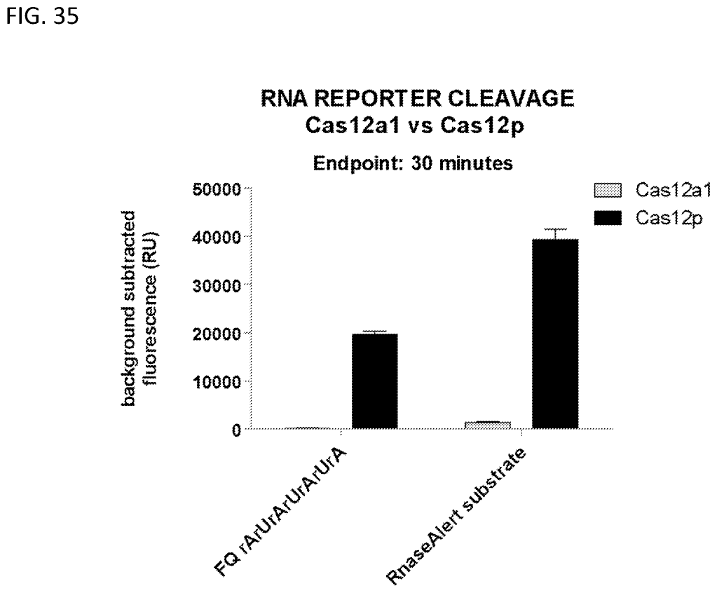

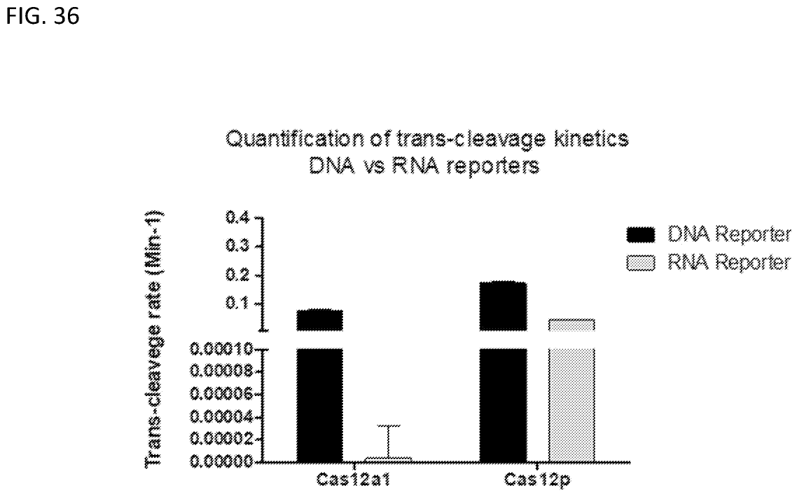

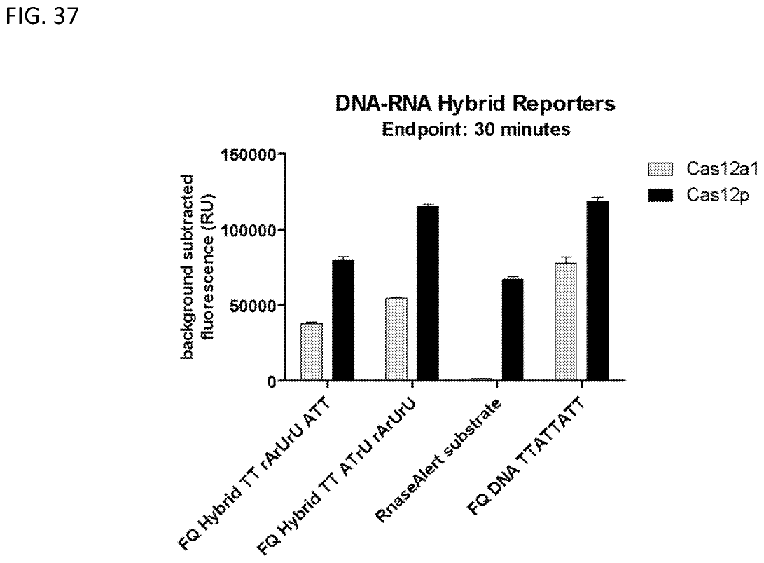

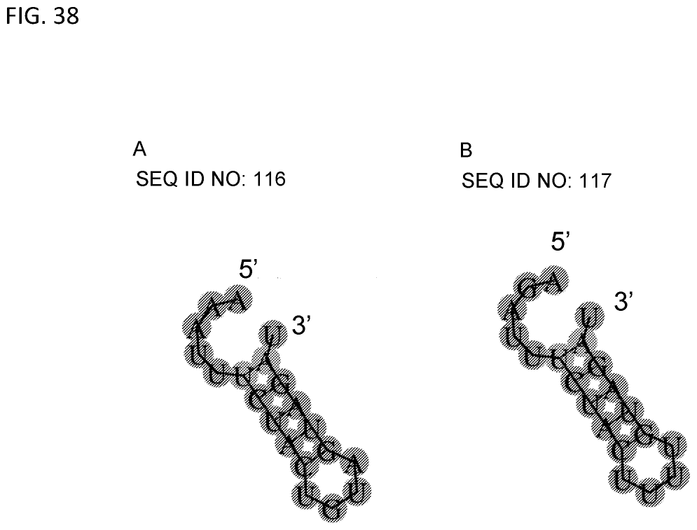

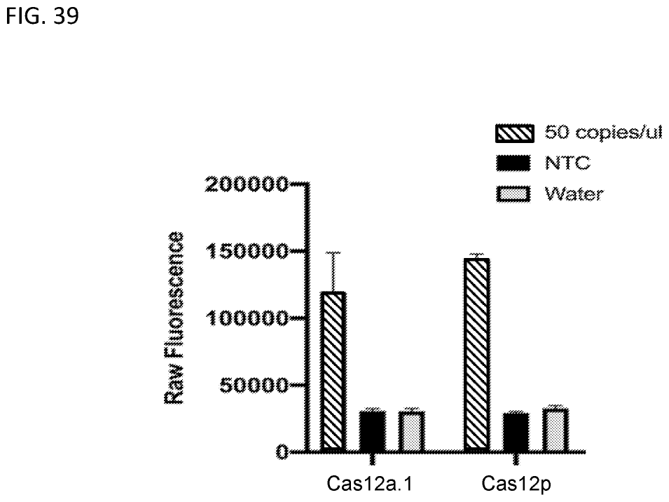

In the following figures, the structure of Cas12p protein was modeled based on Fn Cas12a structure with Swiss Model server. FIG. 6F shows a structural analysis of Cas12p using the Swiss Model server. FIG. 6G shows a spatial prediction of non-conserved amino acid residues in Cas12p. FIG. 6H shows the approximation of charge distribution over the surface of Cas12p. FIG. 6I shows predicted structural differences between Cas12p (SEQ ID NO: 4) and FnCas12a (SEQ ID NO: 221) based on protein sequences. FIG. 6J shows RuvCIII domain structural analysis of Cas12p (SEQ ID NO: 4) and Cas12a proteins (AsCas12a (SEQ ID NO: 223), LbCas12a (SEQ ID NO: 224) and FnCas12a (SEQ ID NO: 221)) based on structural analysis with Swiss Model server.

FIG. 6K shows the amino acid sequence of Cas12q (SEQ ID NO: 5) with the RuvC motifs underlined.

FIGS. 7A, 7B, 7C show predicted RNA secondary structures of non-naturally occurring direct repeats (artificial variants; SEQ ID NOs: 225-239), generated to improve stem-loop stability of guides of the disclosure.

FIG. 8 shows bar graphs for the PAM sequence preferences of Cas12a.1 and Cas12p for the ten PAM motifs, measuring the performance of the Cas12a.1 and the Cas12p using fluorescence assays.

FIG. 9A shows specific cleavage activity of the Ca12a.1 (designated as Cas12.1 in the figure) and Cas12p proteins of the disclosures with an exemplary Hanta virus target. FIG. 9B shows that both Cas12a.1 and Cas12p exhibit collateral activity and can cut non-target containing ssDNA. FIG. 9C shows that Cas12p exhibits both ssDNA and RNA reporter collateral cleavage using as a SARS-CoV-2 inactivated virus as sample as the target.

FIG. 10 shows activity of the novel cas12 proteins at 25° C.

FIG. 11 shows the activity of the novel Cas12 proteins at various salt concentrations.

FIG. 12 shows the performance of the Cas12a.1 and the Cas12p of the disclosure in three different commercial buffers.

FIG. 13 shows sensitivity curves without RPA of the Cas12a.1 and the Cas12p of the disclosure, for various target concentrations measured for 30 minutes.

FIG. 14 shows that the amount of fluorescence detection by Cas12a.1 and Cas12p for a target DNA reverse transcribed from SARS-CoV-2 RNA was equal at both 37° C. and 25° C., indicative of thermostability and function and room temperature.

FIG. 15 shows the differential performance of Cas12p vs. LbCas12a at 25° C.

FIG. 16 shows the differential performance of Cas12p vs. LbCas12a at 25° C., using SARS-CoV-2 as a target, described in Example 10.

FIG. 17 shows the ability of Cas12p to cleave both a ssDNA and RNA reporter.

FIG. 18 shows a schematic workflow for the detection of SARS-CoV-2 described herein.

FIG. 19 shows a schematic workflow for the detection of SARS-CoV-2 described herein, from a sample.

FIG. 20 shows that Cas12p has a minimal background signal after 30-60 minutes of cleavage activity. This provides advantages at low viral concentrations, and indicates stability of the lyophilized format.

FIG. 21 shows that a diagnostics assay using Cas12p at room temperature, can be read out on a paper format.

FIG. 22 shows that a diagnostics assay using Cas12p at room temperature can be read in well plate with a fluorescent detector.

FIG. 23 shows exemplary lyophilized beads of the disclosure.

FIG. 24 shows the results of SARS-CoV-2 detection using a Cas12p/guide, using a RNA reporter from patient samples and negative control samples in lyophilized format.

FIG. 25 shows specific dsDNA cleavage time courses of the Ca12a.1 and Cas12p proteins of the disclosures, complexed with a sgRNA for an exemplary Hanta virus target. Time points: 0, 30, 60 and 90 minutes.

FIG. 26 shows specific ssDNA cleavage time courses of the Ca12a.1 and Cas12p proteins of the disclosures, complexed with a sgRNA for an exemplary Hanta virus target. (S): 3′FAM-ssDNA target substrate. (P): 3′FAM-ssDNA target product. (NTC): ASssDNA non-target control. Time points: 0, 0.5, 1 and 5 minutes.

FIG. 27 shows specific ssRNA cleavage time courses of the Ca12a.1 and Cas12p proteins of the disclosures, complexed with a sgRNA for an exemplary Hanta virus target. (S): ssRNA target substrate. (TC): ssDNA target control. (NTC): ssRNA non-target control. Time points: 0, 1 and 3 h.

FIG. 28 shows the mass spectra data of Cas12p reactions using a DNA oligo as the reporter.

FIG. 29 shows the mass spectra data of Cas12p reactions using a DNA oligo as the reporter.

FIG. 30 shows the mass spectra data of Cas12p reactions using a RNA oligo as the reporter.

FIG. 31 shows the mass spectra data of Cas12p reactions using a RNA oligo as the reporter.

FIG. 32 shows that DNA-RNA chimeric guides enable efficient collateral activity, when used with Cas 12p.

FIG. 33 shows agarose gels demonstrating the collateral activity for Cas12a.1 and Cas12p, for ssDNA, but not dsDNA.

FIG. 34 shows differential efficiency of cleavage of homopolymeric reporters, at 25° C. and 37° C. The results show that Cas12p cleaved poly T, poly A and poly C, whereas Cas12a.1 showed a preference for polyC cleavage.

FIG. 35 shows the collateral cleavage (also referred to herein as trans-cleavage) ability of Cas12p but not of Cas12a.1, to cleave a RNA reporter.

FIG. 36 shows the kinetics of collateral cleavage activity of Cas12p and Cas12a.1, using DNA and RNA as reporters.

FIG. 37 shows the collateral cleavage of Cas12p and Cas12a.1 using a FAMQ DNA-RNA chimeric reporter.

FIG. 38 shows the sequences and secondary structures of mature guide scaffolds for Cas12a.1 (SEQ ID NO: 116) and Cas12p (SEQ ID NO: 117).

FIG. 39 shows the validation of the use of the mature guide scaffolds to detect SARS-CoV-2 using Cas12a.1 and Cas12p, when used in conjunction with a spacer targeting the N gene of SARS-CoV-2.

### DETAILED DESCRIPTION

Provided herein are novel Class 2 Type II and novel Type V CRISPR-Cas RNA-guided systems, methods of making, and methods of use.

### Definitions

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength.

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like).

Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A “vector” or “expression vector” is a replicon, such as plasmid, phage, virus, or cosmid, to which another DNA segment, i.e. an “insert”, may be attached so as to bring about the replication of the attached segment in a cell.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Cas12a.1 protein” includes a plurality of such Cas12a.1 proteins and reference to “the gRNA” or “the guide RNA” includes reference to one or more gRNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

### I. Class 2 Type II CRISPR-Cas RNA-Guided Systems

Provided herein are novel Class 2 Type II CRISPR-Cas RNA-guided proteins and their guide RNAs (a “guide RNA” is interchangeably referred to herein as “gRNA”), constituting the Class 2 Type II CRISPR-Cas RNA-guided systems of the disclosure. As used herein a gRNA may comprise only RNA nucleotides, may comprise RNA and DNA nucleotides, or may comprise only DNA nucleotides, and thus while referred to as a gRNA, may comprise non RNA-nucleotides.

Accordingly, provided herein are systems comprising (a) a Cas9.1, a Cas9.2, a Cas9.3 or a Cas9.4 protein, or a nucleic acid encoding the Cas9.1, the Cas9.2, the Cas9.3 or the Cas9.4 protein; and (b) a Cas9.1, a Cas9.2, a Cas9.3 or a Cas9.4 gRNA, or a nucleic acid encoding the Cas9.1, the Cas9.2, the Cas9.3 or the Cas9.4 molecule RNA, wherein the gRNA and the Cas9.1 the Cas9.2, the Cas9.3 or the Cas9.4 protein do not naturally occur together, wherein the gRNA is capable of hybridizing to a target sequence in a target DNA, and the gRNA is capable of forming a complex with the Cas9.1, the Cas9.2, the Cas9.3 or the Cas9.4 protein. It should be understood that “Cas9.1-Cas9.4” as used herein refers to the following: Cas9.1, Cas9.2, Cas9.3, Cas9.4.

These components are described in turn below.

a. Class 2 Type II CRISPR-Cas RNA-Guided Proteins

Provided herein are novel Class 2 Type II and Type V CRISPR-Cas RNA-guided endonucleases, e.g. novel Cas9 proteins (Cas9 variants) and novel Cas12a proteins (Cas12a variants), and novel Cas12 subtypes.

Table 1 shows the protein sequences for the novel Cas9 proteins of the disclosure. In some embodiments the novel Cas9 proteins of the disclosure have been deduced using bioinformatics methods from metagenomics samples.

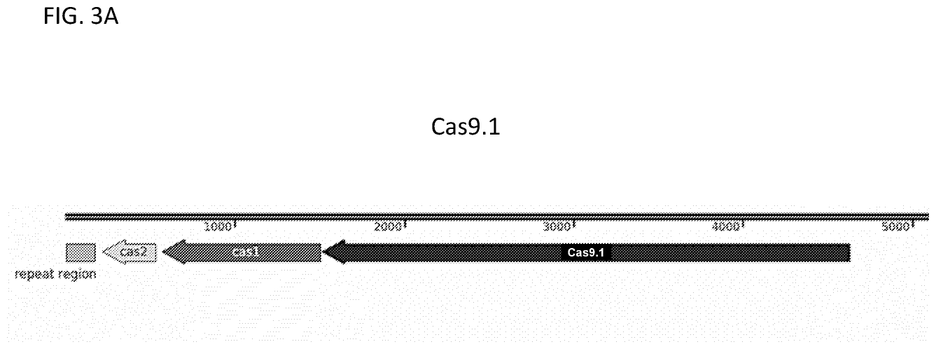

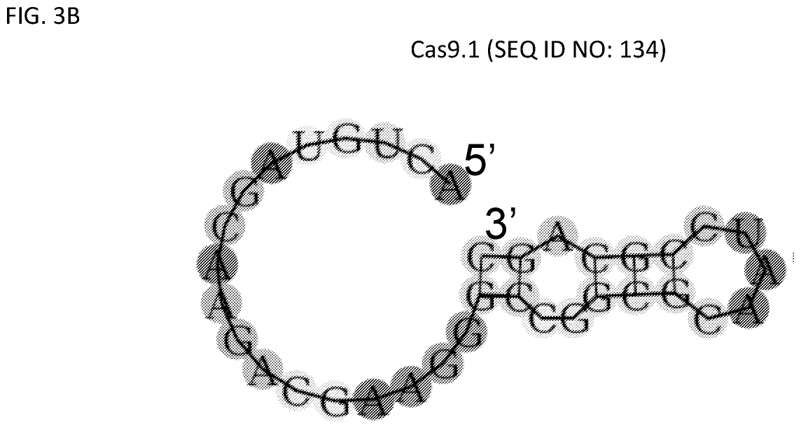

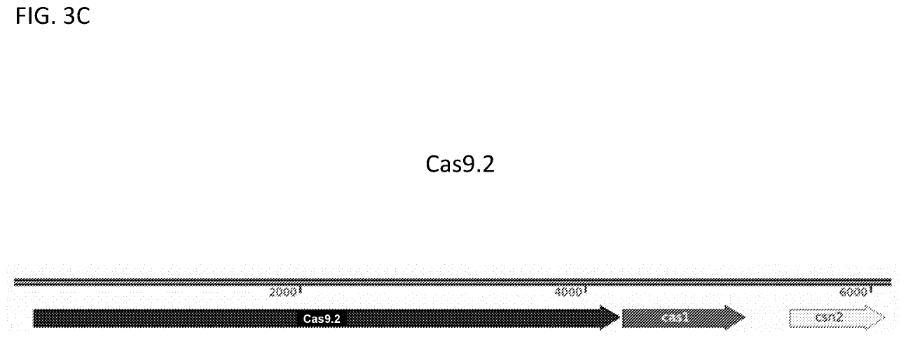

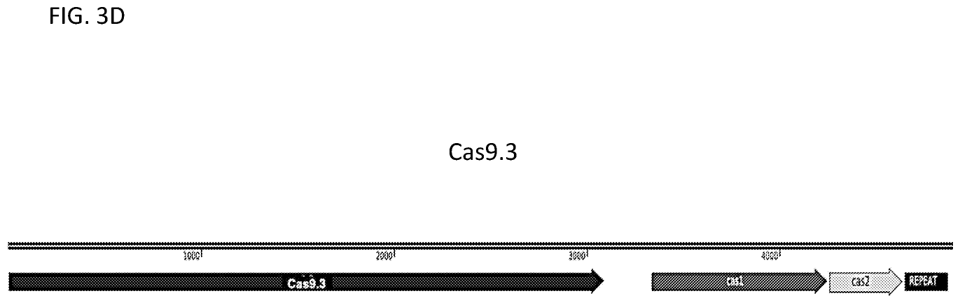

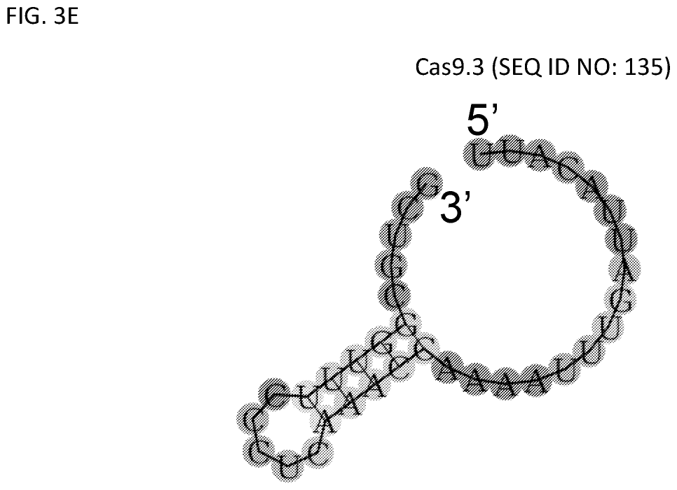

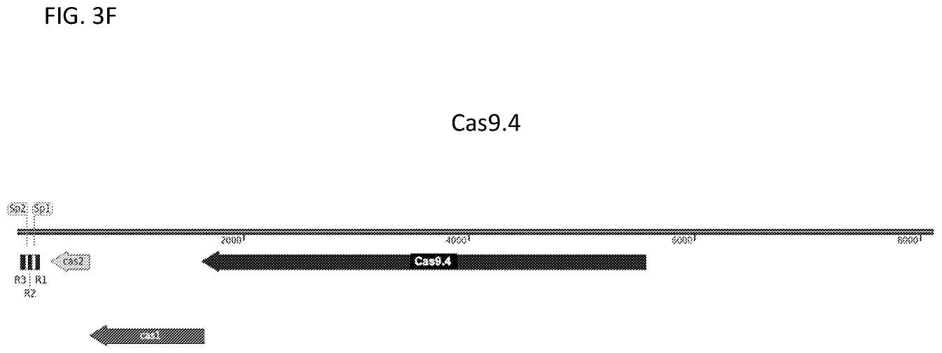

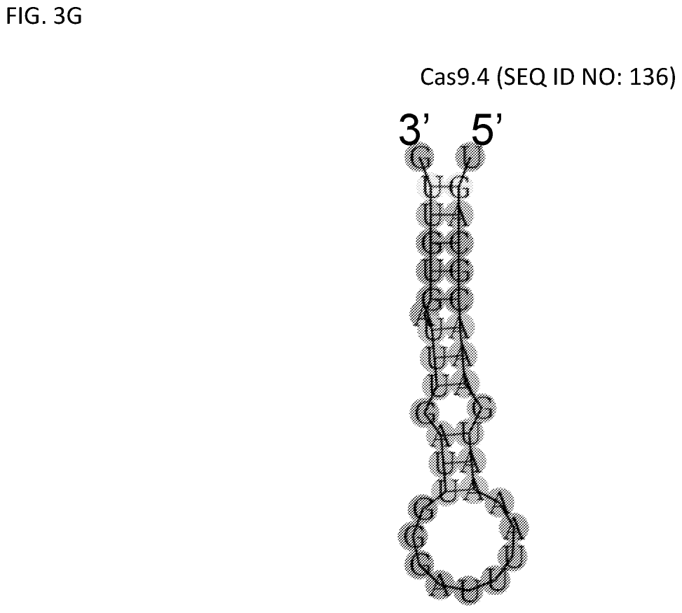

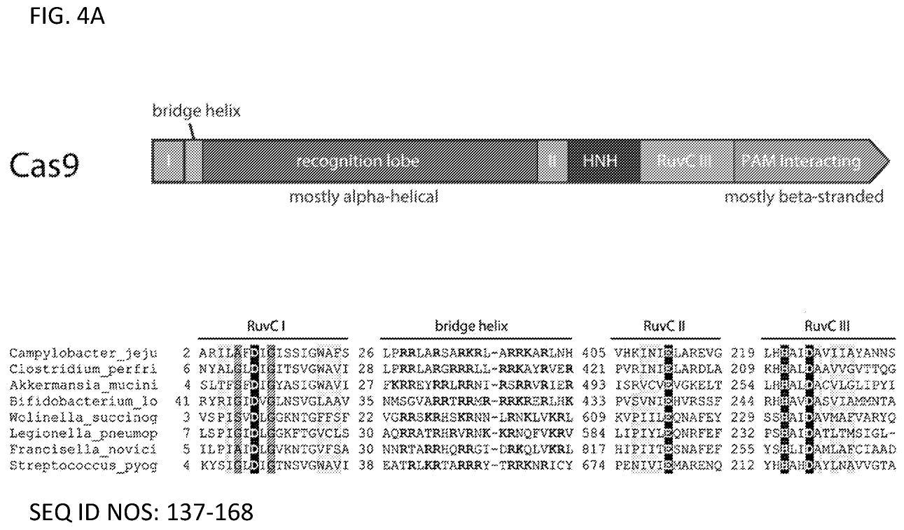

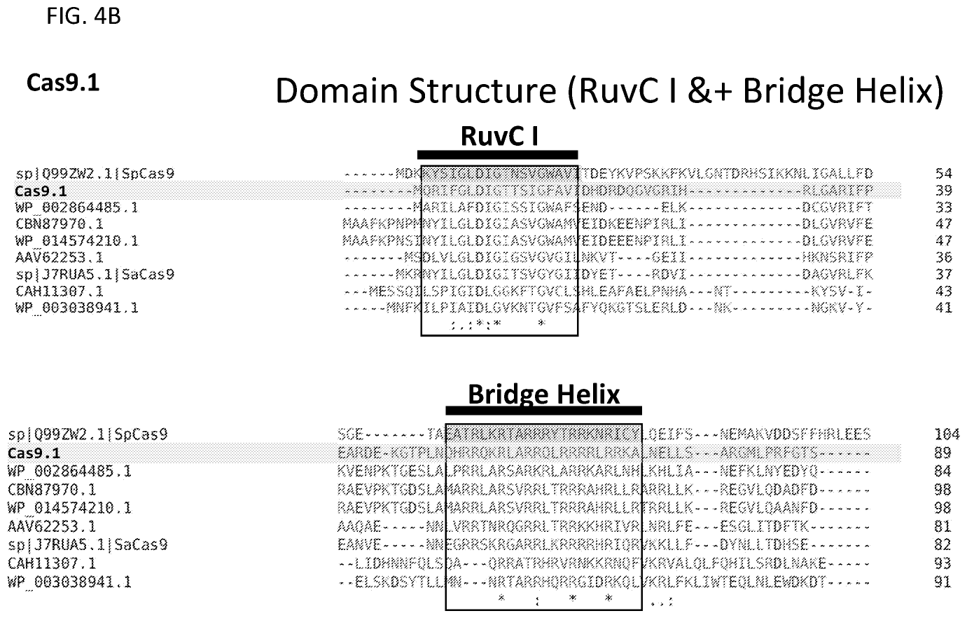

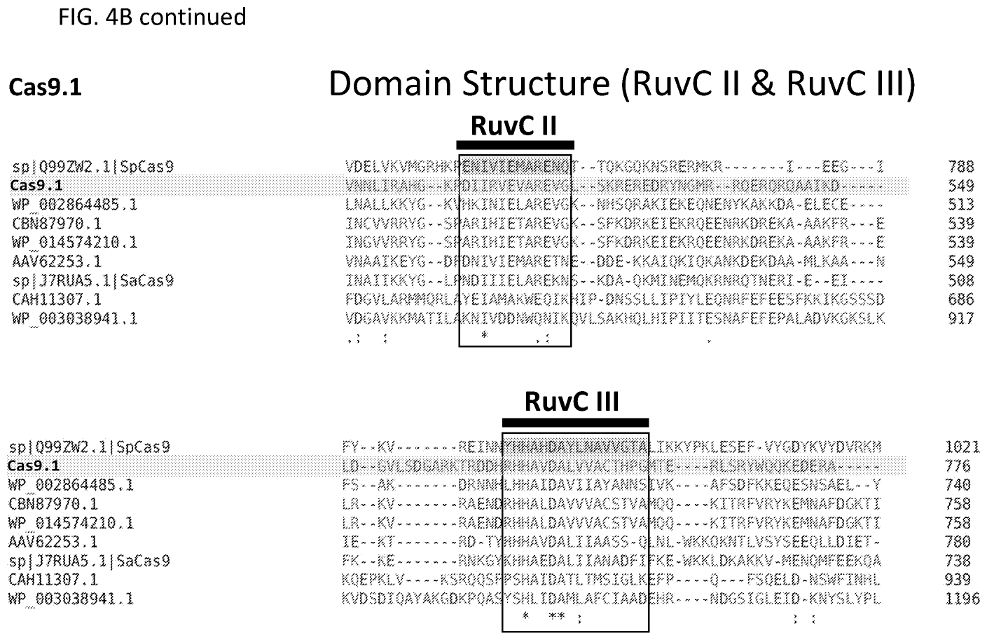

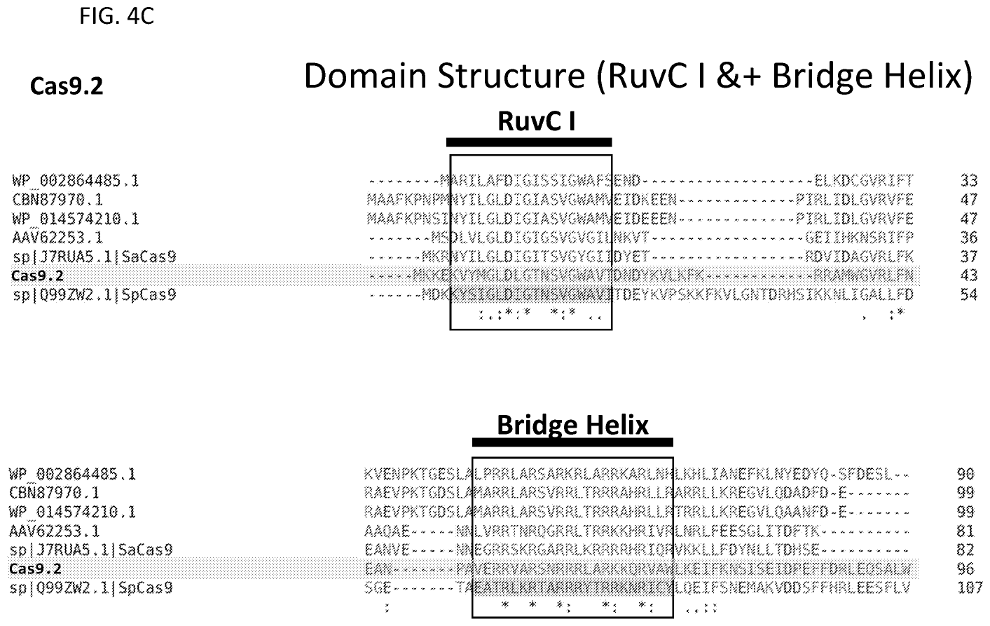

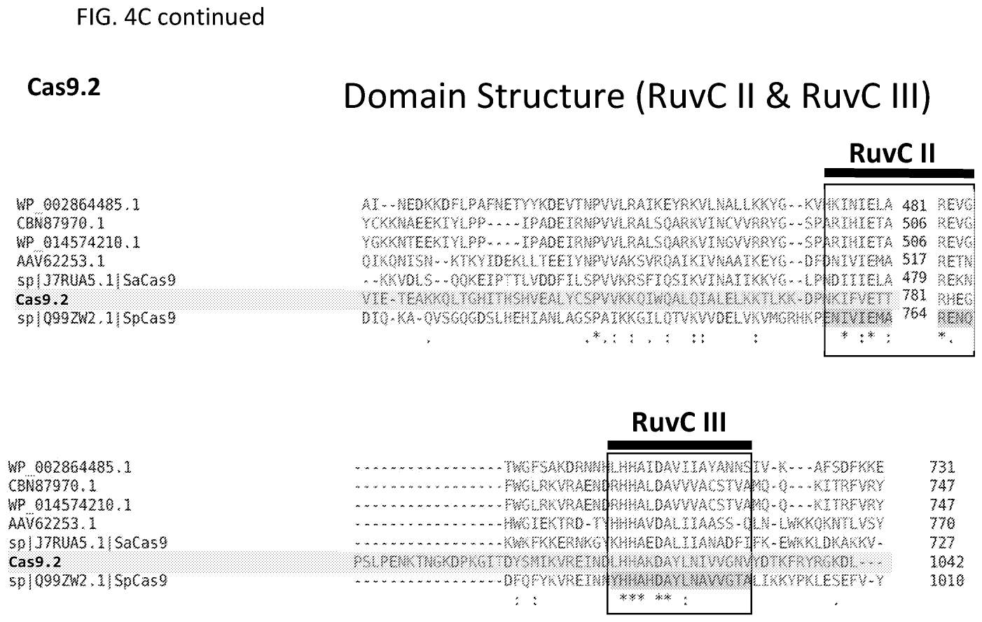

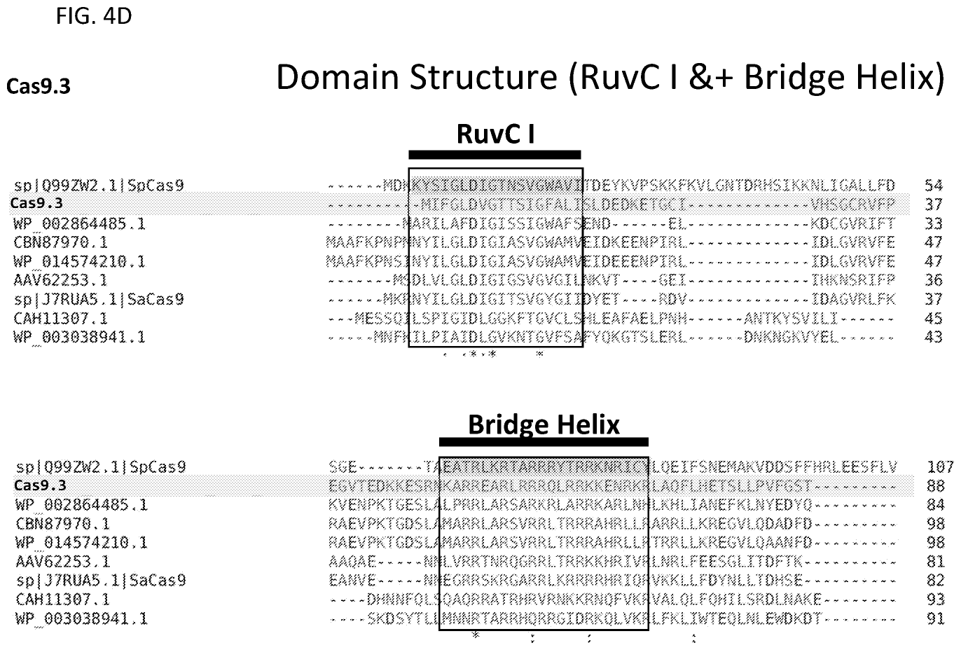

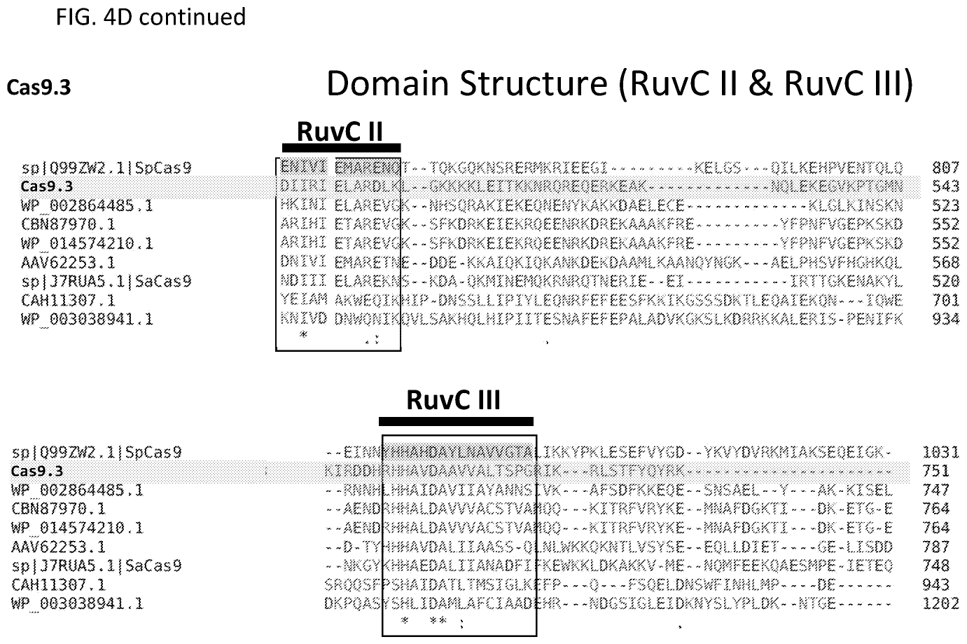

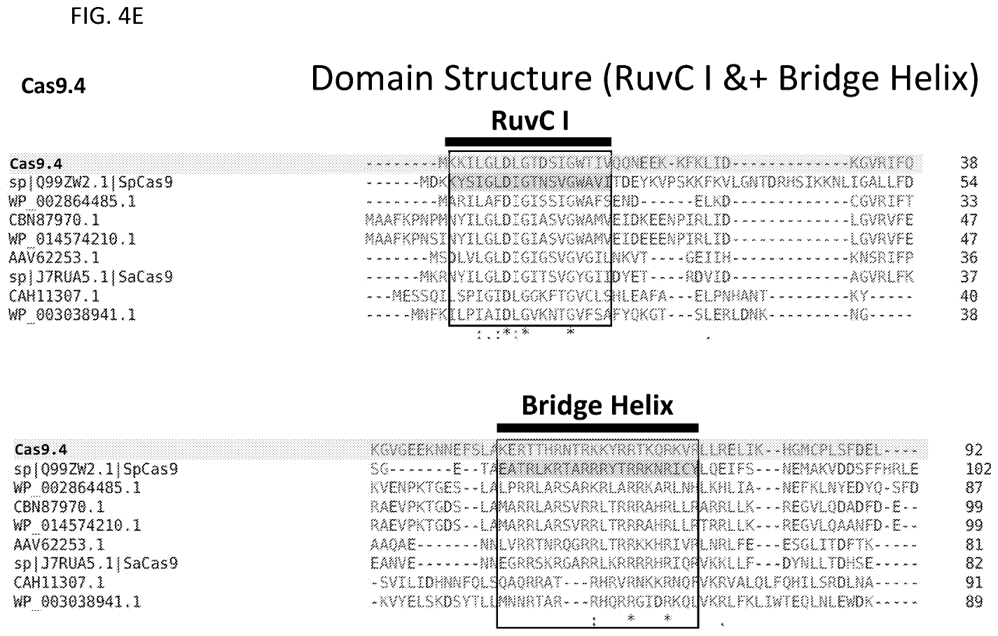

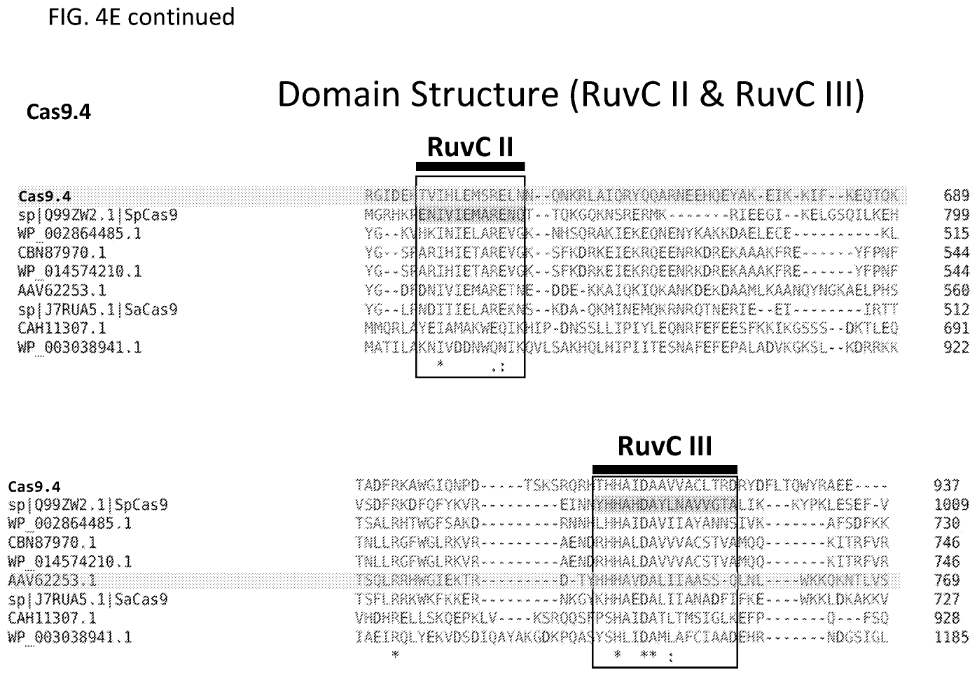

SEQ ID NO: 1 represents a novel Cas9 variant of the disclosure, Cas9.1, (1038 amino acids in length). FIG. 3A is a schematic representation of the CRISPR Cas cluster around the novel Cas9.1 gene. FIG. 4A shows the key catalytic amino acids for Cas9 proteins, and alignments of conserved motifs in selected representatives of the Cas9 protein family. FIG. 4B shows the alignment of RuvC1, Bridge Helix, RuvCII, and RuvCIII domains for Cas9.1 and other selected representatives of the Cas9 protein family.

SEQ ID NO: 2 represents a novel Cas9 variant of the disclosure, Cas9.2, (1375 amino acids in length). FIG. 3C is a schematic representation of the CRISPR Cas cluster around the novel Cas9.2 gene. FIG. 4C shows the alignment of RuvC1, Bridge Helix, RuvCII, and RuvCIII domains for Cas9.2 and other selected representatives of the Cas9 protein family.

SEQ ID NO: 10 represents a novel Cas9 variant of the disclosure, Cas9.3, (1031 amino acids in length). FIG. 3D is a schematic representation of the CRISPR Cas cluster around the novel Cas9.3 gene. FIG. 4D shows the alignment of RuvC1, Bridge Helix, RuvCII, and RuvCIII domains for Cas9.3 and other selected representatives of the Cas9 protein family.

SEQ ID NO: 11 represents a novel Cas9 variant of the disclosure, Cas9.4, (1329 amino acids in length). FIG. 3F is a schematic representation of the CRISPR Cas cluster around the novel Cas9.4 gene. FIG. 4E shows the alignment of RuvC1, Bridge Helix, RuvCII, and RuvCIII domains for Cas9.4 and other selected representatives of the Cas9 protein family.

TABLE 1
Cas9.1
MQRIFGLDIGTTSIGFAVIDHDRDQGVGRIHRLGARIFPEARDEKGTPLN
QHRRQKRLARRQLRRRRLRRKALNELLSARGMLPRFGTSAWHDAMALDPY
ALRARGTEEALQPVEVGRALYHLAQRRHFKPRDEAAEADEQEVGDQEAET
KREKLLQALRRSGRTLGQELAARGPHERKRHEHALRSTVETEFERLLTAQ
ARHHEILRDPEFVEELRETIFAQRPVFWRTSTLGTCPFVPGAPLCPKGAW
LSRQRRMLEQVNNLAITGGNARPLDHEERRAILAVLQTQASMSWGAVRTA
LKPLFKARGEAGAERRLRFNLEEGGGKTLLGNPLEAKLARIFGEAWATHP
HRDAIRETIHDRLFAATYNAKGAQRIVILPASQRAERMRGVIAGLQADFG
LSHEQAMALAELPLTPGWEPYSSEALRALMPKLEEGVRFGALVVAPEWED
WREATFPQRERPTGEVLDLLPSPKCHDESRRQTRLRNPTVLRTQNELRKV
VNNLIRAHGKPDIIRVEVAREVGLSKREREDRYNGMRRQERQRQAAIKDL
QAKGFAEPSRADVEKWLLWKESKETCPYTGDKICFDALFRRGEFQVEHIW
PRSRSFDDSFRNKTLCRRDVNLAKGNQTPFEFFESRPEEWEAVKRRLDGL
QAKRAGGEGMARGKVKRFVASTLPDDFAQRQLNDTGWAAREAVAFLKRLW
PDEGQAAPVRVQAVTGRVTAQLRHLGGLDGVLSDGARKTRDDHRHHAVDA
LVVACTHPGMTERLSRYWQQKEDERAERPQLDPPWPTIRADAEAAKDLIV
VSHRVRKKISGPFHKETVYGATDEREVTRGLEYEKFVTRKRVEDLTKSML
ADIRDDRVRQIVTAWVAERGGDPKKAFPPYPTLGSSGPEIRKVRVLIRRQ
PTLMARAATGFADLGANHHVAIYKTADERFAFEVVSLLEVARRVDRGEPP
VKRQRGDEKLVMSLAQGDLIRFAKTPDAEAAIWRVQKIATKGQISLLHHD
DASPKEPSLFEPMVGGLMARNPEKLAVDPIGRVRKAGD (SEQ ID
NO: 1)
Cas9.2
MKKEKVYMGLDLGTNSVGWAVTDNDYKVLKFKRRAMWGVRLFNEANPAVE
RRVARSNRRRLARKKQRVAWLKEIFKNSISEIDPEFFDRLEQSALWAEDK
NVAGKYSLFNEKKLTDKTFYRKFPTVFHLKKALMDGKIKKPDIRFVYLAL
SHYLQNRGHFLLENELNSVEDIDIRDIFNSLNERIHVLIDSGDDMVPAFD
LTNLDDLKQIATDTNISGKTQEKEAFIKTLLNGAKQPALEAIIKLCTGGS
ANLSKIFGDMFEFESEIKSISFEKANFEDEIAPKLQDCLGDYYQIIELAQ
QIYSWYTLYKVCSGRPSVSHAKVEDYEKHKEQLSHLKVLVRKHFSKNVYR
EIFRKEDDKIHNYVSYISGKKDRDEFYKYLKKTLEKKSTFKKTSEFENIS
RAIEQQNYLPKQRVKDNSVVPQQLYKQEIVKILNNLSSHYPFLSQKTDGI
SNREKIIKIFEYRIPYYVGPLCDIHRAGDDGFSWLVRDCSKKITPWNFEQ
VVDIPQSAENFIKNMTRKCTYLKQYNVLPKNSLLYSEYSVLNELNNVRIK
TKKLTPKLKEKMLNTLFRQKKNISITSLIHWLVSEGVYEKGEIEKSDVSG
VDSNFTSSLSAAISFDRIIGEKMKNKKTQKMVEEIINWLALFSDKKILQQ
KIVEKYQDKVSQEQIGKILRLNLSGWGRLSSEFLQLKNSQPGEHDGKTLI
NIMRQTQMNLMEIIHSPQFSFNTVIETEAKKQLTGHITHSHVEALYCSPV
VKKQIWQALQIALELKKTLKKDPNKIFVETTRHEGEKKRTTSRHKQLLEL
YQAAKSHLPDLTKSIKELNDALKDTEPEKMKRKKLFHYYKQLGRCMYTGR
PISLEDLFTNKYDIDHIYPQSLTKDDSFTNTVLVERLSNAEKSDAFPLDS
KTRKDRQGLWRCLRRNGLITKEKYYRLTRETPLSEEEKAAFIRRQLVETS
QTTKEVIRFLATLFPKSKVVYVKSGNVSDFRRDFSPSLPENKTNGKDPKG
ITDYSMIKVREINDLHHAKDAYLNIVVGNVYDTKFRYRGKDLTAIVREKA
RQYHLSRLFLYSTDGAWIGAADENRGKQRPSIETVIAEMRRNSCQVTWEA
VFKKGQLWDMNAKSKRPGLLPIKKELSDTAKYGGYQGKTASYFVVVEYEN
KKGEREKKLESVPIYVKALSKQKPDAVNSFLRDTLGLEKPSVMVDNIKIG
SIVEINGARMVLTGNNEVLVFGRIASQLILDITMAAYLKRMFKLLADTAK
IKENNVYFKNCGYLDKETNLAVYDTFIAKLKLPRYAQIITHSLYEKMESN
RDVFINLSLADQCNLLAGVLPALQCNSQNADLSLLGEGKAVGNIAFSKNA
ILKKNQVRLVDCSITGLFENSRNMA (SEQ ID NO: 2)
Cas9.3
MIFGLDVGTTSIGFALISLDEDKETGCIVHSGCRVFPEGVTEDKKESRNK
ARREARLRRRQLRRKKENRKRLAQFLHETSLLPVFGSTEWKNLMDNTHSN
PYELRSAALKKQLQPFELGKVIYHLAKHRGFKATKLDELMAESDEKKELG
VVKDGIKELDHKLGDQTLGVYLASIPPSEKKRGRYLGRYMIQEELEQILE
YQKHYNPELITSTFKKHLNSLIFSQRPTFWRLNTLGTCSLEQNESVCPKH
SWIGQQFIMMQKVNDLRIVEPHPRHLTMEERTQLIQGLCKQKIMSFGGIR
KLLHLPKGTVFNFETYQDKEDKRGLPGNAIEAALSTIFGSEWKHLPHKDA
IRSSLSNRIWSISYNRVGNKRIEIRADESYQNQRQTVKQEMMKDWNIAED
QAEQLVQLPIPPQWLRFSEKAIQKLLPDLESGVPLQTAIKEHYPETLKSS
EVEHELLPSSPHLVPELRNPTVNRALNELRKVVNNIIRSYGKPDIIRIEL
ARDLKLGKKKKLEITKKNRQREQERKEAKNQLEKEGVKPTGMNIEKFLLW
QESDGLDLYTGQKISFAALFKQTEYDIEHIIPRSRSFNNTFFNKTLAHNE
INRQKGNMIPKEFFGDGETWHAFVTRVNQSKLPLEKKEKLLIPHYDAIAS
EEMTERQLRDTAYIATEAKTYLQTLGIPVQPTNGRATASLRRVWGINSIW
ATEFGLEEESKKAAGEKIRDDHRHHAVDAAVVALTSPGRIKRLSTFYQYR
KEMKPDDFPLPWETFRADLITSLHKIIISHRVQRKISGPLHEETAYGFTK
KKSETDPTAYYFVTRKTLDKDFKPNKVKDIVDPAVRHLIGEHLQKFDNNP
AVAFAPENRPHMPLRKGGWGPPIKKVRIQIARNPQFMVSRQKNPISYYDS
GDNHHMAIYGTHLDDGTVDPETVSFEVVSRFEVNQRASKNEPLVKPQNEN
GVPLLFTLVKNNVLIWNEPGEEEQMHLVRWTTANKGRIFHKPLWMSGTPP
IEISISVKNLISYGGRKVSVDPIGNIFPCND (SEQ ID NO: 10)
Cas9.4
MKKILGLDLGTDSIGWTIVQQNEEKKFKLIDKGVRIFQKGVGEEKNNEFS
LAKERTTHRNTRKKYRRTKQRKVRLLRELIKHGMCPLSFDELELWSKYRK
GKPYIYPLSNKGFTQWLKLNPYDLRERAIKPDEKLTPLELGRIFYHITQR
RGFKSNRKDNSEDSEGVVKTSISQLREEMEGKTLGQFFNNELKKGNKVRK
KYTAREDYHHEFNEICNIQKIDNKTKAALEREIFFQRALKSQRHLVGKCT
LEPKKPRCPLSAIPYEEFRALQFINSIRIKDAEENLMPLTQKEREVIQSL
FFRKSKPSFPFNDIKKILEKHNGQRLTFNYPEKLQIIGSPTIALLKSVFG
EEWASLSVAYTKKDGTTGTINSEDVWHALFEFEHNDKLEDFLKQRLKLSD
DNIQKLIKGNLKQGYASLSRKAINNILPFLKDGHIYTHAVFLAKIPEIIG
RKQWLHSKDQIVNWFLKSAEELPLKNRLCKIVNNLITEFNETYANADPKY
ILDDSDKKSINRSLQHDFGPKTWNKFSSEKKDELQKETERLFLSQINKGN
ASAPYIKPYRQDEELKQYLIDNFNIKQEEAERIYHPSAIDIFDEAPYNDD
GIKLLQSPRTPSARNPMAMRALHELRYLLNQLLSQRGIDEHTVIHLEMSR
ELNNQNKRLAIQRYQQARNEEHQEYAKEIKKIFKEQTQKEIEPTEADILK
YRLWKEQEHNCLYTGRKIGIADFIGDNSNVDIEHTWPRSKSFDNSTANKT
LCDSHYNRNIKKNKIPYDLPNFKESAIIEGKQYDPIKARLKDWEEKCNHL
KELAAKYRYNAKRASTKEQKDKALQNAHFYQMHHEYWKDKIFRFTGKEIR
NSFKNSQLVDTGIINKYARAYLQTVFNKVFTIKGTLTADFRKAWGIQNPD
TSKSRQRHTHHAIDAAVVACLTRDRYDFLTQWYRAEEKGNERKKHIIQER
MKPWTTFVQDIKAFENSILVSHHTRKTSAKQTRKRLRENGKIVKDPNGNP
IYSKGDTFRNRLHKDTFYGAILRPQIDKEGKTVTDENGNPKLTTQYVVKK
PVTDLKETDIKNIVDSKIKSLFESKKLNEIQKEGISIPPSKPEGKETPIK
SVRLKQPFNPIPLREHTHLSQKPHKQYYHVQNEGNFLMAIYEETSASKKP
EKTFELISNLQAADYYKASNKENREQYPIVPERKFITKRNKEIELPLKQI
IYIGQMVMLYENSPEELKSKNEEELFKCLYKIVGITSMTIQAKYEYGVFI
LKHHAISTPYSELKPKDGDFSWEGNIEAMRKQLHSRIKVVIENLDFKITP
TGKIEWLF (SEQ ID NO: 11)

As used herein, Cas9.1 includes SEQ ID NO: 1 and proteins with at least 70%-99.5% sequence identity thereto. Accordingly, provided herein are proteins comprising the amino acid sequence of SEQ ID NO: 1 and proteins with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity thereto. Also provided herein are nucleic acids encoding the proteins comprising the amino acid sequence of SEQ ID NO: 1 and proteins with at least 70%-99.5% sequence identity thereto.

As used herein, Cas9.2 includes SEQ ID NO: 2 and proteins with at least 70%-99.5% sequence identity thereto. Accordingly, provided herein are proteins comprising the amino acid sequence of SEQ ID NO: 2 and proteins with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity thereto. Also provided herein are nucleic acids encoding the proteins comprising the amino acid sequence of SEQ ID NO: 2 and proteins with at least 70%-99.5% sequence identity thereto.

As used herein, Cas9.3 includes SEQ ID NO: 10 and proteins with at least 70%-99.5% sequence identity thereto. Accordingly, provided herein are proteins comprising the amino acid sequence of SEQ ID NO: 10 and proteins with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity thereto. Also provided herein are nucleic acids encoding the proteins comprising the amino acid sequence of SEQ ID NO: 10 and proteins with at least 70%-99.5% sequence identity thereto.

As used herein, Cas9.4 includes SEQ ID NO: 11 and proteins with at least 70%-99.5% sequence identity thereto. Accordingly, provided herein are proteins comprising the amino acid sequence of SEQ ID NO: 11 and proteins with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity thereto. Also provided herein are nucleic acids encoding the proteins comprising the amino acid sequence of SEQ ID NO: 11 and proteins with at least 70%-99.5% sequence identity thereto.

In some embodiments, the Cas9 protein of the disclosure is a catalytically active Cas9 protein, e.g. a catalytically active Cas9.1, Cas9.2, Cas9.3 or Cas9.4 protein.

In some embodiments, the Cas9 protein of the disclosure cleaves at a site distal to the target sequence, e.g. the Cas9.1, Cas9.2, Cas9.3 or Cas9.4.4 protein cleaves at a site distal to the target sequence.

In some embodiments, the Cas9 protein of the disclosure is a catalytically dead Cas9 protein, e.g. the Cas9.1, Cas9.2, Cas9.3 or Cas9.4 protein is catalytically dead (dCas9.1, dCas9.2, dCas9.3 or dCas9.4 protein).

In some embodiments, the Cas9 protein of the disclosure is a nickase Cas9 protein, e.g. a Cas9.1 nickase, Cas9.2 nickase, Cas9.3 nickase or Cas9.4 nickase protein.

The Cas9 proteins of the disclosure can be modified to include an aptamer.

The Cas9 proteins of the disclosure can be further fused to domains, e.g. catalytic domains to produce dual action Cas proteins. In some embodiments, a Cas9 protein is further fused to a base editor.

b. gRNAs for Class 2 Type II CRISPR-Cas RNA-Guided Proteins

The present disclosure provides DNA-targeting RNAs that direct the activities of the novel Cas9 proteins of the disclosure to a specific target sequence within a target DNA. These DNA-targeting RNAs are referred to herein as “gRNAs” or “gRNAs” Generally, as provided herein, a Cas9 variant gRNA comprises a first segment (also referred to herein as a “targeter-RNA”, a “DNA-targeting segment” or a “DNA-targeting sequence”) and a second segment (also referred to herein as a “activator-RNA”, a “activator-RNA” or a “protein-binding sequence”). Also provided herein are nucleotide sequences encoding the Cas9 gRNAs of the disclosure.

i. Targeter-RNA

The targeter-RNA of a Cas9 variant gRNA of the disclosure comprises a nucleotide sequence that is complementary to a sequence in a target DNA (targeting sequence of the gRNA; DNA-targeting sequence; spacer sequence). The targeter-RNA can interchangeably be referred to as a crRNA. The targeter-RNA of a gRNA interacts with a target DNA in a sequence-specific manner via hybridization (i.e., base pairing). As such, the nucleotide sequence of the targeter-RNA may vary and determines the location within the target DNA that the gRNA and the target DNA will interact. The targeter-RNA of a subject gRNA can be modified (e.g., by genetic engineering) to hybridize to any desired sequence within a target DNA.

The targeter-RNA can have a length of from about 12 nucleotides to about 100 nucleotides. For example, the targeter-RNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the targeter-RNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

Generally, a naturally unprocessed pre-crRNA for Cas9 comprises a direct repeat and an adjacent spacer (the portion of the crRNA that allows for targeting to a DNA molecule). In some embodiments, inclusion of direct repeats, and direct repeat mutations from unprocessed pre-crRNA into the mature gRNA may improve gRNA stability.

Table 2 shows the naturally occurring direct repeat sequences for the naturally occurring crRNAs of the Cas9 variants of the disclosure.

TABLE 2
Direct repeat sequences

| Description | Name | Sequence |
|---|---|---|
| Direct | Cas9.1 | ACTGTAGCAAGACGAAGG |
| Repeat | | GCCGGCGCAATCCGCAGC |
| | | (SEQ ID NO: 9) |

In some embodiments, the gRNAs of the disclosure include non-naturally occurring, engineered direct repeat sequences which can be incorporated into the engineered gRNAs of the disclosure.

ii. Spacer Sequences

gRNAs of the disclosure comprise spacer sequences, complementary to the target DNA. More specifically, the nucleotide sequence of the targeter-RNA that is complementary to a target nucleotide sequence (the DNA-targeting sequence or spacer sequence) of the target DNA can have a length at least about 12 nt. For example, the DNA-targeting sequence of the targeter-RNA that is complementary to a target sequence of the target DNA can have a length at least about 12 nt, at least about 15 nt, at least about 18 nt, at least about 19 nt, at least about 20 nt, at least about 25 nt, at least about 30 nt, at least about 35 nt or at least about 40 nt. For example, the DNA-targeting sequence of the targeter-RNA that is complementary to a target sequence of the target DNA can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. The nucleotide sequence (the DNA-targeting sequence) of the targeter-RNA that is complementary to a nucleotide sequence (target sequence) of the target DNA can have a length at least about 12 nt. In some embodiments, the DNA-targeting sequence of the targeter-RNA that is complementary to a target sequence of the target DNA is 20 nucleotides in length. In some embodiments, the DNA-targeting sequence of the targeter-RNA that is complementary to a target sequence of the target DNA is 19 nucleotides in length.

The percent complementarity between the spacer sequence of the targeter-RNA and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the DNA-targeting sequence of the targeter-RNA and the target sequence of the target DNA is 100% over the 1-25 contiguous 5′-most nucleotides of the target sequence of the complementary strand of the target DNA. In some embodiments, the percent complementarity between the DNA-targeting sequence of the targeter-RNA and the target sequence of the target DNA is at least 60% over about 1-25 contiguous nucleotides. In some embodiments, the percent complementarity between the DNA-targeting sequence of the targeter-RNA and the target sequence of the target DNA is 100% over the 1-25 contiguous 5′-most nucleotides of the target sequence of the complementary strand of the target DNA and as low as 0% over the remainder. In such a case, the DNA-targeting sequence can be considered to be 1-25 nucleotides in length.

In some embodiments the spacer sequence of a Cas9 gRNA of the disclosure is directed to a target sequence in a mammalian organism. In some embodiments the spacer sequence is directed to a target sequence in a non-mammalian organism.

In some embodiments, the spacer sequence of a Cas9 gRNA of the disclosure is directed to a target sequence which is a sequence of a human. In some embodiments, the target sequence is a sequence of a non-human primate.

In some embodiments the spacer sequence of a Cas9 gRNA of the disclosure is directed to a target sequence selected of a therapeutic target.

In some embodiments the spacer sequence of a Cas9 gRNA of the disclosure is directed to a target sequence selected of a diagnostic target—for example in such embodiments a labeled dCas9 of the disclosure and a gRNA directed to a diagnostic target DNA is contacted with the target DNA, or a cell comprising the target DNA, or a sample comprising the target DNA.

iii. Activator-RNA

The activator-RNA of a Cas9 variant gRNA of the disclosure binds with its cognate Cas9 variant of the disclosure. The activator-RNA can interchangeably be referred to as a tracrRNA. The gRNA guides the bound Cas9 protein to a specific nucleotide sequence within target DNA via the above described targeter-RNA. The activator-RNA of a Cas9 variant gRNA comprises two stretches of nucleotides that are complementary to one another.

iv. Dual-Molecule Cas9 gRNAs

In some embodiments, provided herein are dual molecule (two-molecule) Cas9 gRNAs for the novel Cas9 proteins of the disclosure. Such gRNAs comprise two separate RNA molecules (activator RNA-tracRNA; and the targeting RNA-crRNA). Each of the two RNA molecules of a subject double-molecule gRNA comprises a stretch of nucleotides that are complementary to one another such that the complementary nucleotides of the two RNA molecules hybridize to form the double stranded RNA duplex of the gRNA.

A dual-molecule gRNA can be designed to allow for controlled (i.e., conditional) binding of a targeter-RNA with an activator-RNA. Because a dual-molecule gRNA is not functional unless both the activator-RNA and the targeter-RNA are bound in a functional complex with Cas9 variant of the disclosure, a dual-molecule gRNA can be inducible (e.g., drug inducible) by rendering the binding between the activator-RNA and the targeter-RNA to be inducible. As one non-limiting example, RNA aptamers can be used to regulate (i.e., control) the binding of the activator-RNA with the targeter-RNA. Accordingly, the activator-RNA and/or the targeter-RNA can comprise an RNA aptamer sequence.

The dual-molecule guide can be modified to include an aptamer

v. Single-Molecule Cas9 Variant gRNAs

In some embodiments, provided herein are Cas9 gRNAs that comprises a single-molecule gRNA (interchangeably referred to herein as a sgRNA), for the novel Cas9 proteins of the disclosure.

Accordingly provided herein is an engineered single-molecule gRNA, comprising:

a. a targeter-RNA that is capable of hybridizing with a target sequence in a target DNA; and

b. an activator-RNA that is capable of hybridizing with the targeter-RNA to form a double-stranded RNA duplex, the activator-RNA comprising a activator-RNA, wherein the targeter-RNA and the activator-RNA are covalently linked to one another, wherein the single-molecule gRNA is capable of forming a complex with a novel Cas9 protein of the disclosure, and wherein hybridization of the targeter-RNA to the target sequence is capable of targeting the Cas9 protein of the disclosure to the target DNA.

A subject single-molecule gRNA comprises two segments of nucleotides (a targeter-RNA and an activator-RNA) that are complementary to one another, can be covalently linked by intervening nucleotides (“linkers” or “linker nucleotides”), and hybridize to form the double stranded RNA duplex (dsRNA duplex) of the activator-RNA, whereby resulting in a stem-loop structure. In some embodiments, the targeter-RNA and the activator-RNA are covalently linked via the 3′ end of the targeter-RNA and the 5′ end of the activator-RNA. In other embodiments, the activator-RNA is covalently linked via the 5′ end of the targeter-RNA and the 3′ end of the activator-RNA.

In some embodiments, the targeter-RNA and the activator-RNA are arranged in a 5′ to 3′ orientation.

In some embodiments, the activator-RNA and the targeter-RNA are arranged in a 5′ to 3′ orientation.

In some embodiments, the single molecule gRNA comprises one or more sequence modifications compared to a sequence of a corresponding wild type tracrRNA and/or crRNA.

In some embodiments, the targeter-RNA and the activator-RNA are covalently linked to one another via a linker.

When present, the linker of a single-molecule gRNA can have a length of from about 3 nucleotides to about 30 nucleotides. In exemplary embodiments, the linker of a single-molecule gRNA is 4, 5, 6, or 7 nt.

An exemplary single-molecule gRNA comprises two complementary stretches of nucleotides that hybridize to form a dsRNA duplex. In some embodiments, one of the two complementary stretches of nucleotides of the single-molecule gRNA (or the DNA encoding the stretch) is at least about 60% identical to one of the activator-RNA. For example, one of the two complementary stretches of nucleotides of the single-molecule gRNA (or the DNA encoding the stretch) is at least about 65% identical, at least about 70% identical, at least about 75% identical, at least about 80% identical, at least about 85% identical, at least about 90% identical, at least about 95% identical, at least about 98% identical, at least about 99% identical or 100% identical to an activator-RNA.

The activator-RNA and targeter-RNA segments can be engineered, while ensuring that the structure of the protein-binding domain of the gRNA is conserved. Thus, RNA folding structure of a naturally occurring protein-binding domain of a DNA-targeting RNA can be taken into account in order to design artificial protein-binding domains (either dual-molecule or single-molecule versions).

The activator-RNA in a single-molecule gRNA can have a length of from about 10 nucleotides to about 100 nucleotides. For example, the activator-RNA can have a length of from about 15 nucleotides (nt) to about 80 nt, from about 15 nt to about 50 nt, from about 15 nt to about 40 nt, from about 15 nt to about 30 nt or from about 15 nt to about 25 nt.

Also with regard to both the single-molecule and double-molecule gRNAs of the disclosure, the dsRNA duplex of the activator-RNA can have a length from about 6 nucleotides (nt) to about 50 bp. For example, the dsRNA duplex of the activator-RNA can have a length from about 6 nt to about 40 nt, from about 6 nt to about 30 bp, from about 6 nt to about 25 nt, from about 6 nt to about 20 nt, from about 6 nt to about 15 nt, from about 8 nt to about 40 nt, from about 8 nt to about 30 bp, from about 8 nt to about 25 nt, from about 8 nt to about 20 nt or from about 8 nt to about 15 nt. For example, the dsRNA duplex of the activator-RNA can have a length from about from about 8 nt to about 10 nt, from about 10 nt to about 15 nt, from about 15 nt to about 18 nt, from about 18 nt to about 20 nt, from about 20 nt to about 25 nt, from about 25 nt to about 30 nt, from about 30 nt to about 35 nt, from about 35 nt to about 40 nt, or from about 40 nt to about 50 nt. In some embodiments, the dsRNA duplex of the activator-RNA has a length of 8-15 base pairs. The percent complementarity between the nucleotide sequences that hybridize to form the dsRNA duplex of the activator-RNA can be at least about 60%. For example, the percent complementarity between the nucleotide sequences that hybridize to form the dsRNA duplex of the activator-RNA can be at least about 65%, at least about 70%, at least about 75%, at least about 80%, at least about 85%, at least about 90%, at least about 95%, at least about 98%, or at least about 99%. In some embodiments, the percent complementarity between the nucleotide sequences that hybridize to form the dsRNA duplex of the activator-RNA is 100%.

In some embodiments, the spacer sequence of a Cas9 gRNA (whether it is a single molecule gRNA or a dual molecule gRNA) of the disclosure is directed to a target sequence in a mammalian organism, e.g. a human or non-human primate. In some embodiments, the spacer sequence of a Cas9 gRNA of the disclosure is directed to a target sequence in a bacteria.

In some embodiments, the spacer sequence of a Cas9 gRNA of the disclosure is directed to a target sequence in a virus. In some embodiments, the spacer sequence of a Cas9 gRNA of the disclosure is directed to a target sequence in a plant.

In some embodiments, the single-molecule Cas9 gRNAs of the disclosure can be modified to include an aptamer.

vi. gRNA Arrays

The Cas9 gRNAs of the disclosure can be provided as gRNA arrays.

gRNA arrays include more than one gRNA arrayed in tandem, and can be processed into two or more individual gRNAs. Thus, in some embodiments a precursor Cas9 gRNA array comprises two or more (e.g., 3 or more, 4 or more, 5 or more, 2, 3, 4, or 5) gRNAs (e.g., arrayed in tandem as precursor molecules). In some embodiments, two or more gRNAs can be present on an array (a precursor gRNA array). A Cas9 protein of the disclosure can cleave the precursor gRNA array into individual gRNAs.

In some embodiments a Cas9 gRNA array includes 2 or more gRNAs (e.g., 3 or more, 4 or more, 5 or more, 6 or more, or 7 or more, gRNAs). The gRNAs of a given array can target (i.e., can include guide sequences that hybridize to) different target sites of the same target DNA. In some embodiments, two or more gRNAs of a precursor gRNA array have the same guide sequence. In some embodiments, the precursor gRNA array comprises two or more gRNAs that target different target sites within the same target DNA. In some embodiments, the precursor gRNA array comprises two or more gRNAs that target different target DNAs.

### II. Class 2 Type V CRISPR-Cas RNA-Guided Systems

Provided herein are novel Class 2 Type V CRISPR-Cas RNA-guided proteins and their gRNAs, constituting the novel Class 2 Type V CRISPR-Cas RNA-guided systems of the disclosure.

Provided herein are engineered systems comprising: a Class 2 Type V CRISPR-Cas RNA-guided endonuclease protein and a single guide RNA, wherein the gRNA and the Class 2 Type V CRISPR-Cas RNA-guided endonuclease protein do not naturally occur together, wherein the gRNA is capable of hybridizing to a target sequence in a target DNA, wherein the gRNA is capable of forming a complex with the Class 2 Type V CRISPR-Cas RNA-guided endonuclease protein, and wherein the Class 2 Type V CRISPR-Cas RNA-guided endonuclease protein possesses collateral activity and is capable of collaterally cleaving a single stranded polynucleotide comprising RNA, without the use of a tracrRNA. In some embodiments, the Class 2 Type V CRISPR-Cas RNA-guided endonuclease protein comprises the amino acid sequence of SEQ ID NO: 4, or at least 70% sequence identity thereto. In some embodiments, the Class 2 Type V CRISPR-Cas RNA-guided endonuclease protein is capable of collaterally cleaving a single stranded RNA. In some embodiments, the Class 2 Type V CRISPR-Cas RNA-guided endonuclease protein is capable of collaterally cleaving a single stranded DNA/RNA hybrid.

Also provided herein are engineered systems comprising: (a) a Cas12a.1, Cas12p, or Cas12q protein, or a nucleic acid encoding the Cas12a.1, Cas12p, or Cas12q protein; and (b) a Cas12a.1, Cas12p, or Cas12q gRNA, or a nucleic acid encoding a Cas12a.1, Cas12p, or Cas12q gRNA, wherein the gRNA and the Cas12a.1, Cas12p, or Cas12q protein do not naturally occur together, wherein the gRNA is capable of hybridizing to a target sequence in a target DNA, and the gRNA is capable of forming a complex with the Cas12a.1, Cas12p, or Cas12q protein.

The components are described in turn below.

a. Class 2 Type V CRISPR-Cas RNA-Guided Proteins

Provided herein are novel Class 2 Type V CRISPR-Cas RNA-guided endonucleases, e.g. novel Cas12 proteins of the disclosure, including novel Cas12a variants, and novel Cas12 subtypes. In some embodiments the novel Cas12 proteins of the disclosure have been deduced using bioinformatics methods.

Table 3a shows the protein sequences for the novel Cas12 proteins of the disclosure. Table 2b shows the nucleotide sequences encoding the novel Cas12a proteins of the disclosure.

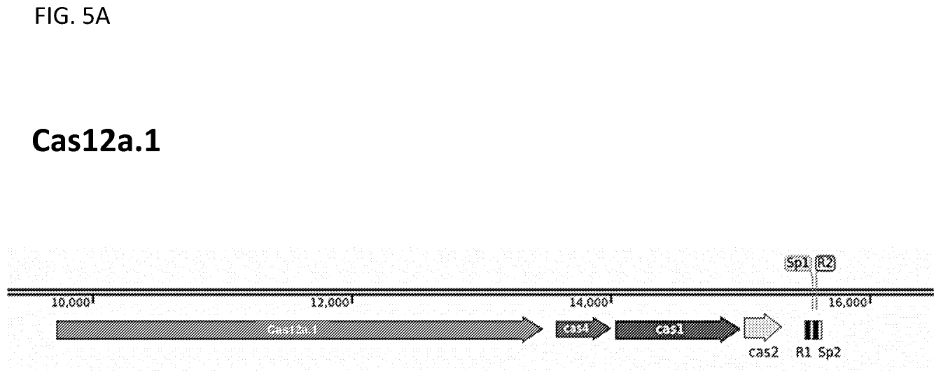

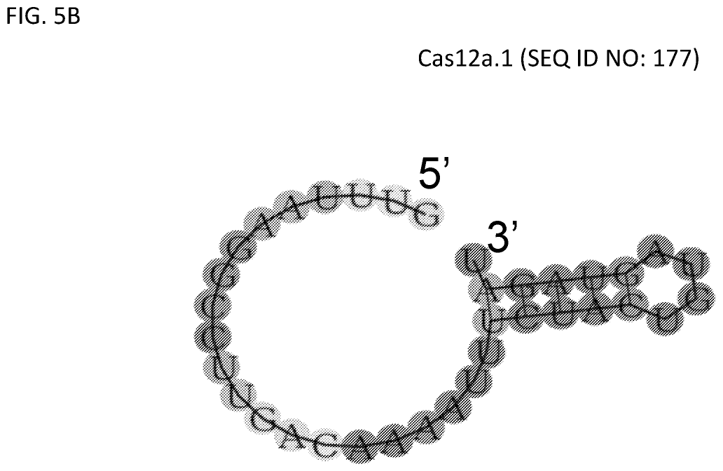

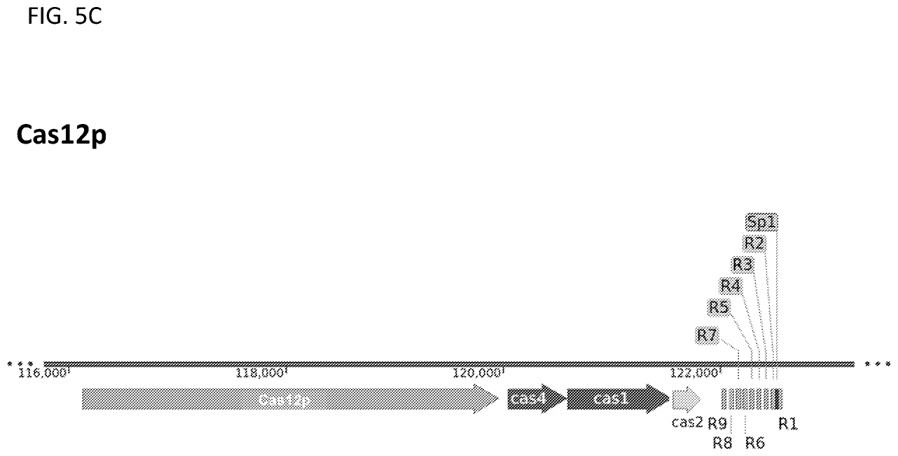

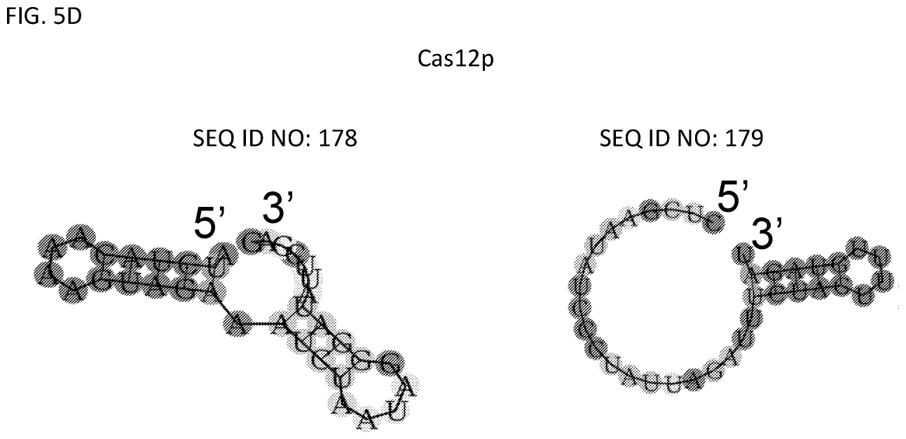

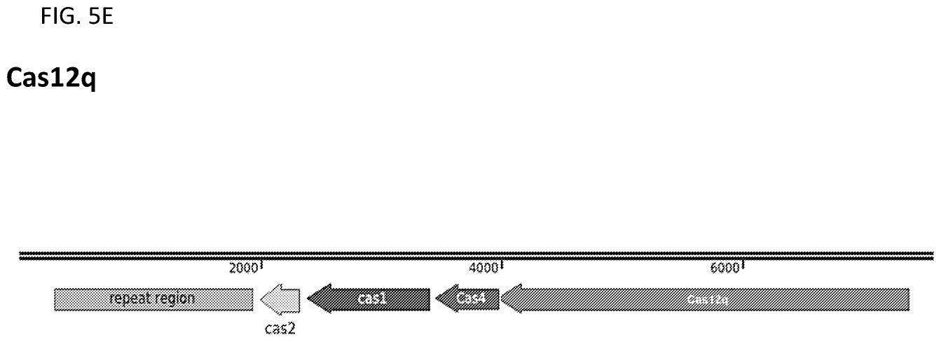

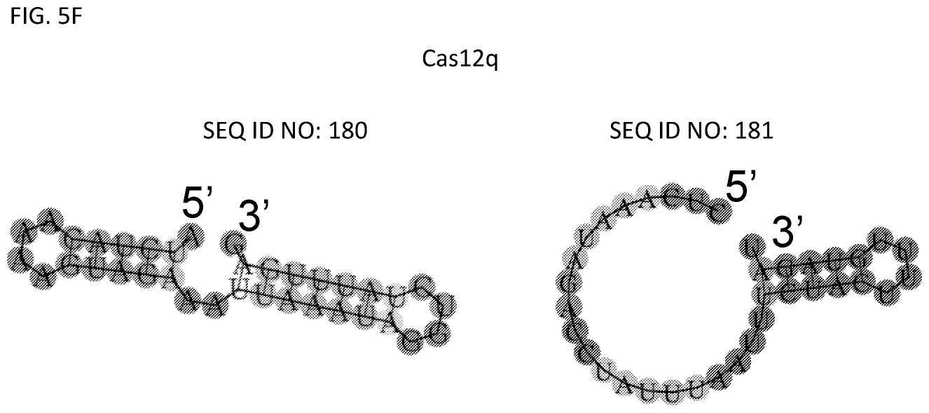

SEQ ID NO: 3 represents a novel Cas12a variant of the disclosure, Cas12a.1 (1254 amino acids in length). Cas12a.1 was isolated from a metagenomics sample and deduced to be from Candidatus Micrarchaeota archaeon. Based on sequence, function, and structural features it is believed that Cas12a.1 is a Cas12a subtype. FIG. 5A is a schematic representation of the CRISPR Cas cluster around the novel Cas12a.1 gene. FIG. 6A shows the key catalytic amino acids for Cas12a proteins, and alignments of conserved motifs in selected representatives of the Cas12a protein family. FIG. 6B shows the alignment of RuvC1, Bridge Helix, RuvCII, and RuvCIII domains for Cas12a.1 and other selected representatives of the Cas12a protein family. SEQ ID NO: 13 shows the nucleotide sequence encoding the Cas12a.1 of the disclosure.

SEQ ID NO: 4 represents a novel Cas12 subtype of the disclosure, Cas12p (1281 amino acids in length). Cas12a.1 was isolated from a metagenomics sample and deduced to be from Candidatus Peregrinibacteria bacterium. Based on sequence, function, and structural features described herein, Cas12p differs from the other members of the Cas12 family identified to date and thus is a novel Cas12 enzyme. This novel Cas12 subtype possesses unique properties, not seen in other Cas12 proteins, for example, the ability to collaterally cleave a RNA or DNA containing sequence, e.g. single stranded DNA, singled stranded RNA, and single stranded chimeric RNA/DNA, without the use of a tracrRNA. It is noted that SEQ ID NO: 222 also in Table 3a is N-terminal truncation of the Cas12p of SEQ ID NO: 4.

SEQ ID NO: 14 provides a nucleotide sequence encoding the Cas12p of the disclosure. FIG. 5C is a schematic representation of the CRISPR Cas cluster around the novel Cas12p gene. FIG. 6B.1 shows the alignment of Cas12a.1 vs. SEQ ID NO: 81 of US20160208243, and has a 46.8% sequence identity; and FIG. 6C shows the alignment of Cas12a.1 vs. SEQ ID NO: 3 of U.S. Pat. No. 10,253,365, and has a 46.5% sequence identity.

FIG. 6D shows the amino acid sequence of Cas12p with the RuvC motifs underlined (SEQ ID NO: 4). The FnCas12a sequence referenced in Shmakov et al., 2015 was used as a reference for identification of the Ruv motifs. FIG. 6E shows the alignment of Cas12p with Cas12g1, another Cas12 enzyme. This figure shows an alignment of Cas12p with Cas12g1. Although Cas12g1 has been reported to possess the ability to collaterally cleave RNA (trans-cleavage), the sequence homology is less than 8.9% as retrieved by the program Clustal Omega. The very low homology between the enzymes and the lack of conserved domains indicate that they are members of different enzyme families. Moreover, Cas12g1 requires the presence of a tracr sequence, Cas12p does now, providing an additional functional distinction.

In the following figures, the structure of Cas12p protein was modeled based on Fn Cas12a structure with Swiss Model server. The sequence identity between the proteins is 38.34%. The model covered the entire sequence of the Cas12p protein. FIG. 6F shows a structural analysis of Cas12p using the Swiss Model server. FIG. 6G shows a spatial prediction of non-conserved amino acid residues in Cas12p. It is seen that the non-conserved residues are located on protein exposed surface. These differences could reflect changes on first contact with substrates and solvent interactions. FIG. 6H shows the approximation of charge distribution over the surface of Cas12p. Using the model showed in FIG. 6F, vacuum electrostatics generated by Pymol software allowed for the modeling of the approximation of charge distribution over the surface of the proteins. The positive to negative charge is represented from white to black, the white zones representing the most positive ones. The white oval highlights the active site groove on both positions. The figure shows a slight increase of positive charges on the active site groove of Cas12p protein in comparison to FnCas12a. An increase of positive charge could be related to a stronger interaction with a negative charge substrate and could explain the increased affinity of Cas12p to RNA and DNA substrates. FIG. 6I shows predicted structural differences between Cas12p and FnCas12a based on protein sequences. On FnCas12a, the region 696-706 on PAM-interacting domain is related to the binding and cleavage of target DNA and the region 842-852 on Wedge III region is related to pre-cRNA processing (Swarts et al, 2017). When compared to Cas12p, the enzyme presents low homology on those regions, given the deletion of the sequences KNGNPQKGY (SEQ ID NO: 113) on position 699 and PAKE (SEQ ID NO: 114) on position 844. Due to the catalytic relevance of those regions, it is possible to relate the sequence changes to changes seen with the catalysis. The deletions are predicted to impact on the secondary structure of Cas12p. The figures show a superimposition of the model of Cas12p (light grey) and the structure of FnCas12a (dark grey), the deleted sequences are shown in black. The lack of the sequence KNGNPQY (SEQ ID NO: 115) is reflected on a loop shortening. The lack of PAKE sequence (SEQ ID NO: 114; added to additional changes on the loop), decrease a loop length and reduce the negative charges on that position of Cas12p. FIG. 6J shows RuvCIII domain structural analysis of Cas12p based on structural analysis with Swiss Model server. The FnCas12a sequence referenced in Shmakov et al., 2015 was used as a reference for identification of the Ruv motifs. Although the RuvCIII region is conserved on Cas12p and the prototypic Cas12a proteins, the Cas12p has several differences on the sequences surrounding the domain. The presence of these changes impact on the secondary structure of Cas12p (as is shown in black) and could account for the differential RNA cleavage activity of this enzyme. In the structural model depicted in the figure, the superimposition of the structure of the RuvCIII region of the studied Cas12a enzymes and the model of Cas12p. Changes on the secondary structure of Cas12p are circled and shown in black. FIGS. 91B, 9C and 17 show the unique collateral activity of this novel Cas12p enzyme.

SEQ ID NO: 5 represents a novel Cas12 of the disclosure, Cas12q (1137 amino acids in length). FIG. 5E is a schematic representation of the CRISPR Cas cluster around the novel Cas12q gene. FIG. 6K shows the Cas12q sequence with RuvC motifs underlined for the novel Cas12 protein of the disclosure, Cas12q. The FnCas12a sequence referenced in Shmakov et al., 2015 was used as a reference for identification of the Ruv motifs. SEQ ID NO: 15 shows the nucleotide sequence encoding the Cas12q of the disclosure.

TABLE 3a
Cas12a.1
MKVSTWDSFTNQYPLTKTLRFELKPVGKTLQKIQDRNLITEDEQRQKDFN
KVKKIMDGYYKQFIEECLEGAKIPLKKLEENNNAYTKLKKDPYNKKLREE
YAKLQKQLRKLIHDEINKKEEFKYLFKKEFIKKILPEWLEKKGKKEELKE
IEKFDKWVTYFSGFFNNRKNVFSSDEISTSMIYRIVNDNLPKFLDDVSRF
GEITRYKEFDANQIEENFESELNGEKLKDFFNLKNFNNCLNQEGIEKFNL
IIGGKSEEGNNKIKGLNELVNELAQKQADKNEQKKVRKLKLAPLFKQILS
DRKSSSFAFEKFEENTEVFDAIDEFYDKISLETLKKIEATLEKLEEKDLE
LVYLKNDRCLTGISQEVFGDRERVLQALREYAKTELGLKTDKKIEKWMKK
GRYSIHEIESGLKKIGSTGHPICNYFSKLEEKKTNLIQEIKKARTEYEKI
SDKKKKLTAESQEPNVARIKALLDSIMRLYHFIKPLNINFKNKKEKDSEA
LETDNDFYNDFDESFAELGNIIPLYNQVRNYVTQKPFSTEKFKLNFENPK
LLSGWDKNKEKDYYSVILRKEESYYLAIMTPKQKNVFDELERLPAGKNYF
EKIDYKLLPTPEKNLPRILFAKKNISFYKPSKEIEAIRNHSAHTKHGNPQ
NGFKKRDFRLSDCHKMIDFYKKSIQKHPEWKEYDFQFKKTEDYVDISEFY
KEVSDQGYKIEFKKISEKYLLDLVEEGKLYLFQIWNKDFSKYSEGRKNLH
TIYWKELFSKENLSDITYKLNGEAEIFYRPKSMERKVTHPKNQKIENKDP
IKGKKFSKFKYDFIKNKRYTEDRFFFHCPITLNFQARDGSKTINKRVNDH
IRETKDDIFVLSIDRGERHLAYYTLLNSKGEIQEQGSFNVISDDKERKRD
YHEKLDEREKERDKARKSWQKIETIKKLKDGYLSQIVHKIAKLAIEKNAI
IVLEDLNLDFKRGRLKIEKQVYQKFEKKLIDKLNYLVFKERTEKEAGGSL
NAYQLTGKFEGFKKLGKETGIIYYVPAAYTSKICPKTGFVNLLRPKFKNI
EKAKEFFKKFNYIKYDSSEGLFEFNFDYSKFIKNGKKETKIIQDNWSVYS
NGTKLVGFRNKNKNNSWDTKEVKPNEKLKILFKEYGVSFQKDENIISQIA
SQNKKAFFENLIKIFKTILMLRNSRKDPEEDYVLSCVKDENGEFFDSRKA
KDNEPKDADANGAYHIGLKGLMLLERIKANKGKKKLDLLISRNDFINFAV
ERSK (SEQ ID NO: 3)
Cas12p
MKKSIFDQFVNQYALSKTLRFELKPVGETGRMLEEAKVFAKDETIKKKYE
ATKPFFNKLHREFVEEALNEVELAGLPEYFEIFKYWKRYKKKFEKDLQKK
EKELRKSVVGFFNAQAKEWAKKYETLGVKKKDVGLLFEENVFAILKERYG
NEEGSQIVDESTGKDVSIFDSWKGFTGYFIKFQETRKNFYKDDGTATALA
TRIIDQNLKRFCDNLLIFESIRDKIDFSEVEQTMGNSIDKVFSVIFYSSC
LLQEGIDFYNCVLGGETLPNGEKRQGINELINLYRQKTSEKVPFLKLLDK
QILSEKEKFMDEIENDEALLDTLKIFRKSAEEKTTLLKNIFGDFVMNQGK
YDLAQIYISRESLNTISRKWTSETDIFEDSLYEVLKKSKIVSASVKKKDG
GYAFPEFIALIYVKSALEQIPTEKFWKERYYKNIGDVLNKGFLNGKEGVW
LQFLLIFDFEFNSLFEREIIDENGDKKVAGYNLFAKGFDDLLNNFKYDQK
AKVVIKDFADEVLHIYQMGKYFAIEKKRSWLADYDIDSFYTDPEKGYLKF
YENAYEEIIQVYNKLRNYLTKKPYSEDKWKLNFENPTLADGWDKNKEADN
STVILKKDGRYYLGLMARGRNKLFDDRNLPKILEGVENGKYEKVVYKYFP
DQAKMFPKVCFSTKGLEFFQPSEEVITIYKNSEFKKGYTFNVRSMQRLID
FYKDCLVRYEGWQCYDFRNLRKTEDYRKNIEEFFSDVAMDGYKISFQDVS
ESYIKEKNQNGDLYLFEIKNKDWNEGANGKKNLHTIYFESLFSADNIAMN
FPVKLNGQAEIFYRPRTEGLEKERIITKKGNVLEKGDKAFHKRRYTENKV
FFHVPITLNRTKKNPFQFNAKINDFLAKNSDINVIGVDRGEKQLAYFSVI
SQRGKILDRGSLNVINGVNYAEKLEEKARGREQARKDWQQIEGIKDLKKG
YISQVVRKLADLAIQYNAIIVFEDLNMRFKQIRGGIEKSVYQQLEKALID
KLTFLVEKEEKDVEKAGHLLKAYQLAAPFETFQKMGKQTGIVFYTQAAYT
SRIDPVTGWRPHLYLKYSSAEKAKADLLKFKKIKFVDGRFEFTYDIKSFR
EQKEHPKATVWTVCSCVERFRWNRYLNSNKGGYDHYSDVTKFLVELFQEY
GIDFERGDIVGQIEVLETKGNEKFFKNFVFFFNLICQIRNTNASELAKKD
GKDDFILSPVEPFFDSRNSEKFGEDLPKNGDDNGAFNIARKGLVIMDKIT
KFADENGGCEKMKWGDLYVSNVEWDNFVANK (SEQ ID NO: 4)
Cas12p truncated
NSIDKVFSVIFYSSCLLQEGIDFYNCVLGGETLPNGEKRQGINELINLYR
QKTSEKVPFLKLLDKQILSEKEKFMDEIENDEALLDTLKIFRKSAEEKTT
LLKNIFGDFVMNQGKYDLAQIYISRESLNTISRKWTSETDIFEDSLYEVL
KKSKIVSASVKKKDGGYAFPEFIALIYVKSALEQIPTEKFWKERYYKNIG
DVLNKGFLNGKEGVWLQFLLIFDFEFNSLFEREIIDENGDKKVAGYNLFA
KGFDDLLNNFKYDQKAKVVIKDFADEVLHIYQMGKYFAIEKKRSWLADYD
IDSFYTDPEKGYLKFYENAYEEIIQVYNKLRNYLTKKPYSEDKWKLNFEN
PTLADGWDKNKEADNSTVILKKDGRYYLGLMARGRNKLFDDRNLPKILEG
VENGKYEKVVYKYFPDQAKMFPKVCFSTKGLEFFQPSEEVITIYKNSEFK
KGYTFNVRSMQRLIDFYKDCLVRYEGWQCYDFRNLRKTEDYRKNIEEFFS
DVAMDGYKISFQDVSESYIKEKNQNGDLYLFEIKNKDWNEGANGKKNLHT
IYFESLFSADNIAMNFPVKLNGQAEIFYRPRTEGLEKERIITKKGNVLEK
GDKAFHKRRYTENKVFFHVPITLNRTKKNPFQFNAKINDFLAKNSDINVI
GVDRGEKQLAYFSVISQRGKILDRGSLNVINGVNYAEKLEEKARGREQAR
KDWQQIEGIKDLKKGYISQVVRKLADLAIQYNAIIVFEDLNMRFKQIRGG
IEKSVYQQLEKALIDKLTFLVEKEEKDVEKAGHLLKAYQLAAPFETFQKM
GKQTGIVFYTQAAYTSRIDPVTGWRPHLYLKYSSAEKAKADLLKFKKIKF
VDGRFEFTYDIKSFREQKEHPKATVWTVCSCVERFRWNRYLNSNKGGYDH
YSDVTKFLVELFQEYGIDFERGDIVGQIEVLETKGNEKFFKNFVFFFNLI
CQIRNTNASELAKKDGKDDFILSPVEPFFDSRNSEKFGEDLPKNGDDNGA
FNIARKGLVIMDKITKFADENGGCEKMKWGDLYVSNVEWDNFVANK
(SEQ ID NO: 222)
Cas12q
MINIDELKNLYKVQKTITFELKNKWENKNDENDRVEFLKTQEWVESLFKV
DEENFDEKESIPNLLDFGQKIASLFYKLSEDIANNQIDTRVLKVSKFLLE
EIDRNQYHEKKNKPTKVKEMNPNTNKSYIKEYKLSDQNTLYVLLKIMEDE
GRGLQKFLYDKADRLNLYNQKVRRDFALKESNEQQKFSGNANYYGNIKLL
IDSLEDAVRIIGYFTFDDQAENAQINEFKSVKQEMNNNEASYQALKDFAI
DNAKKEIELTTLNHRAVNKDPKKIQEQIEEVENFEEDINQLKHQISALND
KKFDVVSRLKHALIKMLPELNLLDAESEQGREVQQIYQDKKNGLELDDFK
FNLLKHHQWQKTIFKYIKLEGLVLPDLYAENKQDKIKVYlENYRQSGERI
SKKAREELGKIDKREEFNGNDELKKAWYEYKDFCRDKRNKSVELGNKKSL
YNAIKREVLRQKMCNHFAVLVSDGEDTSPYYYLILIPNENSDEMNRTFKE
LKASEGNWKMLDYNRLTFKALEKLALLRSSTFEIADQELQEEAKKIWEEY
KEKAYKDFKNKKLLQGLSGRQREEKKQELQKESLNRVINYLIRCIQSLPD
SGKYNFNFKEPHQYQSLEEFAEEIDRQGYHCAWKNVSKDKLMELEAMEKI
KVFKLHNKDFRKVKLNDSKHNPNLFTLYWLDAMNLDKVNVRLLPEVDLYK
RAKETQLKLFERDVKCNINNQKIKSIKEKNRLFQDKLYASFKLEFYPENE
GLGFEQVNDKVNNFCGSDTAYYLGLDRGEKELVTFCLVDSDGRLVKNGDW
TKFKEVNYADKLKQFYYSKGEIESTQQQLLEARDNIKQATNTEDKESMKL
NYKKLELKLKQQNLLAQEFIKKAYCGYLIDSINEILREYPNTYLVLEDLD
IAGKADPESGMTNKEQNLNKTMGASVYQAIENAIVNKFKYRTVKLSDIKG
LQTVPNVVKVEDLREVKEVEDGEHKFGLIRSVKSKDQIGNILFVDEGETS
NTCPNCGFNSDWFKRDVDFDLEIVATVNGQKNAVIEQNDKKYCFPGEIYK
LEIINKEYETNKRNLAMIFKPRAKACRKFINNNLDKNDYFYCPYCAFSSK
NCNNPKLQNGDFVVYSGDDVAAYNVAIRGINLLNNIK (SEQ ID
NO: 5)

As used herein, Cas12a.1 includes SEQ ID NO: 3 and proteins with at least 70%-99.5% sequence identity thereto. Accordingly, provided herein are proteins comprising the amino acid sequence of SEQ ID NO: 3 and proteins with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity thereto. Also provided herein are nucleic acids encoding the proteins comprising the amino acid sequence of SEQ ID NO: 3 and proteins with at least 70%-99.5% sequence identity thereto.

As used herein, Cas12p includes SEQ ID NO: 4 and proteins with at least 70%-99.5% sequence identity thereto. Accordingly, provided herein are proteins comprising the amino acid sequence of SEQ ID NO: 4 and proteins with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity thereto. Also provided herein are nucleic acids encoding the proteins comprising the amino acid sequence of SEQ ID NO: 4 and proteins with at least 70%-99.5% sequence identity thereto.

Also provided herein are proteins comprising the amino acid sequence of SEQ ID NO: 222 and proteins with at least 70%-99.5% sequence identity thereto. Also provided herein are nucleic acids encoding the proteins comprising the amino acid sequence of SEQ ID NO: 222 and proteins with at least 70%-99.5% sequence identity thereto.

As used herein, Cas12q includes SEQ ID NO: 5 and proteins with at least 70%-99.5% sequence identity thereto. Accordingly, provided herein are proteins comprising the amino acid sequence of SEQ ID NO: 5 and proteins with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity thereto. Also provided herein are nucleic acids encoding the proteins comprising the amino acid sequence of SEQ ID NO: 5 and proteins with at least 70%-99.5% sequence identity thereto.

Table 3b shows exemplary nucleotide sequences, and exemplary codon optimized nucleic acid sequences for the novel Cas12 proteins of the disclosure.

TABLE 3b
Cas12a.1
ATGAAGGTCTCGACTTGGGATTCGTTTACAAACCAATACCCCCTAACGAA
AACTCTACGCTTCGAATTAAAGCCAGTCGGCAAAACACTGCAGAAAATTC
AAGATCGCAACCTGATTACAGAAGACGAACAACGCCAAAAAGATTTCAAC
AAAGTCAAAAAAATAATGGACGGATACTACAAGCAATTCATAGAAGAATG
CTTGGAAGGTGCCAAGATACCGTTAAAAAAATTGGAAGAAAACAACAACG
CTTACACGAAACTGAAAAAAGACCCTTACAACAAAAAATTAAGGGAAGAA
TACGCAAAACTCCAAAAACAATTAAGGAAACTAATTCACGACGAAATAAA
TAAAAAAGAAGAATTCAAATACTTGTTCAAGAAAGAATTCATCAAAAAAA
TATTGCCGGAATGGCTCGAAAAAAAAGGGAAAAAAGAGGAACTCAAAGAA
ATCGAAAAATTCGATAAATGGGTTACCTACTTTAGCGGTTTTTTTAACAA
CCGCAAAAACGTTTTTTCAAGCGATGAAATTTCGACGTCAATGATTTACA
GGATAGTCAACGACAACCTACCGAAATTCCTAGATGACGTTTCACGCTTC
GGAGAAATAACCAGATACAAGGAATTTGACGCCAACCAAATAGAAGAAAA
CTTTGAAAGCGAGTTGAACGGAGAGAAATTAAAAGATTTTTTCAACTTGA
AAAACTTCAACAACTGCCTTAACCAAGAAGGAATAGAAAAATTCAACTTA
ATCATAGGAGGCAAAAGCGAAGAAGGCAACAATAAAATAAAGGGCTTAAA
CGAATTAGTCAACGAACTCGCCCAAAAACAAGCGGACAAAAACGAGCAAA
AAAAGGTTAGAAAATTAAAACTCGCGCCGTTATTCAAGCAAATCTTAAGT
GACCGCAAATCCTCCTCGTTCGCATTCGAAAAATTCGAGGAAAATACGGA
GGTATTCGATGCAATAGACGAATTTTACGATAAAATAAGCTTGGAAACAC
TCAAAAAAATAGAAGCGACCCTCGAAAAGCTAGAAGAAAAAGATTTGGAA
TTAGTTTACTTGAAAAACGATAGATGCCTAACAGGAATTTCACAAGAAGT
ATTCGGGGATCGGGAAAGAGTACTTCAAGCCCTAAGGGAATACGCGAAAA
CCGAACTCGGCCTCAAAACCGACAAAAAAATAGAAAAATGGATGAAAAAA
GGCAGGTATTCAATCCACGAAATAGAGAGCGGCCTCAAAAAAATCGGTTC
AACCGGACACCCGATATGTAATTATTTCTCAAAACTAGAAGAAAAAAAGA
CAAACTTGATTCAAGAAATAAAAAAAGCGCGCACTGAATATGAAAAAATA
AGTGACAAAAAAAAGAAATTAACTGCTGAAAGCCAAGAGCCCAACGTCGC
AAGAATAAAGGCGTTACTGGACTCAATAATGCGGCTATACCACTTCATAA
AACCCCTCAACATCAACTTCAAAAACAAGAAAGAAAAGGATTCAGAGGCA
CTTGAAACCGATAACGATTTCTATAACGATTTCGACGAATCGTTTGCGGA
ACTAGGGAATATAATCCCACTATACAATCAAGTCAGAAACTATGTTACGC
AAAAACCGTTCAGCACCGAAAAATTCAAGTTAAACTTTGAAAATCCCAAA
CTCCTAAGCGGCTGGGACAAAAACAAGGAAAAAGACTATTATTCTGTTAT
ATTGAGAAAAGAGGAGTCATACTACTTAGCCATTATGACCCCAAAACAAA
AAAACGTTTTTGACGAACTGGAACGGCTTCCGGCTGGAAAAAATTATTTT
GAAAAAATAGACTACAAATTATTGCCTACCCCAGAAAAAAATCTACCTAG
AATATTATTTGCAAAAAAAAACATTTCATTTTACAAGCCATCAAAAGAAA
TCGAAGCGATTCGTAATCACTCTGCCCACACCAAGCATGGAAACCCACAA
AACGGGTTCAAAAAAAGGGATTTCCGATTAAGCGATTGCCATAAAATGAT
TGACTTTTACAAAAAGAGCATTCAAAAACACCCCGAATGGAAAGAATACG
ATTTCCAATTCAAAAAAACGGAAGATTACGTCGACATATCAGAATTTTAT
AAAGAAGTATCCGACCAAGGCTATAAAATAGAATTCAAAAAAATAAGCGA
AAAATATTTGCTTGACTTGGTCGAAGAAGGAAAACTTTACTTATTCCAAA
TTTGGAACAAGGACTTTTCGAAGTATTCGGAAGGCCGTAAAAACCTGCAC
ACAATTTACTGGAAAGAACTATTCTCCAAAGAAAACCTTTCAGACATAAC
TTACAAATTAAACGGCGAAGCCGAAATATTCTACCGCCCAAAGTCAATGG
AAAGGAAAGTAACTCACCCAAAAAACCAAAAAATAGAAAACAAAGACCCG
ATTAAAGGGAAAAAATTCAGTAAATTCAAATACGACTTTATAAAAAACAA
AAGGTACACCGAAGACCGTTTCTTCTTCCACTGCCCGATAACCTTGAACT
TCCAGGCGCGCGATGGCAGCAAAACGATTAACAAGCGGGTCAACGACCAC
ATACGCGAAACAAAAGATGACATTTTCGTGTTAAGCATTGACCGCGGGGA
AAGGCACTTGGCGTACTACACGCTATTGAATTCAAAAGGAGAAATCCAAG
AACAAGGCTCTTTCAACGTAATCTCGGACGACAAAGAAAGAAAACGTGAT
TACCACGAAAAACTGGATGAACGCGAAAAAGAACGCGACAAAGCAAGGAA
AAGCTGGCAGAAAATCGAGACCATAAAGAAATTGAAGGATGGCTACCTAT
CCCAAATCGTACACAAAATCGCTAAACTCGCAATAGAAAAAAACGCGATA
ATCGTCTTGGAAGACCTGAACTTAGACTTCAAGCGCGGGAGATTAAAAAT
CGAGAAGCAAGTATACCAAAAGTTCGAGAAAAAACTAATAGACAAACTCA
ATTACTTGGTTTTCAAGGAAAGAACCGAAAAAGAAGCCGGCGGATCCCTA
AACGCATACCAACTAACCGGAAAATTTGAAGGATTTAAGAAACTCGGAAA
AGAAACAGGTATAATATACTACGTTCCCGCGGCGTACACCTCGAAGATTT
GCCCGAAAACAGGCTTCGTAAATCTGTTAAGACCTAAATTCAAGAACATA
GAAAAAGCTAAGGAATTCTTCAAAAAATTCAACTACATCAAATACGATTC
GAGCGAAGGCTTATTCGAATTCAACTTCGACTACTCCAAATTCATTAAAA
ACGGAAAAAAAGAAACAAAAATAATTCAAGACAATTGGTCGGTTTACTCG
AACGGAACGAAACTAGTCGGCTTCAGAAACAAGAATAAAAACAATTCATG
GGATACAAAGGAAGTCAAACCGAACGAAAAACTAAAAATATTGTTCAAAG
AATACGGGGTTTCCTTCCAAAAAGACGAAAATATTATAAGCCAAATAGCC
AGCCAAAACAAAAAAGCTTTCTTTGAAAACCTCATTAAAATCTTTAAAAC
GATTTTAATGTTACGCAACTCAAGAAAAGACCCCGAAGAAGATTACGTAC
TTTCCTGCGTAAAAGACGAAAACGGCGAATTCTTCGACTCAAGAAAAGCT
AAAGACAACGAGCCCAAGGACGCCGACGCGAACGGCGCTTACCACATAGG
GTTGAAAGGATTAATGCTCTTGGAAAGAATAAAGGCCAACAAAGGAAAGA
AAAAACTCGATTTACTAATCAGCAGGAACGACTTCATCAACTTCGCAGTT
GAACGGAGCAAGTAA (SEQ ID NO: 13)
Cas12p
ATGAAAAAATCTATTTTTGATCAGTTTGTAAATCAGTATGCTCTTTCTAA
AACGTTGCGGTTTGAATTGAAGCCGGTGGGGGAGACGGGGAGGATGCTTG
AGGAGGCGAAGGTTTTTGCTAAAGATGAAACAATCAAGAAAAAATATGAG
GCAACCAAGCCTTTTTTTAATAAATTGCATCGTGAATTTGTAGAGGAGGC
TTTAAATGAGGTGGAATTAGCTGGTTTGCCTGAATATTTTGAAATATTTA
AATATTGGAAAAGGTATAAAAAGAAGTTTGAAAAGGATTTGCAGAAGAAA
GAAAAAGAATTGCGGAAATCAGTTGTAGGTTTTTTTAATGCACAGGCAAA
GGAATGGGCGAAAAAATATGAAACTTTGGGTGTGAAGAAAAAAGATGTGG
GACTTTTATTTGAAGAAAATGTTTTTGCTATATTGAAGGAAAGGTACGGA
AATGAGGAGGGATCACAAATTGTTGATGAAAGTACAGGAAAAGATGTTTC
GATATTTGATAGTTGGAAGGGCTTTACAGGGTATTTTATTAAATTCCAGG
AAACTCGTAAGAATTTTTATAAGGATGATGGCACGGCTACTGCTTTGGCT
ACAAGGATTATTGATCAAAATTTGAAGCGTTTTTGTGATAATTTACTAAT
ATTTGAAAGTATTAGAGATAAAATTGATTTTTCAGAGGTAGAACAAACTA
TGGGAAACTCTATTGATAAGGTTTTTTCAGTAATTTTTTATAGTTCCTGT
TTACTTCAGGAAGGAATTGATTTTTATAATTGTGTTTTAGGTGGGGAGAC
TCTGCCAAATGGTGAAAAGAGACAGGGAATAAATGAGCTTATTAATCTCT
ATAGGCAAAAAACTAGTGAGAAAGTACCTTTTTTAAAGTTGCTTGATAAG
CAGATTTTGAGTGAAAAAGAGAAGTTTATGGATGAAATTGAAAATGATGA
GGCTCTCTTGGATACTCTTAAAATATTTAGAAAATCGGCTGAAGAAAAAA
CCACTTTGTTAAAAAATATTTTTGGTGATTTTGTTATGAATCAGGGTAAG
TATGATTTAGCGCAGATTTATATTTCCAGAGAATCTTTAAATACTATTTC
ACGGAAATGGACCAGTGAAACAGATATATTTGAGGATTCATTATATGAAG
TGTTAAAGAAATCAAAAATAGTTTCTGCCTCTGTAAAAAAGAAAGATGGA
GGGTACGCTTTCCCTGAGTTTATTGCGCTTATTTATGTGAAAAGTGCTCT
TGAACAAATTCCTACTGAAAAATTTTGGAAGGAGCGATATTATAAAAATA
TTGGAGATGTTTTGAATAAAGGGTTTTTGAATGGTAAGGAAGGTGTCTGG
TTACAATTTTTATTGATTTTTGATTTTGAATTTAATTCTCTTTTTGAAAG
AGAAATAATTGATGAAAATGGAGACAAGAAAGTGGCCGGATATAATTTGT
TTGCCAAGGGTTTTGATGATCTTTTGAATAACTTTAAATATGATCAAAAA
GCTAAGGTTGTTATTAAGGATTTTGCAGATGAGGTTTTACATATTTATCA
GATGGGAAAATATTTTGCTATTGAAAAGAAACGTTCTTGGTTGGCTGATT
ATGATATTGATTCATTTTATACTGATCCTGAAAAAGGTTATTTGAAGTTT
TATGAAAATGCGTATGAAGAGATTATTCAAGTTTATAATAAATTGCGAAA
TTACCTAACGAAGAAACCTTATAGTGAGGATAAATGGAAACTTAATTTTG
AGAATCCAACTTTAGCTGATGGGTGGGACAAAAATAAAGAAGCTGATAAT
TCTACAGTTATTTTGAAAAAGGATGGTCGCTATTATTTAGGGTTGATGGC
TCGCGGGCGAAATAAACTTTTTGATGATAGAAATTTACCAAAAATTTTGG
AGGGCGTTGAGAATGGGAAATATGAGAAAGTTGTATATAAGTATTTTCCG
GATCAGGCAAAAATGTTTCCAAAAGTTTGTTTTTCAACTAAAGGTTTGGA
GTTTTTCCAACCTTCGGAGGAAGTCATTACTATTTACAAAAATTCTGAAT
TCAAAAAAGGGTATACTTTTAATGTAAGGAGTATGCAGAGGCTTATTGAT
TTTTATAAAGATTGTCTTGTTAGATATGAGGGGTGGCAATGTTATGATTT
TAGAAATTTGAGAAAGACAGAAGATTATCGGAAGAATATTGAAGAGTTTT
TCAGCGATGTTGCTATGGATGGGTATAAAATATCCTTTCAGGATGTCTCG
GAAAGTTATATTAAAGAGAAAAATCAGAATGGGGATTTATATTTATTTGA
GATAAAAAATAAAGATTGGAATGAAGGCGCAAATGGAAAGAAAAATTTGC
ACACTATATATTTTGAATCTCTTTTTTCGGCTGATAATATTGCCATGAAT
TTTCCCGTTAAGTTGAATGGACAAGCGGAAATTTTTTATCGGCCAAGAAC
AGAGGGGCTGGAGAAAGAAAGGATAATCACTAAAAAGGGTAATGTTTTGG
AAAAAGGAGATAAAGCTTTTCATAAAAGAAGGTATACGGAAAACAAAGTT
TTTTTTCATGTTCCGATTACACTTAATCGAACAAAAAAAAATCCATTTCA
ATTTAATGCAAAAATTAATGATTTTTTGGCTAAAAATTCTGATATAAATG
TTATTGGGGTCGATCGTGGGGAGAAGCAATTAGCATATTTTTCTGTTATT
TCACAGAGAGGCAAAATTTTGGATAGGGGTAGTTTAAATGTGATAAATGG
AGTTAATTATGCAGAGAAATTAGAAGAAAAAGCTAGAGGGCGTGAGCAGG
CGCGTAAGGATTGGCAGCAGATTGAAGGTATTAAAGATTTAAAGAAGGGA
TATATTTCTCAGGTAGTTAGAAAGCTAGCCGATTTAGCAATTCAGTATAA
TGCGATTATTGTTTTTGAAGATTTGAACATGCGGTTTAAGCAGATTCGTG
GAGGTATTGAAAAAAGTGTTTATCAGCAGTTGGAGAAGGCTTTGATTGAT
AAATTAACTTTTTTGGTTGAAAAGGAAGAAAAAGATGTAGAAAAGGCAGG
TCATTTGTTAAAAGCTTACCAGCTTGCTGCTCCGTTTGAGACTTTTCAGA
AAATGGGTAAACAAACGGGGATTGTTTTTTATACACAGGCTGCATATACT
TCACGAATTGATCCTGTTACAGGTTGGCGGCCTCACTTGTATTTGAAATA
TTCCAGTGCGGAGAAGGCAAAGGCGGATTTATTAAAATTTAAAAAGATAA
AGTTTGTGGATGGCCGGTTTGAGTTTACTTATGATATTAAGAGTTTTCGT
GAACAAAAGGAACATCCAAAGGCGACTGTCTGGACGGTGTGTTCTTGCGT
GGAGAGATTTCGTTGGAATAGATATTTAAATAGCAATAAAGGTGGTTATG
ACCATTACAGTGATGTGACGAAGTTCTTGGTAGAGCTTTTTCAAGAGTAT
GGGATTGATTTTGAAAGAGGGGATATTGTCGGGCAAATTGAGGTTTTGGA
AACGAAGGGAAATGAAAAATTTTTTAAGAATTTCGTTTTTTTCTTTAATT
TGATTTGTCAGATAAGAAATACTAATGCGTCGGAGTTGGCAAAAAAAGAT
GGAAAAGATGATTTTATTCTTTCACCGGTGGAACCGTTTTTTGATAGCAG
AAATTCGGAGAAGTTTGGGGAGGATTTGCCAAAAAATGGGGATGATAATG
GGGCATTTAATATTGCGAGGAAAGGGCTTGTTATTATGGATAAAATTACA
AAATTTGCAGATGAGAATGGTGGGTGCGAGAAGATGAAGTGGGGAGATTT
GTATGTTTCTAATGTGGAGTGGGATAATTTTGTAGCTAATAAATGA
(SEQ ID NO: 14)
Cas12q
ATGATAAATATTGACGAATTAAAAAATTTATATAAAGTTCAAAAAACAAT
TACTTTTGAATTAAAAAATAAATGGGAAAATAAGAATGATGAAAATGATA
GAGTTGAGTTTTTAAAGACTCAAGAATGGGTGGAATCTTTATTCAAAGTT
GATGAGGAGAATTTTGATGAAAAGGAGTCAATTCCGAACTTGTTAGATTT
CGGCCAAAAGATTGCGAGTCTTTTTTATAAGTTGAGTGAAGATATCGCTA
ATAATCAAATTGATACACGGGTTTTAAAAGTGAGCAAGTTTTTGTTGGAG
GAGATCGATAGAAATCAATATCATGAGAAAAAAAATAAACCAACAAAGGT
TAAGGAGATGAATCCAAATACAAATAAGAGTTATATTAAGGAGTATAAGT
TATCAGATCAAAATACATTGTATGTTCTGTTGAAGATAATGGAAGATGAA
GGGCGGGGTTTACAAAAATTTTTATATGATAAGGCAGACAGATTAAATTT
ATATAATCAGAAGGTAAGAAGAGATTTCGCTTTAAAAGAAAGTAACGAAC
AGCAGAAGTTTTCGGGTAACGCTAATTATTACGGAAACATAAAATTGTTG
ATTGATTCATTGGAAGACGCTGTTCGTATTATTGGTTATTTCACGTTTGA
TGATCAAGCAGAAAATGCTCAAATAAATGAATTCAAGAGCGTTAAGCAGG
AAATGAATAACAATGAAGCTTCGTATCAGGCTTTGAAAGATTTTGCTATT
GATAACGCAAAAAAAGAAATTGAACTTACAACTCTAAATCATAGGGCTGT
TAACAAGGATCCAAAAAAGATACAAGAACAGATTGAAGAAGTGGAAAATT
TTGAAGAAGATATAAATCAATTGAAGCACCAAATTTCTGCGCTTAATGAT
AAAAAATTTGATGTAGTGTCAAGATTAAAGCATGCATTAATTAAAATGTT
ACCGGAGTTGAATTTGTTAGATGCTGAAAGCGAGCAAGGTAGAGAGGTTC
AGCAAATATATCAAGATAAAAAGAATGGTTTGGAATTAGACGATTTTAAG
TTCAATTTGCTTAAACATCATCAATGGCAGAAAACCATTTTTAAATACAT
TAAATTAGAGGGTTTGGTTTTACCTGATTTATATGCCGAAAACAAACAAG
ATAAGATTAAAGTGTATATTGAAAATTATCGACAAAGCGGAGAAAGGATA
AGTAAAAAGGCACGCGAGGAGTTGGGCAAGATCGATAAAAGAGAGGAATT
TAATGGTAATGATGAACTAAAGAAAGCGTGGTACGAATACAAAGATTTTT
GCAGAGACAAGCGTAATAAATCCGTGGAATTGGGCAATAAGAAATCACTG
TACAATGCCATCAAGCGTGAGGTTTTAAGGCAGAAAATGTGTAATCATTT
TGCCGTATTGGTGAGTGATGGGGAAGATACATCGCCTTATTATTATTTGA
TATTAATTCCCAATGAAAACAGTGATGAAATGAACAGGACATTCAAAGAG
CTTAAAGCATCCGAAGGAAATTGGAAGATGCTCGATTATAACAGATTAAC
TTTTAAAGCTTTGGAAAAATTGGCATTATTGCGCAGCTCTACATTTGAAA
TTGCAGACCAAGAACTACAAGAAGAAGCTAAAAAAATTTGGGAAGAATAT
AAAGAAAAGGCGTATAAAGATTTTAAGAATAAAAAATTATTACAAGGGCT
ATCCGGTCGCCAAAGAGAAGAAAAAAAACAAGAATTGCAAAAAGAAAGTT
TAAATCGAGTTATAAATTATTTAATTCGTTGCATTCAGTCGTTGCCGGAT
AGCGGTAAATACAATTTTAATTTTAAAGAACCGCATCAATATCAGAGCTT
GGAAGAGTTTGCGGAAGAAATTGATAGACAGGGTTATCATTGCGCTTGGA
AGAATGTAAGCAAAGACAAGCTTATGGAGCTGGAGGCGATGGAAAAAATT
AAAGTATTTAAATTGCATAATAAGGATTTTAGAAAAGTTAAACTTAACGA
TTCGAAACACAATCCGAATCTTTTTACTTTATATTGGCTTGACGCGATGA
ATTTGGATAAAGTCAATGTTCGTTTATTGCCCGAGGTGGATTTATATAAA
AGAGCCAAAGAAACGCAACTAAAATTATTCGAAAGAGATGTAAAGTGCAA
TATTAATAATCAAAAAATAAAATCAATTAAAGAAAAAAATAGATTATTTC
AAGATAAACTTTACGCTTCATTCAAGCTGGAATTTTATCCAGAAAACGAA
GGTTTGGGTTTTGAACAAGTCAATGATAAAGTGAATAATTTTTGCGGAAG
TGATACAGCGTATTATTTGGGTTTGGATAGGGGTGAGAAAGAATTGGTTA
CGTTTTGCTTGGTTGATTCTGATGGGCGGTTGGTTAAGAACGGAGATTGG
ACGAAGTTTAAAGAGGTTAACTATGCGGATAAATTAAAGCAATTTTATTA
TTCAAAAGGTGAAATAGAATCTACTCAACAACAACTTTTGGAAGCTCGAG
ACAATATTAAACAAGCTACTAACACGGAGGATAAAGAATCGATGAAATTA
AACTATAAAAAATTAGAGTTGAAACTAAAACAACAGAATTTGTTAGCGCA
GGAGTTTATTAAAAAAGCTTATTGCGGTTATTTGATAGATTCAATAAATG
AAATATTACGGGAATATCCAAATACGTATCTTGTATTAGAGGATTTGGAT
ATAGCAGGTAAAGCTGACCCCGAAAGCGGCATGACCAATAAAGAACAAAA
TTTAAATAAAACAATGGGTGCCAGCGTTTATCAAGCTATTGAAAATGCCA
TAGTAAATAAGTTTAAATACCGTACTGTTAAATTATCCGATATCAAAGGT
TTGCAAACTGTACCGAATGTAGTGAAGGTGGAAGATTTGCGCGAAGTTAA
GGAAGTGGAAGATGGTGAGCATAAATTTGGTTTGATAAGATCCGTGAAAT
CAAAGGATCAAATTGGCAATATTCTGTTTGTGGATGAAGGAGAAACATCT
AATACTTGCCCGAATTGCGGATTTAACAGCGATTGGTTTAAGCGGGATGT
TGATTTTGATTTGGAGATTGTGGCTACTGTAAACGGTCAGAAAAATGCGG
TTATAGAACAAAACGACAAAAAGTACTGTTTTCCCGGTGAAATTTATAAG
TTAGAAATAATTAATAAAGAATACGAAACAAATAAACGGAATTTAGCCAT
GATTTTTAAACCGCGCGCAAAAGCTTGTAGAAAATTTATAAATAATAATT
TGGATAAGAATGACTATTTTTATTGCCCGTATTGCGCTTTTTCTAGCAAG
AACTGCAATAATCCAAAATTGCAAAACGGTGATTTTGTGGTATATTCGGG
TGATGATGTGGCGGCATACAATGTAGCGATCAGAGGTATTAACCTTTTAA
ACAATATAAAATAG (SEQ ID NO: 15)
Cas12a.1 codon optimized version:
ATGGGGTCAAGTCATCACCACCACCACCACTCAAGTGGACTAGTACCCCG
TGGCAGCATGAAAGTTAGCACCTGGGATAGCTTCACCAACCAGTACCCGC
TGACCAAGACCCTGCGTTTTGAGCTGAAGCCGGTGGGTAAAACCCTGCAG
AAGATCCAAGACCGTAACCTGATTACCGAGGACGAACAGCGTCAAAAGGA
TTTCAACAAGGTTAAGAAAATCATGGATGGTTACTACAAGCAGTTCATCG
AGGAATGCCTGGAAGGCGCGAAGATCCCGCTGAAGAAACTGGAGGAAAAC
AACAACGCGTACACCAAACTGAAGAAAGACCCGTATAACAAGAAACTGCG
TGAGGAATACGCGAAGCTGCAGAAACAACTGCGTAAACTGATCCACGATG
AGATTAACAAGAAAGAGGAATTCAAGTACCTGTTTAAGAAAGAATTCATC
AAGAAAATTCTGCCGGAATGGCTGGAGAAGAAAGGTAAGAAAGAGGAACT
GAAAGAGATCGAAAAGTTCGACAAATGGGTGACCTACTTTAGCGGCTTCT
TTAACAACCGTAAGAACGTTTTCAGCAGCGACGAGATTAGCACCAGCATG
ATCTATCGTATTGTGAACGATAACCTGCCGAAATTCCTGGACGATGTTAG
CCGTTTTGGTGAAATTACCCGTTACAAGGAGTTCGACGCGAACCAGATCG
AGGAAAACTTTGAGAGCGAACTGAACGGTGAAAAACTGAAGGATTTCTTT
AACCTGAAAAACTTCAACAACTGCCTGAACCAAGAAGGCATTGAGAAATT
TAACCTGATCATTGGTGGCAAGAGCGAGGAAGGTAATAACAAAATCAAGG
GCCTGAACGAACTGGTGAACGAGCTGGCGCAAAAACAAGCGGACAAGAAC
GAGCAGAAGAAAGTTCGTAAACTGAAGCTGGCGCCGCTGTTCAAACAAAT
CCTGAGCGATCGTAAGAGCAGCAGCTTTGCGTTCGAAAAATTTGAGGAAA
ACACCGAGGTGTTCGACGCGATCGATGAATTTTATGACAAGATTAGCCTG
GAGACCCTGAAGAAAATCGAAGCGACCCTGGAGAAACTGGAGGAAAAGGA
CCTGGAACTGGTTTACCTGAAAAACGATCGTTGCCTGACCGGTATCAGCC
AGGAAGTGTTCGGCGACCGTGAGCGTGTTCTGCAAGCGCTGCGTGAATAC
GCGAAAACCGAGCTGGGTCTGAAGACCGATAAGAAAATCGAGAAGTGGAT
GAAGAAAGGTCGTTATAGCATCCACGAGATTGAAAGCGGCCTGAAGAAAA
TCGGTAGCACCGGCCACCCGATTTGCAACTACTTCAGCAAACTGGAGGAA
AAGAAAACCAACCTGATCCAGGAAATTAAGAAAGCGCGTACCGAGTATGA
AAAGATCAGCGACAAGAAAAAGAAACTGACCGCGGAAAGCCAAGAGCCGA
ACGTGGCGCGTATCAAAGCGCTGCTGGATAGCATTATGCGTCTGTATCAC
TTCATCAAGCCGCTGAACATCAACTTCAAGAACAAGAAAGAGAAGGACAG
CGAAGCGCTGGAGACCGACAACGATTTTTACAACGACTTCGATGAAAGCT
TTGCGGAGCTGGGCAACATCATTCCGCTGTACAACCAAGTGCGTAACTAT
GTTACCCAAAAACCGTTCAGCACCGAGAAATTCAAGCTGAACTTTGAAAA
CCCGAAGCTGCTGAGCGGTTGGGACAAAAACAAGGAAAAAGATTACTATA
GCGTGATTCTGCGTAAAGAGGAAAGCTACTATCTGGCGATCATGACCCCG
AAGCAGAAAAACGTTTTCGACGAGCTGGAACGTCTGCCGGCGGGCAAAAA
TTACTTCGAGAAGATCGATTACAAGCTGCTGCCGACCCCGGAAAAGAACC
TGCCGCGTATCCTGTTCGCGAAGAAAAACATTAGCTTTTACAAGCCGAGC
AAAGAGATCGAAGCGATTCGTAACCACAGCGCGCACACCAAACACGGTAA
CCCGCAGAACGGCTTCAAGAAACGTGACTTTCGTCTGAGCGATTGCCACA
AGATGATCGACTTCTACAAGAAAAGCATTCAGAAACACCCGGAATGGAAG
GAGTATGATTTTCAATTCAAGAAAACCGAGGACTACGTGGATATCAGCGA
ATTCTATAAAGAGGTTTCTGACCAGGGTTACAAGATCGAATTCAAGAAAA
TTAGCGAGAAATACCTGCTGGACCTGGTGGAGGAAGGTAAACTGTACCTG
TTCCAAATCTGGAACAAGGATTTCAGCAAGTACAGCGAAGGCCGTAAAAA
CCTGCACACCATCTATTGGAAAGAACTGTTCAGCAAGGAGAACCTGAGCG
ATATTACCTATAAGCTGAACGGCGAGGCGGAAATCTTTTACCGTCCGAAA
AGCATGGAGCGTAAGGTTACCCACCCGAAGAACCAGAAAATCGAAAACAA
AGACCCGATCAAGGGTAAGAAATTCAGCAAGTTCAAGTATGACTTCATCA
AGAACAAGCGTTACACCGAGGATCGTTTCTTTTTCCACTGCCCGATCACC
CTGAACTTTCAAGCGCGTGACGGCAGCAAAACCATCAACAAGCGTGTGAA
CGATCACATTCGTGAGACCAAAGACGATATCTTCGTTCTGAGCATTGATC
GTGGTGAACGTCACCTGGCGTACTATACCCTGCTGAACAGCAAGGGTGAA
ATTCAGGAGCAAGGCAGCTTTAACGTGATCAGCGACGATAAGGAGCGTAA
ACGTGACTATCACGAAAAACTGGATGAGCGTGAAAAGGAGCGTGACAAGG
CGCGTAAAAGCTGGCAGAAAATCGAGACCATTAAGAAACTGAAGGATGGC
TACCTGAGCCAAATCGTGCACAAGATTGCGAAACTGGCGATCGAGAAAAA
CGCGATCATTGTTCTGGAAGACCTGAACCTGGATTTCAAGCGTGGTCGTC
TGAAGATTGAGAAACAGGTGTACCAAAAATTCGAAAAGAAACTGATCGAC
AAGCTGAACTATCTGGTTTTTAAAGAACGTACCGAAAAAGAGGCGGGTGG
TAGCCTGAACGCGTATCAGCTGACCGGTAAATTCGAGGGCTTTAAGAAAC
TGGGCAAGGAAACCGGCATCATTTACTATGTGCCGGCGGCGTACACCAGC
AAAATCTGCCCGAAGACCGGCTTCGTTAACCTGCTGCGTCCGAAGTTCAA
GAACATCGAAAAGGCGAAGGAGTTTTTCAAGAAGTTCAACTACATCAAGT
ACGACAGCAGCGAAGGTCTGTTTGAGTTCAACTTCGATTACAGCAAGTTC
ATCAAGAACGGCAAGAAAGAGACCAAAATCATTCAGGACAACTGGAGCGT
GTATAGCAACGGTACCAAGCTGGTTGGCTTCCGTAACAAGAACAAAAACA
ACAGCTGGGATACCAAGGAAGTGAAACCGAACGAGAAGCTGAAAATTCTG
TTCAAAGAGTACGGTGTTAGCTTTCAAAAGGACGAAAACATCATTAGCCA
GATCGCGAGCCAAAACAAGAAAGCGTTTTTCGAGAACCTGATCAAGATTT
TCAAAACCATTCTGATGCTGCGTAACAGCCGTAAAGACCCGGAGGAAGAT
TACGTGCTGAGCTGCGTTAAGGACGAAAACGGCGAGTTTTTCGACAGCCG
TAAGGCGAAAGATAACGAGCCGAAAGACGCGGATGCGAACGGCGCGTACC
ACATTGGTCTGAAGGGCCTGATGCTGCTGGAACGTATCAAGGCGAACAAA
GGTAAGAAAAAGCTGGACCTGCTGATCAGCCGTAACGATTTCATTAACTT
TGCGGTTGAGCGTAGCAAGTAA (SEQ ID NO: 16)
Cas12p codon optimized:
ATGGGATCAAGTCATCACCACCACCACCACTCAAGTGGACTAGTACCCAG
GGGAAGCATGAAGAAGAGCATTTTCGATCAGTTCGTTAACCAGTACGCGC
TGAGCAAGACCCTGCGTTTCGAGCTGAAACCGGTGGGTGAAACCGGCCGT
ATGCTGGAGGAAGCGAAGGTTTTCGCGAAGGATGAAACCATTAAGAAAAA
GTACGAAGCGACCAAGCCGTTCTTTAACAAACTGCACCGTGAATTCGTGG
AGGAAGCGCTGAACGAGGTTGAACTGGCGGGCCTGCCGGAGTACTTCGAA
ATCTTCAAGTACTGGAAGCGTTACAAAAAGAAATTCGAGAAGGACCTGCA
GAAGAAAGAGAAGGAACTGCGTAAAAGCGTGGTTGGTTTCTTTAACGCGC
AAGCGAAGGAGTGGGCGAAGAAATATGAAACCCTGGGCGTGAAGAAAAAG
GATGTTGGTCTGCTGTTCGAGGAAAACGTGTTTGCGATTCTGAAAGAACG
TTACGGTAACGAGGAAGGCAGCCAGATTGTGGACGAGAGCACCGGCAAGG
ATGTTAGCATCTTCGACAGCTGGAAGGGTTTTACCGGCTATTTCATCAAA
TTTCAGGAAACCCGTAAGAACTTCTACAAAGATGATGGTACCGCGACCGC
GCTGGCGACCCGTATCATTGATCAAAACCTGAAACGTTTCTGCGACAACC
TGCTGATCTTTGAGAGCATTCGTGATAAGATCGACTTCAGCGAGGTTGAA
CAGACCATGGGCAACAGCATCGATAAGGTGTTCAGCGTTATCTTTTATAG
CAGCTGCCTGCTGCAAGAAGGTATCGACTTTTACAACTGCGTGCTGGGTG
GTGAAACCCTGCCGAACGGTGAAAAGCGTCAGGGCATTAACGAACTGATC
AACCTGTACCGTCAAAAGACCAGCGAGAAAGTTCCGTTCCTGAAGCTGCT
GGACAAACAGATTCTGAGCGAGAAGGAAAAATTTATGGATGAGATCGAAA
ACGACGAGGCGCTGCTGGATACCCTGAAGATTTTCCGTAAAAGCGCGGAG
GAAAAGACCACCCTGCTGAAAAACATCTTCGGCGATTTTGTGATGAACCA
GGGTAAATATGACCTGGCGCAAATCTACATTAGCCGTGAAAGCCTGAACA
CCATTAGCCGTAAGTGGACCAGCGAAACCGATATCTTCGAAGACAGCCTG
TACGAGGTGCTGAAAAAGAGCAAAATCGTGAGCGCGAGCGTTAAAAAGAA
AGACGGTGGCTACGCGTTCCCGGAGTTTATCGCGCTGATTTATGTTAAAA
GCGCGCTGGAACAGATTCCGACCGAGAAGTTCTGGAAAGAACGTTACTAT
AAGAACATCGGCGATGTGCTGAACAAGGGTTTCCTGAACGGTAAAGAAGG
CGTTTGGCTGCAATTTCTGCTGATCTTTGACTTCGAATTTAACAGCCTGT
TCGAGCGTGAAATCATTGATGAGAACGGCGACAAGAAAGTGGCGGGTTAT
AACCTGTTCGCGAAGGGTTTTGACGATCTGCTGAACAACTTCAAATACGA
CCAGAAGGCGAAAGTGGTTATTAAGGATTTTGCGGACGAAGTTCTGCACA
TTTATCAAATGGGCAAATACTTCGCGATCGAGAAGAAACGTAGCTGGCTG
GCGGACTATGATATTGACAGCTTCTACACCGATCCGGAGAAGGGTTACCT
GAAATTTTATGAAAACGCGTACGAGGAAATCATTCAGGTTTATAACAAGC
TGCGTAACTACCTGACCAAGAAACCGTATAGCGAGGACAAGTGGAAACTG
AACTTCGAAAACCCGACCCTGGCGGATGGTTGGGACAAGAACAAAGAGGC
GGATAACAGCACCGTGATTCTGAAGAAAGACGGTCGTTACTATCTGGGCC
TGATGGCGCGTGGTCGTAACAAGCTGTTCGACGATCGTAACCTGCCGAAA
ATCCTGGAGGGTGTTGAAAACGGCAAGTACGAAAAGGTGGTTTACAAGTA
CTTCCCGGATCAGGCGAAGATGTTCCCGAAAGTGTGCTTTAGCACCAAAG
GCCTGGAATTCTTTCAACCGAGCGAGGAAGTTATCACCATTTACAAGAAC
AGCGAGTTCAAGAAAGGTTATACCTTTAACGTGCGTAGCATGCAGCGTCT
GATTGATTTCTATAAAGACTGCCTGGTTCGTTACGAAGGTTGGCAATGCT
ATGATTTTCGTAACCTGCGTAAGACCGAGGACTACCGTAAAAACATCGAG
GAATTCTTTAGCGATGTGGCGATGGACGGCTACAAGATTAGCTTCCAGGA
CGTTAGCGAGAGCTATATCAAGGAGAAGAACCAAAACGGTGATCTGTACC
TGTTTGAGATCAAGAACAAAGACTGGAACGAAGGTGCGAACGGCAAGAAA
AACCTGCACACCATTTATTTCGAGAGCCTGTTTAGCGCGGATAACATCGC
GATGAACTTCCCGGTGAAACTGAACGGCCAGGCGGAGATCTTTTACCGTC
CGCGTACCGAAGGTCTGGAGAAGGAACGTATCATTACCAAGAAAGGCAAC
GTTCTGGAAAAGGGTGACAAAGCGTTCCACAAGCGTCGTTACACCGAGAA
CAAAGTGTTCTTTCACGTTCCGATTACCCTGAACCGTACCAAGAAAAACC
CGTTCCAATTTAACGCGAAGATCAACGACTTCCTGGCGAAAAACAGCGAT
ATCAACGTGATTGGTGTTGACCGTGGCGAGAAACAGCTGGCGTATTTTAG
CGTGATTAGCCAACGTGGCAAGATCCTGGACCGTGGTAGCCTGAACGTGA
TCAACGGCGTTAACTACGCGGAGAAGCTGGAGGAAAAAGCGCGTGGTCGT
GAACAGGCGCGTAAGGATTGGCAGCAAATCGAGGGCATTAAAGACCTGAA
GAAAGGTTATATTAGCCAGGTGGTTCGTAAACTGGCGGATCTGGCGATCC
AATACAACGCGATCATTGTGTTCGAGGACCTGAACATGCGTTTTAAGCAA
ATTCGTGGTGGCATCGAGAAAAGCGTTTATCAGCAACTGGAAAAGGCGCT
GATCGATAAACTGACCTTCCTGGTGGAGAAGGAAGAAAAGGACGTTGAAA
AGGCGGGTCACCTGCTGAAAGCGTACCAGCTGGCGGCGCCGTTCGAAACC
TTTCAGAAGATGGGTAAACAAACCGGCATTGTGTTTTATACCCAAGCGGC
GTACACCAGCCGTATCGATCCGGTTACCGGCTGGCGTCCGCACCTGTACC
TGAAATATAGCAGCGCGGAAAAGGCGAAAGCGGACCTGCTGAAGTTCAAG
AAAATTAAGTTCGTGGATGGTCGTTTCGAGTTTACCTACGACATCAAGAG
CTTCCGTGAGCAGAAGGAACACCCGAAAGCGACCGTGTGGACCGTTTGCA
GCTGCGTTGAGCGTTTTCGTTGGAACCGTTATCTGAACAGCAACAAAGGT
GGCTACGATCACTATAGCGACGTGACCAAGTTCCTGGTTGAGCTGTTTCA
GGAATACGGCATCGACTTCGAACGTGGTGATATTGTGGGCCAAATCGAGG
TTCTGGAAACCAAGGGTAACGAGAAGTTCTTTAAGAACTTCGTGTTCTTT
TTCAACCTGATCTGCCAGATTCGTAACACCAACGCGAGCGAACTGGCGAA
GAAAGACGGCAAGGACGATTTCATTCTGAGCCCGGTTGAGCCGTTTTTCG
ATAGCCGTAACAGCGAGAAGTTCGGCGAAGACCTGCCGAAAAACGGTGAC
GATAACGGCGCGTTTAACATCGCGCGTAAAGGTCTGGTTATTATGGATAA
GATCACCAAATTCGCGGACGAGAACGGTGGCTGCGAAAAGATGAAATGGG
GTGACCTGTATGTGAGCAATGTGGAGTGGGATAACTTTGTGGCGAATAAA
TAA (SEQ ID NO: 17)
Cas12q codon optimized
ATGGGGTCCTCCCATCATCACCACCACCACTCTTCAGGCTTGGTACCGCG
TGGTTCCATGATCAACATAGACGAATTGAAAAATTTATATAAGGTGCAAA
AGACCATCACTTTCGAACTTAAGAACAAGTGGGAGAACAAAAATGATGAG
AACGACAGAGTAGAGTTCTTGAAGACTCAGGAGTGGGTCGAAAGCCTTTT
CAAGGTCGATGAAGAGAACTTTGATGAGAAAGAGTCTATCCCTAACTTGT
TAGACTTCGGACAGAAGATTGCGTCCTTGTTTTACAAGCTGAGCGAGGAC
ATAGCGAACAACCAAATTGATACGCGGGTATTGAAAGTCTCGAAATTCCT
TTTAGAGGAAATTGATAGAAATCAATACCACGAGAAAAAAAACAAGCCCA
CAAAGGTAAAAGAAATGAATCCCAACACAAACAAAAGTTATATAAAAGAA
TATAAGCTGTCCGACCAAAACACACTGTACGTGTTATTAAAGATAATGGA
AGATGAAGGTCGGGGATTACAAAAATTTTTGTACGATAAAGCGGACCGGT
TAAACCTGTACAATCAAAAAGTTCGGAGAGACTTCGCCTTAAAGGAATCA
AATGAGCAACAAAAATTCTCTGGAAATGCCAACTACTATGGGAATATAAA
GCTGCTTATAGATAGCTTAGAAGATGCAGTCCGGATCATTGGGTATTTCA
CTTTCGACGATCAAGCAGAAAACGCACAAATCAATGAATTTAAGTCCGTT
AAACAGGAAATGAATAATAATGAAGCGTCTTACCAAGCACTGAAAGACTT
CGCTATTGATAACGCAAAAAAAGAGATAGAATTGACGACGTTGAACCACC
GGGCGGTCAACAAGGATCCAAAAAAGATTCAAGAACAGATTGAGGAAGTC
GAAAATTTCGAAGAAGATATTAACCAGTTAAAGCATCAGATATCAGCCTT
GAATGATAAGAAGTTTGACGTGGTTAGCAGATTAAAGCACGCTCTTATAA
AAATGTTACCAGAACTGAATCTTTTGGATGCTGAGTCGGAACAGGGCCGT
GAAGTCCAGCAGATATATCAAGACAAAAAAAACGGGTTGGAGCTTGATGA
CTTTAAATTTAACCTTTTAAAACATCATCAATGGCAAAAAACGATCTTCA
AGTATATTAAGCTTGAGGGCTTAGTTCTGCCAGACCTTTACGCGGAAAAC
AAACAAGATAAAATCAAGGTTTATATTGAGAATTATAGACAGAGTGGTGA
GCGTATTTCTAAGAAGGCGAGAGAGGAATTAGGAAAAATCGATAAACGCG
AAGAGTTCAATGGAAATGACGAACTTAAGAAGGCATGGTATGAGTATAAG
GACTTCTGTAGAGACAAACGTAATAAGAGCGTGGAACTTGGCAATAAGAA
GTCGCTGTACAATGCCATAAAGCGCGAAGTTTTGCGGCAAAAAATGTGCA
ACCATTTCGCTGTGCTGGTGTCCGACGGTGAAGATACTTCCCCTTATTAT
TATCTGATATTAATCCCGAACGAGAACTCCGATGAAATGAATAGAACGTT
CAAGGAATTGAAGGCCTCCGAGGGGAATTGGAAGATGTTGGATTACAATC
GTCTGACCTTCAAAGCCTTGGAGAAATTGGCCCTGTTACGGTCGTCTACC
TTCGAGATAGCGGATCAGGAACTGCAAGAAGAGGCAAAAAAGATCTGGGA
GGAGTACAAGGAAAAGGCGTACAAAGACTTCAAAAACAAAAAGTTATTAC
AGGGTTTATCGGGAAGACAGCGGGAGGAGAAAAAGCAAGAATTGCAAAAG
GAGAGCCTGAATAGAGTAATCAATTACTTGATCAGATGCATTCAGTCATT
GCCCGACAGCGGAAAATACAACTTTAACTTTAAAGAGCCTCATCAATACC
AATCGCTTGAAGAGTTTGCCGAGGAGATTGATCGGCAAGGTTATCACTGT
GCTTGGAAAAACGTTTCTAAAGATAAACTGATGGAATTGGAAGCGATGGA
AAAGATTAAGGTTTTCAAACTTCATAACAAAGACTTTCGCAAGGTAAAAC
TGAACGACTCCAAGCACAACCCTAATCTTTTTACTTTGTACTGGTTAGAC
GCCATGAATTTGGATAAGGTTAACGTCCGCCTGTTACCGGAAGTTGACCT
TTACAAGAGAGCTAAGGAAACACAGCTGAAATTGTTCGAACGTGATGTGA
AATGCAATATCAATAACCAAAAGATTAAATCTATCAAGGAGAAGAATAGA
CTGTTTCAGGACAAGTTGTATGCTAGTTTTAAGTTAGAGTTTTATCCAGA
AAACGAAGGATTAGGTTTCGAGCAGGTAAATGACAAGGTCAATAACTTCT
GCGGTAGCGATACGGCCTATTATCTTGGGCTTGATCGTGGAGAGAAAGAG
CTTGTTACATTCTGCCTGGTGGACTCTGATGGCCGCCTGGTAAAAAACGG
AGACTGGACCAAGTTTAAAGAGGTGAACTATGCCGACAAACTGAAGCAAT
TCTACTACTCAAAAGGCGAAATAGAGAGTACCCAACAACAGCTGTTAGAA
GCCCGGGACAATATTAAACAAGCGACCAACACGGAAGATAAGGAGTCCAT
GAAACTGAATTATAAGAAACTGGAACTGAAGTTAAAACAACAGAATTTGC
TGGCGCAAGAATTCATAAAAAAAGCGTACTGCGGCTACCTTATCGATAGC
ATTAATGAGATTCTGAGAGAATATCCAAATACTTATCTTGTCTTAGAGGA
TTTGGATATCGCGGGTAAAGCGGATCCAGAGTCGGGGATGACTAATAAAG
AGCAGAACTTAAACAAAACGATGGGGGCTTCAGTATACCAGGCCATTGAG
AATGCGATCGTAAATAAATTCAAATATCGCACCGTGAAATTGTCCGATAT
CAAGGGCCTTCAGACTGTACCTAATGTAGTGAAGGTCGAAGACTTACGGG
AAGTGAAAGAGGTTGAAGATGGGGAACACAAGTTCGGGTTAATAAGATCA
GTTAAGAGCAAGGATCAAATCGGTAACATACTTTTTGTCGACGAGGGGGA
GACCAGTAACACTTGTCCGAATTGCGGTTTTAATAGTGATTGGTTTAAAC
GCGATGTTGATTTTGACTTAGAAATAGTCGCTACTGTAAACGGGCAAAAG
AATGCCGTGATTGAGCAAAATGACAAAAAATACTGTTTCCCGGGCGAAAT
ATATAAATTGGAAATCATTAATAAAGAGTACGAAACAAACAAGCGTAATC
TTGCCATGATTTTTAAACCTCGGGCCAAAGCGTGCCGTAAATTTATCAAT
AATAATTTAGATAAGAACGATTATTTCTATTGTCCCTACTGCGCCTTCTC
GTCGAAGAATTGTAACAACCCGAAACTGCAGAACGGCGATTTCGTGGTAT
ATTCAGGAGACGATGTTGCTGCTTACAATGTTGCTATCAGAGGAATTAAC
CTGCTGAACAATATTAAATAG (SEQ ID NO: 18)

Table 4a shows the structural and functional characteristics of the novel Cas12 proteins of the disclosure as exemplified herein. Table 4b shows the number and sequence of the natural spacers of the corresponding CRISPR arrays. Blank cells in the tables do not indicate that no value/property exists, but rather that it has not been exemplified herein.

TABLE 4a

| Structural | Cas12a.1 | Cas12p | Cas12q |
|---|---|---|---|
| Size aa | 1254 | 1281 | 1137 |
| Kda | | | |
| Identity protein seq | 46.7 | 56.3 | 23.7 |
| to NCBI Protein DB | | | |
| Best hit | | | |
| Nuclease domains | yes | yes | yes |
| Bridge Helix | yes | yes | yes |
| Cas gene cluster | yes | yes | yes |
| Cas1 (length in aa | 325 aa | 354 aa | 334 aa |
| of Cas1 proteins | | | |
| encoded in each | | | |
| corresponding | | | |
| cluster) | | | |
| CRISPR array | 806 bp | 551 bp | 1650 bp |
| Repeats | 13 | 9 | 27 |
| (number and sequence | (GTTTAAGGC | (CTCGAATATC | (ATCTACAAA |
| of the natural | CTTGACAAAA | CCTATTAGATT | AGTAGAAATT |
| repeats included in | TTTCTACTGT | TCTACTTTTGT | AAATAGGTCT |
| the corresponding | AGTAGAT) | AGAT) (SEQ | ATTTGAG) |
| CRISPR array) | (SEQ ID | ID NO: 20) | (SEQ ID |
| | NO: 19) | | NO: 21) |
| Target | GTGGCAGCTC | GTGGCAGCTCA | |
| | AAAAATTGGC | AAAATTGGCTA | |
| | TACAAAAC | CAAAAC (SEQ | |
| | (SEQ ID | ID NO: 25) | |
| | NO: 25) | | |
| Collateral Cleavage | yes | yes | |
| shown in examples | | | |

TABLE 4b

| Cas12a.1 | Cas12p | Cas12q |
|---|---|---|
| Repeats: 12 | Repeats: 8 | Repeats: 26 |
| CCCGATTGACGCTATAG | TTCAGATGTTTGCTCTTTG | AATCGTAGCGATAACCGA |
| TAAGCATCGAG (SEQ ID | ACATATCG (SEQ ID NO: | AGAACAAAT (SEQ ID NO: |
| NO: 71) | 82) | 89) |
| GCGTCCCATAAAGGTAT | ATGGTGATTTAAAAACAA | AACCCATATGTTTTATTAT |
| GACTTGTATT (SEQ ID | AACTCGGCGCGA (SEQ ID | CCTGCTGA (SEQ ID NO: |
| NO: 72) | NO: 83) | 90) |
| ACGCACGCAGTATTGAA | CCTTGTGCAAAATAGACA | TACAAAATTAAGGCGGTC |
| TACGCGAATAG (SEQ ID | GGTTAGACCGT (SEQ ID | TAGGAGA (SEQ ID NO: 91) |
| NO: 73) | NO: 84) | |
| GTTCACGTAAAACTTAA | CGAAAATCCAGCTAAACT | TCCATTTGATGATAACCA |
| TCGTTGAACT (SEQ ID | CATTCTCTGATT (SEQ ID | TAAGAAT (SEQ ID NO: 92) |
| NO: 74) | NO: 85) | |
| TGGTAGTGCCAACACGT | TTGATTGGAGGAACAAGC | AAAATAATGTAATATAAT |
| GCGCCCACCA (SEQ ID | TACATAAA (SEQ ID NO: | ACAATAT (SEQ ID NO: 93) |
| NO: 75) | 86) | |
| TCGGTGGTGGGCGAACA | CGTATGGGTGTAATTTAAT | CTCGACACTGGGACAACT |
| TTGACTGTTGGT (SEQ | CGGTTTG (SEQ ID NO: | TCCGTAT (SEQ ID NO: 94) |
| ID NO: 76) | 87) | |
| CCGATTCCTTCTCGTTCG | ATGAATCGTATAAGATAT | CAATAAATACTGATTAGA |
| CCCGTGACCA (SEQ ID | GATCTGAAT (SEQ ID | AGAAGATAT (SEQ ID NO: |
| NO: 77) | NO: 88) | 95) |
| GTTGCGGGAGATACTAC | ATTAAATTACATAATGAG | CTGTCAAAGCCATAGTCT |
| TTCAATATACA (SEQ ID | CCAACACGGCGACC (SEQ | TGATCCAGC (SEQ ID NO: |
| NO: 78) | ID NO: 23 | 96) |
| GGTAGCCGAAATGAATT | | GCGCAAAGCATCAGCGCA |
| CGGTATAACCCG (SEQ | | ATGGCTCG (SEQ ID NO: |
| ID NO: 79) | | 97) |
| AACCAGTATCCTACCGT | | TGGCAGAGTTCGGGCCAA |
| GAAGTTGTCGC (SEQ ID | | GTATCAT (SEQ ID NO: 98) |
| NO: 80) | | |
| GGGGGTTTGAGTGGGCA | | GTAGCGTTCTGTTACGTG |
| ACGCAAGGAA (SEQ ID | | CCAGCGA (SEQ ID NO: 99) |
| NO: 81) | | |
| CGTCGTATTGAGTGCTA | | CGGAATAATGTATGTCTT |
| GTACTGGTTTGAG (SEQ | | ACCGAGGC (SEQ ID NO: |
| ID NO: 22) | | 100) |
| | | CTACGATTACCTTAACGA |
| | | CCCTAAC (SEQ ID NO: |
| | | 101) |
| | | ATGATTGACACAATAATT |
| | | AACTGGTT (SEQ ID NO: |
| | | 102) |
| | | GCTTGGAAATATGTCTTA |
| | | TTTATCA (SEQ ID NO: 12) |
| | | ATACCGTCTGTACCTATTG |
| | | GGGGCA (SEQ ID NO: 103) |
| | | AAAAGTGCTAAAATTCTT |
| | | AACGGAA (SEQ ID NO: |
| | | 104) |
| | | CTTGCTGAATTCGGCTCA |
| | | AGCATCAT (SEQ ID NO: |
| | | 105) |
| | | CAGCATGGGATAGAACGC |
| | | TTCCGAGC (SEQ ID NO: |
| | | 106) |
| | | CCACTAGCATCTCCTAGG |
| | | ATAGTTGGA (SEQ ID NO: |
| | | 107) |
| | | ATATAAGACAGCTCCAAG |
| | | CTCCCGTT (SEQ ID NO: |
| | | 108) |
| | | TACCTCTGGAGTTTAATCT |
| | | TTGATAGA (SEQ ID NO: |
| | | 109) |
| | | AATGAAAAACCAAAATCC |
| | | GCACCTTA (SEQ ID NO: |
| | | 110) |
| | | GACGCATATTGCATAGCG |
| | | GTTTATGC (SEQ ID NO: |
| | | 111) |
| | | ATAAATTCACAAACTAAC |
| | | TTGTAAC (SEQ ID NO: |
| | | 112) |
| | | CTAGCTCCTCTACGTCTTT |
| | | ATTTTCACCCTCAT (SEQ |
| | | ID NO: 24) |

In some embodiments, the Cas12 protein of the disclosure is a catalytically active Cas12 protein, e.g. a catalytically active Cas12a.1, Cas12p, or Cas12q protein.

In some embodiments, the Cas12 protein of the disclosure cleaves at a site distal to the target sequence, e.g. the Cas12a.1, Cas12p, or Cas12q protein cleaves at a site distal to the target sequence.

In some embodiments, the Cas12 protein of the disclosure is a catalytically dead Cas12 protein, e.g. the Cas12a.1, Cas12p, or Cas12q protein is a catalytically dead (dCas12a.1, dCas12p, or a dCas12q protein).

In some embodiments, the Cas12 protein of the disclosure is a nickase Cas12 protein, e.g. a Cas12a.1 nickase, a Cas12p nickase, or a Cas12q nickase protein.

In some embodiments, the Cas12 proteins of the disclosure can be modified to include an aptamer.

In some embodiments, the Cas12 proteins of the disclosure can be further fused to domains, e.g. catalytic domains to produce dual action Cas proteins. In some embodiments, a Cas12a protein is further fused to a base editor.

b. Collateral Activity of Class 2 Type V CRISPR-Cas RNA-Guided Proteins

In addition to the ability to cleave a target sequence in a targeted DNA, the Cas12 proteins of the disclosure also possess collateral (trans-cleavage activity), i.e. the ability to promiscuously cleave non-targeted single stranded DNA (ssDNA) or RNA once activated by detection of a target DNA. Without being bound to any theory or mechanism, generally once a Cas12 protein of the disclosure is activated by a gRNA, which occurs when a sample includes a target sequence to which the gRNA hybridizes (i.e., the sample includes the targeted DNA), the Cas12 can become a nuclease that promiscuously cleaves oligonucleotides (e.g. ssDNAs, RNAs, chimeric RNA/DNAs) not comprising the target sequence of the gRNA (non-target oligonucleotides, to which the guide sequence of the gRNA does not hybridize). Thus, when the targeted DNA (double or single stranded) is present in the sample (e.g., in some embodiments above a threshold amount), the result can be cleavage of single stranded oligonucleotides (e.g. ssDNAs, ssRNAs, single stranded chimeric RNA/DNAs) in the sample, which can be detected using any convenient detection method (e.g., using a labeled detector DNA, RNA, or DNA/RNA chimera).

Accordingly, provided herein are methods and compositions for detecting a target DNA (dsDNA or ssDNA) in a sample. Also provided are methods and compositions for cleaving non-target oligonucleotides, which can be utilized detectors. These embodiments are described in further detail below.

c. gRNAs for Class 2 Type V CRISPR-Cas RNA-Guided Proteins

The present disclosure provides DNA-targeting RNAs that direct the activities of the novel Cas12 proteins of the disclosure to a specific target sequence within a target DNA. As above for the novel Cas9 proteins of the disclosure, these DNA-targeting RNAs are referred to herein as “gRNAs” or “gRNAs” Generally, as provided herein, a Cas12's gRNA comprises a single segment comprising both a spacer (DNA-targeting sequence) and a Cas12a “protein-binding sequence” together referred to as a crRNA. Also provided herein are nucleotide sequences encoding the Cas12a gRNAs of the disclosure.

i. Spacer Sequences

The Cas12 proteins of the disclosure are single crRNA-guided endonucleases (single guide RNA, sgRNA, while the Cas9 proteins of the disclosure are guided by a dual-RNA system consisting of a crRNA and a trans-activating crRNA (tracrRNA). The crRNA of the Cas12 guides of the disclosure comprises a nucleotide sequence that is complementary to a sequence in a target DNA (DNA-targeting sequence or spacer).

The crRNA portion of the Cas12 gRNAs of the disclosure can have a length of from about 25-50 nt. In some embodiments, the length can be about 40-43 nt.

The mature guide scaffolds for Cas12a.1 and Cas12p were deduced in silico from the corresponding CRISPR loci. FIG. 38 shows the secondary structure of the scaffolds for Cas12a.1 (5′ aaauuucuacuguaguagau 3′) (SEQ ID NO: 116; Panel A) and Cas12p (5′ agauuucuacuuuuguagau3′)(SEQ ID NO: 117; Panel B). These mature scaffolds can then be joined with variable targeting spacer sequences, giving rise to a sgRNA. Accordingly, in some embodiments, provided herein is an engineered single-molecule gRNA, comprising the scaffold sequence of SEQ ID NO: 116 or SEQ ID NO: 117 and a spacer sequence that is capable of hybridizing with a target sequence in a target DNA. In some embodiments, the target DNA is viral DNA, plant DNA, fungal DNA, or bacterial DNA. In some embodiments, the target sequence is a sequence of a target provided in any of Tables 6a-6f. In some embodiments, the target is a coronavirus. In some embodiments, the target is a SARS-CoV-2 virus. In some embodiments, the target DNA is cDNA, and has been obtained by reverse transcription.

The DNA-targeting spacer sequence of a Cas12 gRNA generally interacts with a target DNA in a sequence-specific manner via hybridization (i.e., base pairing). As such, the nucleotide sequence of the DNA-targeting sequence may vary and determines the location within the target DNA that the gRNA and the target DNA will interact. The DNA-targeting sequence of a subject Cas12 gRNA can be modified (e.g., by genetic engineering) to hybridize to a desired sequence within a target DNA.

The DNA-targeting sequence of a subject Cas12 gRNA can have a length of from about 8 nucleotides to about 30 nucleotides. For example, the length can be 23 nucleotides.

The percent complementarity between the DNA-targeting spacer sequence of the crRNA and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the DNA-targeting sequence of the crRNA-RNA and the target sequence of the target DNA is 100% over the 1-23 contiguous 5′-most nucleotides of the target sequence of the complementary strand of the target DNA. In some embodiments, the percent complementarity between the DNA-targeting sequence of the crRNA and the target sequence of the target DNA is at least 60% over about 1-23 contiguous nucleotides. In some embodiments, the percent complementarity between the DNA-targeting sequence of the crRNA and the target sequence of the target DNA is 100% over the 1-23 contiguous 5′-most nucleotides of the target sequence of the complementary strand of the target DNA and as low as 0% over the remainder. In such a case, the DNA-targeting sequence can be considered to be 1-23 nucleotides in length.

Generally, a naturally unprocessed pre-crRNA of Cas12 comprises a direct repeat and an adjacent spacer (the portion of the crRNA that allows for targeting to a DNA molecule). In some embodiments, direct repeats, and direct repeat mutations from unprocessed pre-crRNA are included into the Cas12 gRNAs of the disclosure, and improve gRNA stability.

Table 5a shows the predicted (putative) naturally occurring direct repeat sequences in the CRISPR locus, as found in bacterial DNA, of the Cas12 proteins of the disclosure. These are the predicted natural sequences in the CRISPR locus contig, as found in bacterial DNA. The gRNAs of the disclosure have a part of the direct repeat joined to the spacer.

TABLE 5a
Direct repeat sequences

| Description | Name | Sequence |
|---|---|---|
| Direct | Cas12a.1 | GTTTAAGGCCTTGACAAAATTTCTACTG |
| Repeat | | TAGTAGAT (SEQ ID NO: 6) |
| Direct | Cas12p | ATCTACAAAAGTAGAAATCTAATAGGGA |
| Repeat | | TATTCGAG (SEQ ID NO: 7) |
| Direct | Cas12p | CTCGAATATCCCTATTAGATTTCTACTT |
| Repeat | | TTGTAGAT (SEQ ID NO: 26) |
| Direct | Cas12q | ATCTACAAAAGTAGAAATTAAATAGGTC |
| Repeat | | TATTTGAG (SEQ ID NO: 8) |
| | | CTCAAATAGACCTATTTAATTTCTACTT |
| | | TTGTAGAT (SEQ ID NO: 27) |

In some embodiments, the crRNAs include non-naturally occurring, engineered direct repeat sequences. Table 5b shows non-naturally occurring, engineered direct repeat sequences which can be incorporated into the engineered gRNAs of the disclosure.

The predicted RNA secondary structures of non-naturally occurring, engineered direct repeat sequences are shown in FIGS. 7A-7C.

TABLE 5b

| | | Sequence of non-naturally |
|---|---|---|
| | | occurring direct repeat |
| Description | Name | sequences |
| Direct | Cas12a.1 | A) GTTTAAGGCCTTGACAAAATTTCCA |
| Repeat | | CTGTAGTGGAT (SEQ ID NO: 28) |
| | | B) GGTTTAAGGCCTTGACAAAATTTCT |
| | | CCTGTAGGAGAT (SEQ ID NO: 29) |
| | | C)GTTTAAGGCCTTGACAAAATTTCCCC |
| | | TGTAGGGGAT (SEQ ID NO: 30) |
| Direct | Cas12p | A) ATCTACAAAAGTAGAAGTCTAATAG |
| Repeat | | GGACATTCGAG (SEQ ID NO: 31) |
| | | B) ATCTACAAAAGTAGAAAGCTAATAG |
| | | GGCTATTCGAG (SEQ ID NO: 32) |
| | | C) ATCTACAAAAGTAGAAGGCTAATAG |
| | | GGCCATTCGAG (SEQ ID NO: 33) |
| | | D) CTCGAATATCCCTATTAGATTTCGA |
| | | CTTTTGTCGAT (SEQ ID NO: 34) |
| | | E) CTCGAATATCCCTATTAGATTTCTC |
| | | CTTTTGGAGAT (SEQ ID NO: 35) |
| | | F) CTCGAATATCCCTATTAGATTTCGG |
| | | CTTTTGCCGAT (SEQ ID NO: 36) |
| Direct | Cas12q | A) ATCTACAAAAGTAGAAATTGAATAG |
| Repeat | | GTCTATTCGAG (SEQ ID NO: 37) |
| | | B) ATCTACAAAAGTAGAAATTAAAGAG |
| | | GTCTCTTTGAG (SEQ ID NO: 38) |
| | | C)ATCTACAAAAGTAGAAATTGGGTAGG |
| | | TCTACCCGAG (SEQ ID NO: 39) |
| | | D) CTCAAATAGACCTATTTAATTTCCA |
| | | CTTTTGTGGAT (SEQ ID NO: 40) |
| | | E) CTCAAATAGACCTATTTAATTTCTC |
| | | CTTTTGGAGAT (SEQ ID NO: 41) |
| | | F) CTCAAATAGACCTATTTAATTTCCC |
| | | CTTTTGGGGAT (SEQ ID NO: 42) |

In some embodiments the spacer sequence of a Cas12 gRNA of the disclosure is directed to a target sequence in a mammalian organism. In some embodiments the spacer sequence is directed to a target sequence in a non-mammalian organism.

In some embodiments, the spacer sequence of a Cas12 gRNA of the disclosure is directed to a target sequence which is a sequence of a human. In some embodiments, the target sequence is a sequence of a non-human primate.

In some embodiments, the spacer sequence of a Cas12 gRNA of the disclosure is directed to a target sequence in a mammalian organism, e.g. a human or non-human primate.

In some embodiments, the spacer sequence of a Cas12 gRNA of the disclosure is directed to a target sequence in a bacteria.

In some embodiments, the spacer sequence of a Cas12 gRNA of the disclosure is directed to a target sequence in a virus.

In some embodiments, the spacer sequence of a Cas12 gRNA of the disclosure is directed to a target sequence in a plant.

The Cas12 gRNAs of the disclosure can be modified to include an aptamer.

ii. PAM Specificities

TCTN and TGTN are identified to be efficient PAM sequences for Cas12a.1 and Cas12p, respectively.

iii. gRNA Arrays

In some embodiments, the Cas12 gRNAs of the disclosure can be provided as gRNA arrays.

Such gRNA arrays of the disclosure include more than one gRNA arrayed in tandem, and can be processed into two or more individual gRNAs. Thus, in some embodiments a precursor Cas12 gRNA array comprises two or more (e.g., 3 or more, 4 or more, 5 or more, 2, 3, 4, or 5) gRNAs (e.g., arrayed in tandem as precursor molecules). In some embodiments, two or more gRNAs can be present on an array (a precursor gRNA array). A Cas12 protein of the disclosure can cleave the precursor gRNA array into individual gRNAs.

In some embodiments a Cas12 gRNA array includes 2 or more gRNAs (e.g., 3 or more, 4 or more, 5 or more, 6 or more, or 7 or more, gRNAs). The gRNAs of a given array can target (i.e., can include guide sequences that hybridize to) different target sites of the same target DNA. In some embodiments, two or more gRNAs of a precursor gRNA array have the same guide sequence. In some embodiments, the precursor gRNA array comprises two or more gRNAs that target different target sites within the same target DNA. In some embodiments, the precursor gRNA array comprises two or more gRNAs that target different target DNAs.

### III. Methods of Use—Modification and Therapeutics

a. Modification of Target DNA

Provided herein are uses of the novel Cas9 and Cas12 proteins of the disclosure. Accordingly, provided herein is a method of modifying a target DNA, the method comprising contacting the target DNA with any one Cas9 systems or Cas12 systems described herein. Such methods are useful for therapeutic application

In some embodiments, the target DNA is part of a chromosome in vitro. In some embodiments, the target DNA is part of a chromosome in vivo.

In some embodiments, the target DNA is part of a chromosome in a cell.

In some embodiments, the target DNA is extrachromosomal DNA.

In some embodiments, the target DNA is in a cell, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In some embodiments, the target DNA is the DNA of a parasite.

In some embodiments, the target DNA is a viral DNA.

In some embodiments, the target DNA is a bacterial DNA.

In some embodiments, the modifying comprises introducing a double strand break in the target DNA.

In some embodiments, the contacting occurs under conditions that are permissive for non-homologous end joining or homology-directed repair.

In some embodiments, the method comprises contacting the target DNA with a donor polynucleotide, wherein the donor polynucleotide, a portion of the donor polynucleotide, a copy of the donor polynucleotide, or a portion of a copy of the donor polynucleotide integrates into the target DNA.

In some embodiments, the method does not comprise contacting the cell with a donor polynucleotide, wherein the target DNA is modified such that nucleotides within the target DNA are deleted.

b. Therapeutic Applications

The disclosure provides novel Cas9 proteins, novel Cas12a proteins, and novel Cas12 protein subtypes, engineered systems, one or more polynucleotides encoding components of said system, and vector or delivery systems comprising one or more polynucleotides encoding components of said system for use in therapeutic methods. The therapeutic methods may comprise gene or genome editing, or gene therapy. The therapeutic methods comprise use and delivery of the novel Cas9 and Cas12 proteins of the disclosure. Accordingly, in some embodiments, provided herein is a method of modifying a target DNA, the method comprising contacting a target DNA, a cell comprising the target DNA, or a subject with cells with the target DNA, with any one Cas9 systems or Cas12 systems described herein.

In some embodiments, the target DNA is part of a chromosome in vitro. In some embodiments, the target DNA is part of a chromosome in vivo.

In some embodiments, the target DNA is part of a chromosome in a cell.

In some embodiments, the target DNA is extrachromosomal DNA.

In some embodiments, the target DNA is in a cell, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In some embodiments, the target DNA is outside of a cell.

In some embodiments, the target DNA is in vitro inside of a cell.

In some embodiments, the target DNA is in vivo, inside of a cell.

In some embodiments, the modifying comprises introducing a double strand break in the target DNA.

In some embodiments, the contacting occurs under conditions that are permissive for non-homologous end joining or homology-directed repair.

In some embodiments, the method comprises contacting the target DNA with a donor polynucleotide, wherein the donor polynucleotide, a portion of the donor polynucleotide, a copy of the donor polynucleotide, or a portion of a copy of the donor polynucleotide integrates into the target DNA.

In some embodiments, the method does not comprise contacting the cell with a donor polynucleotide, wherein the target DNA is modified such that nucleotides within the target DNA are deleted.

In some embodiments, the therapeutic methods involve modifying a target DNA comprising a target sequence of a gene of interest and/or the regulatory region of the gene of interest, the method comprising delivering to a cell comprising the target DNA, a Cas9 protein of the disclosure and one or more Cas9 gRNAs, a Cas12 protein of the disclosure and one or more Cas12 gRNAs, one or more nucleotides encoding the Cas9 protein of the disclosure and one or more Cas9 gRNAs, or one or more nucleotides encoding a Cas12 protein of the disclosure and one or more Cas12 gRNAs.

In some embodiments, the gene of interest is within a eukaryotic cell, e.g. a human or non-human primate cell.

In some embodiments, the gene of interest is within a plant cell.

In some embodiments, the delivering comprises delivering to the cell a Cas9 protein of the disclosure (or one or more nucleotides encoding the same) and one or more Cas9 gRNAs.

In some embodiments, the delivering comprises delivering to the cell a Cas12 protein of the disclosure (or one or more nucleotides encoding the same) and one or more Cas12 gRNAs.

In some embodiments, the delivering comprises delivering to the cell one or more nucleotides encoding the Cas9 protein of the disclosure and one or more Cas9 gRNAs.

In some embodiments, the delivering comprises delivering to the cell one or more nucleotides encoding a Cas12 protein of the disclosure and one or more Cas12 gRNAs.

Delivery of the Cas9 or Cas12 components to a cell can be achieved by any variety of delivery methods known to those of skill in the art. As a non-limiting example, the components can be combined with a lipid. As another non-limiting example, the components combined with a particle, or formulated into a particle, e.g. a nanoparticle.

Methods of introducing a nucleic acid and/or protein into a host cell are known in the art, and any convenient method can be used to introduce a subject nucleic acid (e.g., an expression construct/vector) into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery and the like.

A gRNA can be introduced, e.g., as a DNA molecule encoding the gRNA, or can be provided directly as an RNA molecule (or a chimeric/hybrid molecule when applicable).

In some embodiments, a Cas9 or Cas12 protein is provided as a nucleic acid (e.g., an mRNA, a DNA, a plasmid, an expression vector, a viral vector, etc.) that encodes the protein.

In some embodiments, the Cas9 or Cas12 protein is provided directly as a protein (e.g., without an associated gRNA or with an associate gRNA, i.e., as a ribonucleoprotein complex RNP). Like a gRNA, a Cas9 or Cas12 protein of the disclosure can be introduced into a cell (provided to the cell) by any convenient method; such methods are known to those of ordinary skill in the art. As an illustrative example, a Cas9 or Cas12 protein of the disclosure can be injected directly into a cell (e.g., with or without a gRNA or nucleic acid encoding a gRNA). As another example, a pre-formed complex of a Cas9 or Cas12 protein and a gRNA can be introduced into a cell (e.g., eukaryotic cell) (e.g., via injection, via nucleofection; via a protein transduction domain (PTD) conjugated to one or more components, e.g., conjugated to the Cas9 or Cas12 protein of the disclosure, conjugated to a gRNA; etc.).

In some embodiments, a nucleic acid (e.g., a gRNA; a nucleic acid comprising a nucleotide sequence encoding a Cas9 or Cas12 protein of the disclosure; etc.) and/or a polypeptide (e.g., a Cas9 or Cas12 protein of the disclosure) is delivered to a cell (e.g., a target host cell) in a particle, or associated with a particle. In some embodiments, the particle is a nanoparticle.

A Cas9 or Cas12 protein of the disclosure (or an mRNA comprising a nucleotide sequence encoding the protein) and/or gRNA (or a nucleic acid such as one or more expression vectors encoding the gRNA) may be delivered simultaneously using particles or lipid envelopes.

i. Target Cells of Interest

Suitable target cells (which can comprise target DNA such as genomic DNA) include, but are not limited to: a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell from a vertebrate animal (e.g., a fish, an amphibian, a reptile, a bird, a mammal); a cell from a mammal (e.g., a cell from a rodent; a cell from a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuna, a sheep, a goat, etc.); a cell of a marine mammal (e.g., a whale, a seal, an elephant seal, a dolphin, a sea lion; etc.) and the like.

Any type of cell may be of interest (e.g. a stem cell, e.g. an embryonic stem (ES) cell, an induced pluripotent stem cell (iPSC), a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Cells may be from cell lines or primary cells. Target cells can be unicellular organisms and/or can be grown in culture. If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. can be conveniently harvested by biopsy.

Because the gRNA provides specificity by hybridizing to target nucleic acid, a mitotic and/or post-mitotic cell of interest in the disclosed methods may include a cell of any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardh, and the like, a fungal cell (e.g., a yeast cell), an animal cell, a cell of an invertebrate animal (e.g. fruit fly, cnidarian, echinoderm, nematode, etc.), a cell of a vertebrate animal (e.g., fish, amphibian, reptile, bird, mammal), a cell of a mammal, a cell of a rodent, a cell of a human, etc.).

Plant cells include cells of a monocotyledon, and cells of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some embodiments, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be and in vivo cell (e.g., a cell in an individual). A cell can be an isolated cell. A cell can be a cell inside of an organism. A cell can be an organism.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some embodiments, the cell is an immune cell, a neuron, an epithelial cell, and endothelial cell, or a stem cell. In some embodiments, the immune cell is a T cell, a B cell, a monocyte, a natural killer cell, a dendritic cell, or a macrophage. In some embodiments, the immune cell is a cytotoxic T cell. In some embodiments, the immune cell is a helper T cell. In some embodiments, the immune cell is a regulatory T cell (Treg).

In some embodiments, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some embodiments, the stem cell is a human stem cell. In some embodiments, the stem cell is a rodent (e.g., a mouse; a rat) stem cell. In some embodiments, the stem cell is a non-human primate stem cell.

ii. Targets

Any gene of interest can serve as a target for modification.

In particular embodiments, the target is a gene implicated in cancer. In particular embodiments, the target is a gene implicated in an immune disease, e.g. an autoimmune disease.

In particular embodiments, the target is a gene implicated in a neurodegenerative disease. In particular embodiments, the target is a gene implicated in a neuropsychiatric disease. In particular embodiments, the target is a gene implicated in a muscular disease. In particular embodiments, the target is a gene implicated in a cardiac disease. In particular embodiments, the target is a gene implicated in diabetes. In particular embodiments, the target is a gene implicated in kidney disease.

iii. Precursor gRNA Arrays

The therapeutic methods provided herein can include delivery of precursor gRNA arrays. A Cas9 or Cas12 protein of the disclosure can cleave a precursor gRNA into a mature gRNA, e.g., by endoribonucleolytic cleavage of the precursor. A Cas9 or Cas12 protein of the disclosure can cleave a precursor gRNA array (that includes more than one gRNA arrayed in tandem) into two or more individual gRNAs.

### IV. Methods of Use—Detection and Diagnostic Applications

In addition to the ability to cleave a target sequence in a targeted DNA, the Cas12 proteins of the disclosure also possess collateral (trans-cleavage activity), i.e. the ability to promiscuously cleave non-targeted oligonucleotides (ssDNA, RNA, DNA/RNA hybrids) once activated by detection of a target DNA. Without being bound to any theory or mechanism, generally once a Cas12 protein of the disclosure is activated by a gRNA, which occurs when a sample includes a target sequence to which the gRNA hybridizes (i.e., the sample includes the targeted DNA), the Cas12 becomes a nuclease that promiscuously cleaves single stranded oligonucleotides (i.e., non-target single stranded oligonucleotides, i.e., single stranded oligonucleotides to which the guide sequence of the gRNA does not hybridize). Thus, when the targeted DNA (double or single stranded) is present in the sample (e.g., in some embodiments above a threshold amount), the result can be cleavage (collateral) of oligonucleotides in the sample, which can be detected using any convenient detection method (e.g., using a labeled single stranded detector DNA, labeled detector RNA, or labeled detector DNA/RNA chimeric oligonucleotides).

Accordingly, provided herein are methods and compositions for detecting a target DNA (dsDNA or ssDNA) in a sample. Also provided are methods and compositions for cleaving non-target oligonucleotides (e.g. used as detectors).

As used herein a “detector” comprises a oligonucleotide of any nature, single or double stranded and does not hybridize with the guide sequence of the gRNA (i.e., the detector oligonucleotide that is a non-target). Exemplary detectors include, but are not limited to ssDNA, dsDNA, ssRNA, ss DNA/RNA chimeras, dsRNA, RNA comprising ss and ds regions, and ss or ds oligonucleotides containing RNA and DNA nucleotides (as used herein ss=single stranded; and ds=double stranded).

The detection methods based on the collateral activity of the Cas12 proteins of the disclosure can include:

(a) contacting the sample with: (i) a Cas12 protein of the disclosure; (ii) a gRNA comprising: a region that binds to the Cas12 protein, and a guide sequence that hybridizes with the target DNA; and (iii) a detector that does not hybridize with the guide sequence of the gRNA; and

(b) measuring a detectable signal produced by cleavage of the detector by the Cas12 protein, thereby detecting the target DNA.

Once a subject Cas12 protein is activated by a gRNA, which can occur when the sample includes a target DNA to which the gRNA hybridizes (i.e., the sample includes the targeted sequence in the target DNA), the Cas12 can be activated to function as an endoribonuclease that non-specifically cleaves detector oligonucleotides (including non-target ss oligonucleotides) present in the sample. Thus, when the target DNA is present in the sample, the result is cleavage of a detector oligonucleotide in the sample, which can be detected using any convenient detection method (e.g., using a labeled detector oligonucleotides).

Also provided are methods and compositions for cleaving detector oligonucleotides (e.g., ssDNAs, ssRNAs, ssDNA/RNA chimeras or detectors comprising ss and ds regions). Such methods can include contacting a population of nucleic acids, wherein said population comprises a target DNA and a plurality of non-target ss oligonucleotides, with: (i) a Cas12 protein of the disclosure; and (ii) a gRNA comprising: a region that binds to the Cas12 effector protein, and a guide sequence that hybridizes with the target DNA, wherein the Cas12 protein cleaves non-target ss oligonucleotides

Accordingly, provided herein is a method of detecting a target DNA in a sample, the method comprising:

(a) contacting the sample with:

(i) a Cas12 protein of the disclosure (e.g. Cas12a.1, Cas12p, or Cas12q protein);

(ii) a gRNA comprising a spacer sequence that is capable of hybridizing with a target sequence in a target DNA; and

(iii) a labeled detector oligonucleotide that does not hybridize with the spacer sequence of the gRNA; and

(b) measuring a detectable signal produced by cleavage of the labeled detector oligonucleotide by the Cas12 protein, thereby detecting the target oligonucleotide.

In some embodiments, the method further comprises the above along with detecting a positive control target DNA in a positive control sample, the detecting comprising the additional steps of:

(c) contacting the positive control sample with:

(i) a Cas12 protein of the disclosure (e.g. Cas12a.1, Cas12p, or Cas12q protein);

(ii) a positive control gRNA comprising: a region that binds to the Cas12a.1, Cas12p, or Cas12q protein, and a positive control spacer sequence that hybridizes with the positive control target DNA; and

(iii) a labeled detector oligonucleotide that does not hybridize with the positive control spacer sequence of the positive control gRNA; and

(d) measuring a detectable signal produced by cleavage of the labeled detector by the Cas12 protein, thereby detecting the positive control target DNA.

In some embodiments, the contacting step can be carried out in an acellular environment, e.g., outside of a cell. In other embodiments, contacting step can be carried out inside a cell. The contacting step can be carried out in a cell in vitro. The contacting step can be carried out in a cell in vivo. The contacting step of a detection method can be carried out in a composition comprising divalent metal ions.

The gRNA can be provided as RNA or as a nucleic acid encoding the gRNA (e.g., a DNA such as a recombinant expression vector), described herein.

The contacting, prior to the measuring step, can last for any period of time, e.g from 5 seconds to 2 hours or more, prior to the measuring step. In some embodiments the sample is contacted for 45 minutes or less prior to the measuring step. In some embodiments the sample is contacted for 30 minutes or less prior to the measuring step. In some embodiments the sample is contacted for 10 minutes or less prior to the measuring step. In some embodiments the sample is contacted for 5 minutes or less prior to the measuring step. In some embodiments the sample is contacted for 1 minute or less prior to the measuring step. In some embodiments the sample is contacted for from 50 seconds to 60 seconds prior to the measuring step. In some embodiments the sample is contacted for from 40 seconds to 50 seconds prior to the measuring step. In some embodiments the sample is contacted for from 30 seconds to 40 seconds prior to the measuring step. In some embodiments the sample is contacted for from 20 seconds to 30 seconds prior to the measuring step. In some embodiments the sample is contacted for from 10 seconds to 20 seconds prior to the measuring step.

The detection methods provided herein can detect a target DNA with a high degree of sensitivity. Accordingly, in some embodiments, the detection methods of the disclosure can be used to detect a target DNA present in a sample comprising a plurality of DNAs (including the target DNA and a plurality of non-target DNAs), where the target DNA is present at one or more copies per 5 to 10\hat{ }9 copies of the non-target DNAs

In some embodiments, the threshold of detection, for a detection method of detecting a target DNA in a sample, is 10 nM or less. The term “threshold of detection” is used herein to describe the minimal amount of target DNA that must be present in a sample in order for detection to occur. Thus, as an illustrative example, when a threshold of detection is 10 nM, then a signal can be detected when a target DNA is present in the sample at a concentration of 10 nM or more. In some embodiments, a subject composition or method exhibits an attomolar (aM) sensitivity of detection. In some embodiments, a subject composition or method exhibits a femtomolar (fM) sensitivity of detection. In some embodiments, a subject composition or method exhibits a picomolar (pM) sensitivity of detection. In some embodiments, a subject composition or method exhibits a nanomolar (nM) sensitivity of detection.

a. Target DNA

A target DNA can be single stranded (ssDNA) or double stranded (dsDNA). There need not be any preference or requirement for a PAM sequence in a single stranded target DNA.

The source of the target DNA can be any source. In some embodiments the target DNA is a viral or bacterial DNA (e.g., a genomic DNA of a DNA virus or bacteria). As such, detection method can be for detecting the presence of a viral or bacterial DNA amongst a population of nucleic acids (e.g., in a sample). In the case of a RNA-carrying organism, for example, a RNA virus (e.g. a coronavirus)—it is understood that a step such as reverse transcription may be carried out on a sample comprising the RNA-carrying organism to generated cDNA, and the cDNA is then the target DNA, for the purposes of this disclosure.

Exemplary non-limiting sources for target DNA are provided in Tables 6a-6f.

TABLE 6a
Bacterial Resistance Gene Targets

KPC: carbapenem-hydrolyzing class A beta-lactamase
NDM: metallo-beta-lactamase
OXA: oxacillin-hydrolyzing class D beta-lactamase
MecA: PBP2a family beta-lactam-resistant
peptidoglycan transpeptidase
vanA/B: Vancomycin resistance

TABLE 6b
Virus Genome Targets

Dengue (DENV) fever virus (subtypes 1,2, 3 and 4)
Zika Virus
Chikungunya virus
Coronoavirus

### Respiratory Targets

DNA obtained from viruses and bacteria related to respiratory infections may also be targeted. A list of targets of interest may include the examples shown in Table 6c.

TABLE 6c
Respiratory Targets

Adenovirus
Coronoavirus
SARS-CoV
SARS-CoV-2
MERS-CoV
Coronavirus HKU1
Coronavirus NL63
Coronavirus 229E
Coronavirus OC43
Coronovirus HKU1
Human Metapneumovirus
Human Rhinovirus/Enterovirus
Influenza A
Influenza A/H1
Influenza A/H3
Influenza A/H1-2009
Influenza B
Parainfluenza Virus 1
Parainfluenza Virus 2
Parainfluenza Virus 3
Parainfluenza Virus 4
Respiratory Syncytial Virus
BACTERIA:
Bordetella parapertussis
Bordetella pertussis
Chlamydia pneumoniae
Mycoplasma pneumoniae

### Sexually Transmitted Disease Targets

DNA obtained from viruses and bacteria related to sexually transmitted diseases may also be targeted. A list of targets of interest may include the examples shown in Table 6d.

TABLE 6d
Sexually Transmitted Disease Targets

HIV (Type 1 and type 2)
Herpes Simplex Virus 1 (HSV-1)
Herpes Simplex Virus 2 (HSV-2)
Hepatitis A
Hepatitis B
Hepatitis C
BACTERIA
Treponema pallidum
Chlamydia
Neisseria gonorrhoeae

### Other Targets

Other DNAs may also be targeted. As another example, male genes to determine the sex of the embryo of a pregnant woman/animal, and the male genes to determine the sex of plants and seeds may also be targeted. Examples of further targets of interest may include the following shown in Table 6e.

TABLE 6e
Viral
Papovavirus (e.g., human papillomavirus (HPV), polyomavirus)
Hepadnavirus (e.g., Hepatitis B Virus (HBV))
Herpesvirus (e.g., herpes simplex virus (HSV)
Varicella zoster virus (VZV)
Epstein-barr virus (EBV)
Cytomegalovirus (CMV)
Herpes lymphotropic virus, Pityriasis Rosea, kaposi's sarcoma-associated herpesvirus);
Adenovirus (e.g., atadenovirus, aviadenovirus, ichtadenovirus, mastadenovirus, siadenovirus)
Poxvirus (e.g., smallpox, vaccinia virus, cowpox virus, monkeypox virus, orf virus,
pseudocowpox, bovine papular stomatitis virus; tanapox virus, yaba monkey tumor virus;
molluscum contagiosum virus (MCV))
Parvovirus (e.g., adeno-associated virus (AAV), Parvovirus B19, human bocavirus,
bufavirus, human parv4 G1); Geminiviridae; Nanoviridae; Phycodnaviridae; and the like.
Dengue fever virus (subtypes 1, 2, 3, and 4)
Zika virus
Hantavirus
Chikungunya virus

Other miscellaneous targets of interest that provide sources for DNA targets are shown in Table 6f.

TABLE 6f

Sex determination targets
SRY genes of mammals and non-mammal animals
Other miscellaneous targets of interest
hHPRT1 (hypoxanthine phosphoribosyltransferase 1)
16S E. coli

A list of non-limiting exemplary target sequences is provided in Tables 6g.

TABLE 6g

| Description | Name | Sequence | Targets |
|---|---|---|---|
| Target | KPC 1 | TTGCTGAAGGAGTTGGGCGGC | KPC |
| sequence | | CC (SEQ ID NO: 51) | |
| Target | NDM 1 | GCGATCTGGTTTTCCGCCAGC | NDM |
| sequence | | TC (SEQ ID NO: 52) | |
| Target | Ctrol + | GGTTAAAGATGGTTAAATGAT | hHPRTl |
| sequence | hHPRT1 1 | (SEQ ID NO: 53) | |
| Target | S16 cntl | CAGTAGTTATCCCCCTCCATC | 16S |
| sequence | E coli 1 | AG (SEQ ID NO: 54) | E. coli |
| Target | DENV1 | CTTCTGTCCAGTGAGCATGGT | Dengue |
| sequence | | CT (SEQ ID NO: 55) | virus |
| Target | DENV2 | TGGTTCAAAGAGAGCTGGTAT | Dengue |
| sequence | | AA (SEQ ID NO: 56) | Virus |
| Target | ZIK1 | GGCATGTGCGTCCTTGAACTC | Zika |
| sequence | | TA (SEQ ID NO: 57) | |
| Target | ZIK2 | CCTTTTGGCATGTGCGTCCTT | Zika |
| sequence | | GA (SEQ ID NO: 58) | |
| Target | OXA1 | AGCCCGAATAATATAGTCACC | Oxa-1 |
| sequence | | AT (SEQ ID NO: 59) | |
| Target | OXA1b | AGCCCGAATAATATAGTCGCC | Oxa-1 |
| sequence | | AT (SEQ ID NO: 60) | |
| Target | HANTAndes1 | GTGGCAGCTCAAAAATTGGCT | Hanta- |
| sequence | | AC (SEQ ID NO: 61) | virus |
| Target | HANTAndes2 | GATGATCATCAGGCTCAAGCC | Hanta- |
| sequence | | CT (SEQ ID NO: 62) | virus |
| Target | MecAl | TCTTTTTGCCAACCTTTACCA | MecA1 |
| sequence | | TC (SEQ ID NO: 63) | |
| Target | SARS-CoV-2 | GATCGCGCCCCACTGCGTTCT | SARS- |
| sequence | | CC (SEQ ID NO: 120) | CoV-2 |

b. Samples

The term “sample” is used herein to mean any sample that includes DNA (e.g., in order to determine whether a target DNA is present among a population of DNAs). As noted above, the DNA can be single stranded DNA, double stranded DNA, complementary DNA, and the like.

A sample intended for detection comprises a plurality of nucleic acids. Thus, in some embodiments a sample includes two or more (e.g., 3 or more, 5 or more, 10 or more, 20 or more, 50 or more, 100 or more, 500 or more, 1,000 or more, or 5,000 or more) nucleic acids (e.g., DNAs). A detection method can be used as a very sensitive way to detect a target DNA present in a sample (e.g., in a complex mixture of nucleic acids such as DNAs).

In some embodiments the sample includes 5 or more DNAs (e.g., 10 or more, 20 or more, 50 or more, 100 or more, 500 or more, 1,000 or more, or 5,000 or more DNAs) that differ from one another in sequence. In some embodiments, the sample includes 10 or more, 20 or more, 50 or more, 100 or more, 500 or more, 10\hat{ }3 or more, 5×10\hat{ }3 or more, 10\hat{ }4 or more, 5×10\hat{ }4 or more, 10\hat{ }5 or more, 5×10\hat{ }5 or more, 10\hat{ }6 or more 5×10\hat{ }6 or more, or 10\hat{ }7 or more, DNAs. In some embodiments, the sample comprises from 10 to 20, from 20 to 50, from 50 to 100, from 100 to 500, from 500 to 10\hat{ }3, from 10\hat{ }3 to 5×10\hat{ }3, from 5×10\hat{ }3 to 10\hat{ }4, from 10\hat{ }4 to 5×10\hat{ }4, from 5×10\hat{ }4 to 10\hat{ }5, from 10\hat{ }5 to 5×10\hat{ }5, from 5×10\hat{ }5 to 10\hat{ }6, from 10\hat{ }6 to 5×10\hat{ }6, or from 5×10\hat{ }6 to 10\hat{ }7, or more than 10\hat{ }7, DNAs. In some embodiments, the sample comprises from 5 to 10\hat{ }7 DNAs (e.g., that differ from one another in sequence) (e.g., from 5 to 10\hat{ }6, from 5 to 10\hat{ }5, from 5 to 50,000, from 5 to 30,000, from 10 to 10\hat{ }6, from 10 to 10\hat{ }5, from 10 to 50,000, from 10 to 30,000, from 20 to 10\hat{ }6, from 20 to 10\hat{ }5, from 20 to 50,000, or from 20 to 30,000 DNAs).

In some embodiments the sample includes 20 or more DNAs that differ from one another in sequence. In some embodiments, the sample includes DNAs from a cell lysate (e.g., a eukaryotic cell lysate, a mammalian cell lysate, a human cell lysate, a prokaryotic cell lysate, a plant cell lysate, and the like). For example, in some embodiments the sample includes DNA from a cell such as a eukaryotic cell, e.g., a mammalian cell such as a human cell.

The sample can be derived from any source, e.g., the sample can be a synthetic combination of purified DNAs; the sample can be a cell lysate, a DNA-enriched cell lysate, or DNAs isolated and/or purified from a cell lysate. The sample can be from a patient (e.g., for the purpose of diagnosis). The sample can be from permeabilized cells. The sample can be from crosslinked cells. The sample can be in tissue sections.

A sample can include a target DNA and a plurality of non-target DNAs. In some embodiments, the target DNA is present in the sample at one or more copies per 5 to 10\hat{ }9 copies of the non-target DNAs.

Suitable samples include but are not limited to urine, blood, serum, plasma, lymphatic fluid, cerebrospinal fluid, saliva, nasopharyngeal, oropharyngeal, nasopharyngeal/oropharyngeal, aspirate, or biopsy sample. Thus, the term “sample” with respect to a patient encompasses blood and other liquid samples of biological origin, solid tissue samples such as a biopsy specimen or tissue cultures or cells derived therefrom and the progeny thereof. Samples also can be samples that have been manipulated in any way after their procurement, such as by treatment with reagents; washed; or enrichment for certain cell populations, such as cancer cells. The samples can be obtained by use of a swab, for example, a nasopharyngeal swab, an oropharyngeal swab, or a nasopharyngeal/oropharyngeal swab. Samples also can be samples that have been enriched for particular types of molecules, e.g., DNAs. Samples encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising DNAs that is obtained from such cells (e.g., a cell lysate or other cell extract comprising DNAs).

A sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal; a cell, tissue, fluid, or organ from a mammal (e.g., a human; a non-human primate; an ungulate; a feline; a bovine; an ovine; a caprine; etc.). Suitable sample sources include nematodes, protozoans, and the like. Suitable sample sources include parasites such as helminths, malarial parasites, etc.

Suitable sample sources include a cell, tissue, or organism of any of the six kingdoms.

Suitable sources of a sample include cells, fluid, tissue, or organ taken from an organism; from a particular cell or group of cells isolated from an organism; etc. For example, where the organism is a plant, suitable sources include xylem, the phloem, the cambium layer, leaves, roots, etc. Where the organism is an animal, suitable sources include particular tissues (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.).

In some embodiments, the source of the sample is a (or is suspected of being a diseased cell, fluid, tissue, or organ.

In some embodiments, the source of the sample is a normal (non-diseased) cell, fluid, tissue, or organ.

In some embodiments, the source of the sample is a (or is suspected of being a pathogen-infected cell, tissue, or organ. For example, the source of a sample can be an individual who may or may not be infected—and the sample could be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. In some embodiments, the sample is a cell-free liquid sample.

In some embodiments, the sample is a liquid sample that can comprise cells (urine, blood, serum, plasma, lymphatic fluid, cerebrospinal fluid, saliva, nasopharyngeal, oropharyngeal, nasopharyngeal/oropharyngeal, aspirate, and biopsy). Pathogens include viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include RNA or DNA viruses, e.g., coronavirus (e.g. SARS-CoV, SARS-CoV-2, MERS-CoV); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogenic viruses can include DNA viruses such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Pathogens can include, e.g., DNAviruses [e.g.: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like], Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

c. Measuring a Detectable Signal

The detection method generally includes a step of measuring (e.g., measuring a detectable signal produced by the Cas12 of the disclosure. A detectable signal can be any signal that is produced when ss oliogonucleotide is cleaved. The step of detection can involve a fluorescence-based detection. The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), the presence or absence of (or a particular amount of) a magnetic signal and the presence or absence of (or a particular amount of) an electrical signal.

The measuring can in some embodiments be quantitative, e.g., in the sense that the amount of signal detected can be used to determine the amount of target DNA present in the sample. The measuring can in some embodiments be qualitative, e.g., in the sense that the presence or absence of detectable signal can indicate the presence or absence of targeted DNA (e.g., virus, SNP, etc.). In some embodiments, a detectable signal will not be present (e.g., above a given threshold level) unless the targeted DNA(s) (e.g., virus, SNP, etc.) is present above a particular threshold concentration. In some embodiments, the threshold of detection can be titrated by modifying the amount of the Cas12 protein provided.

The compositions and methods of this disclosure can be used to detect any DNA target.

In some embodiments, the detection methods of the disclosure can be used to determine the amount of a target DNA in a sample (e.g., a sample comprising the target DNA and a plurality of non-target DNAs). Determining the amount of a target DNA in a sample can comprise comparing the amount of detectable signal generated from a test sample to the amount of detectable signal generated from a reference sample. Determining the amount of a target DNA in a sample can comprise: measuring the detectable signal to generate a test measurement; measuring a detectable signal produced by a reference sample to generate a reference measurement; and comparing the test measurement to the reference measurement to determine an amount of target DNA present in the sample.

In some embodiments, the detectable signal is detectable in less than 1, 2, 3, 4, 5, 10, 15, 20, 30, 60, 90, 120, 150, 180, 210, or 240 minutes.

In some embodiments, sensitivity of a subject composition and/or method (e.g., for detecting the presence of a target DNA, such as viral DNA or a SNP, in cellular genomic DNA) can be increased by coupling detection with nucleic acid amplification.

In some embodiments, the nucleic acids in a sample are amplified prior to contact with a Cas12; in particular embodiments, the Cas12 remains in an inactive state until amplification has concluded. In some embodiments, the nucleic acids in a sample are amplified simultaneous with contact with Cas12. Amplification can be carried out using primers. As it relates to the overall processing time for the detection method, amplification can occur for 5 seconds or more, up to 240 minutes or more.

Various amplification methods and components will be known to one of ordinary skill in the art and any convenient method can be used.

Nucleic acid amplification can comprise polymerase chain reaction (PCR), reverse transcription PCR (RT-PCR), quantitative PCR (qPCR), reverse transcription qPCR (RT-qPCR), isothermal PCR, nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, and thermal asymmetric interlaced PCR (TAIL-PCR).

In some embodiments the amplification is isothermal amplification. Isothermal nucleic acid amplification methods can therefore be carried out inside or outside of a laboratory environment. Examples of isothermal amplification methods include but are not limited to: loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

d. Detector Oligonucleotides

The novel Cas12 proteins of the disclosure possess collateral cleavage (trans-cleavage) activity. As in the case of Cas12a.1, the protein possesses the ability to collaterally cleave ssDNAs upon the binding of the DNA targeted by the guide. In the case of Cas12p, the protein possesses the dual ability to collaterally cleave all types of oligonucleotides inclusive of ssDNAs, ssRNAs, chimeric ss DNA/RNAs, and other oligonucleotides comprising RNAs. These characteristics are taken into account when designing the detector oligonucleotides when using the assay.

In some embodiments, a detection method includes contacting a sample (e.g., a sample comprising a target DNA and a plurality of non-target ssDNAs) with: i) a Cas12 protein of the disclosure; ii) a gRNA (or precursor gRNA array); and iii) a detector that does not hybridize with the guide sequence of the gRNA. For example, in some embodiments, a detection method includes contacting a sample with a labeled detector (detector ssDNA in the case of Cas12a.1 or a detector comprising RNA, DNA, and combinations of the same in the case of Cas12p) that includes a fluorescence-emitting dye pair; the Cas12 protein of the disclosure has the ability to cleave the labeled detector after it is activated (by gRNA hybridizing to a target DNA); and the detectable signal that is measured is produced by the fluorescence-emitting dye pair. For example, in some embodiments, a detection method includes contacting a sample with a labeled detector comprising a fluorescence resonance energy transfer (FRET) pair or a quencher/fluor pair, or both. In some embodiments, a detection method includes contacting a sample with a labeled detector comprising a FRET pair. In some embodiments, a detection method includes contacting a sample with a labeled detector comprising a fluor/quencher pair.

Fluorescence-emitting dye pairs comprise a FRET pair or a quencher/fluor pair. In both embodiments of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair”. The term “fluorescence-emitting dye pair” is used interchangeably with the phrase “a FRET pair and/or a quencher/fluor pair.”

In some embodiments (e.g., when the detector includes a FRET pair) the labeled detector produces an amount of detectable signal prior to being cleaved, and the amount of detectable signal that is measured is reduced when the labeled detector is cleaved. In some embodiments, the labeled detector produces a first detectable signal prior to being cleaved (e.g., from a FRET pair) and a second detectable signal when the labeled detector is cleaved (e.g., from a quencher/fluor pair). As such, in some embodiments, the labeled detector comprises a FRET pair and a quencher/fluor pair.

In some embodiments, the labeled detector comprises a FRET pair.

FRET donor and acceptor moieties (FRET pairs) will be known to one of ordinary skill in the art and any convenient FRET pair (e.g., any convenient donor and acceptor moiety pair) can be used. Examples of suitable FRET pairs include but are not limited to those presented in Table 7. FRET pairs provided in U.S. Pat. No. 10,253,365 are incorporate by reference herein in their entirety. In some embodiments, the FRET pair is 5′ 6-FAM and 3IABkFQ (Iowa Black (Registred)-FQ).

TABLE 7
Examples of FRET pairs (donor and and acceptor pairs)

| Donor | Acceptor |
|---|---|
| Tryptophan | Dansyl |
| IAEDANS (1) | DDPM (2) |
| BFP | DsRFP |
| Dansyl | Fluorescein |
| | isothiocyanate (FITC) |
| Dansyl | Octadecylrhodamine |
| Cyan fluorescent | Green fluorescent protein |
| protein (CFP) | (GFP) |
| CF (3) | Texas Red |
| Fluorescein | Tetramethylrhodamine |
| Cy3 | Cy5 |
| GFP | Yellow fluorescent |
| | protein (YFP) |
| BODIPY FL (4) | BODIPY FL (4) |
| Rhodamine 110 | Cy3 |
| Rhodamine 6G | Malachite Green |
| FITC | Eosin Thiosemicarbazide |
| B-Phycoerythrin | Cy5 |
| Cy5 | Cy5.5 |

(1) 5-(2-iodoacetylaminoethyl)aminonaphthalene-1-sulfonic acid
(2) N-(4-dimethylamino-3,5-dinitrophenyl)maleimide
(3) carboxyfluorescein succinimidyl ester
(4) 4,4-difluoro-4-bora-3a,4a-diaza-s-indacene

In some embodiments, a detectable signal is produced when the labeled detector is cleaved (e.g., in some embodiments, the labeled detector comprises a quencher/fluor pair).

Any fluorescent label can be utilized. Examples of fluorescent labels include, but are not limited to: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), fluorescein amidite (FAM), tetramethylrhodamine (TRITC), Texas Red, Oregon Green, Pacific Blue, Pacific Green, Pacific Orange, quantum dots, and a tethered fluorescent protein.

Examples of quencher moieties include, but are not limited to: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In some embodiments, a quencher moiety is selected from: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and a metal cluster.

In some embodiments, cleavage of a labeled detector can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some embodiments, cleavage of a subject labeled detector can be detected by a color-shift. Such a shift can be expressed as a loss of an amount of signal of one color (wavelength), a gain in the amount of another color, a change in the ration of one color to another, and the like.

As provided herein, a labeled detector can be a nucleic acid mimetic. Polynucleotide mimics include PNAs, LNAs, CeNAs, and morpholino nucleic acids.

A labeled detector can also include one or more substituted sugar moieties.

A labeled detector may also include modified nucleotides.

e. Positive Controls

The detection methods provided herein can also include a positive control target DNA. In some embodiments, the methods include using a positive control gRNA that comprises a nucleotide sequence that hybridizes to a control target DNA. In some embodiments, the positive control target DNA is provided in various amounts. In some embodiments, the positive control target DNA is provided in various known concentrations, along with control non-target DNAs.

f. gRNA Arrays

In some embodiments, the method comprises contacting the sample with a precursor gRNA array, wherein the novel Cas12 protein of the disclosure cleaves the precursor gRNA array to produce said gRNA.

In some embodiments a such a gRNA array includes 2 or more gRNAs (e.g., 3 or more, 4 or more, 5 or more, 6 or more, or 7 or more, gRNAs). The gRNAs of a given array can target (i.e., can include guide sequences that hybridize to) different target sites of the same target DNA (e.g., which can increase sensitivity of detection) and/or can target different target DNAs (e.g., single nucleotide polymorphisms (SNPs), different strains of a particular virus, etc.), and such could be used for example to detect multiple strains of a virus. In some embodiments, each gRNA of a precursor gRNA array has a different guide sequence.

In some embodiments, the precursor gRNA array comprises two or more gRNAs that target different target sites within the same target DNA. For example, such a scenario can in some embodiments increase sensitivity of detection by activating Cas9 or Cas12 protein of the disclosure when either one hybridizes to the target DNA. As such, in some embodiments as subject composition (e.g., kit) or method includes two or more gRNAs (in the context of a precursor gRNA array, or not in the context of a precursor gRNA array, e.g., the gRNAs can be mature gRNAs).

In some embodiments, the precursor gRNA array comprises two or more gRNAs that target different target DNAs. For example, such a scenario can result in a positive signal when any one of a family of potential target DNAs is present. Such an array could be used for targeting a family of transcripts, e.g., based on variation such as single nucleotide polymorphisms (SNPs) (e.g., for diagnostic purposes). Such could also be useful for detecting whether any one of a number of different strains of virus is present. Such could also be useful for detecting whether any one of a number of different species, strains, isolates, or variants of a bacterium or virus is present As such, in some embodiments as subject composition (e.g., kit) or method includes two or more gRNAs (in the context of a precursor gRNA array, or not in the context of a precursor gRNA array, e.g., the gRNAs can be mature gRNAs).

### V. Compositions of Matter

Provided herein are compositions and pharmaceutical compositions comprising the Cas9 proteins and/or the Cas9 gRNAs of the disclosure, which can optionally include a pharmaceutically acceptable carrier and/or a protein stabilizing buffer, and/or a nucleic acid stabilizing buffer. In some embodiments, the Cas9 proteins and/or the Cas9 gRNAs are provided in a lyophilized form.

Provided herein are compositions and pharmaceutical compositions comprising the Cas12 proteins and/or the Cas12 gRNAs of the disclosure, which can optionally include a pharmaceutically acceptable carrier and/or a protein stabilizing buffer, and/or a nucleic acid stabilizing buffer. In some embodiments, the Cas12 proteins and/or the Cas12 gRNAs are provided in a lyophilized form.

Provided herein are compositions comprising gRNAs and/or gRNA arrays of the disclosure (compatible for use with Cas9 proteins of the disclosure, and/or Cas12 proteins of the disclosure), and optionally a protein stabilizing buffer.

Provided herein are proteins comprising an amino acid sequence with 70%-99.5% homology to SEQ ID NO: 1, 2, 3, 4, 222, 5, 10, 11, or 12. Provided herein are compositions comprising these proteins, and optionally a pharmaceutically acceptable carrier. Provided herein are these proteins and optionally a protein stabilizing buffer.

Provided herein are DNA polynucleotides encoding a sequence that encodes any of the Cas9 or Cas12 proteins of the disclosure. Also provided are recombinant expression vectors comprising such DNA polynucleotides. In some embodiments, a nucleotide sequence encoding a Cas9 or Cas12 of the disclosure is operably linked to a promoter. In some embodiments, the nucleic acid encoding the Cas9 or Cas12 further comprises a nuclear localization signal (NLS), useful for expression in eukaryotic systems.

Provided herein are DNA polynucleotides or RNAs comprising a sequence that encodes any of the gRNAs of the disclosure. Also provided are recombinant expression vectors comprising such DNA polynucleotides. In some embodiments, a nucleotide sequence encoding a gRNA of the disclosure is operably linked to a promoter.

Also provided herein are host cells comprising any of the recombinant vectors provided herein.

### VI. Kits

Provided herein are kits comprising one or more components of the Cas9 and Cas12 engineered systems described herein, useful for a variety of applications including, but not limited to, therapeutic and diagnostic applications.

In some embodiments provided herein is a kit comprising: (a) a Cas9.1, Cas9.2, Cas9.3 or Cas9.4 protein, or a nucleic acid encoding the Cas9.1, Cas9.2, Cas9.3 or Cas9.4 protein; and (b) a Cas9.1, Cas9.2, Cas9.3 or Cas9.4 gRNA, or a nucleic acid encoding the Cas9.1, Cas9.2, Cas9.3 or Cas9.4 gRNA, wherein the gRNA and the Cas9.1, Cas9.2, Cas9.3 or Cas9.4 protein do not naturally occur together, wherein the gRNA is capable of hybridizing to a target sequence in a target DNA, and the gRNA is capable of forming a complex with the Cas9.1, Cas9.2, Cas9.3 or Cas9.4 protein.

In some embodiments provided herein is a kit comprising: (a) a Cas12a.1, Cas12p, or Cas12q protein, or a nucleic acid encoding the Cas12a.1, Cas12p, or Cas12q protein; and (b) a Cas12a.1, Cas12p, or Cas12q gRNA, or a nucleic acid encoding a Cas12a.1, Cas12p, or Cas12q gRNA, wherein the gRNA and the Cas12a.1, Cas12p, or Cas12q protein do not naturally occur together, wherein the gRNA is capable of hybridizing to a target sequence in a target DNA, and the gRNA is capable of forming a complex with the Cas12a.1, Cas12p, or Cas12q protein.

In exemplary embodiments, provided herein are diagnostic kits. In exemplary embodiments, the reagent components are provided in lyophilized form. In some embodiments, the reagent components are provided individually (either lyophilized or not lyophilized), in other embodiments, the reagent components are provided in a pre-mixed format (either lyophilized or not lyophilized).

The following are exemplary kit reagent components useful for the detection of SARS-CoV-2, a RNA virus, using one of the novel Cas12 proteins of the disclosure (Cas12a.1, Cas12p, and Cas12q), exemplified in Example 10.

(1) Lyophilized reaction mix containing reagents, SARS-CoV-2 primer sets and enzymes for reverse transcription and loop-mediated isothermal amplification (RT-LAMP) of a gene of diseasSARS-CoV-2 genome.

(2) Lyophilized reaction mix containing reagents, control RNAse P primer sets and enzymes for reverse transcription and RT-LAMP amplification of human housekeeping gene RNAse P.

(3) Lyophilized reaction mix containing reagents and Cas12p-gRNA RNP complexes for detection of a SARS-CoV-2 amplification product. Such mix may also include a labeled reporter, e.g. a 5′FAM-3′Quencher ssRNA-based oligonucleotide reporter, or a 5′FAM-3′Quencher single stranded DNA/RNA chimera-based oligonucleotide reporter.

(4) Lyophilized reaction mix containing reagents and Cas12p-gRNA RNP complexes for detection of RNAse P amplification product. Such mix may also include a labeled reporter, e.g. a 5′FAM-3′Quencher RNA-based oligonucleotide reporter.

FIG. 23 shows an exemplary strip of lyophilized beads of the disclosure included in exemplary kits. Each bead can be resuspended with water, and used for a detection assay. Exemplary beads each comprise a CRISPR protein (e.g. Cas12p), a gRNA for a desired target (e.g. gRNA for SARS-CoV-2), a labeled reporter, a buffer, and nuclease free water.

### VII. Enumerated Embodiments

Provided herein are illustrative, non-limiting, enumerated embodiments of the disclosure.

Embodiment 1. An engineered system comprising:

a. a Cas9.1, Cas9.2, Cas9.3, or Cas9.4 protein, or a nucleic acid encoding the a Cas9.1, Cas9.2, Cas9.3, or Cas9.4 protein; and

b. a Cas9.1, Cas9.2, Cas9.3, or Cas9.4 guide RNA (gRNA), or a nucleic acid encoding the Cas9.1, Cas9.2, Cas9.3, or Cas9.4 gRNA, wherein the gRNA and the Cas9.1, Cas9.2, Cas9.3, or Cas9.4 protein do not naturally occur together, wherein the gRNA is capable of hybridizing to a target sequence in a target DNA, and the gRNA is capable of forming a complex with the Cas9.1, Cas9.2, Cas9.3, or Cas9.45 protein.

Embodiment 2. The system of embodiment 1, comprising:

a. a Cas9.1, Cas9.2, Cas9.3, Cas9.4 protein; and

b. a Cas9.1, Cas9.2, Cas9.3, or Cas9.4 gRNA.

Embodiment 3. The system of embodiment 1, comprising:

a. a nucleic acid encoding the Cas9.1, Cas9.2, Cas9.3, or Cas9.4 protein; and

b. a nucleic acid encoding the Cas9.1, Cas9.2, Cas9.3, or Cas9.4 gRNA.

Embodiment 4. The system of any one of embodiments 1 to 3, wherein the gRNA is a single-molecule gRNA.

Embodiment 5. The system of any one of embodiments 1 to 3, wherein the gRNA is a dual-molecule gRNA.

Embodiment 6. The system of any one of embodiments 1 to 5, wherein the Cas9.1 protein comprises the amino acid sequence of SEQ ID NO: 1, or at least 70% sequence identity thereto.

Embodiment 7. The system of any one of embodiments 1 to 5, wherein the Cas9.2 protein comprises the amino acid sequence of SEQ ID NO: 2 or at least 70% sequence identity thereto.

Embodiment 8. The system of any one of embodiments 1 to 5, wherein the Cas9.3 protein comprises the amino acid sequence of SEQ ID NO: 10, or at least 70% sequence identity thereto.

Embodiment 9. The system of any one of embodiments 1 to 5, wherein the Cas9.4 protein comprises the amino acid sequence of SEQ ID NO: 11, or at least 70% sequence identity thereto.

Embodiment 10. The system of any one of embodiments 1 to 7, wherein the target sequence is a sequence of a target provided in any of Tables 6a-6f.

Embodiment 11. The system of any one of embodiments 1 to 7, wherein the target sequence is a sequence of a human.

Embodiment 12. The system of any one of embodiments 1 to 7, wherein the target sequence is a sequence of a non-human primate.

Embodiment 13. The system of any one of embodiments 1 to 12, wherein the Cas9.1, Cas9.2, Cas9.3, or Cas9.4 protein is a catalytically active protein.

Embodiment 14. The system of embodiment 13, wherein the Cas9.1, Cas9.2, Cas9.3, or Cas9.4 protein cleaves at a site distal to the target sequence.

Embodiment 15. The system of any one of embodiments 1 to 12, wherein the Cas9.1, Cas9.2, Cas9.3, or Cas9.4 protein is a catalytically dead protein.

Embodiment 16. The system of any one of embodiments 1 to 12, wherein the Cas9.1, Cas9.2, Cas9.3, or Cas9.4 protein comprises nickase activity.

Embodiment 17. An engineered system comprising:

a. a Class 2 Type V CRISPR-Cas RNA-guided endonuclease protein; and

b. a single guide RNA (gRNA),

wherein the gRNA and the Class 2 Type V CRISPR-Cas RNA-guided endonuclease protein do not naturally occur together, wherein the gRNA is capable of hybridizing to a target sequence in a target DNA, wherein the gRNA is capable of forming a complex with the Class 2 Type V CRISPR-Cas RNA-guided endonuclease protein, and wherein the Class 2 Type V CRISPR-Cas RNA-guided endonuclease protein possesses collateral activity and is capable of collaterally cleaving a single stranded polynucleotide comprising RNA without a tracrRNA.

Embodiment 18. The system of embodiment 17, wherein the Class 2 Type V CRISPR-Cas RNA-guided endonuclease protein comprises the amino acid sequence of SEQ ID NO: 4, or at least 70% sequence identity thereto.

Embodiment 19. The system of any one of embodiments 17 to 18, wherein the target sequence is a sequence of a target provided in any of Tables 6a-6f.

Embodiment 20. The system of any one of embodiments 17 to 18, wherein the target sequence is a sequence of a human.

Embodiment 21. The system of any one of embodiments 17 to 18, wherein the target sequence is a sequence of a non-human primate.

Embodiment 22. The system of any one of embodiments 17 to 18, wherein the target sequence is a bacterial or viral sequence.

Embodiment 23. The system of any one of embodiments 17 to 22, wherein the Class 2 Type V CRISPR-Cas RNA-guided endonuclease protein is capable of collaterally cleaving a single stranded RNA.

Embodiment 24. The system of any one of embodiments 17 to 22, wherein the Class 2 Type V CRISPR-Cas RNA-guided endonuclease protein is capable of collaterally cleaving a single stranded DNA/RNA hybrid.

Embodiment 25. An engineered system comprising:

a. a Cas12a.1, Cas12p, or Cas12q protein, or a nucleic acid encoding the Cas12a.1, Cas12p, or Cas12q protein; and

b. a Cas12a.1, Cas12p, or Cas12q gRNA, or a nucleic acid encoding a Cas12a.1, Cas12p, or Cas12q gRNA,

wherein the gRNA and the Cas12a.1, Cas12p, or Cas12q protein do not naturally occur together, wherein the gRNA is capable of hybridizing to a target sequence in a target DNA, and the gRNA is capable of forming a complex with the Cas12a.1, Cas12p, or Cas12q protein.

Embodiment 26. The system of embodiment 25, comprising:

a. a Cas12a.1, Cas12p, or Cas12q protein; and

b. a Cas12a.1, Cas12p, or Cas12q gRNA.

Embodiment 27. The system of embodiment 25, comprising:

a. a nucleic acid encoding the Cas12a.1, Cas12p, or Cas12q protein; and

b. a nucleic acid encoding a Cas12a.1, Cas12p, or Cas12q gRNA.

Embodiment 28. The system of any one of embodiments 25 to 27, wherein the Cas12a.1 protein comprises the amino acid sequence of SEQ ID NO: 3, or at least 70% sequence identity thereto.

Embodiment 29. The system of any one of embodiments 25 to 27, wherein the Cas12p protein comprises the amino acid sequence of SEQ ID NO: 4, or at least 70% sequence identity thereto.

Embodiment 30. The system of any one of embodiments 25 to 27, wherein the Cas12q protein comprises the amino acid sequence of SEQ ID NO: 222, or at least 70% sequence identity thereto.

Embodiment 31. The system of any one of embodiments 25 to 27, wherein the Cas12q protein comprises the amino acid sequence of SEQ ID NO: 5, or at least 70% sequence identity thereto.

Embodiment 32. The system of any one of embodiments 25 to 31, wherein the target sequence is a sequence of a target provided in any of Tables 6a-6f.

Embodiment 33. The system of any one of embodiments 25 to 31, wherein the target sequence is a sequence of a human.

Embodiment 34. The system of any one of embodiments 25 to 31, wherein the target sequence is a sequence of a non-human primate.

Embodiment 35. The system of any one of embodiments 25 to 31, wherein the target sequence is a bacterial or viral sequence.

Embodiment 36. The system of any one of embodiments 25 to 34, wherein the Cas12a.1, Cas12p, or Cas12q protein is a catalytically active Cas12a.1, Cas12p, or Cas12q protein.

Embodiment 37. The system of embodiment 36, wherein the Cas12a.1, Cas12p, or Cas12q protein cleaves at a site distal to the target sequence.

Embodiment 38. The system of any one of embodiments 25 to 34, wherein the Cas12a.1, Cas12p, or Cas12q protein is a catalytically dead Cas12a.1, Cas12p, or Cas12q protein.

Embodiment 39. The system of any one of embodiments 25 to 34, wherein the Cas12a.1, Cas12p, or Cas12q protein comprises nickase activity.

Embodiment 40. An engineered single-molecule gRNA, comprising:

a. a targeter-RNA comprising a spacer sequence that is capable of hybridizing with a target sequence in a target DNA; and

b. an activator-RNA that is capable of hybridizing with the targeter-RNA to form a double-stranded RNA duplex, the activator-RNA comprising a activator-RNA,

wherein the targeter-RNA and the activator-RNA are covalently linked to one another, wherein the single-molecule gRNA is capable of forming a complex with a Cas9.1, Cas9.2, Cas9.3, or Cas9.4 protein, and wherein hybridization of the spacer sequence to the target sequence is capable of targeting the Cas9.1, Cas9.2, Cas9.3, or Cas9.4 protein to the target DNA.

Embodiment 41. The gRNA of embodiment 40, wherein the targeter-RNA and the activator-RNA are arranged in a 5′ to 3′ orientation.

Embodiment 42. The gRNA of embodiment 40, wherein the activator-RNA and the targeter-RNA are arranged in a 5′ to 3′ orientation.

Embodiment 43. The gRNA of any one of embodiments 40 to 42, wherein the targeter-RNA and the activator-RNA are covalently linked to one another via a linker.

Embodiment 44. The gRNA of ay one of embodiments 40 to 43, wherein the single-molecule gRNA comprises one or more sequence modifications compared to a sequence of a corresponding wild type tracrRNA and/or crRNA.

Embodiment 45. The gRNA of ay one of embodiments 40 to 44, wherein the targeter-RNA comprises a spacer sequence of about 10-50 nucleotides that have 100% complementarity to a sequence in the target DNA.

Embodiment 46. The gRNA of any one of embodiments 40 to 44, wherein the targeter-RNA comprises a spacer sequence of about 10-50 nucleotides that have less than 100% complementarity to a sequence in the target DNA.

Embodiment 47. The gRNA of any one of embodiments 40 to 46, wherein the target sequence is a sequence of a target provided in any of Tables 6a-6f.

Embodiment 48. The gRNA of any one of embodiments 40 to 47, wherein the Cas9.1 protein comprises the sequence of SEQ ID NO: 1 or a sequence with at least 70% sequence identity thereto.

Embodiment 49. The gRNA of any one of embodiments 40 to 47, wherein the Cas9.2 protein comprises the sequence of SEQ ID NO: 2 or a sequence with at least 70% sequence identity thereto.

Embodiment 50. The gRNA of any one of embodiments 40 to 47, wherein the Cas9.3 protein comprises the sequence of SEQ ID NO: 10 or a sequence with at least 70% sequence identity thereto.

Embodiment 51. The gRNA of any one of embodiments 40 to 47, wherein the Cas9.4 protein comprises the sequence of SEQ ID NO: 11 or a sequence with at least 70% sequence identity thereto.

Embodiment 52. An engineered single-molecule gRNA, comprising the scaffold sequence of SEQ ID NO: 116 or SEQ ID NO: 117 and a spacer sequence that is capable of hybridizing with a target sequence in a target DNA.

Embodiment 53. The gRNA of embodiment 52, wherein the target DNA comprises viral DNA, plant DNA, fungal DNA, or bacterial DNA.

Embodiment 54. The gRNA of embodiment 52, wherein the target sequence is a sequence of a target provided in any of Tables 6a-6f.

Embodiment 55. The gRNA of embodiment 52, wherein the target is a coronavirus.

Embodiment 56. The gRNA of embodiment 52, wherein the target is a SARS-CoV-2 virus.

Embodiment 57. The gRNA of embodiment 52, wherein the target DNA is cDNA, and has been obtained by reverse transcription.

Embodiment 58. A method of modifying a target DNA, the method comprising contacting the target DNA with any one of the systems of embodiments 1 to 39, wherein the gRNA hybridizes with the target sequence whereby modification of the target DNA occurs.

Embodiment 59. The method of embodiment 58, wherein the target DNA is extrachromosomal DNA.

Embodiment 60. The method of embodiment 58, wherein the target DNA is part of a chromosome.

Embodiment 61. The method of embodiment 58, wherein the target DNA is part of a chromosome in vitro.

Embodiment 62. The method of embodiment 58, wherein the target DNA is part of a chromosome in vivo.

Embodiment 63. The method of embodiment 58, wherein the target DNA is outside a cell.

Embodiment 64. The method of embodiment 58, wherein the target DNA is inside a cell.

Embodiment 65. The method of embodiment 64, wherein the target DNA comprises a gene and/or its regulatory region.

Embodiment 66. The method of embodiment 64 or 65, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

Embodiment 67. The method of any of the embodiments of 58 to 66, wherein the modifying comprises introducing a double strand break in the target DNA.

Embodiment 68. The method of any of the embodiments of 58 to 67, wherein the contacting occurs under conditions that are permissive for non-homologous end joining or homology-directed repair.

Embodiment 69. The method of any of the embodiments of 58 to 67, wherein the contacting the target DNA with a donor polynucleotide, wherein the donor polynucleotide, a portion of the donor polynucleotide, a copy of the donor polynucleotide, or a portion of a copy of the donor polynucleotide integrates into the target DNA.

Embodiment 70. The method of any of the embodiments of 58 to 67, wherein the method does not comprise contacting the cell with a donor polynucleotide, or wherein the target DNA is modified such that nucleotides within the target DNA are deleted.

Embodiment 71. A method of detecting a target DNA in a sample, the method comprising:

a. contacting the sample with:
i. a Cas12a.1, Cas12p, or Cas12q protein;
ii. a Cas12a.1, Cas12p, or Cas12q gRNA comprising a spacer sequence that is capable of hybridizing with a target sequence in a target DNA; and
iii. a labeled detector that does not hybridize with the spacer sequence of the gRNA; and

b. measuring a detectable signal produced by cleavage of the labeled detector by the Cas12a.1, Cas12p, or Cas12q protein, thereby detecting the target DNA.

Embodiment 72. The method of embodiment 71, wherein the labeled detector comprises a labeled single stranded DNA.

Embodiment 73. The method of embodiment 71, wherein the labeled detector comprises a labeled RNA.

Embodiment 74. The method of embodiment 72, wherein the labeled RNA is a single stranded RNA.

Embodiment 75. The method of embodiment 71, wherein the labeled detector comprises a labeled single stranded DNA/RNA chimera.

Embodiment 76. The method of any one of embodiments 71 to 75, wherein the labeled detector comprises one or more modified nucleotides.

Embodiment 77. The method of any one of embodiments 71 to 76, comprising contacting the sample with a precursor gRNA array, wherein the Cas12a.1, Cas12p, or Cas12q protein cleaves the precursor gRNA array to produce said gRNA.

Embodiment 78. The method of any one of embodiments 71 to 77, wherein the target DNA is single stranded.

Embodiment 79. The method of any one of embodiments 71 to 78, wherein the target DNA is double stranded.

Embodiment 80. The method of any one of embodiments 71 to 79, wherein the target DNA is viral DNA, plant DNA, fungal DNA, or bacterial DNA.

Embodiment 81. The method of embodiment 80, wherein the target sequence is a sequence of a target provided in any of Tables 6a-6f.

Embodiment 82. The method of embodiment 81, wherein the target is a coronavirus.

Embodiment 83. The method of embodiment 82, wherein the target is a SARS-CoV-2 virus.

Embodiment 84. The method of any one of embodiments 71 to 83, wherein the target DNA is cDNA, and has been obtained by reverse transcription.

Embodiment 85. The method of any one of embodiments 71 to 79, wherein the target DNA is from a human cell.

Embodiment 86. The method of embodiment 85, wherein the target DNA is human fetal or cancer cell DNA.

Embodiment 87. The method of any one of embodiments 71 to 86, wherein the protein is Cas12a.1 comprising the amino acid sequence of SEQ ID NO: 3, or at least 70% sequence identity thereto.

Embodiment 88. The method of any one of embodiments 71 to 86, wherein the protein is Cas12p comprising the amino acid sequence of SEQ ID NO: 4, or at least 70% sequence identity thereto.

Embodiment 89. The method of any one of embodiments 71 to 86, wherein the protein is Cas12p comprising the amino acid sequence of SEQ ID NO: 222, or at least 70% sequence identity thereto.

Embodiment 90. The method of any one of embodiments 71 to 86, wherein the protein is Cas12q comprising the amino acid sequence of SEQ ID NO: 5, or at least 70% sequence identity thereto.

Embodiment 91. The method of any one of embodiments 71 to 87, wherein the sample comprises DNA from a cell lysate.

Embodiment 92. The method of any one of embodiments 71 to 87, wherein the sample comprises cells.

Embodiment 93. The method of any one of embodiments 71 to 87, wherein the sample is a urine, blood, serum, plasma, lymphatic fluid, cerebrospinal fluid, saliva, nasopharyngeal, oropharyngeal, nasopharyngeal/oropharyngeal, aspirate, or biopsy sample.

Embodiment 94. The method of any one of embodiments 71 to 93, comprising determining an amount of the target DNA present in the sample.

Embodiment 95. The method of embodiment 94, wherein said measuring a detectable signal comprises one or more of: visual based detection, sensor based detection, color detection, gold nanoparticle based detection, fluorescence polarization, colloid phase transition/dispersion, electrochemical detection, and semiconductor-based sensing.

Embodiment 96. The method of any one of embodiments 71 to 95, wherein the labeled detector comprises a modified nucleobase, a modified sugar moiety, and/or a modified nucleic acid linkage.

Embodiment 97. The method of any one of embodiments 71 to 96, further comprising detecting a positive control target DNA in a positive control sample, the detecting comprising:

a. contacting the positive control sample with:
i. the Cas12a.1, Cas12p, or Cas12q protein;
ii. a positive control gRNA comprising: a region that binds to the Cas12a.1, Cas12p, or Cas12q protein, and a positive control spacer sequence that hybridizes with the positive control target DNA; and
iii. a labeled detector that does not hybridize with the positive control spacer sequence of the positive control gRNA; and

b. measuring a detectable signal produced by cleavage of the labeled detector by the Cas12a.1, Cas12p, or Cas12q protein, thereby detecting the positive control target DNA.

Embodiment 98. The method of any one of embodiments 71 to 97, wherein the detectable signal is detectable in less than 15, 30, 45, 60, 90, 120, 150, 180, 210, or 240 minutes.

Embodiment 99. The method of any one of embodiments 71 to 98, further comprising amplifying the target DNA in the sample by loop-mediated isothermal amplification (LAMP), helicase-dependent amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), or isothermal multiple displacement amplification (IMDA).

Embodiment 100. The method of any one of embodiments 71 to 99, wherein target DNA in the sample is present at a concentration of less than 100 uM.

Embodiment 101. A protein comprising an amino acid sequence with 70%-99.5% homology to SEQ ID NO: 1, 2, 3, 4, 5, 10, 11, or 222.

Embodiment 102. A protein of embodiment 101, wherein the sequence of the protein has been deduced bioinformatically.

Embodiment 103. A composition comprising any of the proteins of embodiment 101, and optionally a pharmaceutically acceptable carrier.

Embodiment 104. A composition comprising any of the proteins of embodiment 101, optionally comprising a pharmaceutically acceptable carrier, a nucleic acid stabilizing buffer and/or or a protein stabilizing buffer.

Embodiment 105. A composition comprising any of the proteins of embodiment 101, wherein the protein is lyophilized, and optionally further comprises any one or more of a labeled detector, a reverse transcriptase enzyme, and reagents for loop-mediated isothermal amplification.

Embodiment 106. A DNA polynucleotide comprising a nucleotide sequence that encodes any of the proteins of embodiment 101.

Embodiment 107. A recombinant expression vector comprising the DNA polynucleotide of embodiment 106.

Embodiment 108. The recombinant expression vector of embodiment 107, wherein the nucleotide sequence encoding the single protein is operably linked to a promoter.

Embodiment 109. A host cell comprising the DNA polynucleotide of any one of embodiments 106 to 108.

Embodiment 110. A pharmaceutical composition comprising any of the engineered systems of embodiments 1 to 39, and optionally a pharmaceutically acceptable carrier.

Embodiment 111. A composition comprising any of the engineered systems of embodiments 1 to 39, and optionally comprising a nucleic acid stabilizing buffer and/or or a protein stabilizing buffer.

Embodiment 112. A pharmaceutical composition comprising any of the single molecule gRNAs of embodiments 40 to 57, and optionally pharmaceutically acceptable carrier.

Embodiment 113. A composition comprising any of the singe molecule gRNAs of embodiments 40 to 51, and optionally a nucleic acid stabilizing buffer and/or or a protein stabilizing buffer.

Embodiment 114. A DNA polynucleotide comprising a nucleotide sequence that encodes any of the nucleic acids of embodiments 3, 27, or the gRNAs of embodiments 40 to 51.

Embodiment 115. A recombinant expression vector comprising the DNA polynucleotide of embodiment 114.

Embodiment 116. The recombinant expression vector of embodiment 115, wherein the nucleotide sequence encoding the single gRNA is operably linked to a promoter.

Embodiment 117. A host cell comprising the DNA polynucleotide of any one of embodiments 114 to 116.

Embodiment 118. A kit comprising one or more components of any of the engineered systems of embodiments 1 to 39.

Embodiment 119. The kit of embodiment 118, wherein one or more components are lyophilized.

Embodiment 120. The kit of any one of embodiments 118 to 119, wherein the one or more components comprise Cas12p, a labeled RNA reporter, and a gRNA directed to SARS-CoV-2.

Embodiment 121. A method of isolating a Class 2 Type II or Class 2 Type V CRISPR-Cas protein from a metagenomics sample comprising the use of a bioinformatics-based method.

Embodiment 122. The method of embodiment 121, wherein the Class 2 Type II or Class 2 Type V CRISPR-Cas protein is selected from the group consisting of SEQ ID NO: 1, 2, 3, 4, 5, 10, 11, and 222.

### EXAMPLES

The following examples are included for illustrative purposes and are not intend to limit the scope of the invention.

### Example 1: Identification and Validation of Novel Class II Type II and V Endonucleases

### Class 2 Type II and Type V CRISPR-Cas Loci Identification

Metagenome sequences were obtained from NCBI, and compiled to construct a database of putative CRISPR-Cas loci. CRISPR arrays were identified using CrisprCasFinder software. The criteria of filtering were putative Class II type II and V effectors >500 aa, which were adjacent to cas genes and CRISPR arrays. Sequences were aligned with Clustal Omega using HMM profiles. The novel Cas9.1, Cas9.2, Cas9.3, Cas9.4, Cas12a.1, Cas12p and Cas12q proteins described herein were identified.

### Generation of Expression Plasmids and Non-Coding Elements

Minimal conditions to validate the Cas proteins were established into a cloning strategy. Minimal CRISPR loci were designed by removing acquisition proteins and generating minimal arrays with a single spacer (Sp1). The natural Sp1 sequence was replaced by a known specific target sequence with the length of the naturally occurring sequence (GTGGCAGCTCAAAAATTGGCTACAAAACCAGTT; SEQ ID NO: 118) for target detection and PAM screening assays. The E. coli codon-optimized protein sequences of CRISPR effectors and/or accessory proteins were placed under the transcriptional control of lac and IPTG-inducible T7 promoters into a pET-based expression vector (EMD-Millipore).

### Artificial Synthesis

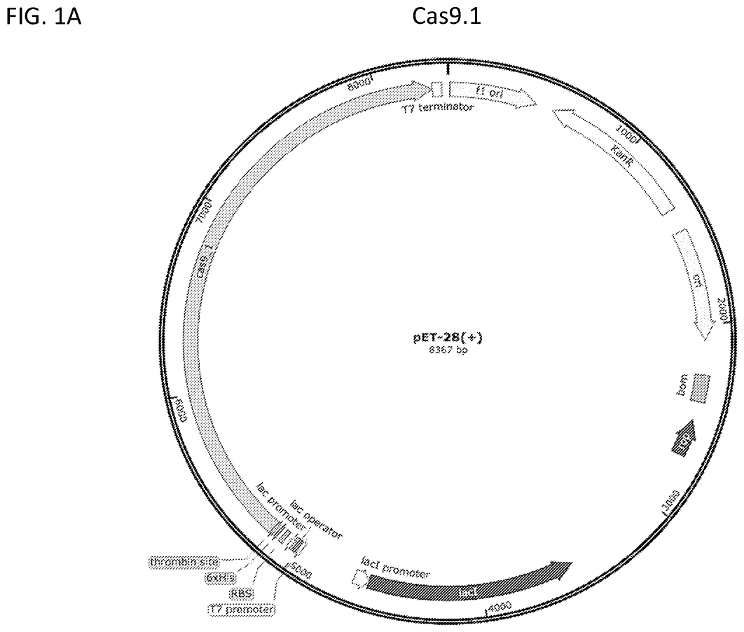

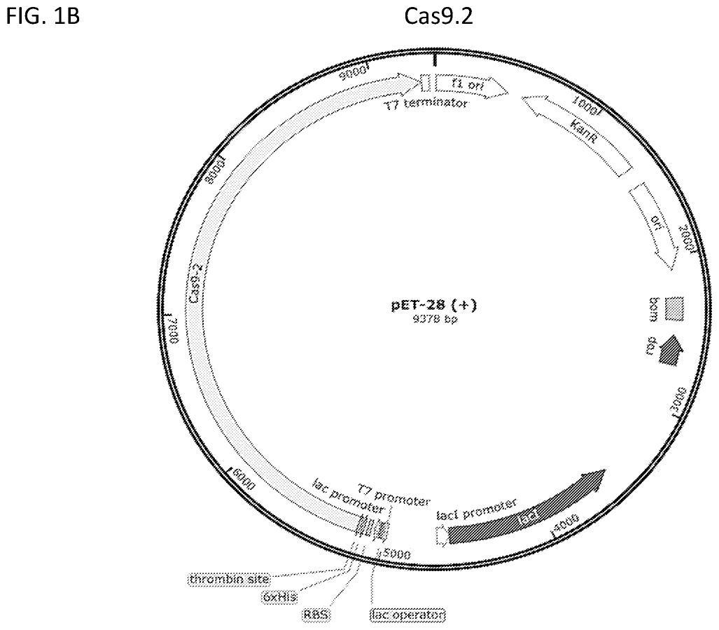

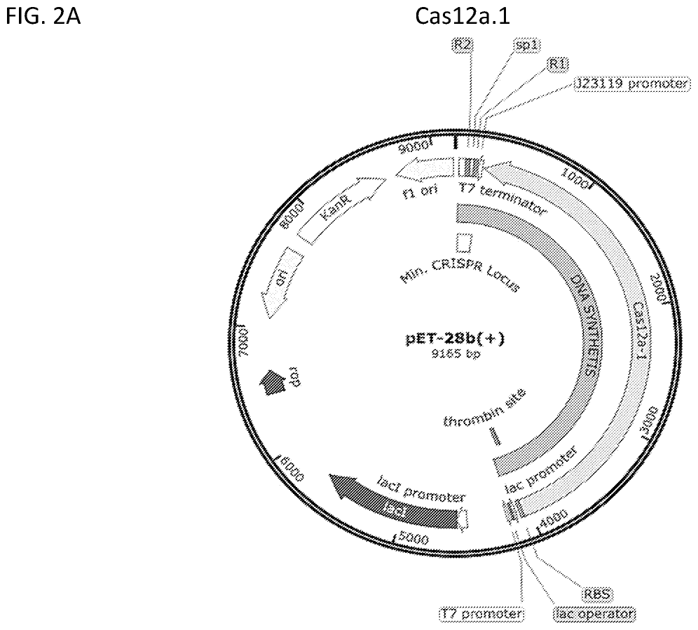

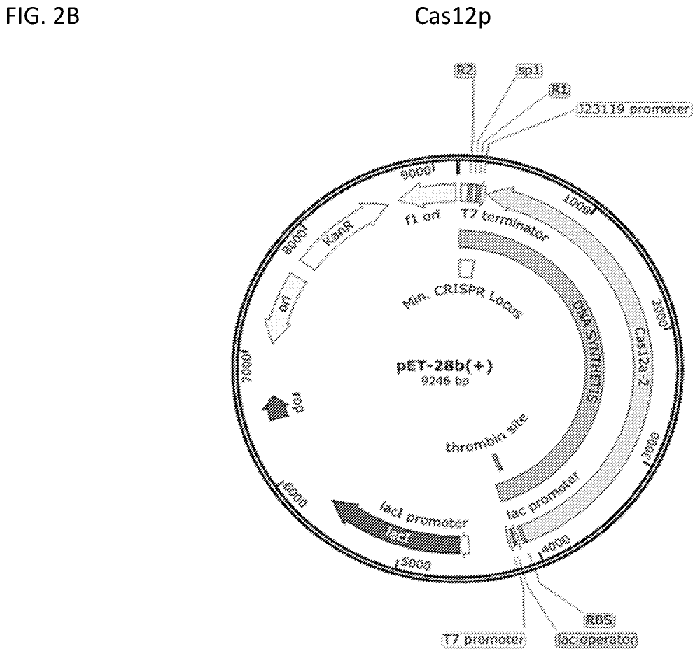

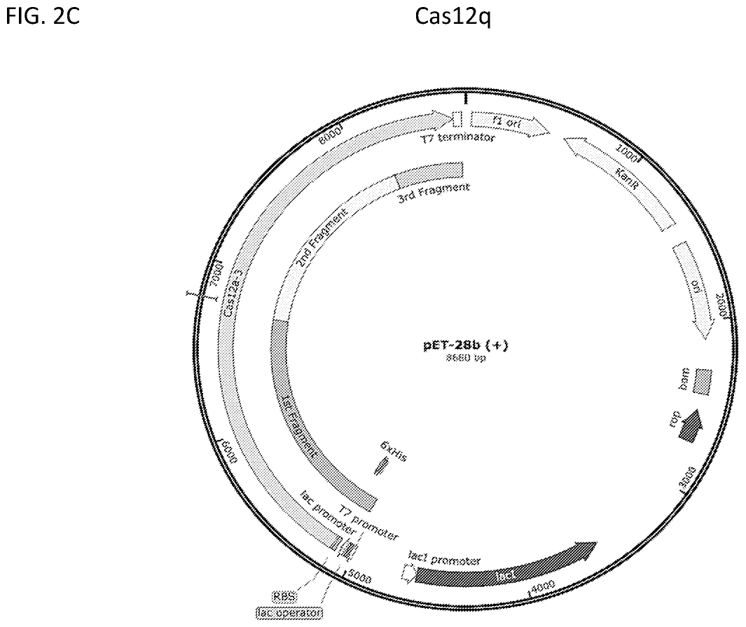

For Cas12a.1, Cas12p, Cas9.1 and Cas9.2, expression vectors were artificially synthesized. The effector plasmid codon optimization, synthesis, and cloning were generated by a provider (GeneScript). To consider both putative transcription directions, flanking restriction sites were added in the CRISPR array to clone a DNA fragment (IDT). This was done with the same element in the opposite direction to create a second construct variant. FIGS. 1A-1B show expression vector maps for Cas9.1 and Cas9.2. FIGS. 2A-2C show expression vector maps for Cas12a.1, Cas12p, and Cas12q. Vector sequences are provided in Table 8.

TABLE 8
Expression vector sequences

| Protein | Vector Sequence |
|---|---|
| Cas12a.1 | TGGCGAATGGGACGCGCCCTGTAGCGGCGCATTAAGCGCG |
| | GCGGGTGTGGTGGTTACGCGCAGCGTGACCGCTACACTTG |
| | CCAGCGCCCTAGCGCCCGCTCCTTTCGCTTTCTTCCCTTC |
| | CTTTCTCGCCACGTTCGCCGGCTTTCCCCGTCAAGCTCTA |
| | AATCGGGGGCTCCCTTTAGGGTTCCGATTTAGTGCTTTAC |
| | GGCACCTCGACCCCAAAAAACTTGATTAGGGTGATGGTTC |
| | ACGTAGTGGGCCATCGCCCTGATAGACGGTTTTTCGCCCT |
| | TTGACGTTGGAGTCCACGTTCTTTAATAGTGGACTCTTGT |
| | TCCAAACTGGAACAACACTCAACCCTATCTCGGTCTATTC |
| | TTTTGATTTATAAGGGATTTTGCCGATTTCGGCCTATTGG |
| | TTAAAAAATGAGCTGATTTAACAAAAATTTAACGCGAATT |
| | TTAACAAAATATTAACGTTTACAATTTCAGGTGGCACTTT |
| | TCGGGGAAATGTGCGCGGAACCCCTATTTGTTTATTTTTC |
| | TAAATACATTCAAATATGTATCCGCTCATGAATTAATTCT |
| | TAGAAAAACTCATCGAGCATCAAATGAAACTGCAATTTAT |
| | TCATATCAGGATTATCAATACCATATTTTTGAAAAAGCCG |
| | TTTCTGTAATGAAGGAGAAAACTCACCGAGGCAGTTCCAT |
| | AGGATGGCAAGATCCTGGTATCGGTCTGCGATTCCGACTC |
| | GTCCAACATCAATACAACCTATTAATTTCCCCTCGTCAAA |
| | AATAAGGTTATCAAGTGAGAAATCACCATGAGTGACGACT |
| | GAATCCGGTGAGAATGGCAAAAGTTTATGCATTTCTTTCC |
| | AGACTTGTTCAACAGGCCAGCCATTACGCTCGTCATCAAA |
| | ATCACTCGCATCAACCAAACCGTTATTCATTCGTGATTGC |
| | GCCTGAGCGAGACGAAATACGCGATCGCTGTTAAAAGGAC |
| | AATTACAAACAGGAATCGAATGCAACCGGCGCAGGAACAC |
| | TGCCAGCGCATCAACAATATTTTCACCTGAATCAGGATAT |
| | TCTTCTAATACCTGGAATGCTGTTTTCCCGGGGATCGCAG |
| | TGGTGAGTAACCATGCATCATCAGGAGTACGGATAAAATG |
| | CTTGATGGTCGGAAGAGGCATAAATTCCGTCAGCCAGTTT |
| | AGTCTGACCATCTCATCTGTAACATCATTGGCAACGCTAC |
| | CTTTGCCATGTTTCAGAAACAACTCTGGCGCATCGGGCTT |
| | CCCATACAATCGATAGATTGTCGCACCTGATTGCCCGACA |
| | TTATCGCGAGCCCATTTATACCCATATAAATCAGCATCCA |
| | TGTTGGAATTTAATCGCGGCCTAGAGCAAGACGTTTCCCG |
| | TTGAATATGGCTCATAACACCCCTTGTATTACTGTTTATG |
| | TAAGCAGACAGTTTTATTGTTCATGACCAAAATCCCTTAA |
| | CGTGAGTTTTCGTTCCACTGAGCGTCAGACCCCGTAGAAA |
| | AGATCAAAGGATCTTCTTGAGATCCTTTTTTTCTGCGCGT |
| | AATCTGCTGCTTGCAAACAAAAAAACCACCGCTACCAGCG |
| | GTGGTTTGTTTGCCGGATCAAGAGCTACCAACTCTTTTTC |
| | CGAAGGTAACTGGCTTCAGCAGAGCGCAGATACCAAATAC |
| | TGTCCTTCTAGTGTAGCCGTAGTTAGGCCACCACTTCAAG |
| | AACTCTGTAGCACCGCCTACATACCTCGCTCTGCTAATCC |
| | TGTTACCAGTGGCTGCTGCCAGTGGCGATAAGTCGTGTCT |
| | TACCGGGTTGGACTCAAGACGATAGTTACCGGATAAGGCG |
| | CAGCGGTCGGGCTGAACGGGGGGTTCGTGCACACAGCCCA |
| | GCTTGGAGCGAACGACCTACACCGAACTGAGATACCTACA |
| | GCGTGAGCTATGAGAAAGCGCCACGCTTCCCGAAGGGAGA |
| | AAGGCGGACAGGTATCCGGTAAGCGGCAGGGTCGGAACAG |
| | GAGAGCGCACGAGGGAGCTTCCAGGGGGAAACGCCTGGTA |
| | TCTTTATAGTCCTGTCGGGTTTCGCCACCTCTGACTTGAG |
| | CGTCGATTTTTGTGATGCTCGTCAGGGGGGCGGAGCCTAT |
| | GGAAAAACGCCAGCAACGCGGCCTTTTTACGGTTCCTGGC |
| | CTTTTGCTGGCCTTTTGCTCACATGTTCTTTCCTGCGTTA |
| | TCCCCTGATTCTGTGGATAACCGTATTACCGCCTTTGAGT |
| | GAGCTGATACCGCTCGCCGCAGCCGAACGACCGAGCGCAG |
| | CGAGTCAGTGAGCGAGGAAGCGGAAGAGCGCCTGATGCGG |
| | TATTTTCTCCTTACGCATCTGTGCGGTATTTCACACCGCA |
| | TATATGGTGCACTCTCAGTACAATCTGCTCTGATGCCGCA |
| | TAGTTAAGCCAGTATACACTCCGCTATCGCTACGTGACTG |
| | GGTCATGGCTGCGCCCCGACACCCGCCAACACCCGCTGAC |
| | GCGCCCTGACGGGCTTGTCTGCTCCCGGCATCCGCTTACA |
| | GACAAGCTGTGACCGTCTCCGGGAGCTGCATGTGTCAGAG |
| | GTTTTCACCGTCATCACCGAAACGCGCGAGGCAGCTGCGG |
| | TAAAGCTCATCAGCGTGGTCGTGAAGCGATTCACAGATGT |
| | CTGCCTGTTCATCCGCGTCCAGCTCGTTGAGTTTCTCCAG |
| | AAGCGTTAATGTCTGGCTTCTGATAAAGCGGGCCATGTTA |
| | AGGGCGGTTTTTTCCTGTTTGGTCACTGATGCCTCCGTGT |
| | AAGGGGGATTTCTGTTCATGGGGGTAATGATACCGATGAA |
| | ACGAGAGAGGATGCTCACGATACGGGTTACTGATGATGAA |
| | CATGCCCGGTTACTGGAACGTTGTGAGGGTAAACAACTGG |
| | CGGTATGGATGCGGCGGGACCAGAGAAAAATCACTCAGGG |
| | TCAATGCCAGCGCTTCGTTAATACAGATGTAGGTGTTCCA |
| | CAGGGTAGCCAGCAGCATCCTGCGATGCAGATCCGGAACA |
| | TAATGGTGCAGGGCGCTGACTTCCGCGTTTCCAGACTTTA |
| | CGAAACACGGAAACCGAAGACCATTCATGTTGTTGCTCAG |
| | GTCGCAGACGTTTTGCAGCAGCAGTCGCTTCACGTTCGCT |
| | CGCGTATCGGTGATTCATTCTGCTAACCAGTAAGGCAACC |
| | CCGCCAGCCTAGCCGGGTCCTCAACGACAGGAGCACGATC |
| | ATGCGCACCCGTGGGGCCGCCATGCCGGCGATAATGGCCT |
| | GCTTCTCGCCGAAACGTTTGGTGGCGGGACCAGTGACGAA |
| | GGCTTGAGCGAGGGCGTGCAAGATTCCGAATACCGCAAGC |
| | GACAGGCCGATCATCGTCGCGCTCCAGCGAAAGCGGTCCT |
| | CGCCGAAAATGACCCAGAGCGCTGCCGGCACCTGTCCTAC |
| | GAGTTGCATGATAAAGAAGACAGTCATAAGTGCGGCGACG |
| | ATAGTCATGCCCCGCGCCCACCGGAAGGAGCTGACTGGGT |
| | TGAAGGCTCTCAAGGGCATCGGTCGAGATCCCGGTGCCTA |
| | ATGAGTGAGCTAACTTACATTAATTGCGTTGCGCTCACTG |
| | CCCGCTTTCCAGTCGGGAAACCTGTCGTGCCAGCTGCATT |
| | AATGAATCGGCCAACGCGCGGGGAGAGGCGGTTTGCGTAT |
| | TGGGCGCCAGGGTGGTTTTTCTTTTCACCAGTGAGACGGG |
| | CAACAGCTGATTGCCCTTCACCGCCTGGCCCTGAGAGAGT |
| | TGCAGCAAGCGGTCCACGCTGGTTTGCCCCAGCAGGCGAA |
| | AATCCTGTTTGATGGTGGTTAACGGCGGGATATAACATGA |
| | GCTGTCTTCGGTATCGTCGTATCCCACTACCGAGATATCC |
| | GCACCAACGCGCAGCCCGGACTCGGTAATGGCGCGCATTG |
| | CGCCCAGCGCCATCTGATCGTTGGCAACCAGCATCGCAGT |
| | GGGAACGATGCCCTCATTCAGCATTTGCATGGTTTGTTGA |
| | AAACCGGACATGGCACTCCAGTCGCCTTCCCGTTCCGCTA |
| | TCGGCTGAATTTGATTGCGAGTGAGATATTTATGCCAGCC |
| | AGCCAGACGCAGACGCGCCGAGACAGAACTTAATGGGCCC |
| | GCTAACAGCGCGATTTGCTGGTGACCCAATGCGACCAGAT |
| | GCTCCACGCCCAGTCGCGTACCGTCTTCATGGGAGAAAAT |
| | AATACTGTTGATGGGTGTCTGGTCAGAGACATCAAGAAAT |
| | AACGCCGGAACATTAGTGCAGGCAGCTTCCACAGCAATGG |
| | CATCCTGGTCATCCAGCGGATAGTTAATGATCAGCCCACT |
| | GACGCGTTGCGCGAGAAGATTGTGCACCGCCGCTTTACAG |
| | GCTTCGACGCCGCTTCGTTCTACCATCGACACCACCACGC |
| | TGGCACCCAGTTGATCGGCGCGAGATTTAATCGCCGCGAC |
| | AATTTGCGACGGCGCGTGCAGGGCCAGACTGGAGGTGGCA |
| | ACGCCAATCAGCAACGACTGTTTGCCCGCCAGTTGTTGTG |
| | CCACGCGGTTGGGAATGTAATTCAGCTCCGCCATCGCCGC |
| | TTCCACTTTTTCCCGCGTTTTCGCAGAAACGTGGCTGGCC |
| | TGGTTCACCACGCGGGAAACGGTCTGATAAGAGACACCGG |
| | CATACTCTGCGACATCGTATAACGTTACTGGTTTCACATT |
| | CACCACCCTGAATTGACTCTCTTCCGGGCGCTATCATGCC |
| | ATACCGCGAAAGGTTTTGCGCCATTCGATGGTGTCCGGGA |
| | TCTCGACGCTCTCCCTTATGCGACTCCTGCATTAGGAAGC |
| | AGCCCAGTAGTAGGTTGAGGCCGTTGAGCACCGCCGCCGC |
| | AAGGAATGGTGCATGCAAGGAGATGGCGCCCAACAGTCCC |
| | CCGGCCACGGGGCCTGCCACCATACCCACGCCGAAACAAG |
| | CGCTCATGAGCCCGAAGTGGCGAGCCCGATCTTCCCCATC |
| | GGTGATGTCGGCGATATAGGCGCCAGCAACCGCACCTGTG |
| | GCGCCGGTGATGCCGGCCACGATGCGTCCGGCGTAGAGGA |
| | TCGAGATCTCGATCCCGCGAAATTAATACGACTCACTATA |
| | GGGGAATTGTGAGCGGATAACAATTCCCCTCTAGATTTAC |
| | ACTTTATGCTTCCGGCTCGTATGTTTCTAGAGAATTCAAA |
| | TAATTTTGTTTAACTTTAAGAAGGAGATTTAAATATGGGG |
| | TCAAGTCATCACCACCACCACCACTCAAGTGGACTAGTAC |
| | CCCGTGGCAGCATGAAAGTTAGCACCTGGGATAGCTTCAC |
| | CAACCAGTACCCGCTGACCAAGACCCTGCGTTTTGAGCTG |
| | AAGCCGGTGGGTAAAACCCTGCAGAAGATCCAAGACCGTA |
| | ACCTGATTACCGAGGACGAACAGCGTCAAAAGGATTTCAA |
| | CAAGGTTAAGAAAATCATGGATGGTTACTACAAGCAGTTC |
| | ATCGAGGAATGCCTGGAAGGCGCGAAGATCCCGCTGAAGA |
| | AACTGGAGGAAAACAACAACGCGTACACCAAACTGAAGAA |
| | AGACCCGTATAACAAGAAACTGCGTGAGGAATACGCGAAG |
| | CTGCAGAAACAACTGCGTAAACTGATCCACGATGAGATTA |
| | ACAAGAAAGAGGAATTCAAGTACCTGTTTAAGAAAGAATT |
| | CATCAAGAAAATTCTGCCGGAATGGCTGGAGAAGAAAGGT |
| | AAGAAAGAGGAACTGAAAGAGATCGAAAAGTTCGACAAAT |
| | GGGTGACCTACTTTAGCGGCTTCTTTAACAACCGTAAGAA |
| | CGTTTTCAGCAGCGACGAGATTAGCACCAGCATGATCTAT |
| | CGTATTGTGAACGATAACCTGCCGAAATTCCTGGACGATG |
| | TTAGCCGTTTTGGTGAAATTACCCGTTACAAGGAGTTCGA |
| | CGCGAACCAGATCGAGGAAAACTTTGAGAGCGAACTGAAC |
| | GGTGAAAAACTGAAGGATTTCTTTAACCTGAAAAACTTCA |
| | ACAACTGCCTGAACCAAGAAGGCATTGAGAAATTTAACCT |
| | GATCATTGGTGGCAAGAGCGAGGAAGGTAATAACAAAATC |
| | AAGGGCCTGAACGAACTGGTGAACGAGCTGGCGCAAAAAC |
| | AAGCGGACAAGAACGAGCAGAAGAAAGTTCGTAAACTGAA |
| | GCTGGCGCCGCTGTTCAAACAAATCCTGAGCGATCGTAAG |
| | AGCAGCAGCTTTGCGTTCGAAAAATTTGAGGAAAACACCG |
| | AGGTGTTCGACGCGATCGATGAATTTTATGACAAGATTAG |
| | CCTGGAGACCCTGAAGAAAATCGAAGCGACCCTGGAGAAA |
| | CTGGAGGAAAAGGACCTGGAACTGGTTTACCTGAAAAACG |
| | ATCGTTGCCTGACCGGTATCAGCCAGGAAGTGTTCGGCGA |
| | CCGTGAGCGTGTTCTGCAAGCGCTGCGTGAATACGCGAAA |
| | ACCGAGCTGGGTCTGAAGACCGATAAGAAAATCGAGAAGT |
| | GGATGAAGAAAGGTCGTTATAGCATCCACGAGATTGAAAG |
| | CGGCCTGAAGAAAATCGGTAGCACCGGCCACCCGATTTGC |
| | AACTACTTCAGCAAACTGGAGGAAAAGAAAACCAACCTGA |
| | TCCAGGAAATTAAGAAAGCGCGTACCGAGTATGAAAAGAT |
| | CAGCGACAAGAAAAAGAAACTGACCGCGGAAAGCCAAGAG |
| | CCGAACGTGGCGCGTATCAAAGCGCTGCTGGATAGCATTA |
| | TGCGTCTGTATCACTTCATCAAGCCGCTGAACATCAACTT |
| | CAAGAACAAGAAAGAGAAGGACAGCGAAGCGCTGGAGACC |
| | GACAACGATTTTTACAACGACTTCGATGAAAGCTTTGCGG |
| | AGCTGGGCAACATCATTCCGCTGTACAACCAAGTGCGTAA |
| | CTATGTTACCCAAAAACCGTTCAGCACCGAGAAATTCAAG |
| | CTGAACTTTGAAAACCCGAAGCTGCTGAGCGGTTGGGACA |
| | AAAACAAGGAAAAAGATTACTATAGCGTGATTCTGCGTAA |
| | AGAGGAAAGCTACTATCTGGCGATCATGACCCCGAAGCAG |
| | AAAAACGTTTTCGACGAGCTGGAACGTCTGCCGGCGGGCA |
| | AAAATTACTTCGAGAAGATCGATTACAAGCTGCTGCCGAC |
| | CCCGGAAAAGAACCTGCCGCGTATCCTGTTCGCGAAGAAA |
| | AACATTAGCTTTTACAAGCCGAGCAAAGAGATCGAAGCGA |
| | TTCGTAACCACAGCGCGCACACCAAACACGGTAACCCGCA |
| | GAACGGCTTCAAGAAACGTGACTTTCGTCTGAGCGATTGC |
| | CACAAGATGATCGACTTCTACAAGAAAAGCATTCAGAAAC |
| | ACCCGGAATGGAAGGAGTATGATTTTCAATTCAAGAAAAC |
| | CGAGGACTACGTGGATATCAGCGAATTCTATAAAGAGGTT |
| | TCTGACCAGGGTTACAAGATCGAATTCAAGAAAATTAGCG |
| | AGAAATACCTGCTGGACCTGGTGGAGGAAGGTAAACTGTA |
| | CCTGTTCCAAATCTGGAACAAGGATTTCAGCAAGTACAGC |
| | GAAGGCCGTAAAAACCTGCACACCATCTATTGGAAAGAAC |
| | TGTTCAGCAAGGAGAACCTGAGCGATATTACCTATAAGCT |
| | GAACGGCGAGGCGGAAATCTTTTACCGTCCGAAAAGCATG |
| | GAGCGTAAGGTTACCCACCCGAAGAACCAGAAAATCGAAA |
| | ACAAAGACCCGATCAAGGGTAAGAAATTCAGCAAGTTCAA |
| | GTATGACTTCATCAAGAACAAGCGTTACACCGAGGATCGT |
| | TTCTTTTTCCACTGCCCGATCACCCTGAACTTTCAAGCGC |
| | GTGACGGCAGCAAAACCATCAACAAGCGTGTGAACGATCA |
| | CATTCGTGAGACCAAAGACGATATCTTCGTTCTGAGCATT |
| | GATCGTGGTGAACGTCACCTGGCGTACTATACCCTGCTGA |
| | ACAGCAAGGGTGAAATTCAGGAGCAAGGCAGCTTTAACGT |
| | GATCAGCGACGATAAGGAGCGTAAACGTGACTATCACGAA |
| | AAACTGGATGAGCGTGAAAAGGAGCGTGACAAGGCGCGTA |
| | AAAGCTGGCAGAAAATCGAGACCATTAAGAAACTGAAGGA |
| | TGGCTACCTGAGCCAAATCGTGCACAAGATTGCGAAACTG |
| | GCGATCGAGAAAAACGCGATCATTGTTCTGGAAGACCTGA |
| | ACCTGGATTTCAAGCGTGGTCGTCTGAAGATTGAGAAACA |
| | GGTGTACCAAAAATTCGAAAAGAAACTGATCGACAAGCTG |
| | AACTATCTGGTTTTTAAAGAACGTACCGAAAAAGAGGCGG |
| | GTGGTAGCCTGAACGCGTATCAGCTGACCGGTAAATTCGA |
| | GGGCTTTAAGAAACTGGGCAAGGAAACCGGCATCATTTAC |
| | TATGTGCCGGCGGCGTACACCAGCAAAATCTGCCCGAAGA |
| | CCGGCTTCGTTAACCTGCTGCGTCCGAAGTTCAAGAACAT |
| | CGAAAAGGCGAAGGAGTTTTTCAAGAAGTTCAACTACATC |
| | AAGTACGACAGCAGCGAAGGTCTGTTTGAGTTCAACTTCG |
| | ATTACAGCAAGTTCATCAAGAACGGCAAGAAAGAGACCAA |
| | AATCATTCAGGACAACTGGAGCGTGTATAGCAACGGTACC |
| | AAGCTGGTTGGCTTCCGTAACAAGAACAAAAACAACAGCT |
| | GGGATACCAAGGAAGTGAAACCGAACGAGAAGCTGAAAAT |
| | TCTGTTCAAAGAGTACGGTGTTAGCTTTCAAAAGGACGAA |
| | AACATCATTAGCCAGATCGCGAGCCAAAACAAGAAAGCGT |
| | TTTTCGAGAACCTGATCAAGATTTTCAAAACCATTCTGAT |
| | GCTGCGTAACAGCCGTAAAGACCCGGAGGAAGATTACGTG |
| | CTGAGCTGCGTTAAGGACGAAAACGGCGAGTTTTTCGACA |
| | GCCGTAAGGCGAAAGATAACGAGCCGAAAGACGCGGATGC |
| | GAACGGCGCGTACCACATTGGTCTGAAGGGCCTGATGCTG |
| | CTGGAACGTATCAAGGCGAACAAAGGTAAGAAAAAGCTGG |
| | ACCTGCTGATCAGCCGTAACGATTTCATTAACTTTGCGGT |
| | TGAGCGTAGCAAGTAATAAGGATCCCTCGAGTTGACAGCT |
| | AGCTCAGTCCTAGGTATAATGCTAGCGTTTAAGGCCTTGA |
| | CAAAATTTCTACTGTAGTAGATGTGGCAGCTCAAAAATTG |
| | GCTACAAAACGTTTAAGGCCTTGACAAAATTTCTACTGTA |
| | GTAGATCTAGCATAACCCCTTGGGGCCTCTAAACGGGTCT |
| | TGAGGGGTTTTTTGCATATGCTGAAAGGAGGAACTATATC |
| | CGGAT (SEQ ID NO: 64) |
| Cas12p | TGGCGAATGGGACGCGCCCTGTAGCGGCGCATTAAGCGCG |
| | GCGGGTGTGGTGGTTACGCGCAGCGTGACCGCTACACTTG |
| | CCAGCGCCCTAGCGCCCGCTCCTTTCGCTTTCTTCCCTTC |
| | CTTTCTCGCCACGTTCGCCGGCTTTCCCCGTCAAGCTCTA |
| | AATCGGGGGCTCCCTTTAGGGTTCCGATTTAGTGCTTTAC |
| | GGCACCTCGACCCCAAAAAACTTGATTAGGGTGATGGTTC |
| | ACGTAGTGGGCCATCGCCCTGATAGACGGTTTTTCGCCCT |
| | TTGACGTTGGAGTCCACGTTCTTTAATAGTGGACTCTTGT |
| | TCCAAACTGGAACAACACTCAACCCTATCTCGGTCTATTC |
| | TTTTGATTTATAAGGGATTTTGCCGATTTCGGCCTATTGG |
| | TTAAAAAATGAGCTGATTTAACAAAAATTTAACGCGAATT |
| | TTAACAAAATATTAACGTTTACAATTTCAGGTGGCACTTT |
| | TCGGGGAAATGTGCGCGGAACCCCTATTTGTTTATTTTTC |
| | TAAATACATTCAAATATGTATCCGCTCATGAATTAATTCT |
| | TAGAAAAACTCATCGAGCATCAAATGAAACTGCAATTTAT |
| | TCATATCAGGATTATCAATACCATATTTTTGAAAAAGCCG |
| | TTTCTGTAATGAAGGAGAAAACTCACCGAGGCAGTTCCAT |
| | AGGATGGCAAGATCCTGGTATCGGTCTGCGATTCCGACTC |
| | GTCCAACATCAATACAACCTATTAATTTCCCCTCGTCAAA |
| | AATAAGGTTATCAAGTGAGAAATCACCATGAGTGACGACT |
| | GAATCCGGTGAGAATGGCAAAAGTTTATGCATTTCTTTCC |
| | AGACTTGTTCAACAGGCCAGCCATTACGCTCGTCATCAAA |
| | ATCACTCGCATCAACCAAACCGTTATTCATTCGTGATTGC |
| | GCCTGAGCGAGACGAAATACGCGATCGCTGTTAAAAGGAC |
| | AATTACAAACAGGAATCGAATGCAACCGGCGCAGGAACAC |
| | TGCCAGCGCATCAACAATATTTTCACCTGAATCAGGATAT |
| | TCTTCTAATACCTGGAATGCTGTTTTCCCGGGGATCGCAG |
| | TGGTGAGTAACCATGCATCATCAGGAGTACGGATAAAATG |
| | CTTGATGGTCGGAAGAGGCATAAATTCCGTCAGCCAGTTT |
| | AGTCTGACCATCTCATCTGTAACATCATTGGCAACGCTAC |
| | CTTTGCCATGTTTCAGAAACAACTCTGGCGCATCGGGCTT |
| | CCCATACAATCGATAGATTGTCGCACCTGATTGCCCGACA |
| | TTATCGCGAGCCCATTTATACCCATATAAATCAGCATCCA |
| | TGTTGGAATTTAATCGCGGCCTAGAGCAAGACGTTTCCCG |
| | TTGAATATGGCTCATAACACCCCTTGTATTACTGTTTATG |
| | TAAGCAGACAGTTTTATTGTTCATGACCAAAATCCCTTAA |
| | CGTGAGTTTTCGTTCCACTGAGCGTCAGACCCCGTAGAAA |
| | AGATCAAAGGATCTTCTTGAGATCCTTTTTTTCTGCGCGT |
| | AATCTGCTGCTTGCAAACAAAAAAACCACCGCTACCAGCG |
| | GTGGTTTGTTTGCCGGATCAAGAGCTACCAACTCTTTTTC |
| | CGAAGGTAACTGGCTTCAGCAGAGCGCAGATACCAAATAC |
| | TGTCCTTCTAGTGTAGCCGTAGTTAGGCCACCACTTCAAG |
| | AACTCTGTAGCACCGCCTACATACCTCGCTCTGCTAATCC |
| | TGTTACCAGTGGCTGCTGCCAGTGGCGATAAGTCGTGTCT |
| | TACCGGGTTGGACTCAAGACGATAGTTACCGGATAAGGCG |
| | CAGCGGTCGGGCTGAACGGGGGGTTCGTGCACACAGCCCA |
| | GCTTGGAGCGAACGACCTACACCGAACTGAGATACCTACA |
| | GCGTGAGCTATGAGAAAGCGCCACGCTTCCCGAAGGGAGA |
| | AAGGCGGACAGGTATCCGGTAAGCGGCAGGGTCGGAACAG |
| | GAGAGCGCACGAGGGAGCTTCCAGGGGGAAACGCCTGGTA |
| | TCTTTATAGTCCTGTCGGGTTTCGCCACCTCTGACTTGAG |
| | CGTCGATTTTTGTGATGCTCGTCAGGGGGGCGGAGCCTAT |
| | GGAAAAACGCCAGCAACGCGGCCTTTTTACGGTTCCTGGC |
| | CTTTTGCTGGCCTTTTGCTCACATGTTCTTTCCTGCGTTA |
| | TCCCCTGATTCTGTGGATAACCGTATTACCGCCTTTGAGT |
| | GAGCTGATACCGCTCGCCGCAGCCGAACGACCGAGCGCAG |
| | CGAGTCAGTGAGCGAGGAAGCGGAAGAGCGCCTGATGCGG |
| | TATTTTCTCCTTACGCATCTGTGCGGTATTTCACACCGCA |
| | TATATGGTGCACTCTCAGTACAATCTGCTCTGATGCCGCA |
| | TAGTTAAGCCAGTATACACTCCGCTATCGCTACGTGACTG |
| | GGTCATGGCTGCGCCCCGACACCCGCCAACACCCGCTGAC |
| | GCGCCCTGACGGGCTTGTCTGCTCCCGGCATCCGCTTACA |
| | GACAAGCTGTGACCGTCTCCGGGAGCTGCATGTGTCAGAG |
| | GTTTTCACCGTCATCACCGAAACGCGCGAGGCAGCTGCGG |
| | TAAAGCTCATCAGCGTGGTCGTGAAGCGATTCACAGATGT |
| | CTGCCTGTTCATCCGCGTCCAGCTCGTTGAGTTTCTCCAG |
| | AAGCGTTAATGTCTGGCTTCTGATAAAGCGGGCCATGTTA |
| | AGGGCGGTTTTTTCCTGTTTGGTCACTGATGCCTCCGTGT |
| | AAGGGGGATTTCTGTTCATGGGGGTAATGATACCGATGAA |
| | ACGAGAGAGGATGCTCACGATACGGGTTACTGATGATGAA |
| | CATGCCCGGTTACTGGAACGTTGTGAGGGTAAACAACTGG |
| | CGGTATGGATGCGGCGGGACCAGAGAAAAATCACTCAGGG |
| | TCAATGCCAGCGCTTCGTTAATACAGATGTAGGTGTTCCA |
| | CAGGGTAGCCAGCAGCATCCTGCGATGCAGATCCGGAACA |
| | TAATGGTGCAGGGCGCTGACTTCCGCGTTTCCAGACTTTA |
| | CGAAACACGGAAACCGAAGACCATTCATGTTGTTGCTCAG |
| | GTCGCAGACGTTTTGCAGCAGCAGTCGCTTCACGTTCGCT |
| | CGCGTATCGGTGATTCATTCTGCTAACCAGTAAGGCAACC |
| | CCGCCAGCCTAGCCGGGTCCTCAACGACAGGAGCACGATC |
| | ATGCGCACCCGTGGGGCCGCCATGCCGGCGATAATGGCCT |
| | GCTTCTCGCCGAAACGTTTGGTGGCGGGACCAGTGACGAA |
| | GGCTTGAGCGAGGGCGTGCAAGATTCCGAATACCGCAAGC |
| | GACAGGCCGATCATCGTCGCGCTCCAGCGAAAGCGGTCCT |
| | CGCCGAAAATGACCCAGAGCGCTGCCGGCACCTGTCCTAC |
| | GAGTTGCATGATAAAGAAGACAGTCATAAGTGCGGCGACG |
| | ATAGTCATGCCCCGCGCCCACCGGAAGGAGCTGACTGGGT |
| | TGAAGGCTCTCAAGGGCATCGGTCGAGATCCCGGTGCCTA |
| | ATGAGTGAGCTAACTTACATTAATTGCGTTGCGCTCACTG |
| | CCCGCTTTCCAGTCGGGAAACCTGTCGTGCCAGCTGCATT |
| | AATGAATCGGCCAACGCGCGGGGAGAGGCGGTTTGCGTAT |
| | TGGGCGCCAGGGTGGTTTTTCTTTTCACCAGTGAGACGGG |
| | CAACAGCTGATTGCCCTTCACCGCCTGGCCCTGAGAGAGT |
| | TGCAGCAAGCGGTCCACGCTGGTTTGCCCCAGCAGGCGAA |
| | AATCCTGTTTGATGGTGGTTAACGGCGGGATATAACATGA |
| | GCTGTCTTCGGTATCGTCGTATCCCACTACCGAGATATCC |
| | GCACCAACGCGCAGCCCGGACTCGGTAATGGCGCGCATTG |
| | CGCCCAGCGCCATCTGATCGTTGGCAACCAGCATCGCAGT |
| | GGGAACGATGCCCTCATTCAGCATTTGCATGGTTTGTTGA |
| | AAACCGGACATGGCACTCCAGTCGCCTTCCCGTTCCGCTA |
| | TCGGCTGAATTTGATTGCGAGTGAGATATTTATGCCAGCC |
| | AGCCAGACGCAGACGCGCCGAGACAGAACTTAATGGGCCC |
| | GCTAACAGCGCGATTTGCTGGTGACCCAATGCGACCAGAT |
| | GCTCCACGCCCAGTCGCGTACCGTCTTCATGGGAGAAAAT |
| | AATACTGTTGATGGGTGTCTGGTCAGAGACATCAAGAAAT |
| | AACGCCGGAACATTAGTGCAGGCAGCTTCCACAGCAATGG |
| | CATCCTGGTCATCCAGCGGATAGTTAATGATCAGCCCACT |
| | GACGCGTTGCGCGAGAAGATTGTGCACCGCCGCTTTACAG |
| | GCTTCGACGCCGCTTCGTTCTACCATCGACACCACCACGC |
| | TGGCACCCAGTTGATCGGCGCGAGATTTAATCGCCGCGAC |
| | AATTTGCGACGGCGCGTGCAGGGCCAGACTGGAGGTGGCA |
| | ACGCCAATCAGCAACGACTGTTTGCCCGCCAGTTGTTGTG |
| | CCACGCGGTTGGGAATGTAATTCAGCTCCGCCATCGCCGC |
| | TTCCACTTTTTCCCGCGTTTTCGCAGAAACGTGGCTGGCC |
| | TGGTTCACCACGCGGGAAACGGTCTGATAAGAGACACCGG |
| | CATACTCTGCGACATCGTATAACGTTACTGGTTTCACATT |
| | CACCACCCTGAATTGACTCTCTTCCGGGCGCTATCATGCC |
| | ATACCGCGAAAGGTTTTGCGCCATTCGATGGTGTCCGGGA |
| | TCTCGACGCTCTCCCTTATGCGACTCCTGCATTAGGAAGC |
| | AGCCCAGTAGTAGGTTGAGGCCGTTGAGCACCGCCGCCGC |
| | AAGGAATGGTGCATGCAAGGAGATGGCGCCCAACAGTCCC |
| | CCGGCCACGGGGCCTGCCACCATACCCACGCCGAAACAAG |
| | CGCTCATGAGCCCGAAGTGGCGAGCCCGATCTTCCCCATC |
| | GGTGATGTCGGCGATATAGGCGCCAGCAACCGCACCTGTG |
| | GCGCCGGTGATGCCGGCCACGATGCGTCCGGCGTAGAGGA |
| | TCGAGATCTCGATCCCGCGAAATTAATACGACTCACTATA |
| | GGGGAATTGTGAGCGGATAACAATTCCCCTCTAGATTTAC |
| | ACTTTATGCTTCCGGCTCGTATGTTTCTAGAGAATTCAAA |
| | TAATTTTGTTTAACTTTAAGAAGGAGATTTAAATATGGGA |
| | TCAAGTCATCACCACCACCACCACTCAAGTGGACTAGTAC |
| | CCAGGGGAAGCATGAAGAAGAGCATTTTCGATCAGTTCGT |
| | TAACCAGTACGCGCTGAGCAAGACCCTGCGTTTCGAGCTG |
| | AAACCGGTGGGTGAAACCGGCCGTATGCTGGAGGAAGCGA |
| | AGGTTTTCGCGAAGGATGAAACCATTAAGAAAAAGTACGA |
| | AGCGACCAAGCCGTTCTTTAACAAACTGCACCGTGAATTC |
| | GTGGAGGAAGCGCTGAACGAGGTTGAACTGGCGGGCCTGC |
| | CGGAGTACTTCGAAATCTTCAAGTACTGGAAGCGTTACAA |
| | AAAGAAATTCGAGAAGGACCTGCAGAAGAAAGAGAAGGAA |
| | CTGCGTAAAAGCGTGGTTGGTTTCTTTAACGCGCAAGCGA |
| | AGGAGTGGGCGAAGAAATATGAAACCCTGGGCGTGAAGAA |
| | AAAGGATGTTGGTCTGCTGTTCGAGGAAAACGTGTTTGCG |
| | ATTCTGAAAGAACGTTACGGTAACGAGGAAGGCAGCCAGA |
| | TTGTGGACGAGAGCACCGGCAAGGATGTTAGCATCTTCGA |
| | CAGCTGGAAGGGTTTTACCGGCTATTTCATCAAATTTCAG |
| | GAAACCCGTAAGAACTTCTACAAAGATGATGGTACCGCGA |
| | CCGCGCTGGCGACCCGTATCATTGATCAAAACCTGAAACG |
| | TTTCTGCGACAACCTGCTGATCTTTGAGAGCATTCGTGAT |
| | AAGATCGACTTCAGCGAGGTTGAACAGACCATGGGCAACA |
| | GCATCGATAAGGTGTTCAGCGTTATCTTTTATAGCAGCTG |
| | CCTGCTGCAAGAAGGTATCGACTTTTACAACTGCGTGCTG |
| | GGTGGTGAAACCCTGCCGAACGGTGAAAAGCGTCAGGGCA |
| | TTAACGAACTGATCAACCTGTACCGTCAAAAGACCAGCGA |
| | GAAAGTTCCGTTCCTGAAGCTGCTGGACAAACAGATTCTG |
| | AGCGAGAAGGAAAAATTTATGGATGAGATCGAAAACGACG |
| | AGGCGCTGCTGGATACCCTGAAGATTTTCCGTAAAAGCGC |
| | GGAGGAAAAGACCACCCTGCTGAAAAACATCTTCGGCGAT |
| | TTTGTGATGAACCAGGGTAAATATGACCTGGCGCAAATCT |
| | ACATTAGCCGTGAAAGCCTGAACACCATTAGCCGTAAGTG |
| | GACCAGCGAAACCGATATCTTCGAAGACAGCCTGTACGAG |
| | GTGCTGAAAAAGAGCAAAATCGTGAGCGCGAGCGTTAAAA |
| | AGAAAGACGGTGGCTACGCGTTCCCGGAGTTTATCGCGCT |
| | GATTTATGTTAAAAGCGCGCTGGAACAGATTCCGACCGAG |
| | AAGTTCTGGAAAGAACGTTACTATAAGAACATCGGCGATG |
| | TGCTGAACAAGGGTTTCCTGAACGGTAAAGAAGGCGTTTG |
| | GCTGCAATTTCTGCTGATCTTTGACTTCGAATTTAACAGC |
| | CTGTTCGAGCGTGAAATCATTGATGAGAACGGCGACAAGA |
| | AAGTGGCGGGTTATAACCTGTTCGCGAAGGGTTTTGACGA |
| | TCTGCTGAACAACTTCAAATACGACCAGAAGGCGAAAGTG |
| | GTTATTAAGGATTTTGCGGACGAAGTTCTGCACATTTATC |
| | AAATGGGCAAATACTTCGCGATCGAGAAGAAACGTAGCTG |
| | GCTGGCGGACTATGATATTGACAGCTTCTACACCGATCCG |
| | GAGAAGGGTTACCTGAAATTTTATGAAAACGCGTACGAGG |
| | AAATCATTCAGGTTTATAACAAGCTGCGTAACTACCTGAC |
| | CAAGAAACCGTATAGCGAGGACAAGTGGAAACTGAACTTC |
| | GAAAACCCGACCCTGGCGGATGGTTGGGACAAGAACAAAG |
| | AGGCGGATAACAGCACCGTGATTCTGAAGAAAGACGGTCG |
| | TTACTATCTGGGCCTGATGGCGCGTGGTCGTAACAAGCTG |
| | TTCGACGATCGTAACCTGCCGAAAATCCTGGAGGGTGTTG |
| | AAAACGGCAAGTACGAAAAGGTGGTTTACAAGTACTTCCC |
| | GGATCAGGCGAAGATGTTCCCGAAAGTGTGCTTTAGCACC |
| | AAAGGCCTGGAATTCTTTCAACCGAGCGAGGAAGTTATCA |
| | CCATTTACAAGAACAGCGAGTTCAAGAAAGGTTATACCTT |
| | TAACGTGCGTAGCATGCAGCGTCTGATTGATTTCTATAAA |
| | GACTGCCTGGTTCGTTACGAAGGTTGGCAATGCTATGATT |
| | TTCGTAACCTGCGTAAGACCGAGGACTACCGTAAAAACAT |
| | CGAGGAATTCTTTAGCGATGTGGCGATGGACGGCTACAAG |
| | ATTAGCTTCCAGGACGTTAGCGAGAGCTATATCAAGGAGA |
| | AGAACCAAAACGGTGATCTGTACCTGTTTGAGATCAAGAA |
| | CAAAGACTGGAACGAAGGTGCGAACGGCAAGAAAAACCTG |
| | CACACCATTTATTTCGAGAGCCTGTTTAGCGCGGATAACA |
| | TCGCGATGAACTTCCCGGTGAAACTGAACGGCCAGGCGGA |
| | GATCTTTTACCGTCCGCGTACCGAAGGTCTGGAGAAGGAA |
| | CGTATCATTACCAAGAAAGGCAACGTTCTGGAAAAGGGTG |
| | ACAAAGCGTTCCACAAGCGTCGTTACACCGAGAACAAAGT |
| | GTTCTTTCACGTTCCGATTACCCTGAACCGTACCAAGAAA |
| | AACCCGTTCCAATTTAACGCGAAGATCAACGACTTCCTGG |
| | CGAAAAACAGCGATATCAACGTGATTGGTGTTGACCGTGG |
| | CGAGAAACAGCTGGCGTATTTTAGCGTGATTAGCCAACGT |
| | GGCAAGATCCTGGACCGTGGTAGCCTGAACGTGATCAACG |
| | GCGTTAACTACGCGGAGAAGCTGGAGGAAAAAGCGCGTGG |
| | TCGTGAACAGGCGCGTAAGGATTGGCAGCAAATCGAGGGC |
| | ATTAAAGACCTGAAGAAAGGTTATATTAGCCAGGTGGTTC |
| | GTAAACTGGCGGATCTGGCGATCCAATACAACGCGATCAT |
| | TGTGTTCGAGGACCTGAACATGCGTTTTAAGCAAATTCGT |
| | GGTGGCATCGAGAAAAGCGTTTATCAGCAACTGGAAAAGG |
| | CGCTGATCGATAAACTGACCTTCCTGGTGGAGAAGGAAGA |
| | AAAGGACGTTGAAAAGGCGGGTCACCTGCTGAAAGCGTAC |
| | CAGCTGGCGGCGCCGTTCGAAACCTTTCAGAAGATGGGTA |
| | AACAAACCGGCATTGTGTTTTATACCCAAGCGGCGTACAC |
| | CAGCCGTATCGATCCGGTTACCGGCTGGCGTCCGCACCTG |
| | TACCTGAAATATAGCAGCGCGGAAAAGGCGAAAGCGGACC |
| | TGCTGAAGTTCAAGAAAATTAAGTTCGTGGATGGTCGTTT |
| | CGAGTTTACCTACGACATCAAGAGCTTCCGTGAGCAGAAG |
| | GAACACCCGAAAGCGACCGTGTGGACCGTTTGCAGCTGCG |
| | TTGAGCGTTTTCGTTGGAACCGTTATCTGAACAGCAACAA |
| | AGGTGGCTACGATCACTATAGCGACGTGACCAAGTTCCTG |
| | GTTGAGCTGTTTCAGGAATACGGCATCGACTTCGAACGTG |
| | GTGATATTGTGGGCCAAATCGAGGTTCTGGAAACCAAGGG |
| | TAACGAGAAGTTCTTTAAGAACTTCGTGTTCTTTTTCAAC |
| | CTGATCTGCCAGATTCGTAACACCAACGCGAGCGAACTGG |
| | CGAAGAAAGACGGCAAGGACGATTTCATTCTGAGCCCGGT |
| | TGAGCCGTTTTTCGATAGCCGTAACAGCGAGAAGTTCGGC |
| | GAAGACCTGCCGAAAAACGGTGACGATAACGGCGCGTTTA |
| | ACATCGCGCGTAAAGGTCTGGTTATTATGGATAAGATCAC |
| | CAAATTCGCGGACGAGAACGGTGGCTGCGAAAAGATGAAA |
| | TGGGGTGACCTGTATGTGAGCAATGTGGAGTGGGATAACT |
| | TTGTGGCGAATAAATAATAAGGATCCCTCGAGTTGACAGC |
| | TAGCTCAGTCCTAGGTATAATGCTAGCATCTACAAAAGTA |
| | GAAATCTAATAGGGATATTCGAGGTGGCAGCTCAAAAATT |
| | GGCTACAAAACATCTACAAAAGTAGAAATCTAATAGGGAT |
| | ATTCGAGCTAGCATAACCCCTTGGGGCCTCTAAACGGGTC |
| | TTGAGGGGTTTTTTGCATATGCTGAAAGGAGGAACTATAT |
| | CCGGAT (SEQ ID NO: 65) |
| Cas12q | TGGCGAATGGGACGCGCCCTGTAGCGGCGCATTAAGCGCG |
| | GCGGGTGTGGTGGTTACGCGCAGCGTGACCGCTACACTTG |
| | CCAGCGCCCTAGCGCCCGCTCCTTTCGCTTTCTTCCCTTC |
| | CTTTCTCGCCACGTTCGCCGGCTTTCCCCGTCAAGCTCTA |
| | AATCGGGGGCTCCCTTTAGGGTTCCGATTTAGTGCTTTAC |
| | GGCACCTCGACCCCAAAAAACTTGATTAGGGTGATGGTTC |
| | ACGTAGTGGGCCATCGCCCTGATAGACGGTTTTTCGCCCT |
| | TTGACGTTGGAGTCCACGTTCTTTAATAGTGGACTCTTGT |
| | TCCAAACTGGAACAACACTCAACCCTATCTCGGTCTATTC |
| | TTTTGATTTATAAGGGATTTTGCCGATTTCGGCCTATTGG |
| | TTAAAAAATGAGCTGATTTAACAAAAATTTAACGCGAATT |
| | TTAACAAAATATTAACGTTTACAATTTCAGGTGGCACTTT |
| | TCGGGGAAATGTGCGCGGAACCCCTATTTGTTTATTTTTC |
| | TAAATACATTCAAATATGTATCCGCTCATGAATTAATTCT |
| | TAGAAAAACTCATCGAGCATCAAATGAAACTGCAATTTAT |
| | TCATATCAGGATTATCAATACCATATTTTTGAAAAAGCCG |
| | TTTCTGTAATGAAGGAGAAAACTCACCGAGGCAGTTCCAT |
| | AGGATGGCAAGATCCTGGTATCGGTCTGCGATTCCGACTC |
| | GTCCAACATCAATACAACCTATTAATTTCCCCTCGTCAAA |
| | AATAAGGTTATCAAGTGAGAAATCACCATGAGTGACGACT |
| | GAATCCGGTGAGAATGGCAAAAGTTTATGCATTTCTTTCC |
| | AGACTTGTTCAACAGGCCAGCCATTACGCTCGTCATCAAA |
| | ATCACTCGCATCAACCAAACCGTTATTCATTCGTGATTGC |
| | GCCTGAGCGAGACGAAATACGCGATCGCTGTTAAAAGGAC |
| | AATTACAAACAGGAATCGAATGCAACCGGCGCAGGAACAC |
| | TGCCAGCGCATCAACAATATTTTCACCTGAATCAGGATAT |
| | TCTTCTAATACCTGGAATGCTGTTTTCCCGGGGATCGCAG |
| | TGGTGAGTAACCATGCATCATCAGGAGTACGGATAAAATG |
| | CTTGATGGTCGGAAGAGGCATAAATTCCGTCAGCCAGTTT |
| | AGTCTGACCATCTCATCTGTAACATCATTGGCAACGCTAC |
| | CTTTGCCATGTTTCAGAAACAACTCTGGCGCATCGGGCTT |
| | CCCATACAATCGATAGATTGTCGCACCTGATTGCCCGACA |
| | TTATCGCGAGCCCATTTATACCCATATAAATCAGCATCCA |
| | TGTTGGAATTTAATCGCGGCCTAGAGCAAGACGTTTCCCG |
| | TTGAATATGGCTCATAACACCCCTTGTATTACTGTTTATG |
| | TAAGCAGACAGTTTTATTGTTCATGACCAAAATCCCTTAA |
| | CGTGAGTTTTCGTTCCACTGAGCGTCAGACCCCGTAGAAA |
| | AGATCAAAGGATCTTCTTGAGATCCTTTTTTTCTGCGCGT |
| | AATCTGCTGCTTGCAAACAAAAAAACCACCGCTACCAGCG |
| | GTGGTTTGTTTGCCGGATCAAGAGCTACCAACTCTTTTTC |
| | CGAAGGTAACTGGCTTCAGCAGAGCGCAGATACCAAATAC |
| | TGTCCTTCTAGTGTAGCCGTAGTTAGGCCACCACTTCAAG |
| | AACTCTGTAGCACCGCCTACATACCTCGCTCTGCTAATCC |
| | TGTTACCAGTGGCTGCTGCCAGTGGCGATAAGTCGTGTCT |
| | TACCGGGTTGGACTCAAGACGATAGTTACCGGATAAGGCG |
| | CAGCGGTCGGGCTGAACGGGGGGTTCGTGCACACAGCCCA |
| | GCTTGGAGCGAACGACCTACACCGAACTGAGATACCTACA |
| | GCGTGAGCTATGAGAAAGCGCCACGCTTCCCGAAGGGAGA |
| | AAGGCGGACAGGTATCCGGTAAGCGGCAGGGTCGGAACAG |
| | GAGAGCGCACGAGGGAGCTTCCAGGGGGAAACGCCTGGTA |
| | TCTTTATAGTCCTGTCGGGTTTCGCCACCTCTGACTTGAG |
| | CGTCGATTTTTGTGATGCTCGTCAGGGGGGCGGAGCCTAT |
| | GGAAAAACGCCAGCAACGCGGCCTTTTTACGGTTCCTGGC |
| | CTTTTGCTGGCCTTTTGCTCACATGTTCTTTCCTGCGTTA |
| | TCCCCTGATTCTGTGGATAACCGTATTACCGCCTTTGAGT |
| | GAGCTGATACCGCTCGCCGCAGCCGAACGACCGAGCGCAG |
| | CGAGTCAGTGAGCGAGGAAGCGGAAGAGCGCCTGATGCGG |
| | TATTTTCTCCTTACGCATCTGTGCGGTATTTCACACCGCA |
| | TATATGGTGCACTCTCAGTACAATCTGCTCTGATGCCGCA |
| | TAGTTAAGCCAGTATACACTCCGCTATCGCTACGTGACTG |
| | GGTCATGGCTGCGCCCCGACACCCGCCAACACCCGCTGAC |
| | GCGCCCTGACGGGCTTGTCTGCTCCCGGCATCCGCTTACA |
| | GACAAGCTGTGACCGTCTCCGGGAGCTGCATGTGTCAGAG |
| | GTTTTCACCGTCATCACCGAAACGCGCGAGGCAGCTGCGG |
| | TAAAGCTCATCAGCGTGGTCGTGAAGCGATTCACAGATGT |
| | CTGCCTGTTCATCCGCGTCCAGCTCGTTGAGTTTCTCCAG |
| | AAGCGTTAATGTCTGGCTTCTGATAAAGCGGGCCATGTTA |
| | AGGGCGGTTTTTTCCTGTTTGGTCACTGATGCCTCCGTGT |
| | AAGGGGGATTTCTGTTCATGGGGGTAATGATACCGATGAA |
| | ACGAGAGAGGATGCTCACGATACGGGTTACTGATGATGAA |
| | CATGCCCGGTTACTGGAACGTTGTGAGGGTAAACAACTGG |
| | CGGTATGGATGCGGCGGGACCAGAGAAAAATCACTCAGGG |
| | TCAATGCCAGCGCTTCGTTAATACAGATGTAGGTGTTCCA |
| | CAGGGTAGCCAGCAGCATCCTGCGATGCAGATCCGGAACA |
| | TAATGGTGCAGGGCGCTGACTTCCGCGTTTCCAGACTTTA |
| | CGAAACACGGAAACCGAAGACCATTCATGTTGTTGCTCAG |
| | GTCGCAGACGTTTTGCAGCAGCAGTCGCTTCACGTTCGCT |
| | CGCGTATCGGTGATTCATTCTGCTAACCAGTAAGGCAACC |
| | CCGCCAGCCTAGCCGGGTCCTCAACGACAGGAGCACGATC |
| | ATGCGCACCCGTGGGGCCGCCATGCCGGCGATAATGGCCT |
| | GCTTCTCGCCGAAACGTTTGGTGGCGGGACCAGTGACGAA |
| | GGCTTGAGCGAGGGCGTGCAAGATTCCGAATACCGCAAGC |
| | GACAGGCCGATCATCGTCGCGCTCCAGCGAAAGCGGTCCT |
| | CGCCGAAAATGACCCAGAGCGCTGCCGGCACCTGTCCTAC |
| | GAGTTGCATGATAAAGAAGACAGTCATAAGTGCGGCGACG |
| | ATAGTCATGCCCCGCGCCCACCGGAAGGAGCTGACTGGGT |
| | TGAAGGCTCTCAAGGGCATCGGTCGAGATCCCGGTGCCTA |
| | ATGAGTGAGCTAACTTACATTAATTGCGTTGCGCTCACTG |
| | CCCGCTTTCCAGTCGGGAAACCTGTCGTGCCAGCTGCATT |
| | AATGAATCGGCCAACGCGCGGGGAGAGGCGGTTTGCGTAT |
| | TGGGCGCCAGGGTGGTTTTTCTTTTCACCAGTGAGACGGG |
| | CAACAGCTGATTGCCCTTCACCGCCTGGCCCTGAGAGAGT |
| | TGCAGCAAGCGGTCCACGCTGGTTTGCCCCAGCAGGCGAA |
| | AATCCTGTTTGATGGTGGTTAACGGCGGGATATAACATGA |
| | GCTGTCTTCGGTATCGTCGTATCCCACTACCGAGATATCC |
| | GCACCAACGCGCAGCCCGGACTCGGTAATGGCGCGCATTG |
| | CGCCCAGCGCCATCTGATCGTTGGCAACCAGCATCGCAGT |
| | GGGAACGATGCCCTCATTCAGCATTTGCATGGTTTGTTGA |
| | AAACCGGACATGGCACTCCAGTCGCCTTCCCGTTCCGCTA |
| | TCGGCTGAATTTGATTGCGAGTGAGATATTTATGCCAGCC |
| | AGCCAGACGCAGACGCGCCGAGACAGAACTTAATGGGCCC |
| | GCTAACAGCGCGATTTGCTGGTGACCCAATGCGACCAGAT |
| | GCTCCACGCCCAGTCGCGTACCGTCTTCATGGGAGAAAAT |
| | AATACTGTTGATGGGTGTCTGGTCAGAGACATCAAGAAAT |
| | AACGCCGGAACATTAGTGCAGGCAGCTTCCACAGCAATGG |
| | CATCCTGGTCATCCAGCGGATAGTTAATGATCAGCCCACT |
| | GACGCGTTGCGCGAGAAGATTGTGCACCGCCGCTTTACAG |
| | GCTTCGACGCCGCTTCGTTCTACCATCGACACCACCACGC |
| | TGGCACCCAGTTGATCGGCGCGAGATTTAATCGCCGCGAC |
| | AATTTGCGACGGCGCGTGCAGGGCCAGACTGGAGGTGGCA |
| | ACGCCAATCAGCAACGACTGTTTGCCCGCCAGTTGTTGTG |
| | CCACGCGGTTGGGAATGTAATTCAGCTCCGCCATCGCCGC |
| | TTCCACTTTTTCCCGCGTTTTCGCAGAAACGTGGCTGGCC |
| | TGGTTCACCACGCGGGAAACGGTCTGATAAGAGACACCGG |
| | CATACTCTGCGACATCGTATAACGTTACTGGTTTCACATT |
| | CACCACCCTGAATTGACTCTCTTCCGGGCGCTATCATGCC |
| | ATACCGCGAAAGGTTTTGCGCCATTCGATGGTGTCCGGGA |
| | TCTCGACGCTCTCCCTTATGCGACTCCTGCATTAGGAAGC |
| | AGCCCAGTAGTAGGTTGAGGCCGTTGAGCACCGCCGCCGC |
| | AAGGAATGGTGCATGCAAGGAGATGGCGCCCAACAGTCCC |
| | CCGGCCACGGGGCCTGCCACCATACCCACGCCGAAACAAG |
| | CGCTCATGAGCCCGAAGTGGCGAGCCCGATCTTCCCCATC |
| | GGTGATGTCGGCGATATAGGCGCCAGCAACCGCACCTGTG |
| | GCGCCGGTGATGCCGGCCACGATGCGTCCGGCGTAGAGGA |
| | TCGAGATCTCGATCCCGCGAAATTAATACGACTCACTATA |
| | GGGGAATTGTGAGCGGATAACAATTCCCCTCTAGATTTAC |
| | ACTTTATGCTTCCGGCTCGTATGTTTCTAGAGAATTCAAA |
| | TAATTTTGTTTAACTTTAAGAAGGAGATTTAAATATGGGG |
| | TCCTCCCATCATCACCACCACCACTCTTCAGGCTTGGTAC |
| | CGCGTGGTTCCATGATCAACATAGACGAATTGAAAAATTT |
| | ATATAAGGTGCAAAAGACCATCACTTTCGAACTTAAGAAC |
| | AAGTGGGAGAACAAAAATGATGAGAACGACAGAGTAGAGT |
| | TCTTGAAGACTCAGGAGTGGGTCGAAAGCCTTTTCAAGGT |
| | CGATGAAGAGAACTTTGATGAGAAAGAGTCTATCCCTAAC |
| | TTGTTAGACTTCGGACAGAAGATTGCGTCCTTGTTTTACA |
| | AGCTGAGCGAGGACATAGCGAACAACCAAATTGATACGCG |
| | GGTATTGAAAGTCTCGAAATTCCTTTTAGAGGAAATTGAT |
| | AGAAATCAATACCACGAGAAAAAAAACAAGCCCACAAAGG |
| | TAAAAGAAATGAATCCCAACACAAACAAAAGTTATATAAA |
| | AGAATATAAGCTGTCCGACCAAAACACACTGTACGTGTTA |
| | TTAAAGATAATGGAAGATGAAGGTCGGGGATTACAAAAAT |
| | TTTTGTACGATAAAGCGGACCGGTTAAACCTGTACAATCA |
| | AAAAGTTCGGAGAGACTTCGCCTTAAAGGAATCAAATGAG |
| | CAACAAAAATTCTCTGGAAATGCCAACTACTATGGGAATA |
| | TAAAGCTGCTTATAGATAGCTTAGAAGATGCAGTCCGGAT |
| | CATTGGGTATTTCACTTTCGACGATCAAGCAGAAAACGCA |
| | CAAATCAATGAATTTAAGTCCGTTAAACAGGAAATGAATA |
| | ATAATGAAGCGTCTTACCAAGCACTGAAAGACTTCGCTAT |
| | TGATAACGCAAAAAAAGAGATAGAATTGACGACGTTGAAC |
| | CACCGGGCGGTCAACAAGGATCCAAAAAAGATTCAAGAAC |
| | AGATTGAGGAAGTCGAAAATTTCGAAGAAGATATTAACCA |
| | GTTAAAGCATCAGATATCAGCCTTGAATGATAAGAAGTTT |
| | GACGTGGTTAGCAGATTAAAGCACGCTCTTATAAAAATGT |
| | TACCAGAACTGAATCTTTTGGATGCTGAGTCGGAACAGGG |
| | CCGTGAAGTCCAGCAGATATATCAAGACAAAAAAAACGGG |
| | TTGGAGCTTGATGACTTTAAATTTAACCTTTTAAAACATC |
| | ATCAATGGCAAAAAACGATCTTCAAGTATATTAAGCTTGA |
| | GGGCTTAGTTCTGCCAGACCTTTACGCGGAAAACAAACAA |
| | GATAAAATCAAGGTTTATATTGAGAATTATAGACAGAGTG |
| | GTGAGCGTATTTCTAAGAAGGCGAGAGAGGAATTAGGAAA |
| | AATCGATAAACGCGAAGAGTTCAATGGAAATGACGAACTT |
| | AAGAAGGCATGGTATGAGTATAAGGACTTCTGTAGAGACA |
| | AACGTAATAAGAGCGTGGAACTTGGCAATAAGAAGTCGCT |
| | GTACAATGCCATAAAGCGCGAAGTTTTGCGGCAAAAAATG |
| | TGCAACCATTTCGCTGTGCTGGTGTCCGACGGTGAAGATA |
| | CTTCCCCTTATTATTATCTGATATTAATCCCGAACGAGAA |
| | CTCCGATGAAATGAATAGAACGTTCAAGGAATTGAAGGCC |
| | TCCGAGGGGAATTGGAAGATGTTGGATTACAATCGTCTGA |
| | CCTTCAAAGCCTTGGAGAAATTGGCCCTGTTACGGTCGTC |
| | TACCTTCGAGATAGCGGATCAGGAACTGCAAGAAGAGGCA |
| | AAAAAGATCTGGGAGGAGTACAAGGAAAAGGCGTACAAAG |
| | ACTTCAAAAACAAAAAGTTATTACAGGGTTTATCGGGAAG |
| | ACAGCGGGAGGAGAAAAAGCAAGAATTGCAAAAGGAGAGC |
| | CTGAATAGAGTAATCAATTACTTGATCAGATGCATTCAGT |
| | CATTGCCCGACAGCGGAAAATACAACTTTAACTTTAAAGA |
| | GCCTCATCAATACCAATCGCTTGAAGAGTTTGCCGAGGAG |
| | ATTGATCGGCAAGGTTATCACTGTGCTTGGAAAAACGTTT |
| | CTAAAGATAAACTGATGGAATTGGAAGCGATGGAAAAGAT |
| | TAAGGTTTTCAAACTTCATAACAAAGACTTTCGCAAGGTA |
| | AAACTGAACGACTCCAAGCACAACCCTAATCTTTTTACTT |
| | TGTACTGGTTAGACGCCATGAATTTGGATAAGGTTAACGT |
| | CCGCCTGTTACCGGAAGTTGACCTTTACAAGAGAGCTAAG |
| | GAAACACAGCTGAAATTGTTCGAACGTGATGTGAAATGCA |
| | ATATCAATAACCAAAAGATTAAATCTATCAAGGAGAAGAA |
| | TAGACTGTTTCAGGACAAGTTGTATGCTAGTTTTAAGTTA |
| | GAGTTTTATCCAGAAAACGAAGGATTAGGTTTCGAGCAGG |
| | TAAATGACAAGGTCAATAACTTCTGCGGTAGCGATACGGC |
| | CTATTATCTTGGGCTTGATCGTGGAGAGAAAGAGCTTGTT |
| | ACATTCTGCCTGGTGGACTCTGATGGCCGCCTGGTAAAAA |
| | ACGGAGACTGGACCAAGTTTAAAGAGGTGAACTATGCCGA |
| | CAAACTGAAGCAATTCTACTACTCAAAAGGCGAAATAGAG |
| | AGTACCCAACAACAGCTGTTAGAAGCCCGGGACAATATTA |
| | AACAAGCGACCAACACGGAAGATAAGGAGTCCATGAAACT |
| | GAATTATAAGAAACTGGAACTGAAGTTAAAACAACAGAAT |
| | TTGCTGGCGCAAGAATTCATAAAAAAAGCGTACTGCGGCT |
| | ACCTTATCGATAGCATTAATGAGATTCTGAGAGAATATCC |
| | AAATACTTATCTTGTCTTAGAGGATTTGGATATCGCGGGT |
| | AAAGCGGATCCAGAGTCGGGGATGACTAATAAAGAGCAGA |
| | ACTTAAACAAAACGATGGGGGCTTCAGTATACCAGGCCAT |
| | TGAGAATGCGATCGTAAATAAATTCAAATATCGCACCGTG |
| | AAATTGTCCGATATCAAGGGCCTTCAGACTGTACCTAATG |
| | TAGTGAAGGTCGAAGACTTACGGGAAGTGAAAGAGGTTGA |
| | AGATGGGGAACACAAGTTCGGGTTAATAAGATCAGTTAAG |
| | AGCAAGGATCAAATCGGTAACATACTTTTTGTCGACGAGG |
| | GGGAGACCAGTAACACTTGTCCGAATTGCGGTTTTAATAG |
| | TGATTGGTTTAAACGCGATGTTGATTTTGACTTAGAAATA |
| | GTCGCTACTGTAAACGGGCAAAAGAATGCCGTGATTGAGC |
| | AAAATGACAAAAAATACTGTTTCCCGGGCGAAATATATAA |
| | ATTGGAAATCATTAATAAAGAGTACGAAACAAACAAGCGT |
| | AATCTTGCCATGATTTTTAAACCTCGGGCCAAAGCGTGCC |
| | GTAAATTTATCAATAATAATTTAGATAAGAACGATTATTT |
| | CTATTGTCCCTACTGCGCCTTCTCGTCGAAGAATTGTAAC |
| | AACCCGAAACTGCAGAACGGCGATTTCGTGGTATATTCAG |
| | GAGACGATGTTGCTGCTTACAATGTTGCTATCAGAGGAAT |
| | TAACCTGCTGAACAATATTAAATAGCTAGCATAACCCCTT |
| | GGGGCCTCTAAACGGGTCTTGAGGGGTTTTTTGCTGAAAG |
| | GAGGAACTATATCCGGAT (SEQ ID NO: 66) |
| Cas9.1 | TGGCGAATGGGACGCGCCCTGTAGCGGCGCATTAAGCGCG |
| | GCGGGTGTGGTGGTTACGCGCAGCGTGACCGCTACACTTG |
| | CCAGCGCCCTAGCGCCCGCTCCTTTCGCTTTCTTCCCTTC |
| | CTTTCTCGCCACGTTCGCCGGCTTTCCCCGTCAAGCTCTA |
| | AATCGGGGGCTCCCTTTAGGGTTCCGATTTAGTGCTTTAC |
| | GGCACCTCGACCCCAAAAAACTTGATTAGGGTGATGGTTC |
| | ACGTAGTGGGCCATCGCCCTGATAGACGGTTTTTCGCCCT |
| | TTGACGTTGGAGTCCACGTTCTTTAATAGTGGACTCTTGT |
| | TCCAAACTGGAACAACACTCAACCCTATCTCGGTCTATTC |
| | TTTTGATTTATAAGGGATTTTGCCGATTTCGGCCTATTGG |
| | TTAAAAAATGAGCTGATTTAACAAAAATTTAACGCGAATT |
| | TTAACAAAATATTAACGTTTACAATTTCAGGTGGCACTTT |
| | TCGGGGAAATGTGCGCGGAACCCCTATTTGTTTATTTTTC |
| | TAAATACATTCAAATATGTATCCGCTCATGAATTAATTCT |
| | TAGAAAAACTCATCGAGCATCAAATGAAACTGCAATTTAT |
| | TCATATCAGGATTATCAATACCATATTTTTGAAAAAGCCG |
| | TTTCTGTAATGAAGGAGAAAACTCACCGAGGCAGTTCCAT |
| | AGGATGGCAAGATCCTGGTATCGGTCTGCGATTCCGACTC |
| | GTCCAACATCAATACAACCTATTAATTTCCCCTCGTCAAA |
| | AATAAGGTTATCAAGTGAGAAATCACCATGAGTGACGACT |
| | GAATCCGGTGAGAATGGCAAAAGTTTATGCATTTCTTTCC |
| | AGACTTGTTCAACAGGCCAGCCATTACGCTCGTCATCAAA |
| | ATCACTCGCATCAACCAAACCGTTATTCATTCGTGATTGC |
| | GCCTGAGCGAGACGAAATACGCGATCGCTGTTAAAAGGAC |
| | AATTACAAACAGGAATCGAATGCAACCGGCGCAGGAACAC |
| | TGCCAGCGCATCAACAATATTTTCACCTGAATCAGGATAT |
| | TCTTCTAATACCTGGAATGCTGTTTTCCCGGGGATCGCAG |
| | TGGTGAGTAACCATGCATCATCAGGAGTACGGATAAAATG |
| | CTTGATGGTCGGAAGAGGCATAAATTCCGTCAGCCAGTTT |
| | AGTCTGACCATCTCATCTGTAACATCATTGGCAACGCTAC |
| | CTTTGCCATGTTTCAGAAACAACTCTGGCGCATCGGGCTT |
| | CCCATACAATCGATAGATTGTCGCACCTGATTGCCCGACA |
| | TTATCGCGAGCCCATTTATACCCATATAAATCAGCATCCA |
| | TGTTGGAATTTAATCGCGGCCTAGAGCAAGACGTTTCCCG |
| | TTGAATATGGCTCATAACACCCCTTGTATTACTGTTTATG |
| | TAAGCAGACAGTTTTATTGTTCATGACCAAAATCCCTTAA |
| | CGTGAGTTTTCGTTCCACTGAGCGTCAGACCCCGTAGAAA |
| | AGATCAAAGGATCTTCTTGAGATCCTTTTTTTCTGCGCGT |
| | AATCTGCTGCTTGCAAACAAAAAAACCACCGCTACCAGCG |
| | GTGGTTTGTTTGCCGGATCAAGAGCTACCAACTCTTTTTC |
| | CGAAGGTAACTGGCTTCAGCAGAGCGCAGATACCAAATAC |
| | TGTCCTTCTAGTGTAGCCGTAGTTAGGCCACCACTTCAAG |
| | AACTCTGTAGCACCGCCTACATACCTCGCTCTGCTAATCC |
| | TGTTACCAGTGGCTGCTGCCAGTGGCGATAAGTCGTGTCT |
| | TACCGGGTTGGACTCAAGACGATAGTTACCGGATAAGGCG |
| | CAGCGGTCGGGCTGAACGGGGGGTTCGTGCACACAGCCCA |
| | GCTTGGAGCGAACGACCTACACCGAACTGAGATACCTACA |
| | GCGTGAGCTATGAGAAAGCGCCACGCTTCCCGAAGGGAGA |
| | AAGGCGGACAGGTATCCGGTAAGCGGCAGGGTCGGAACAG |
| | GAGAGCGCACGAGGGAGCTTCCAGGGGGAAACGCCTGGTA |
| | TCTTTATAGTCCTGTCGGGTTTCGCCACCTCTGACTTGAG |
| | CGTCGATTTTTGTGATGCTCGTCAGGGGGGCGGAGCCTAT |
| | GGAAAAACGCCAGCAACGCGGCCTTTTTACGGTTCCTGGC |
| | CTTTTGCTGGCCTTTTGCTCACATGTTCTTTCCTGCGTTA |
| | TCCCCTGATTCTGTGGATAACCGTATTACCGCCTTTGAGT |
| | GAGCTGATACCGCTCGCCGCAGCCGAACGACCGAGCGCAG |
| | CGAGTCAGTGAGCGAGGAAGCGGAAGAGCGCCTGATGCGG |
| | TATTTTCTCCTTACGCATCTGTGCGGTATTTCACACCGCA |
| | TATATGGTGCACTCTCAGTACAATCTGCTCTGATGCCGCA |
| | TAGTTAAGCCAGTATACACTCCGCTATCGCTACGTGACTG |
| | GGTCATGGCTGCGCCCCGACACCCGCCAACACCCGCTGAC |
| | GCGCCCTGACGGGCTTGTCTGCTCCCGGCATCCGCTTACA |
| | GACAAGCTGTGACCGTCTCCGGGAGCTGCATGTGTCAGAG |
| | GTTTTCACCGTCATCACCGAAACGCGCGAGGCAGCTGCGG |
| | TAAAGCTCATCAGCGTGGTCGTGAAGCGATTCACAGATGT |
| | CTGCCTGTTCATCCGCGTCCAGCTCGTTGAGTTTCTCCAG |
| | AAGCGTTAATGTCTGGCTTCTGATAAAGCGGGCCATGTTA |
| | AGGGCGGTTTTTTCCTGTTTGGTCACTGATGCCTCCGTGT |
| | AAGGGGGATTTCTGTTCATGGGGGTAATGATACCGATGAA |
| | ACGAGAGAGGATGCTCACGATACGGGTTACTGATGATGAA |
| | CATGCCCGGTTACTGGAACGTTGTGAGGGTAAACAACTGG |
| | CGGTATGGATGCGGCGGGACCAGAGAAAAATCACTCAGGG |
| | TCAATGCCAGCGCTTCGTTAATACAGATGTAGGTGTTCCA |
| | CAGGGTAGCCAGCAGCATCCTGCGATGCAGATCCGGAACA |
| | TAATGGTGCAGGGCGCTGACTTCCGCGTTTCCAGACTTTA |
| | CGAAACACGGAAACCGAAGACCATTCATGTTGTTGCTCAG |
| | GTCGCAGACGTTTTGCAGCAGCAGTCGCTTCACGTTCGCT |
| | CGCGTATCGGTGATTCATTCTGCTAACCAGTAAGGCAACC |
| | CCGCCAGCCTAGCCGGGTCCTCAACGACAGGAGCACGATC |
| | ATGCGCACCCGTGGGGCCGCCATGCCGGCGATAATGGCCT |
| | GCTTCTCGCCGAAACGTTTGGTGGCGGGACCAGTGACGAA |
| | GGCTTGAGCGAGGGCGTGCAAGATTCCGAATACCGCAAGC |
| | GACAGGCCGATCATCGTCGCGCTCCAGCGAAAGCGGTCCT |
| | CGCCGAAAATGACCCAGAGCGCTGCCGGCACCTGTCCTAC |
| | GAGTTGCATGATAAAGAAGACAGTCATAAGTGCGGCGACG |
| | ATAGTCATGCCCCGCGCCCACCGGAAGGAGCTGACTGGGT |
| | TGAAGGCTCTCAAGGGCATCGGTCGAGATCCCGGTGCCTA |
| | ATGAGTGAGCTAACTTACATTAATTGCGTTGCGCTCACTG |
| | CCCGCTTTCCAGTCGGGAAACCTGTCGTGCCAGCTGCATT |
| | AATGAATCGGCCAACGCGCGGGGAGAGGCGGTTTGCGTAT |
| | TGGGCGCCAGGGTGGTTTTTCTTTTCACCAGTGAGACGGG |
| | CAACAGCTGATTGCCCTTCACCGCCTGGCCCTGAGAGAGT |
| | TGCAGCAAGCGGTCCACGCTGGTTTGCCCCAGCAGGCGAA |
| | AATCCTGTTTGATGGTGGTTAACGGCGGGATATAACATGA |
| | GCTGTCTTCGGTATCGTCGTATCCCACTACCGAGATATCC |
| | GCACCAACGCGCAGCCCGGACTCGGTAATGGCGCGCATTG |
| | CGCCCAGCGCCATCTGATCGTTGGCAACCAGCATCGCAGT |
| | GGGAACGATGCCCTCATTCAGCATTTGCATGGTTTGTTGA |
| | AAACCGGACATGGCACTCCAGTCGCCTTCCCGTTCCGCTA |
| | TCGGCTGAATTTGATTGCGAGTGAGATATTTATGCCAGCC |
| | AGCCAGACGCAGACGCGCCGAGACAGAACTTAATGGGCCC |
| | GCTAACAGCGCGATTTGCTGGTGACCCAATGCGACCAGAT |
| | GCTCCACGCCCAGTCGCGTACCGTCTTCATGGGAGAAAAT |
| | AATACTGTTGATGGGTGTCTGGTCAGAGACATCAAGAAAT |
| | AACGCCGGAACATTAGTGCAGGCAGCTTCCACAGCAATGG |
| | CATCCTGGTCATCCAGCGGATAGTTAATGATCAGCCCACT |
| | GACGCGTTGCGCGAGAAGATTGTGCACCGCCGCTTTACAG |
| | GCTTCGACGCCGCTTCGTTCTACCATCGACACCACCACGC |
| | TGGCACCCAGTTGATCGGCGCGAGATTTAATCGCCGCGAC |
| | AATTTGCGACGGCGCGTGCAGGGCCAGACTGGAGGTGGCA |
| | ACGCCAATCAGCAACGACTGTTTGCCCGCCAGTTGTTGTG |
| | CCACGCGGTTGGGAATGTAATTCAGCTCCGCCATCGCCGC |
| | TTCCACTTTTTCCCGCGTTTTCGCAGAAACGTGGCTGGCC |
| | TGGTTCACCACGCGGGAAACGGTCTGATAAGAGACACCGG |
| | CATACTCTGCGACATCGTATAACGTTACTGGTTTCACATT |
| | CACCACCCTGAATTGACTCTCTTCCGGGCGCTATCATGCC |
| | ATACCGCGAAAGGTTTTGCGCCATTCGATGGTGTCCGGGA |
| | TCTCGACGCTCTCCCTTATGCGACTCCTGCATTAGGAAGC |
| | AGCCCAGTAGTAGGTTGAGGCCGTTGAGCACCGCCGCCGC |
| | AAGGAATGGTGCATGCAAGGAGATGGCGCCCAACAGTCCC |
| | CCGGCCACGGGGCCTGCCACCATACCCACGCCGAAACAAG |
| | CGCTCATGAGCCCGAAGTGGCGAGCCCGATCTTCCCCATC |
| | GGTGATGTCGGCGATATAGGCGCCAGCAACCGCACCTGTG |
| | GCGCCGGTGATGCCGGCCACGATGCGTCCGGCGTAGAGGA |
| | TCGAGATCTCGATCCCGCGAAATTAATACGACTCACTATA |
| | GGGGAATTGTGAGCGGATAACAATTCCCCTCTAGATTTAC |
| | ACTTTATGCTTCCGGCTCGTATGTTTCTAGAGAATTCAAA |
| | TAATTTTGTTTAACTTTAAGAAGGAGATTTAAATATGGGC |
| | AGCAGCCATCATCATCATCATCACAGCAGCGGCCTGGTGC |
| | CGCGCGGCAGCATGCAGAGGATTTTCGGCCTCGATATCGG |
| | CACCACGTCCATCGGCTTTGCGGTCATCGACCACGACCGC |
| | GACCAAGGCGTCGGCCGCATCCACCGGCTGGGCGCGCGCA |
| | TCTTCCCGGAAGCGCGCGACGAGAAGGGAACACCGCTCAA |
| | CCAGCATCGGCGGCAAAAGCGTCTCGCGCGCCGCCAATTG |
| | CGCCGGCGCCGGCTTCGGCGCAAGGCGCTCAACGAACTGC |
| | TTTCGGCCCGCGGGATGCTGCCGCGCTTCGGCACGTCCGC |
| | TTGGCACGACGCGATGGCGCTCGACCCTTACGCGCTCCGT |
| | GCACGGGGTACGGAGGAGGCGTTGCAGCCGGTAGAGGTCG |
| | GTCGGGCTCTCTATCACCTCGCCCAGCGTCGCCACTTCAA |
| | GCCACGGGACGAGGCTGCGGAAGCCGACGAGCAGGAGGTG |
| | GGCGATCAGGAGGCCGAGACCAAGCGTGAGAAGCTGCTGC |
| | AGGCGTTGCGCCGCAGCGGTCGAACGCTGGGCCAGGAACT |
| | GGCGGCGCGCGGTCCGCACGAGCGCAAGCGGCACGAGCAC |
| | GCTTTGCGCTCGACCGTCGAGACCGAGTTCGAGCGGCTCC |
| | TCACCGCGCAAGCGCGGCATCACGAGATCCTTCGCGATCC |
| | CGAGTTCGTCGAGGAACTGAGAGAGACCATCTTCGCGCAA |
| | CGGCCCGTCTTTTGGCGGACGAGCACGCTCGGCACGTGCC |
| | CGTTCGTTCCAGGCGCACCGTTGTGCCCGAAGGGTGCTTG |
| | GCTCTCCCGCCAGCGGCGCATGCTGGAGCAGGTCAACAAC |
| | CTCGCCATCACCGGCGGCAACGCGCGTCCGCTCGACCACG |
| | AGGAGCGACGAGCGATCCTCGCCGTCTTACAGACGCAGGC |
| | CAGCATGAGCTGGGGCGCGGTCCGAACCGCGCTTAAGCCG |
| | CTCTTCAAGGCACGCGGCGAGGCGGGCGCCGAGCGTCGGC |
| | TCCGGTTCAATCTCGAAGAGGGCGGCGGTAAGACGCTGCT |
| | CGGGAACCCGCTGGAAGCGAAGCTCGCCCGGATCTTCGGC |
| | GAAGCCTGGGCCACGCACCCTCACCGCGACGCGATCCGTG |
| | AGACGATCCATGACCGCCTTTTCGCCGCGACCTATAACGC |
| | GAAGGGCGCGCAGCGCATCGTCATCCTTCCGGCATCCCAA |
| | CGCGCTGAACGGATGCGGGGGGTCATCGCCGGCCTCCAAG |
| | CGGATTTCGGCCTTTCCCACGAGCAGGCGATGGCGCTTGC |
| | GGAGCTGCCGCTGACGCCCGGCTGGGAACCCTATTCGAGC |
| | GAAGCCCTTCGCGCGTTAATGCCGAAGCTGGAGGAAGGCG |
| | TGCGCTTCGGCGCCCTCGTCGTGGCCCCTGAATGGGAAGA |
| | TTGGCGCGAGGCCACCTTCCCCCAGCGCGAGCGGCCGACC |
| | GGCGAGGTGCTCGACCTCTTGCCTTCACCGAAATGCCACG |
| | ATGAGAGCCGCCGGCAGACGCGGCTGCGGAACCCGACGGT |
| | GCTGCGCACGCAGAACGAGCTGCGCAAGGTCGTCAACAAC |
| | CTGATCCGGGCGCACGGCAAGCCCGACATCATCCGCGTCG |
| | AGGTCGCCCGCGAGGTGGGGCTTTCCAAGCGCGAGCGTGA |
| | AGATCGCTACAACGGGATGCGGCGCCAGGAGCGCCAGCGG |
| | CAAGCGGCGATCAAAGACCTCCAAGCCAAGGGCTTCGCCG |
| | AGCCGTCGCGCGCCGACGTCGAGAAGTGGCTTTTGTGGAA |
| | GGAGAGCAAGGAGACCTGCCCTTACACGGGGGACAAGATC |
| | TGCTTCGACGCTCTGTTTCGCCGCGGTGAGTTTCAAGTGG |
| | AGCACATCTGGCCGCGCTCGCGCTCGTTCGACGACAGCTT |
| | CCGCAACAAGACCCTGTGTCGGCGCGACGTGAACCTCGCC |
| | AAGGGTAACCAAACGCCCTTCGAGTTCTTCGAGAGCCGAC |
| | CCGAGGAGTGGGAGGCCGTGAAGCGCCGCCTCGATGGCTT |
| | GCAGGCCAAGCGGGCAGGCGGTGAGGGGATGGCGCGCGGC |
| | AAGGTGAAGCGCTTCGTCGCGAGCACGTTGCCGGACGATT |
| | TCGCGCAGCGTCAGCTCAACGACACGGGCTGGGCGGCGCG |
| | CGAGGCGGTGGCCTTCCTCAAGCGGCTGTGGCCGGACGAG |
| | GGGCAAGCCGCGCCGGTCCGCGTCCAGGCGGTCACGGGGC |
| | GGGTGACGGCGCAGCTTCGCCACCTGGGGGGCCTCGATGG |
| | CGTGCTGTCGGACGGTGCTCGAAAGACGCGTGACGACCAC |
| | CGCCATCACGCCGTCGATGCGCTGGTCGTCGCCTGCACGC |
| | ATCCGGGCATGACCGAGCGGCTCAGCCGCTACTGGCAGCA |
| | GAAGGAGGACGAGCGCGCCGAACGACCGCAGCTGGACCCA |
| | CCGTGGCCCACGATCCGAGCGGACGCCGAGGCGGCCAAGG |
| | ACTTAATCGTCGTCTCGCACCGGGTGCGCAAGAAGATCTC |
| | GGGACCGTTCCACAAGGAAACCGTCTATGGCGCGACCGAC |
| | GAGCGCGAGGTCACGCGCGGGCTTGAGTACGAGAAATTCG |
| | TCACGCGGAAGCGCGTCGAGGACCTGACGAAATCCATGCT |
| | CGCCGACATCCGCGACGACAGGGTGCGGCAAATTGTGACG |
| | GCGTGGGTGGCCGAGCGCGGCGGCGACCCGAAGAAGGCGT |
| | TTCCGCCCTATCCGACGCTGGGGTCGAGCGGACCCGAGAT |
| | CCGCAAGGTGCGCGTTCTGATCCGCCGGCAGCCCACCTTG |
| | ATGGCACGGGCAGCGACGGGCTTCGCTGATCTCGGAGCGA |
| | ACCACCATGTCGCCATCTACAAGACCGCCGACGAGCGATT |
| | CGCCTTCGAGGTCGTCAGCTTGCTGGAGGTCGCCAGGCGC |
| | GTCGACCGCGGTGAACCGCCCGTGAAGAGACAGCGAGGCG |
| | ACGAGAAGCTCGTGATGTCTTTGGCGCAGGGCGATCTGAT |
| | ACGGTTCGCCAAAACGCCCGATGCGGAAGCAGCAATTTGG |
| | CGTGTTCAGAAAATCGCAACTAAAGGTCAGATATCGCTCC |
| | TTCACCACGATGACGCTTCGCCGAAGGAGCCGAGTCTCTT |
| | TGAACCGATGGTTGGTGGGTTGATGGCTCGGAACCCGGAG |
| | AAGCTGGCAGTCGATCCCATCGGCCGAGTGCGCAAGGCAG |
| | GCGACTGACTAGCATAACCCCTTGGGGCCTCTAAACGGGT |
| | CTTGAGGGGTTTTTTGCATATGCTGAAAGGAGGAACTATA |
| | TCCGGAT (SEQ ID NO: 67) |
| Cas9.2 | TGGCGAATGGGACGCGCCCTGTAGCGGCGCATTAAGCGCG |
| | GCGGGTGTGGTGGTTACGCGCAGCGTGACCGCTACACTTG |
| | CCAGCGCCCTAGCGCCCGCTCCTTTCGCTTTCTTCCCTTC |
| | CTTTCTCGCCACGTTCGCCGGCTTTCCCCGTCAAGCTCTA |
| | AATCGGGGGCTCCCTTTAGGGTTCCGATTTAGTGCTTTAC |
| | GGCACCTCGACCCCAAAAAACTTGATTAGGGTGATGGTTC |
| | ACGTAGTGGGCCATCGCCCTGATAGACGGTTTTTCGCCCT |
| | TTGACGTTGGAGTCCACGTTCTTTAATAGTGGACTCTTGT |
| | TCCAAACTGGAACAACACTCAACCCTATCTCGGTCTATTC |
| | TTTTGATTTATAAGGGATTTTGCCGATTTCGGCCTATTGG |
| | TTAAAAAATGAGCTGATTTAACAAAAATTTAACGCGAATT |
| | TTAACAAAATATTAACGTTTACAATTTCAGGTGGCACTTT |
| | TCGGGGAAATGTGCGCGGAACCCCTATTTGTTTATTTTTC |
| | TAAATACATTCAAATATGTATCCGCTCATGAATTAATTCT |
| | TAGAAAAACTCATCGAGCATCAAATGAAACTGCAATTTAT |
| | TCATATCAGGATTATCAATACCATATTTTTGAAAAAGCCG |
| | TTTCTGTAATGAAGGAGAAAACTCACCGAGGCAGTTCCAT |
| | AGGATGGCAAGATCCTGGTATCGGTCTGCGATTCCGACTC |
| | GTCCAACATCAATACAACCTATTAATTTCCCCTCGTCAAA |
| | AATAAGGTTATCAAGTGAGAAATCACCATGAGTGACGACT |
| | GAATCCGGTGAGAATGGCAAAAGTTTATGCATTTCTTTCC |
| | AGACTTGTTCAACAGGCCAGCCATTACGCTCGTCATCAAA |
| | ATCACTCGCATCAACCAAACCGTTATTCATTCGTGATTGC |
| | GCCTGAGCGAGACGAAATACGCGATCGCTGTTAAAAGGAC |
| | AATTACAAACAGGAATCGAATGCAACCGGCGCAGGAACAC |
| | TGCCAGCGCATCAACAATATTTTCACCTGAATCAGGATAT |
| | TCTTCTAATACCTGGAATGCTGTTTTCCCGGGGATCGCAG |
| | TGGTGAGTAACCATGCATCATCAGGAGTACGGATAAAATG |
| | CTTGATGGTCGGAAGAGGCATAAATTCCGTCAGCCAGTTT |
| | AGTCTGACCATCTCATCTGTAACATCATTGGCAACGCTAC |
| | CTTTGCCATGTTTCAGAAACAACTCTGGCGCATCGGGCTT |
| | CCCATACAATCGATAGATTGTCGCACCTGATTGCCCGACA |
| | TTATCGCGAGCCCATTTATACCCATATAAATCAGCATCCA |
| | TGTTGGAATTTAATCGCGGCCTAGAGCAAGACGTTTCCCG |
| | TTGAATATGGCTCATAACACCCCTTGTATTACTGTTTATG |
| | TAAGCAGACAGTTTTATTGTTCATGACCAAAATCCCTTAA |
| | CGTGAGTTTTCGTTCCACTGAGCGTCAGACCCCGTAGAAA |
| | AGATCAAAGGATCTTCTTGAGATCCTTTTTTTCTGCGCGT |
| | AATCTGCTGCTTGCAAACAAAAAAACCACCGCTACCAGCG |
| | GTGGTTTGTTTGCCGGATCAAGAGCTACCAACTCTTTTTC |
| | CGAAGGTAACTGGCTTCAGCAGAGCGCAGATACCAAATAC |
| | TGTCCTTCTAGTGTAGCCGTAGTTAGGCCACCACTTCAAG |
| | AACTCTGTAGCACCGCCTACATACCTCGCTCTGCTAATCC |
| | TGTTACCAGTGGCTGCTGCCAGTGGCGATAAGTCGTGTCT |
| | TACCGGGTTGGACTCAAGACGATAGTTACCGGATAAGGCG |
| | CAGCGGTCGGGCTGAACGGGGGGTTCGTGCACACAGCCCA |
| | GCTTGGAGCGAACGACCTACACCGAACTGAGATACCTACA |
| | GCGTGAGCTATGAGAAAGCGCCACGCTTCCCGAAGGGAGA |
| | AAGGCGGACAGGTATCCGGTAAGCGGCAGGGTCGGAACAG |
| | GAGAGCGCACGAGGGAGCTTCCAGGGGGAAACGCCTGGTA |
| | TCTTTATAGTCCTGTCGGGTTTCGCCACCTCTGACTTGAG |
| | CGTCGATTTTTGTGATGCTCGTCAGGGGGGCGGAGCCTAT |
| | GGAAAAACGCCAGCAACGCGGCCTTTTTACGGTTCCTGGC |
| | CTTTTGCTGGCCTTTTGCTCACATGTTCTTTCCTGCGTTA |
| | TCCCCTGATTCTGTGGATAACCGTATTACCGCCTTTGAGT |
| | GAGCTGATACCGCTCGCCGCAGCCGAACGACCGAGCGCAG |
| | CGAGTCAGTGAGCGAGGAAGCGGAAGAGCGCCTGATGCGG |
| | TATTTTCTCCTTACGCATCTGTGCGGTATTTCACACCGCA |
| | TATATGGTGCACTCTCAGTACAATCTGCTCTGATGCCGCA |
| | TAGTTAAGCCAGTATACACTCCGCTATCGCTACGTGACTG |
| | GGTCATGGCTGCGCCCCGACACCCGCCAACACCCGCTGAC |
| | GCGCCCTGACGGGCTTGTCTGCTCCCGGCATCCGCTTACA |
| | GACAAGCTGTGACCGTCTCCGGGAGCTGCATGTGTCAGAG |
| | GTTTTCACCGTCATCACCGAAACGCGCGAGGCAGCTGCGG |
| | TAAAGCTCATCAGCGTGGTCGTGAAGCGATTCACAGATGT |
| | CTGCCTGTTCATCCGCGTCCAGCTCGTTGAGTTTCTCCAG |
| | AAGCGTTAATGTCTGGCTTCTGATAAAGCGGGCCATGTTA |
| | AGGGCGGTTTTTTCCTGTTTGGTCACTGATGCCTCCGTGT |
| | AAGGGGGATTTCTGTTCATGGGGGTAATGATACCGATGAA |
| | ACGAGAGAGGATGCTCACGATACGGGTTACTGATGATGAA |
| | CATGCCCGGTTACTGGAACGTTGTGAGGGTAAACAACTGG |
| | CGGTATGGATGCGGCGGGACCAGAGAAAAATCACTCAGGG |
| | TCAATGCCAGCGCTTCGTTAATACAGATGTAGGTGTTCCA |
| | CAGGGTAGCCAGCAGCATCCTGCGATGCAGATCCGGAACA |
| | TAATGGTGCAGGGCGCTGACTTCCGCGTTTCCAGACTTTA |
| | CGAAACACGGAAACCGAAGACCATTCATGTTGTTGCTCAG |
| | GTCGCAGACGTTTTGCAGCAGCAGTCGCTTCACGTTCGCT |
| | CGCGTATCGGTGATTCATTCTGCTAACCAGTAAGGCAACC |
| | CCGCCAGCCTAGCCGGGTCCTCAACGACAGGAGCACGATC |
| | ATGCGCACCCGTGGGGCCGCCATGCCGGCGATAATGGCCT |
| | GCTTCTCGCCGAAACGTTTGGTGGCGGGACCAGTGACGAA |
| | GGCTTGAGCGAGGGCGTGCAAGATTCCGAATACCGCAAGC |
| | GACAGGCCGATCATCGTCGCGCTCCAGCGAAAGCGGTCCT |
| | CGCCGAAAATGACCCAGAGCGCTGCCGGCACCTGTCCTAC |
| | GAGTTGCATGATAAAGAAGACAGTCATAAGTGCGGCGACG |
| | ATAGTCATGCCCCGCGCCCACCGGAAGGAGCTGACTGGGT |
| | TGAAGGCTCTCAAGGGCATCGGTCGAGATCCCGGTGCCTA |
| | ATGAGTGAGCTAACTTACATTAATTGCGTTGCGCTCACTG |
| | CCCGCTTTCCAGTCGGGAAACCTGTCGTGCCAGCTGCATT |
| | AATGAATCGGCCAACGCGCGGGGAGAGGCGGTTTGCGTAT |
| | TGGGCGCCAGGGTGGTTTTTCTTTTCACCAGTGAGACGGG |
| | CAACAGCTGATTGCCCTTCACCGCCTGGCCCTGAGAGAGT |
| | TGCAGCAAGCGGTCCACGCTGGTTTGCCCCAGCAGGCGAA |
| | AATCCTGTTTGATGGTGGTTAACGGCGGGATATAACATGA |
| | GCTGTCTTCGGTATCGTCGTATCCCACTACCGAGATATCC |
| | GCACCAACGCGCAGCCCGGACTCGGTAATGGCGCGCATTG |
| | CGCCCAGCGCCATCTGATCGTTGGCAACCAGCATCGCAGT |
| | GGGAACGATGCCCTCATTCAGCATTTGCATGGTTTGTTGA |
| | AAACCGGACATGGCACTCCAGTCGCCTTCCCGTTCCGCTA |
| | TCGGCTGAATTTGATTGCGAGTGAGATATTTATGCCAGCC |
| | AGCCAGACGCAGACGCGCCGAGACAGAACTTAATGGGCCC |
| | GCTAACAGCGCGATTTGCTGGTGACCCAATGCGACCAGAT |
| | GCTCCACGCCCAGTCGCGTACCGTCTTCATGGGAGAAAAT |
| | AATACTGTTGATGGGTGTCTGGTCAGAGACATCAAGAAAT |
| | AACGCCGGAACATTAGTGCAGGCAGCTTCCACAGCAATGG |
| | CATCCTGGTCATCCAGCGGATAGTTAATGATCAGCCCACT |
| | GACGCGTTGCGCGAGAAGATTGTGCACCGCCGCTTTACAG |
| | GCTTCGACGCCGCTTCGTTCTACCATCGACACCACCACGC |
| | TGGCACCCAGTTGATCGGCGCGAGATTTAATCGCCGCGAC |
| | AATTTGCGACGGCGCGTGCAGGGCCAGACTGGAGGTGGCA |
| | ACGCCAATCAGCAACGACTGTTTGCCCGCCAGTTGTTGTG |
| | CCACGCGGTTGGGAATGTAATTCAGCTCCGCCATCGCCGC |
| | TTCCACTTTTTCCCGCGTTTTCGCAGAAACGTGGCTGGCC |
| | TGGTTCACCACGCGGGAAACGGTCTGATAAGAGACACCGG |
| | CATACTCTGCGACATCGTATAACGTTACTGGTTTCACATT |
| | CACCACCCTGAATTGACTCTCTTCCGGGCGCTATCATGCC |
| | ATACCGCGAAAGGTTTTGCGCCATTCGATGGTGTCCGGGA |
| | TCTCGACGCTCTCCCTTATGCGACTCCTGCATTAGGAAGC |
| | AGCCCAGTAGTAGGTTGAGGCCGTTGAGCACCGCCGCCGC |
| | AAGGAATGGTGCATGCAAGGAGATGGCGCCCAACAGTCCC |
| | CCGGCCACGGGGCCTGCCACCATACCCACGCCGAAACAAG |
| | CGCTCATGAGCCCGAAGTGGCGAGCCCGATCTTCCCCATC |
| | GGTGATGTCGGCGATATAGGCGCCAGCAACCGCACCTGTG |
| | GCGCCGGTGATGCCGGCCACGATGCGTCCGGCGTAGAGGA |
| | TCGAGATCTCGATCCCGCGAAATTAATACGACTCACTATA |
| | GGGGAATTGTGAGCGGATAACAATTCCCCTCTAGATTTAC |
| | ACTTTATGCTTCCGGCTCGTATGTTTCTAGAGAATTCAAA |
| | TAATTTTGTTTAACTTTAAGAAGGAGATTTAAATATGGGC |
| | AGCAGCCATCATCATCATCATCACAGCAGCGGCCTGGTGC |
| | CGCGCGGCAGCATGAAGAAAGAAAAAGTGTACATGGGGCT |
| | AGATCTTGGCACGAACTCTGTCGGCTGGGCGGTAACGGAT |
| | AACGACTATAAGGTGCTCAAGTTTAAACGGCGCGCTATGT |
| | GGGGGGTTCGGCTCTTTAATGAAGCCAATCCGGCTGTCGA |
| | GAGGCGTGTTGCCCGTTCAAATCGCCGTCGCCTGGCCCGA |
| | AAAAAACAACGCGTGGCTTGGCTGAAAGAAATATTTAAGA |
| | ATTCCATTAGCGAAATTGATCCGGAATTTTTCGACCGTCT |
| | TGAACAAAGCGCGCTTTGGGCAGAAGACAAAAATGTCGCC |
| | GGAAAATACTCCCTTTTTAATGAGAAAAAATTAACCGATA |
| | AGACATTCTATCGGAAATTTCCCACCGTTTTTCACCTAAA |
| | AAAAGCGCTTATGGACGGCAAAATAAAAAAACCTGATATT |
| | CGCTTTGTATATCTTGCCTTGTCCCACTATCTGCAAAACA |
| | GAGGCCATTTTCTCTTGGAAAATGAGCTGAACAGTGTTGA |
| | AGATATAGACATTCGGGATATTTTTAACAGTCTTAATGAA |
| | AGAATTCATGTTCTTATTGACAGCGGTGATGATATGGTTC |
| | CTGCTTTTGATTTGACAAACCTTGATGATTTGAAACAAAT |
| | TGCCACAGACACAAATATATCCGGGAAAACGCAGGAAAAA |
| | GAAGCCTTTATAAAAACCCTGTTAAATGGGGCCAAACAGC |
| | CTGCCTTAGAGGCAATTATTAAATTATGTACAGGCGGCTC |
| | GGCTAATTTATCAAAAATCTTTGGTGATATGTTTGAATTT |
| | GAAAGTGAAATCAAATCAATATCATTCGAAAAGGCCAACT |
| | TCGAAGATGAAATCGCTCCCAAGCTGCAAGATTGTCTGGG |
| | AGATTACTATCAGATTATTGAGCTGGCTCAGCAGATTTAC |
| | AGCTGGTACACGCTTTATAAGGTATGCAGCGGTCGACCGT |
| | CGGTCTCTCACGCCAAAGTGGAGGATTACGAAAAACACAA |
| | AGAACAGCTGTCCCACCTAAAAGTGCTGGTAAGAAAACAC |
| | TTTTCGAAAAATGTCTACCGGGAAATATTCCGAAAAGAAG |
| | ACGACAAAATCCATAACTATGTATCCTACATATCCGGCAA |
| | AAAGGACCGCGACGAATTTTATAAATATCTCAAAAAAACG |
| | TTAGAAAAAAAATCTACATTCAAGAAAACGTCTGAATTTG |
| | AGAATATTTCTCGCGCCATTGAACAGCAAAACTACCTGCC |
| | GAAACAACGGGTCAAAGACAACTCTGTGGTGCCTCAGCAG |
| | CTATACAAACAAGAAATCGTAAAAATCCTCAACAACCTTT |
| | CATCACACTACCCCTTTTTATCACAAAAAACAGACGGGAT |
| | CAGCAATCGAGAAAAGATTATCAAAATCTTTGAATACCGC |
| | ATCCCATACTATGTCGGTCCTCTTTGCGATATCCATCGTG |
| | CGGGGGATGACGGGTTCTCCTGGCTGGTTCGTGACTGCAG |
| | TAAAAAGATTACTCCTTGGAACTTCGAGCAAGTCGTCGAT |
| | ATCCCCCAGTCTGCTGAAAATTTCATTAAGAACATGACCC |
| | GTAAATGCACCTATTTAAAACAGTATAATGTGCTGCCGAA |
| | AAATTCTCTCCTCTATAGCGAGTATAGCGTACTAAATGAA |
| | CTTAACAATGTGCGCATCAAAACTAAAAAGCTGACCCCTA |
| | AGCTAAAAGAAAAAATGCTCAACACATTATTTCGCCAAAA |
| | GAAGAATATTTCGATAACGAGCTTGATTCATTGGCTTGTC |
| | AGTGAAGGAGTGTATGAGAAAGGGGAGATTGAAAAATCAG |
| | ACGTCAGCGGTGTTGATTCCAATTTTACCAGCTCTCTTTC |
| | TGCAGCCATTTCTTTTGATCGTATCATTGGTGAAAAGATG |
| | AAAAACAAAAAAACCCAAAAAATGGTCGAGGAGATCATAA |
| | ACTGGCTCGCCCTTTTTTCGGACAAAAAAATACTACAACA |
| | AAAGATTGTAGAGAAATATCAAGATAAAGTCTCGCAAGAA |
| | CAAATCGGAAAAATTCTGCGCCTCAACCTAAGCGGATGGG |
| | GACGACTTTCTTCGGAGTTTCTGCAACTGAAAAACTCCCA |
| | ACCGGGAGAACACGACGGAAAAACGCTCATCAATATCATG |
| | CGGCAGACCCAGATGAATCTGATGGAGATTATTCACTCTC |
| | CCCAGTTCAGTTTCAATACCGTTATTGAAACGGAGGCCAA |
| | AAAACAGCTAACGGGACACATTACCCACAGTCATGTTGAG |
| | GCGCTGTACTGCTCTCCTGTGGTCAAGAAACAGATATGGC |
| | AGGCCCTGCAAATCGCCCTGGAGCTAAAGAAAACCTTAAA |
| | GAAAGACCCGAACAAAATTTTTGTGGAGACAACCCGGCAT |
| | GAAGGGGAGAAAAAACGGACCACAAGCCGTCACAAACAAC |
| | TACTCGAGTTATACCAAGCCGCCAAGTCCCATCTGCCCGA |
| | CCTGACGAAAAGTATAAAGGAACTAAACGATGCGCTAAAA |
| | GATACAGAGCCGGAGAAGATGAAACGGAAAAAACTGTTTC |
| | ACTACTACAAACAACTGGGACGTTGTATGTATACAGGCAG |
| | GCCCATCAGTCTAGAGGATCTGTTTACCAATAAATATGAC |
| | ATTGATCATATTTATCCCCAGAGTTTAACCAAAGATGACA |
| | GTTTTACTAATACTGTACTGGTGGAACGGCTATCAAACGC |
| | GGAGAAATCAGACGCATTCCCTCTTGACAGTAAAACAAGA |
| | AAAGACCGTCAAGGACTGTGGCGCTGTTTACGACGGAACG |
| | GACTAATTACCAAAGAAAAGTACTACCGCTTAACACGGGA |
| | AACACCTCTAAGCGAAGAAGAAAAAGCGGCCTTTATTCGT |
| | CGTCAGCTGGTGGAAACCAGCCAGACAACCAAGGAAGTAA |
| | TCCGATTTCTGGCGACCCTTTTCCCAAAGTCAAAAGTTGT |
| | GTATGTAAAGAGCGGCAACGTCAGCGACTTTCGCCGTGAC |
| | TTTTCCCCGTCCCTGCCCGAAAACAAAACTAACGGCAAAG |
| | ACCCCAAGGGGATAACCGACTACAGCATGATTAAAGTGAG |
| | GGAAATCAATGATTTGCACCACGCGAAAGACGCGTATTTA |
| | AACATCGTGGTCGGCAATGTCTACGACACCAAATTTCGCT |
| | ACCGAGGCAAAGACCTCACGGCCATAGTGCGCGAAAAAGC |
| | GAGGCAGTACCATTTATCCCGTTTGTTTCTTTACTCTACC |
| | GACGGCGCCTGGATCGGAGCGGCTGATGAAAACAGAGGGA |
| | AGCAACGACCGAGTATTGAAACCGTGATCGCGGAAATGCG |
| | GCGAAATAGCTGTCAGGTAACGTGGGAAGCCGTCTTTAAA |
| | AAAGGGCAGCTGTGGGACATGAACGCCAAAAGTAAGCGGC |
| | CGGGACTGTTGCCGATCAAGAAAGAACTATCCGATACGGC |
| | AAAATATGGAGGGTACCAGGGGAAGACCGCGTCTTATTTT |
| | GTGGTCGTTGAGTATGAGAATAAAAAAGGCGAACGTGAAA |
| | AAAAACTGGAATCGGTCCCGATTTATGTGAAAGCGCTCAG |
| | TAAACAAAAGCCGGACGCTGTCAATTCTTTCCTACGGGAT |
| | ACACTGGGTCTGGAGAAACCAAGCGTCATGGTCGACAACA |
| | TCAAAATCGGCTCCATCGTCGAGATCAACGGGGCCCGAAT |
| | GGTCCTTACGGGGAATAATGAAGTTCTAGTATTTGGGCGT |
| | ATCGCGTCCCAACTGATCCTGGATATAACGATGGCCGCCT |
| | ATCTAAAACGAATGTTTAAGCTGCTTGCTGACACAGCCAA |
| | GATCAAAGAGAACAATGTCTACTTTAAAAACTGCGGCTAT |
| | CTGGATAAGGAGACGAACCTGGCAGTATACGATACGTTTA |
| | TTGCCAAGCTGAAACTGCCCCGGTATGCTCAGATTATCAC |
| | CCATAGCCTATATGAGAAGATGGAAAGCAATCGTGATGTG |
| | TTTATCAACCTTTCACTGGCCGACCAGTGTAATCTGCTGG |
| | CCGGCGTACTGCCTGCGCTACAGTGTAACAGCCAAAATGC |
| | CGATCTGTCTCTTCTTGGTGAAGGTAAAGCGGTCGGAAAT |
| | ATCGCATTTTCAAAAAACGCGATCCTGAAAAAGAATCAGG |
| | TCCGTCTTGTTGATTGCTCCATTACCGGGCTCTTCGAAAA |
| | CAGCAGAAATATGGCATAACTAGCATAACCCCTTGGGGCC |
| | TCTAAACGGGTCTTGAGGGGTTTTTTGCATATGCTGAAAG |
| | GAGGAACTATATCCGGAT (SEQ ID NO: 68) |

### Protein Expression and Purification

Cas12 coding sequences were codon-optimized and synthesized by GeneScript and then cloned into pET28a (Novagen) with N-terminal 6×His tagging. Cas12 expression plasmids were transformed into E. coli NiCo21 (DE3) (NEB). For protein expression, a single clone was first cultured overnight in 5-mL liquid LB tubes and then inoculated into 400 ml of fresh liquid LB (OD 600 0.1). Cells were grown with shaking at 200 rpm and 37° C. until the OD 600 reached 0.8, and IPTG was then added to a final concentration of 0.1 mM followed by further culture of the cells at 37° C. for about 2 h before the cell harvesting. Cells were resuspended in 20 mL of buffer A (50 mM Tris-HCl pH 8.0, 0.5 M NaCl, 1 mM DTT and 5% glycerol) with protease inhibitor cocktail (Promega) and 5 mg/ml lysozyme. After a 15 min incubation at 37° C., cells were lysed by sonication for 10 minutes with 10 s on and 10 s off cycle. Cell debris and insoluble particles were removed by centrifugation (15,000 rpm for 30 min). After centrifuging, the supernatant was loaded onto a 5 mL Crude HisTrap column (GE Healthcare) equilibrated in buffer A with 20 mM imidazol on an AKTA Pure 25L device (GE Healthcare Life Sciences). The elution was performed by a step gradient of buffer B (buffer A plus 0.5 M imidazole). The elution was dialysed with dialysis buffer (50 mM Tris-HCl pH 8.0, 200 mM NaCl, 1 mM DTT and 5% glycerol).

Guide RNAs (gRNAs) and Variants

Inclusion of direct repeat mutations may improve gRNA stability. The direct repeats from the three CRISPR Cas12 systems provided herein have two A:U base pairs within the stem-loop region. Increasing the thermal stability of the stem-loop is expected to increase the fraction of properly folded crRNA for loading into its cognate Cas12 and thereby nuclease activity (Pengpeng et al., 2019). Those A:U base pairs were replaced with C:G in the direct repeats of the CRISPR systems of the disclosure to create new, more stable non-naturally occurring variants based on the minimum free energy prediction for the RNA folding.

The predicted (putative) naturally occurring direct repeat sequences in the CRISPR locus, as found in bacterial DNA, of the Cas proteins of the disclosure are shown in Table 2 and 5a, above (shown as DNA sequences). Novel variants are shown in Table 5b above (represented as DNA sequences). The predicted secondary structure are shown in FIGS. 7A-7C. The entire direct repeat sequence, or part of the direct repeat sequence is expected to form a functional non-naturally occurring gRNA, and bind to a Cas protein of the disclosure. RNAs forming the direct repeat variants and spacers used in this example were synthesized by Synthego.

FIGS. 3B, 3E, 3G, 5B, 5D, and 5F shows the predicted secondary structures (folding) of the repeat sequence for the Cas9.1, Cas9.3, Cas9.4, Cas12a.1, Cas12p, and Cas12q pre-crRNA. To assemble these predictions, the openly available RNAfold webserver tool was used.

### In Vitro Transcription Reaction (IVT)

In Vitro Transcription was carried out using MEGAscript™ T7 Transcription Kit (Ambion, Invitrogen) according to the manufacturer's instructions and were cleaned with Monarch® RNA Cleanup Kit (New England Biolab) according the manufacturer's instructions. RNAs were visualized in a 2% agarose gel using Gel Loading Buffer II (Ambion, Invitrogen).

### In Vitro Target Cleavage Assays

The following template sequences used for in vitro target cleavage assays are shown in Table 9.

TABLE 9

| | | Nucleic |
|---|---|---|
| Name | Sequence | acid |
| KPC | TTCAAGGGCTTTCTTGCTGCCGCTGTGCTGGCTCGCAG | DNA |
| gB template 1 | CCAGCAGCAGGCCGGCTTGCTGGACACACCCATCCGT | |
| | TACGGCAAAAATGCGCTGGTTCCGTGGTCACCCATCTC | |
| | GGAAAAATATCTGACAACAGGCATGACGGTGGCGGAG | |
| | CTGTCCGCGGCCGCCGTGCAATACAGTGATAACGCCG | |
| | CCGCCAATTTGTTGCTGAAGGAGTTGGGCGGCCCGGC | |
| | CGGGCTGACGGCCTTCATGCGCTCTATCGGCGATACCA | |
| | CGTTCCGTCTGGACCGCTGGGAGCTGGAGCTGAACTC | |
| | CGCCATCCCAGGCGATGCGCGCGATACCTCATCGCCG | |
| | CGCGCCGTGACGGAAAGCTTACAAAAACTGACACTGG | |
| | GCTCTGCACTGGCTGCGCCGCAGCGGCAGCAGTTTGTT | |
| | GATTGGCTAAAGGGAAACACGACCGGCAACCACCGCA | |
| | TCCGCGCGGCGGTGCCGGCAGACTGGGCAGTCGGAGA | |
| | CA (SEQ ID NO: 43) | |
| NDM | CCAAATTAAGATCATCTATTTACTAGGCCTCGCATTTG | DNA |
| gB template 1 | CGGGGTTTTTAATGCTGAATAAAAGGAAAACTTGATG | |
| | GAATTGCCCAATATTATGCACCCGGTCGCGAAGCTGA | |
| | GCACCGCATTAGCCGCTGCATTGATGCTGAGCGGGTG | |
| | CATGCCCGGTGAAATCCGCCCGACGATTGGCCAGCAA | |
| | ATGGAAACTGGCGACCAACGGTTTGGCGATCTGGTTTT | |
| | CCGCCAGCTCGCACCGAATGTCTGGCAGCACACTTCCT | |
| | ATCTCGACATGCCGGGTTTCGGGGCAGTCGCTTCCAAC | |
| | GGTTTGATCGTCAGGGATGGCGGCCGCGTGCTGGTGG | |
| | TCGATACCGCCTGGACCGATGACCAGACCGCCCAGAT | |
| | CCTCAACTGGATCAAGCAGGAGATCAACCTGCCGGTC | |
| | GCGCTGGCGGTGGTGACTCACGCGCATCAGGACAAGA | |
| | TGGGCGGTATGGACGCGCTGCATGCGGCGGGG (SEQ | |
| | ID NO: 44) | |
| OXA | CGAAGCCAATGGTGACTATATTATTCGGGCTAAAACT | DNA |
| gBlock 1 | GGATACTCGACTAGAATCGAACCTAAGATTGGCTGGT | |
| | GGGTCGGTTGGGTTGAACTTGATGATAATGTGTGGTTT | |
| | TTTGCGATGAATA (SEQ ID NO: 45) | |
| MecA | TACAACTTCACCAGGTTCAACTCAAAAAATATTAACA | |
| gBlock1 | GCAATGATTGGGTTAAATAACAAAACATTAGACGATA | |
| | AAACAAGTTATAAAATCGATGGTAAAGGTTGGCAAAA | |
| | AGATAAATCTTGGGGTGGTTACAACGTTACAAGATAT | |
| | GAAGTGGTAAATGGTAATATCGACTTAAAACAAGCAA | |
| | TAGAATCATCAGATAACATTTTCTTTGCTAGAGTAGCA | |
| | CTCGAATTAGGCAGTAAGAAATTTGAAAAAGGCATGA | |
| | AAAAACTAGGTGTTGGTGAAGATATACCAAGTGATTA | |
| | TCCATTTTATAATGCTCAAATTTCAAACAAAAATTTAG | |
| | ATAATGAAATATTATTAGCTGATTCAGGTTACGGACAA | |
| | GGTGAAATACTGATTAACCCAGTACAGATCCTTTCAAT | |
| | CTATAGCGCATTAGAAAATAATGGCAATATTAACGCA | |
| | CCTCACTTATTAAAAGACACGAAAAACAAAGTTTGGA | |
| | AGAAA (SEQ ID NO: 46) | |
| hHPRT1 | CTCTGTATGTTATATGTCACATTTTGTAATTAACAGCT | DNA |
| | TGCTGGTGAAAAGGACCCCACGAAGTGTTGGATATAAG | |
| | CCAGACTGTAAGTGAATTACTTTTTTTGTCAATCATTT | |
| | AACCATCTTTAACCTAAAAGAGTTTTATGTGAAATGGC | |
| | TTATAATTGCTTAGAGAATATTTGTAGAGAGGCACATT | |
| | TGCCAGTATTAGATTTAAAAGTGATGTTTTCTTTATCT | |
| | AAAT (SEQ ID NO: 47) | |
| DENV ssRNA | UGACGAAGACCAUGCUCACUGGACAGAAGCAAAAAU | RNA |
| target | GCUGCUGGACAACAUCAACACACCAGAAGGGAUUAU | |
| | ACCAGCUCUCUUUGAACCAGAAAGGGAG | |
| | (SEQ ID NO: 48) | |
| ZIK ssRNA | CCACACUGGAACAACAAAGAAGCACUGGUAGAGUUC | RNA |
| target | AAGGACGCACAUGCCAAAAGGCAAACUGUCGUGGUU | |
| | CUAGGGAGUCAAGAAGGAGCAGUUCACA | |
| | (SEQ ID NO: 49) | |
| HANT ssRNA | AGAGGCAACUUGCAGAUUUGGUGGCAGCUCAAAAAU | RNA |
| target | UGGCUACAAAACCAGUUGAUCCAACAGGGCUUGAGC | |
| | CUGAUGAUCAUCUAAAGGAAAAAUCAUC | |
| | (SEQ ID NO: 50) | |

gBlocks (in Table 9) are double stranded DNA templates synthetize by IDT of about 100-500 nt, whose sequences include the target of interest. The specific cleavage assay containing 1 ug of gBlock target sequences is conducted in buffer NEB 3 with 30 nM Cas (Cas9.1, Cas9.2, Cas9.3, Cas9.4 Cas12a.1, Cas12p, Cas12q), 30 nM crRNA against the specific sequences, during 2 h at 37° C. Reactions are stopped by 10 min at 70° C. The products are cleaned up using PCR purification columns (QIAGEN) and visualized in 1% agarose gel pre-stained with SYBER Gold (Invitrogen). To identify the type of cut (staggered/blunt) aliquots of digestion products are run in 1% of agarose gel and bands corresponding to cleaved target were gel extracted using DNA Clean & Concentrator kit (Zymo Research). The purified products were sequenced using specific primers and analyzed by DNASTART. For collateral activity assays, we used buffer NEB 3 with 30 nM Cas (Cas9.1, Cas9.2, Cas9.3, Cas9.4, Cas12a.1, Cas12p, Cas12q), 30 nM crRNA and 1 nM ssDNA activator containing the target sequence during 10, 20, 40 and 60 min at 37 C. The reactions were initiated by addition of 250 nM M13 ssDNA or M13 dsDNA plasmid (NEB). Reactions were stopped by 10 min at 70° C. Products were separated by 2% agarose gel pre-stained with SYBER Gold (Invitrogen)

### Fluorescence Detection of Collateral Activity

Fluorescence detection can be conducted to determine collateral activity. 30 nM Cas12 was complexed with 30 nM crRNA and 50 nM DNaseAlert™ substrate (IDT) in Buffer NEB 2.1 at 37° C. in a 40 μl reaction final volume. The reaction can be monitored in a fluorescence plate reader for up to 30 min at 37° C. with fluorescence measurements taken every 2 min in HEX channel (λex: 536 nm; λem: 556 nm). The resulting data can be background-corrected using the readings obtained in the absence of target. For the FQ detection of collateral cleavage of dsDNA/ssDNA and dsRNA/ssRNA DNaseAlert™ (IDT) and RNaseAlert®-1 was used respectively.

### Velocity of Cleavage Cis and Trans

The initial velocity (VO) can be calculated by fitting a linear regression and plotted against the substrate concentration to determine the Michaelis-Menten constants (GraphPad Software), according to the following equation: Y=(Vmax×X)/(Km+X), where X is the substrate concentration and Y is the enzyme velocity. The turnover number (kcat) is determined by the following equation: kcat=Vmax/Et, where Et=0.1 nM.

### Example 2: Determination of Endonuclease Activity

It was investigated whether the novel Cas12a.1 and the Cas12p of the disclosure supplied only with crRNA could cleave target DNA in vitro. The Cas12a.1 and the Cas12p were designed, overexpressed, purified in vitro and used to form a complex with a crRNA against a specific target. It was found that the presence of the Cas12 protein and the cRNA are sufficient for forming an active complex for mediating DNA cleavage.

### Example 3: Determination of PAM Sequence Specificities

To demonstrate a PAM sequence cleavage-dependent action of the Cas12a.1 and the Cas12p of the disclosure, ten different PAM motifs were designed, following a specific target sequence. Using these, of the ten motifs tested, TCTN and TGTN were identified as efficient PAM sequences for Cas12a.1 and Cas12p, respectively. FIG. 8 shows bar graphs for the PAM sequence preferences of Cas12a.1 and Cas12p for the ten PAM motifs, measuring the performance of the Cas12a.1 and the Cas12p using fluorescence assays. The resulting fluorescence data were background-subtracted.

### Example 4: Demonstration of Collateral Activity of Cas12a.1 and Cas12p, and their Ability to Cut ssDNA and RNA Reporters

It was investigated whether the Cas12a.1 and the Cas12p proteins of the disclosure were able to cut dsDNA or RNA. Cas12a.1-gRNA or Casp-gRNA complexes were mixed with sample (positive and negative) and a reporter to react in presence of a target. In these examples, a custom ssDNA fluorescently labeled reporter (5′ FAM-TTATTATT-3IABkFQ 3′-IDT) (SEQ ID NO: 121) and a commercial fluorescently labeled reporter RNA reporter (Cat N 11-04-03-03-IDT) were used.

FIG. 9B shows collateral activity of the Cas12a.1 and Cas12p proteins of the disclosure, using the Hanta virus as an exemplary target. Cas12a.1 and Cas12p were incubated with their respective gRNAs to target Hanta to form a 1 uM complex and were exposed to the DNA target at concentration of 10 nM; added to the mix were fluorescently labeled ssDNA or RNA reporters, at a concentration between 1 and 0.5 uM. Controls did not contain the specific DNA target. Collateral activity was observed only in the presence of target. Cas12a.1 shows ssDNA collateral cleavage for ssDNA but not for RNA, under these conditions. On the other hand, Cas12p exhibited collateral cleavage activity of both ssDNA and RNA reporters. The RNA substrate used for this and other examples provided herein was RNaseAlert®-1 Substrate (25 single use tubes. Catalog No. 11-04-03-03-IDT). The exemplary ssDNA reporter used for this and other examples provided herein was (5′ FAM-TTATTATT-3IABkFQ 3′-IDT) (SEQ ID NO: 121).

FIG. 9C shows that Cas12p exhibits both ssDNA and RNA reporter collateral cleavage using as a SARS-CoV-2 inactivated virus as sample as the target.

### Example 5: Testing of Thermostability

The activities of Cas12a.1 and Cas12p were tested at different temperatures.

FIG. 10 shows activity of the Cas12a.1 and Cas12p proteins at 25° C., using 1 uM complex, 300 nM Reporter SARS-CoV-2 (Spn2 target) at 1 minute and 5 minutes as endpoint for the readout.

FIGS. 10 and 14 shows that Cas12p perform equally well at 25° C. as it does at 37° C.

FIG. 15 shows the differential performance of Cas12p vs. LbCas12a in producing a fluorescence signal by reporter cleavage at 25° C. LbCas12a and Cas12p were incubated with their respective gRNAs to target N gene of SARS-CoV-2 to form a 1 uM complex. The target was the same for both and was provided at a concentration of 10 nM. 600 nM ssDNA reporter was added into the reaction mix (50 mM NaCl, 10 mM Tris-HCl, 10 mM MgCl2 and 100 μg/ml BSA). Collateral cleavage was measured by fluorescence and the readout was performed in real time. FIG. 16 shows the differential performance of Cas12p vs. LbCas12a at 25° C., using SARS-CoV-2 as a target, described in Example 10.

### Example 6: Testing of Various Salt Concentrations

The activities of Cas12a.1 and Cas12p were tested at various NaCl concentrations; Cas12a.1 and Cas12p and were shown to maintain functionality, FIG. 11 shows the activity of the two proteins at various NaCl concentrations. The resulting fluorescence data was background-subtracted.

### Example 7: Testing of Various Commercial Buffers

In various commercial buffers, the Cas12a.1 and the Cas12p of the disclosure showed different performances. FIG. 12 shows the performance of the Cas12a.1 and the Cas12p of the disclosure in three different commercial buffers. The resulting fluorescence data was background-subtracted.

### Example 8: Use of Cas12a.1 and Cas12p for the Detection of Hantavirus

Hantaviruses are a family of viruses spread mainly by rodents and can cause various disease symptoms in people worldwide. Infection with any hantavirus can produce hantavirus disease in people. Described below is the use of the novel Cas12a.1 and Cas12p proteins of the disclosure for the detection of Hantavirus.

### Primer Design and CRISPR RNA Guide Selection

Provided below is the Hantavirus genome, Andes virus segment S complete sequence

(NCBI Reference Sequence: NC_003466.1)
(SEQ ID NO: 69)
TAGTAGTAGACTCCTTGAGAAGCTACTGCTGCGAAA
GCTGGAATGAGCACCCTCCAAGAATTGCAGGAAAA
CATCACAGCACACGAACAACAGCTCGTGACTGCTC
GGCAAAAGCTTAAGGATGCCGAGAAGGCAGTGGAG
GTGGACCCGGATGACGTTAACAAGAGCACACTACA
AAGTAGACGGGCAGCTGTGTCTACATTGGAGACCA
AACTCGGAGAACTTAAGAGGCAACTTGCAGATTTG
GTGGCAGCTCAAAAATTGGCTACAAAACCAGTTGA
TCCAACAGGGCTTGAGCCTGATGATCATCTAAAGG
AAAAATCATCTCTGAGATATGGGAATGTCCTGGAT
GTTAATTCAATTGATTTGGAAGAACCGAGTGGACA
GACTGCTGATTGGAAGGCTATAGGAGCATACATCT
TAGGGTTTGCAATTCCGATCATCCTAAAGGCCTTA
TACATGCTGTCAACCCGTGGGAGACAAACTGTGAA
AGACAACAAAGGGACCAGGATAAGGTTTAAGGATG
ATTCTTCCTTTGAAGAAGTCAATGGGATACGTAAA
CCAAAACACCTTTACGTCTCAATGCCAACTGCACA
GTCCACTATGAAGGCTGAAGAAATCACGCCAGGAC
GATTTAGGACAATTGCTTGTGGCCTTTTTCCAGCA
CAGGTCAAAGCCCGAAATATAATAAGTCCTGTAAT
GGGAGTAATTGGATTTGGCTTCTTTGTAAAGGATT
GGATGGATCGGATAGAAGAGTTTCTGGCTGCAGAG
TGTCCATTCTTACCTAAGCCAAAGGTCGCCTCAGA
AGCCTTCATGTCTACCAATAAGATGTATTTTCTGA
ACAGACAGAGACAAGTCAATGAATCTAAGGTTCAA
GATATTATCGATTTGATAGACCATGCTGAGACCGA
GTCTGCTACCTTGTTTACAGAGATTGCAACACCCC
ATTCAGTCTGGGTGTTTGCATGTGCACCTGACCGG
TGCCCTCCAACTGCATTGTATGTTGCAGGGGTACC
GGAACTTGGTGCATTTTTTTCTATCCTTCAGGACA
TGCGTAATACCATCATGGCATCTAAATCTGTAGGG
ACTGCAGAAGAGAAGCTAAAGAAAAAATCTGCCTT
CTACCAATCATACCTAAGAAGGACACAATCTATGG
GAATCCAACTGGACCAGAAGATCATAATCCTTTAC
ATGCTATCATGGGGTAAAGAAGCTGTGAATCACTT
CCATCTTGGTGATGATATGGACCCTGAACTCAGGC
AGCTAGCACAATCTCTGATCGATACTAAGGTGAAG
GAGATCTCCAACCAAGAGCCACTTAAGTTGTAGGT
GCTTAATGAAATCATGATTGAAGAAAGACTTTCCG
GGCTTGTGCCACATATTAATCATCTCAGGACCTAT
CCTTAATGTGATTAATAGGGTTTTATTATAAGGGC
AGTTAATGGGGTTGGTTACTAACTATGGGTAAGGG
TTCATTACCATTTTTGCACTAGGGTTAAAGGGCCA
CTACATTGTATTTGCACTAAGGGAAATGGGAGGTG
GGTTAGTTTGTATTTAGTTGTTAAGTTTTTTATAA
TCATATGTTAATGAGGAATTAGCTATATGATATCA
CTGATTGATTGGCTATTTTTAGGTTAAGTAATTGT
AGTTAAATAGTTGTGTTAAGTTAGTATGTTAAGGT
TTATAGGTTAAGATTTACTAACAATCATATTATGT
CATTAGATGTAAATTTCATTCCTGGCTTGCTTCTG
CTTTCGCATTGCTAACCTACAACAAGACTACCTCA
CCCACTACCCCTCCCCTATTCTACCTCAACACATA
CTACCTCACATTTGATTTTTCTTGATTGCTTTTCA
AGGAGCATACTACTA

The following exemplary sequence was selected as the target of the spacer gRNA, for Hantavirus detection: GTGGCAGCTCAAAAATTGGCTAC (SEQ ID NO: 70) (underlined above). Other sequences can be selected for targeting.

### CRISPR Guide Design and Synthesis

A gRNA was designed, with a spacer specific to the Hantavirus target sequence. Shown below is the guide (includes direct repeat (single underline)+target complementary sequence (double underline)): AAATTTCTACTGTAGTAGAT GTGGCAGCTCAAAAATTGGCTAC (SEQ ID NO: 249)

For natural expression and processing of the gRNA, a minimal array with direct repeat from Cas12a.1 and Cas12p and the target complementary sequence was cloned in the Cas expression vector. The CRISPR complex was formed in vivo in the expressing bacteria NiCo21(DE3) Competent E. coli and purified from bacteria extracts. In other variations, the guide can be synthesized and complexed with a Cas protein in vitro.

The complex was added to a mix which contained a molecular reporter with a fluorochrome. The sample to be tested was added to the mix. The sample to be tested may be: a sample directly obtained from a subject; a sample obtained from a subject and then diluted and/or treated; DNA (may be amplified) or RNA from a sample taken from a subject; or the sample to be tested may be cDNA made from RNA from the sample. The sample may be further amplified, for example using RPA (Recombinase Polymerase Amplification, e.g. using RPA TwistAmp Basic (TABAS03)).

The components for formation of the CRISPR complex is shown in Table 10, mixed in that order. The complex was made, and allowed to incubate for 10 minutes at room temperature.

TABLE 10

| Component | [Stock] | [Final] | Volume (1X) |
|---|---|---|---|
| Nuclease free water | | | 15.05 |
| Buffer NEB 2.1 | 10X | IX | 2.0 |
| RNA guide Work solution | 300 nM | 30 nM | 2.2 |
| Cas12a.1 Work solution | 1 uM | 30 nM | 0.8 |
| TOTAL | | | 20.0 |

The components for formation of the CRISPR mix is shown in Table 11, mixed in that order.

TABLE 11

| Component | [Stock] | [Final] | Volume (1X) |
|---|---|---|---|
| Nuclease free water | | | 16.00 |
| Buffer NEB 3.1 | 10X | 1X | 2.0 |
| CRISPR complex | 300 nM | 30 nM | 20.0 |
| Molecular reporter FAM | 50 uM | 2.5 uM | 2.0 |
| DNA background | 400 ng/ul | 200 ng | 0.5 |
| Amplified Sample | * | | 4.0 |
| TOTAL | | | 40.0 |

### Results Readout

The reaction was monitored in a fluorescence plate reader for up to 30 min at 37° C. with fluorescence measurements taken every 2 min or in the final endpoint in HEX channel (λex: 536 nm; λem: 556 nm). The resulting data are background-corrected using the readings obtained in the absence of target.

FIG. 9A shows specific cleavage activity of the Ca12a.1 and Cas12p proteins of the disclosures with the Hanta target. A pGEM plasmid was cloned with the Hanta target (pGEM-Hanta) and used to demonstrate specific cleavage activity of Cas12a.1 and Cas12p. Cas12a.1 and Cas12p were incubated with their respective gRNAs to target the Hanta target and exposed to gGEM-Hanta plasmid or gGEM plasmid without target for 2 hours at 37° C. Arrows shows that pGEM-Hanta plasmid is cut but pGEM is not, demonstrating that the cleavage is specific to the Hanta target.

Using collateral activity, Hantavirus RNA was able to be detected in a picomolar concentration in less than one hour, as shown in FIG. 13. FIG. 13 shows sensitivity curves without RPA of the Cas12a.1 and the Cas12p of the disclosure, for various target concentrations measured for 30 minutes.

### Example 9: Cas12p Characterization

Cas12p was further characterized and compared to LbCas12a (SEQ ID NO: 122 (SEQ ID NO: 242 from U.S. Pat. No. 9,790,490)) to support the characteristics of this novel Cas12 subtype.

FIG. 14 shows that the amount of fluorescence detection by Cas12p for a target DNA reverse transcribed from SARS-CoV-2 RNA was equal at both 37° C. and 25° C., indicative of thermostability and function and room temperature.

FIG. 15 and the below show the kinetic performance of Cas12p vs. LbCas12a at room temperature.

| Cas12a2 vs LbCas12a 1 uM | Vmax Points = 38 | | | | | | |
|---|---|---|---|---|---|---|---|
| complex 600 nM Reporter | Well | ◯ G1 | □ G3 | Δ G5 | ⋄ M1 | ● M3 | ▪ M5 |
| | Vmax | 6.17e6 | 6.74e6 | 5.81e6 | 2.88e6 | 3.59e6 | 3.52e6 |
| Spn2 40′ RT | R\hat{ }2 | 0.997 | 1.000 | 0.996 | 0.979 | 0.993 | 0.988 |

FIG. 16 further shows the differential performance of Cas12p vs. LbCas12a at room temperature.

As noted above, FIG. 9A shows specific cleavage activity of the Ca12a.1 and Cas12p proteins of the disclosures with an exemplary Hanta virus target, as described in the above example. FIG. 9B shows collateral activity of the Cas12a.1 and Cas12p proteins of the disclosure, using the Hanta virus as an exemplary target, as described in the above example. FIG. 9C shows collateral activity of the novel Cas12p protein for SARS-CoV-2 target described in Example 10.

FIG. 17 shows the ability of Cas12p to cleave both a ssDNA and RNA reporter, as tested across various targets as exemplary (Hanta virus, SARS-CoV-2). Cas12p was incubated with a gRNAs directed to the Hanta virus or SARS-CoV-2 virus to form a 1 uM complex and was exposed to the DNA target at 10 nM concentration adding into the mix a ssDNA or RNA fluorescence marked reporter at a concentration between 1 and 0.5 uM. Controls did not have the specific DNA target. Collateral activity is seen only in the presence of target for both ssDNA and RNA.

### Example 10: Use of Cas12a.1 for the Detection of SARS-CoV-2

Provided here is an example of the use of Cas12p for the detection of SARS-CoV-2 in upper respiratory specimens during the acute phase of infections. Positive results are indicative of the presentence of SARS-CoV-2 RNA. Further clinical correlation with patient history and other diagnostic information could be utilized to determine patient infection status.

### Assay

RNA was purified from 140 μl of nasopharyngeal/oropharyngeal sample using QIAmp Viral RNA Mini Kit (QIAGEN) as instructed in the user guide and eluting in 60 μl. If RNA was not tested immediately, the RNA was stored at <−70° C.

After RNA purification, detection of SARS-CoV-2 genomic RNA using CASPR Lyo-CRISPR SARS-CoV-2 kit was carried out using a two-step procedure as summarized in FIG. 18 and outlined below.

Step 1: The purified RNA was subject to reverse transcription and amplification. Reverse transcription and amplification of 5 μl of purified RNA using reverse transcription loop-mediated isothermal amplification (RT-LAMP) with primer sets specifically designed to target a highly conserved N gene of the SARS-CoV-2 viral genome were carried out.

The RT-LAMP reaction was based on a total of three (3) pair of primers that amplify a specific sequence in the N gene of SARS-CoV-2 RNA.

The RT-LAMP reaction was performed by incubating the reaction mix at 62° C. for 30 minutes.

Step 2: Following the RT-LAMP reaction, the detection of amplified viral target was carried out using a Cas12a.1 ribonucleoprotein complex (RNP complex) comprising Cas12a.1+a gRNA (single molecule guide) targeting the amplified viral N gene sequences from Step 1. The sequence targeted by the gRNA in the cDNA made from the viral RNA was as follows:

(SEQ ID NO: 119)
GATCGCGCCCCACTGCGTTCTCC

If the SARS-CoV-2 genomic RNA was present in the sample and was amplified during the RT-LAMP reaction, the gRNA from the RNP complex can bind to the DNA target and trigger the collateral cleavage activity of Cas12a.1, which degrades a 5′FAM-3′Quencher single stranded DNA (ss-DNA) reporter molecule causing the emission of fluorescence. Fluorescence measurements can be performed in standard plate readers with fluorescence capabilities.

The assay was carried out in less than 60 minutes from start to finish—from obtaining the sample to a readout of the results. FIG. 18 shows a schematic workflow for the detection of SARS-CoV-2 described in this example.

The additional negative, positive, and extraction controls were included.

Negative Control: Nuclease-free water was used to identify any potential contamination of the assay run.

Positive Control: A synthetic sequence identical to the target sequence was provided at a concentration of 2000 cp/ml, in a separate vial. The positive control verified that the assay was performing as expected.

Extraction controls: Primer sets that target human housekeeping gene RNAse P (for example) were included in the RT-LAMP reaction mix to ensure the proper performance of extraction procedure.

The reagents used were provided in lyophilized form, reducing manual sources of operator error.

### Results

For negative controls (NTC) a ratio value was calculated between fluorescence (IF) measured at end-point (t=20 min) over fluorescence at the beginning of the run (t=0 min)

$$Ratio Value ( X ) = \frac{{IF}_{NTC} {t}^{20 min}}{{IF}_{NTC} {t}^{0 min}}$$

For positive control and clinical samples, a ratio value was calculated between sample reaction fluorescence (IF) measured at end-point (t=20 min) over the corresponding valid negative template control reaction fluorescence measurement at 20 minutes.
For positive control (PC)

$$Ratio Value ( A ) = \frac{{IF}_{PC} {t}^{20 min}}{{IF}_{NTC} {t}^{20 min}}$$

For clinical samples

$$Ratio Value ( A ) = \frac{{IF}_{Sample} {t}^{20 min}}{{IF}_{NTC} {t}^{20 min}}$$

Once ratio values for controls and samples were calculated results were calculated according to the following criteria for control assays:

| | | Cutoff | Result | |
|---|---|---|---|---|
| Control Assay | Target | Value | Valid | Invalid |
| NTC | SARS-CoV-2 | 2 | If ratio if <2 | If ratio > 2 |
| Positive Control | SARS-CoV-2 | 3 | If ratio if >3 | If ratio < 3 |

In this example, for unknown clinical samples: a Positive sample would have a ratio value >3 (a minimum 3-fold increase in fluorescence emission between sample reaction and negative control reaction at t=20 min).

In this example, a Negative sample would have a ratio value <3 (less than 3-fold increase in fluorescence emission between a sample reaction and negative control reaction at t=20 min). To confirm a negative result, RNAse P would be expected to have a value >3 (an increase in fluorescence emission between sample reaction and negative control reaction at t=20 min).

### Performance Evaluation—Analytical Sensitivity, Limit of Detection

The Limit of Detection (LoD) study established the lowest concentration of SARS-CoV-2 (genome copies(cp)/μL of input) that could be detected at least 95% of the time.

To determine LoD, serial dilutions of whole inactivated SARS-CoV-2 were spiked into negative nasopharyngeal samples and processed according to the procedure described above.

A LoD was determined by testing three (3) replicates of three (3) different dilutions (10 copies/μl, 5 copies/μl, 2.5 copies/μl) and corresponded to the lowest concentration (5 copies/μl) at which 3/3 replicates were tested positive. This preliminary LoD (5 copies/μl) was confirmed by testing at 0.5×-1×-1.5×-2× of the preliminary LoD in twenty (20) replicates for each concentration. The LoD was the lowest concentration at which at least 19/20 replicates were tested positive for the target.

LoD was confirmed at 7.5 copies/μL with a detection rate of 95% (19/20). Results are summarized in the following table:

TABLE 12

| | Ratio | |
|---|---|---|
| Replicates | Value | Result |
| 1 | >3 | Positive |
| 2 | >3 | Positive |
| 3 | >3 | Positive |
| 4 | >3 | Positive |
| 5 | >3 | Positive |
| 6 | >3 | Positive |
| 7 | >3 | Positive |
| 8 | >3 | Positive |
| 9 | >3 | Positive |
| 10 | >3 | Positive |
| 11 | >3 | Positive |
| 12 | <3 | Negative |
| 13 | >3 | Positive |
| 14 | >3 | Positive |
| 15 | >3 | Positive |
| 16 | >3 | Positive |
| 17 | >3 | Positive |
| 18 | >3 | Positive |
| 19 | >3 | Positive |
| 20 | >3 | Positive |

### Performance Evaluation—Analytical Sensitivity, Inclusivity

Inclusivity was demonstrated by comparing the SARS-CoV-2 assay primers and gRNA to an alignment of 4703 SARS-CoV-2 sequences available in GISAID as of May 16, 2020. The dataset was further refined by considering only whole genome sequences (>29000 bp) and by removing low-quality sequences with ambiguous sequencing data (N's) and animal origin. This in-silico analysis indicated that the that primers and gRNA sequences utilized have a 99.9% homology to all available circulating SARS-CoV-2 sequences.

### Performance Evaluation—Analytical Specificity

The assay 2 was based on a set of primers and a unique gRNA designed for specific detection of SARS-CoV-2.

To evaluate the analytical specificity, an in-silico analysis using NCBI Blast tool was first performed to confirm the absence of any potential cross-reactivity between any of the primer/gRNA sequences with normal and pathogenic organisms of the respiratory tract.

Results are summarized in Table 13:

TABLE 13

| | % homology | % homology |
|---|---|---|
| | for SARS- | for SARS- |
| Pathogen | CoV-2 gRNA | CoV-2 primers |
| Human coronavirus 229E | <80% | <80% |
| Human coronavirus HKU1 | <80% | <80% |
| Human coronavirus NL63 | <80% | <80% |
| Human coronavirus OC43 | <80% | <80% |
| MERS-coronavirus | <80% | <80% |
| SARS-coronavirus | >80% | >80% |
| Adenovirus | <80% | <80% |
| Human Metapneumovirus | <80% | <80% |
| (hMPV) | | |
| Parainfluenza virus 1-4 | <80% | <80% |
| Influenza A & B | <80% | <80% |
| Respiratory syncytial virus | <80% | <80% |
| Enterovirus | <80% | <80% |
| Rhinovirus | <80% | <80% |
| Chlamydia pneumoniae | <80% | >80% |
| Haemophilus influenzae | <80% | <80% |
| Legionella pneumophila | <80% | >80% |
| Mycobacterium tuberculosis | <80% | >80% |
| Streptococcus pneumoniae | <80% | <80% |
| Streptococcus pyogenes | <80% | <80% |
| Bordetella pertussis | <80% | >80% |
| Mycoplasma pneumoniae | <80% | <80% |
| Pneumocystis jirovecii | <80% | <80% |
| Candida albicans | <80% | <80% |
| Pseudomonas aeruginosa | <80% | >80% |
| Staphylococcus epidermidis | <80% | <80% |
| Streptococcus salivarius | >80% | <80% |

These results showed that only a few microorganisms have >80% homology between their genome sequences and at least one of the SARS-CoV-2 primers or gRNA included in the assay.

To confirm the in-silico evaluation, the same pathogens were in vitro to check for both potential cross-reactivity and interference.

The analysis was performed on a total of 22 pathogens by spiking either genomic DNA/RNA or inactivated strains into the SARS-CoV-2 negative nasopharyngeal sample at the concentration indicated in Table 15 during the lysis step of the extraction procedure and tested using the assay described herein. Each pathogen was tested in triplicate. To discard any false negative results an RNAseP assay was run in parallel to each sample,

An interference analysis was also evaluated on the microorganisms that showed >80% homology with either the SARS-CoV-2 primers or the gRNA included in the kit. To detect any potential interference the analysis was performed by following the same protocol used for cross-reactivity testing in presence of 3× LoD SARS-CoV-2 (22.5 cp/μl).

All negative results for pathogens tested were confirmed by a positive result in the RNAseP assay.

TABLE 14

| | | Tested | Cross- | | |
|---|---|---|---|---|---|
| Pathogens | Source | Concentration | Reactivity | Interference | RNAse P |
| Human coronavirus 229E | 0810229CFHI | 10\hat{ }5 TCID50/mL | 0/3 | N/A | 3/3 |
| Human coronavirus OC43 | 0810024CFHI | 10\hat{ }5 TCID50/mL | 0/3 | N/A | 3/3 |
| Human coronavirus HKU1 | ATCC VR- | 10\hat{ }5 copies/mL | 0/3 | N/A | 3/3 |
| | 1580DQ | | | | |
| Human coronavirus NL63 | ATCC ® 3263 SD | 10\hat{ }5 copies/mL | 0/3 | N/A | 3/3 |
| SARS-coronavirus | NATSARS-ST | 10\hat{ }5 TCID50/mL | 0/3 | 0/3 | 3/3 |
| Respiratory syncytial virus | ATCC ® VR- | 10\hat{ }5 copies/mL | 0/3 | N/A | 3/3 |
| | 1580DQ | | | | |
| Influenza A | VR-95DQ | 10\hat{ }5 copies/mL | 0/3 | N/A | 3/3 |
| Influenza B | VR-1885DQ | 10\hat{ }5 copies/mL | 0/3 | N/A | 3/3 |
| Mycobacterium | NR-14867 | 10\hat{ }6 copies/mL | 0/3 | 0/3 | 3/3 |
| tuberculosis | | | | | |
| Candida albicans | ATCC ® | 10\hat{ }6 copies/mL | 0/3 | N/A | 3/3 |
| | 10231D-5 | | | | |
| Pseudomonas aeruginosa | ATCC ® | 10\hat{ }6 copies/mL | 0/3 | 0/3 | 3/3 |
| | 27853D-5 | | | | |
| Staphylococcus epidermis | ATCC ® | 10\hat{ }6 copies/mL | 0/3 | N/A | 3/3 |
| | 12228D-5 | | | | |

In conclusion, based on in-silico and in vitro analysis it was anticipated no cross-reactivity nor interference between primers/gRNA included in the assay and most common pathogens in the respiratory tract.

### Clinical Evaluation

Clinical evaluation of the assay was performed using nasopharyngeal swabs as clinical samples from male and female adult patients with signs and symptoms of an upper respiratory infection.

A total of 30 positive samples and 30 negative samples were collected to assess the performance and tested using QIAmp Viral RNA Mini kit for RNA extraction followed by the procedure as described (noted as “Cas12a.1-Based Assay” in Table 15). All samples were also tested using an RT-PCR Test as a comparison method to obtain positive and negative percent agreement values. Results are presented in Table 15 and show 100% positive percent agreement (PPA) and 100% negative percent agreement (NPA) with comparator method.

TABLE 15

| Cas12a.1-Based | | FDA EUA RT-PCR Test | | | |
|---|---|---|---|---|---|
| Assay | | (Comparator) | | PPA | NPA |
| Positive | Negative | Positive | Negative | (95% CI) | (95% CI) |
| 30/30 | 30/30 | 30/30 | 30/30 | 100% | 100% |
| | | | | (90.55-100) | (90.55-100) |

### Example 11: Use of Cas12p for the Detection of SARS-CoV-2

Provided here is an example of the use of Cas12p for the detection of SARS-CoV-2 in upper respiratory specimens during the acute phase of infections. Positive results are indicative of the presence of SARS-CoV-2 RNA. Further clinical correlation with patient history and other diagnostic information could be utilized to determine patient infection status.

### Assay

Nasopharyngeal/nasal swab is inserted in 500 uL of Lysis Buffer, vortex is applied for 2 minutes and 100 uL lysed sample is transported into 1.5 mL capacity tube and heated at 95 C for 5 minutes.

After sample treatment, detection of SARS-CoV-2 genomic RNA using CASPR Direct Lyo-CRISPR SARS-CoV-2 kit was carried out using a two-step procedure as summarized in FIG. 19 and outlined below.

Step 1: The lysed sample was subject to reverse transcription and amplification. Reverse transcription and amplification of 10 μl of lysed sample using reverse transcription loop-mediated isothermal amplification (RT-LAMP) with primer sets specifically designed to target two highly conserved N gene and one highly conserved ORF1ab gene of the SARS-CoV-2 viral genome were carried out.

The RT-LAMP reaction was based on a total of three (9) pair of primers that amplify two specific sequences in the N gene and one specific sequence in the ORF1ab gene of SARS-CoV-2 RNA.

The RT-LAMP reaction was performed by incubating the reaction mix at 62□C for 60 minutes.

Step 2: Following the RT-LAMP reaction, the detection of amplified viral target was carried out using a Cas12p ribonucleoprotein complex (RNP complex) comprising Cas12p+three gRNAs (single molecule guide) targeting the amplified viral N and ORF1ab gene sequences from Step 1. The sequences targeted by the gRNAs in the cDNA made from the viral RNA were as follows: GATCGCGCCCCACTGCGTTCTCC (SEQ ID NO: 119), AUGGCACCUGUGUAGGUCAACCA (SEQ ID NO:120) and UGUGCUGACUCUAUCAUUAUUGG (SEQ ID NO:123).

If the SARS-CoV-2 genomic RNA was present in the sample and was amplified during the RT-LAMP reaction, the gRNA from the RNP complex can bind to the DNA target and trigger the collateral cleavage activity of Cas12p, which degrades a 5′FAM-3′Quencher single stranded reporter molecule causing the emission of fluorescence. Fluorescence measurements can be performed in standard plate readers with fluorescence capabilities.

The assay was carried out in less than 75 minutes from start to finish—from obtaining the sample to a readout of the results. FIG. 18 and FIG. 19 show a schematic workflow for the detection of SARS-CoV-2.

The additional negative, positive, and extraction controls were included.

Negative Control: Nuclease-free water was used to identify any potential contamination of the assay run.

Positive Control: A synthetic sequence identical to the target sequences was provided at a concentration of 2000 cp/ml, in a separate vial. The positive control verified that the assay was performing as expected.

Extraction controls: Primer sets that target human housekeeping gene RNAse P (for example) were included in the RT-LAMP reaction mix to ensure the proper performance of extraction procedure.

The reagents used were provided in lyophilized form, reducing manual sources of operator error.

### Results

For negative controls (NTC) a ratio value was calculated between fluorescence (IF) measured at end-point (t=5 min) over fluorescence at the beginning of the run (t=0 min).

$$Ratio Value ( X ) = \frac{{IF}_{NTC} {t}^{5 min}}{{IF}_{NTC} {t}^{0 min}}$$

For positive control and clinical samples, a ratio value was calculated between sample reaction fluorescence (IF) measured at end-point (t=5 min) over the corresponding valid negative template control reaction fluorescence measurement at 5 minutes.

$$Ratio Value ( A ) = \begin{matrix}\frac{{IF}_{PC} {t}^{5 min}}{{IF}_{NTC} {t}^{5 min}} & \frac{{IF}_{Sample} {t}^{5 min}}{{IF}_{NTC} {t}^{5 min}}\end{matrix}$$

Once ratio values for controls and samples were calculated results were calculated according to the following criteria for control assays:

TABLE 16

| | | Cutoff | Result | |
|---|---|---|---|---|
| Control Assay | Target | Value | Valid | Invalid |
| NTC | SARS-CoV-2 | 2 | If ratio if ≤2.5 | If ratio > 2.5 |
| Positive Control | SARS-CoV-2 | 3 | If ratio if ≥2.5 | If ratio < 2.5 |

In this example, for unknown clinical samples: a Positive sample would have a ratio value ≥2.5 (a minimum 2.5-fold increase in fluorescence emission between sample reaction and negative control reaction at t=5 min).

In this example, a Negative sample would have a ratio value ≤2.5 (less than 2.5-fold increase in fluorescence emission between a sample reaction and negative control reaction at t=5 min). To confirm a negative result, RNAse P would be expected to have a value ≥2.5 (an increase in fluorescence emission between sample reaction and negative control reaction at t=5 min).

### Performance Evaluation—Analytical Sensitivity, Limit of Detection

The Limit of Detection (LoD) study established the lowest concentration of SARS-CoV-2 (genome copies(cp)/μL of input) that could be detected at least 95% of the time.

To determine LoD, serial dilutions of whole inactivated SARS-CoV-2 were spiked into lysis buffer with negative nasal matrix and processed according to the procedure described above.

A LoD was determined by testing three (5) replicates of three (3) different dilutions (25 copies/μl, 12.5 copies/μl, 6.125 copies/μl) and corresponded to the lowest concentration (25 copies/μl) at which 3/3 replicates were tested positive. This preliminary LoD (25 copies/μl) was confirmed in twenty (20) replicates. The LoD was the lowest concentration at which at least 20/20 replicates were tested positive for the target.

LoD was confirmed at 25 copies/μL with a detection rate of 100% (20/20). Results are summarized in the following table:

TABLE 17

| | Ratio | |
|---|---|---|
| Replicates | Value | Result |
| 1 | >2.5 | Positive |
| 2 | >2.5 | Positive |
| 3 | >2.5 | Positive |
| 4 | >2.5 | Positive |
| 5 | >2.5 | Positive |
| 6 | >2.5 | Positive |
| 7 | >2.5 | Positive |
| 8 | >2.5 | Positive |
| 9 | >2.5 | Positive |
| 10 | >2.5 | Positive |
| 11 | >2.5 | Positive |
| 12 | >2.5 | Positive |
| 13 | >2.5 | Positive |
| 14 | >2.5 | Positive |
| 15 | >2.5 | Positive |
| 16 | >2.5 | Positive |
| 17 | >2.5 | Positive |
| 18 | >2.5 | Positive |
| 19 | >2.5 | Positive |
| 20 | >2.5 | Positive |

### Performance Evaluation—Analytical Sensitivity, Inclusivity

Inclusivity was demonstrated by comparing the SARS-CoV-2 assay primers and gRNAs to an alignment of 4703 SARS-CoV-2 sequences available in GISAID as of May 16, 2020. The dataset was further refined by considering only whole genome sequences (>29000 bp) and by removing low-quality sequences with ambiguous sequencing data (N's) and animal origin. This in-silico analysis indicated that the that primers and gRNA sequences overall utilized have a 100% homology to all available circulating SARS-CoV-2 sequences.

### Performance Evaluation—Analytical Specificity

The assay 2 was based on a set of primers and gRNAs designed for specific detection of SARS-CoV-2.

To evaluate the analytical specificity, an in-silico analysis using NCBI Blast tool was first performed to confirm the absence of any potential cross-reactivity between any of the primer/gRNA sequences with normal and pathogenic organisms of the respiratory tract.

Results are summarized in Table 18:

TABLE 18

| | Target 1 (N) | | Target 2 (N) | | Target 3 (Orf1ab) | |
|---|---|---|---|---|---|---|
| | % | | % | | % | |
| | Homology | % | Homology | % | Homology | % |
| | with | Homology | with | Homology | with | Homology |
| Pathogen | sgRNA | primers | sgRNA | primers | sgRNAs | primers |
| Coronavirus | <80 | <80 | <80 | <80 | <80 | <80 |
| 229E | | | | | | |
| Coronavirus | <80 | <80 | <80 | <80 | <80 | <80 |
| HKU1 | | | | | | |
| Coronavirus | <80 | <80 | <80 | <80 | <80 | <80 |
| NL63 | | | | | | |
| Coronavirus | <80 | <80 | <80 | <80 | <80 | <80 |
| OC43 | | | | | | |
| MERS- | <80 | <80 | <80 | <80 | <80 | <80 |
| coronavirus | | | | | | |
| SARS- | >80 | >80 | <80 | <80 | >80 | <80 |
| coronavirus | | | | | | |
| Adenovirus | <80 | <80 | <80 | <80 | <80 | <80 |
| Human | <80 | <80 | <80 | <80 | <80 | <80 |
| Metapneumovirus | | | | | | |
| (hMPV) | | | | | | |
| Parainfluenza | <80 | <80 | <80 | <80 | <80 | <80 |
| virus 1-4 | | | | | | |
| Influenza A & B | <80 | <80 | <80 | <80 | <80 | <80 |
| Respiratory | <80 | <80 | <80 | <80 | <80 | <80 |
| syncytial virus | | | | | | |
| Enterovirus | <80 | <80 | <80 | <80 | <80 | <80 |
| Rhinovirus | <80 | <80 | <80 | <80 | <80 | <80 |
| Chlamydia | <80 | >80 | <80 | <80 | <80 | <80 |
| pneumoniae | | | | | | |
| Haemophilus | <80 | <80 | <80 | <80 | <80 | <80 |
| influenzae | | | | | | |
| Legionella | <80 | >80 | <80 | <80 | >80 | <80 |
| pneumophila | | | | | | |
| Mycobacterium | <80 | >80 | <80 | <80 | <80 | <80 |
| tuberculosis | | | | | | |
| Streptococcus | <80 | <80 | <80 | <80 | <80 | <80 |
| pneumoniae | | | | | | |
| Streptococcus | <80 | <80 | <80 | <80 | <80 | >80 |
| pyogenes | | | | | | |
| Bordetella | <80 | >80 | <80 | <80 | <80 | <80 |
| pertussis | | | | | | |
| Mycoplasma | <80 | <80 | <80 | <80 | <80 | <80 |
| pneumoniae | | | | | | |
| Pneumocystis | <80 | <80 | <80 | <80 | <80 | <80 |
| jirovecii | | | | | | |
| Candida albicans | <80 | <80 | <80 | <80 | <80 | >80 |
| Pseudomonas | <80 | >80 | <80 | <80 | <80 | <80 |
| aeruginosa | | | | | | |
| Staphylococcus | <80 | <80 | <80 | <80 | <80 | <80 |
| epidermidis | | | | | | |
| Streptococcus | >80 | <80 | <80 | >80 | <80 | <80 |
| salivarius | | | | | | |

These results showed that only a few microorganisms have >80% homology between their genome sequences and at least one of the SARS-CoV-2 primers or gRNA included in the assay.

To confirm the in-silico evaluation, the same pathogens were in vitro to check for both potential cross-reactivity and interference.

The analysis was performed on a total of 22 pathogens by spiking either genomic DNA/RNA or inactivated strains into the SARS-CoV-2 negative lysed sample at the concentration indicated in Table 19 and tested using the assay described herein. Each pathogen was tested in triplicate. To discard any false negative results an RNAseP assay was run in parallel to each sample,

An interference analysis was also evaluated on the microorganisms that showed >80% homology with either the SARS-CoV-2 primers or the gRNA included in the kit. To detect any potential interference the analysis was performed by following the same protocol used for cross-reactivity testing in presence of 3×LoD SARS-CoV-2 (75 cp/μl).

All negative results for pathogens tested were confirmed by a positive result in the RNAseP assay.

TABLE 19

| | | Tested | Cross- | | |
|---|---|---|---|---|---|
| Pathogens | Source | Concentration | Reactivity | Interference | RNAse P |
| Human coronavirus 229E | 0810229CFHI | 10\hat{ }5 | 0/3 | N/A | 3/3 |
| | | TCID50/mL | | | |
| Human coronavirus OC43 | 0810024CFHI | 10\hat{ }5 | 0/3 | N/A | 3/3 |
| | | TCID50/mL | | | |
| Human coronavirus | ATCC VR- | 10\hat{ }5 copies/mL | 0/3 | N/A | 3/3 |
| HKU1 | 1580DQ | | | | |
| Human coronavirus NL63 | ATCC ® | 10\hat{ }5 copies/mL | 0/3 | N/A | 3/3 |
| | 3263SD | | | | |
| SARS-coronavirus | NATSARS-ST | 10\hat{ }5 | 0/3 | 3/3 | 3/3 |
| | | TCID50/mL | | | |
| Respiratory syncytial | ATCC ® VR- | 10\hat{ }5 copies/mL | 0/3 | N/A | 3/3 |
| virus | 1580DQ | | | | |
| Influenza A | VR-95DQ | 10\hat{ }5 copies/mL | 0/3 | N/A | 3/3 |
| Influenza B | VR-1885DQ | 10\hat{ }5 copies/mL | 0/3 | N/A | 3/3 |
| Mycobacterium | NR-14867 | 10\hat{ }6 copies/mL | 0/3 | 3/3 | 3/3 |
| tuberculosis | | | | | |
| Candida albicans | ATCC ® | 10\hat{ }6 copies/mL | 0/3 | 3/3 | 3/3 |
| | 10231D-5 | | | | |
| Pseudomonas aeruginosa | ATCC ® | 10\hat{ }6 copies/mL | 0/3 | 3/3 | 3/3 |
| | 27853D-5 | | | | |
| Staphylococcus epidermis | ATCC ® | 10\hat{ }6 copies/mL | 0/3 | N/A | 3/3 |
| | 12228D-5 | | | | |
| Streptococcus salivarius | HM-121D | 10\hat{ }6 copies/mL | 0/3 | 3/3 | 3/3 |
| Pooled human nasal fluid | 991-13-P-1 | N/A | 0/3 | N/A | 3/3 |
| Legionella pneumophila | ATCC ® | 10\hat{ }6 copies/mL | 0/3 | 3/3 | 3/3 |
| | 33152D-5 | | | | |
| Haemophilus influenzae | ATCC ® | 10\hat{ }5 copies/mL | 0/3 | N/A | 3/3 |
| | 51907D-5 ™ | | | | |
| Streptococcus pyogenes | ATCC ® | 10\hat{ }6 copies/mL | 0/3 | 3/3 | 3/3 |
| | 12344D-5 ™ | | | | |
| Streptococcus | ATCC ® | 10\hat{ }6 copies/mL | 0/3 | N/A | 3/3 |
| pneumoniae | 700669D-5 ™ | | | | |
| MERS-CoV | NR-45843 | 10\hat{ }5 copies/mL | 0/3 | N/A | 3/3 |
| Rhinovirus | NR-51453 | 10\hat{ }5 copies/mL | 0/3 | N/A | 3/3 |
| Chlamydia pneumoniae | 10\hat{ }6 | 10\hat{ }6 copies/mL | 0/3 | 3/3 | 3/3 |
| | copies/mL | | | | |
| Bordetella pertussis | 9797D-5 | 10\hat{ }6 copies/mL | 0/3 | 3/3 | 3/3 |

In conclusion, based on in-silico and in vitro analysis it was anticipated no cross-reactivity nor interference between primers/gRNAs included in the assay and most common pathogens in the respiratory tract.

### Clinical Evaluation

Clinical evaluation of the assay was performed using nasopharyngeal swabs as clinical samples from male and female adult patients with signs and symptoms of an upper respiratory infection.

A total of 47 positive samples and 43 negative samples were collected to assess the performance. All samples were also tested using an RT-PCR Test as a comparison method to obtain positive and negative percent agreement values. Results are presented in Table 20 and show 97.9% positive percent agreement (PPA) and 100% negative percent agreement (NPA) with comparator method.

TABLE 20

| | | RT-PCR Reference Method | | |
|---|---|---|---|---|
| | | Positive | Negative | Total |
| Direct CASPR | Positive | 46 | 0 | 46 |
| Lyo-CRISPR | Negative | 1 | 43 | 44 |
| SARS-CoV-2 | Total | 47 | 43 | 90 |

FIG. 20 shows that Cas12p has a minimal background signal after 30-60 minutes of cleavage activity. This provides advantages at low viral concentrations, and indicates stability of the lyophilized format. FIG. 21 shows that a diagnostics assay using Cas12p at room temperature, can be read out on a paper format. FIG. 22 shows that a diagnostics assay using Cas12p at room temperature can be read in well plate with a fluorescent detector.

### Example 12: SARS-CoV-2 Detection Using a Cas12p and a RNA Guide

Lyophilized beads with a RNA based reporter were used to detect SARS-CoV-2 RNA in patient and control samples. A subset of the samples described in Example 11 were used for this example. Cas12p was pre-incubated with their respective sgRNA and labeled RNA reporter was added before the lyophilization process. Pre amplified RT-LAMP product was used as input. Input for the RT-LAMP reaction were lysed sample from patient and negative control nasopharyngeal swabs. FIG. 19 shows the workflow for SARS-CoV-2 detection using a Cas12p/guide complex, using a RNA reporter, from a sample. FIG. 24 shows the results of SARS-CoV-2 detection using a Cas12p and a RNA reporter from patient samples and negative control samples in lyophilized format, at 30 minutes at 37° C. (n=16).

### Example 13: Testing Specific Cleavage Activity

FIG. 25: It was investigated whether the Cas12a.1 and the Cas12p of the disclosure are able to cut dsDNA when complexed with its guide. In these examples, the target was a Hanta virus dsDNA sequence (100 pb) cloned into the commercial pGEM®-T Easy vector from Promega (Cat. #A1360). Negative controls included the empty pGEM®-T Easy vector. The positive control included the pGEM®-T Easy vector/Hanta dsDNA target linearized by cut with NdeI restriction endonuclease from NEB (Cat. #R0111L). The procedure was as follows: 100 nM of Cas12a.1 or Cas12p were complexed with 100 nM of sgRNA to target the Hanta sequence, in a commercial NEBuffer™ 2.1 (Cat. #B7202S) for 15 min at RT. Controls with Cas enzyme not complexed with its guide were included. Then, 5 ng/uL of target was added, in a final reaction volume of 20 uL. Reactions were incubated at 37 or 25° C. for 0, 30, 60 or 90 min, and ended by addition of 50 mM EDTA. Then, the samples were centrifuged at 12000 g for 10 min and mixed with 6× Gel Loading Dye from NEB (Cat #B7024S). Samples were analyzed in a 0.8% TBE-agarose gel. Fast DNA Ladder from NEB (Cat. #N3238S) was used to assess the molecular weight of the species. After electrophoresis the gel was stained for 30 min with a fresh solution of SYBR™ Gold Nucleic Acid Gel Stain from Invitrogen (Cat #S11494) and imaged on VersaDoc™ Imaging System (Bio-Rad). FIG. 1 shows the results of the assay. Cas12a.1 could linearize the totality of the plasmid after 90 min at 37° C., while Cas12p lasted only 60 min to achieve comparable results.

FIG. 26: It was investigated whether the Cas12a.1 and Cas12p of the disclosure are able to cut ssDNA when complexed with its guide. In these examples, the target consisted of a custom ssDNA fluorescence marked sequence (3′FAM-ssDNA) of 70 nucleotide length from IDT (5′-TCA TTT AGA AAG TAG ATA TTG ATT GAT TTT AGC GAA AGC CAA TTT TTG AGC TGC CAC TGA TGT AAA AGT T-3′-6-FAM; SEQ ID NO: 124) targeted to Hanta virus. Negative control included a custom anti-sense ssDNA sequence (ASssDNA) of 120 nucleotide length from IDT (5′-GCT ATC TTA ATC CTT AAT CTA TCC TCA AAC GTT CTA TTA ATG GCC GTG TCA ATC AAT ATC TAC TTT CTA AAT GAA ACT TTT ACA TCA GTG GCA GCT CAA AAA TTG GCT TTC GCT AAA ATC-3′; SEQ ID NO: 125) also targeted to Hanta virus. The procedure was as follows: 10 pmol of Cas12a.1 or Cas12p, were complexed with 10 pmol of sgRNA to target Hanta sequence, in commercial NEBuffer™ 2.1 (Cat. #B7202S) for 15 min at RT. Controls with Cas enzyme not complexed with its guide were included. Then, 10 pmol of 3′FAM-ssDNA or alternatively ASssDNA was added, in a final reaction volume of 10 uL. Reactions were incubated at 37° C. for 0, 0.5, 1 or 5 min, and ended by addition of 2× Novex™ TBE-Urea Sample Buffer from Invitrogen (Cat #LC6876) followed by heating at 95° C. for 3 min. Samples were centrifuged at 12000 g for 10 min and analyzed on 15% Mini-PROTEAN© TBE-Urea Gel from Bio-Rad (Cat. #4566056). Oligo length standards from IDT (Cat. #51-05-15-02) was used to assess the molecular weight of the species. Gels were first imaged on VersaDoc™ Imaging System (Bio-Rad) and then were stained for 30 min with a fresh solution of SYBR™ Gold Nucleic Acid Gel Stain from Invitrogen (Cat #S11494) to visualize the non-fluorescence marked sequence of ASssDNA and the non-fluorescence marked ladder. FIG. 2 shows the results of the assay. Cas12a.1 and Cas12p demonstrated specific ssDNA cleavage of the 3′FAM-ssDNA substrate (S), with the production of a ˜40 nucleotide length product (P). The two Cas enzymes were unable to cut the ASssDNA sequence (NTC). The reactions took place in the timeframe of seconds to few minutes.

FIG. 27: It was investigated whether the Cas12a.1 and Cas12p of the disclosure are able to cut ssRNA, when complexed with its guide. In these examples, the target consisted of a ssRNA sequence obtained by in vitro transcription (IVT) and targeted to Hanta virus. Negative control included a custom non-target ssRNA sequence of 65 nucleotide length from IDT (5′-TAA GCG CCC TTG CGC TTT CCC CAG CCT TCG GGT TGG TTG CCT TTT AGT GCA AGG GCG CGA TTA TT-3′; SEQ ID NO: 126). Positive control included a custom ssDNA sequence of 120 nucleotide length from IDT (5′-GAT TTT AGC GAA AGC CAA TTT TTG AGC TGC CAC TGA TGT AAA AGT TTC ATT TAG AAA GTA GAT ATT GAT TGA CAC GGC CAT TAA TAG AAC GTT TGA GGA TAG ATT AAG GAT TAA GAT AGC-3′; SEQ ID NO: 127), targeted to Hanta Virus. The procedure was as follows: 150 nM of Cas12a.1 or Cas12p were complexed with 150 nM of sgRNA to target Hanta sequence, in commercial NEBuffer™ 2.1 (Cat. #B7202S) for 15 min at RT. Controls with Cas enzyme not complexed with its guide were included. Then, 5 ng/uL of ssRNA or alternatively non-target ssRNA or ssDNA was added, in a final reaction volume of 10 uL. Reactions were incubated at 37° C. for 0, 1 or 3 h, and ended by addition of 2× Novex™ TBE-Urea Sample Buffer from Invitrogen (Cat #LC6876) followed by heating at 65° C. for 3 min. Samples were centrifuged at 12000 g for 10 min and analyzed on 15% Mini-PROTEAN© TBE-Urea Gel from Bio-Rad (Cat. #4566056). Low Range ssRNA Ladder from NEB (Cat. #N0364S) was used to assess the molecular weight of the species. Gels were stained for 30 min with a fresh solution of SYBR™ Gold Nucleic Acid Gel Stain from Invitrogen (Cat #S11494) and imaged on VersaDoc™ Imaging System (Bio-Rad). FIG. 3 shows the results of the assay. Neither Cas12a.1 nor Cas12p demonstrated specific ssRNA cleavage activity.

### Example 14: Characterization of Non-Specific Nuclease Activity of Cas12p

MALDI-TOF MS experiment description: Matrix-assisted laser desorption/ionization-time of flight mass spectrometry (MALDI-TOF MS) was employed to monitor the products generated by the unspecific nuclease activity of Cas12p enzyme. Protected DNA (C* C* C* C* C* C* C* C* C* C* C* C* C* C* C* C* C* C*TTATT; SEQ ID NO: 128) and RNA (rC* rC* rC* rC* rC* rC* rC* rC* rC* rC* rC* rC* rC* rC* rC* rC* rC* rC*rUrUrArUrU; SEQ ID NO: 129) reporters were used to ensure a minimal length and minimize the number of possible hydrolysis products. The symbol (*) on C and rC bases indicates the presence of phosphorothioate bonds that are resistant to nuclease degradation. CRISPR reactions with the corresponding reporter were performed with complexes to a final concentration of 75 nM Cas12p:75 nM sgRNA:20 nM activator:2.5 uM DNA reporter or 75 nM Cas12p:75 nM sgRNA:10 nM activator:1.25 uM RNA reporter in a solution containing 1×Binding Buffer (50 mM NaCl, 10 mM Tris-HCl, 10 mM MgCl2, 1 mM DTT, 100 g/ml BSA, pH 7.9). The reactions were incubated during 1 h at 25° C. for DNA reporter or 6 h at 37° C. for RNA reporter (T1 of reaction, FIG. 28 and FIG. 30). The time zero (T0, FIG. 29 and FIG. 31) of reaction was made as a negative control by heating Crispr reaction before reporter addition. The reactions were purified and analyzed on a PerSeptive Biosystems (ABI)-Voyager-DE RP-MALDI-TOF mass spectrometer, Stanford University. For each reaction, a list was generated with the predicted m/z (mass to charge ratio) of all the possible DNA/RNA cleavage products and all the expected overhangs, as was proposed by Joyner et al. 2012. The observed m/z were correlated to the aforementioned list by the use of a Perl script. Relative intensity of peaks was calculated in relation to the predominant signal of the reaction. DNA reporter hydrolysis gave rise to a unique cleavage product (FIG. 28) meanwhile the hydrolysis of RNA reporter generated multiple fragments, including both 3′ hydroxide and phosphate ends (FIG. 30). In all cases, the predominant hydrolysis species was the one that contained two nucleotides after the protected sequence and 3′ hydroxide ends.

FIG. 28-29 show the mass spectra data of Cas12p reactions using a DNA oligo as the reporter. FIG. 30-31 shows the mass spectra data of Cas12p reactions using a RNA oligo as the reporter.

### Example 15: Characterization of the Use of Chimeric (Hybrid) Guides

Guide sequences (hybrid guides, chimeric guides) partially composed of DNA and RNA nucleotides were tested and determined that they can support efficient collateral Cas12p activity. Partial replacement with DNA nucleotides at 3′ of sgRNA (Hybrid 4 DNA; 5′AGAUUUCUACUUUUGUAGAUGUGGCAGCUCAAAAAU(TGGC)3′; SEQ ID NO: 130) or a replacement with DNA nucleotides at both 5′ and 3′ (Hybrid 3/4 DNA; 5′(AGA)UUUCUACUUUUGUAGAU GUGGCAGCUCAAAAAU(TGGC)3′; SEQ ID NO: 131) maintained its activity compared to the unmodified guide sequence (sgRNA; 5′AGAUUUCUACUUUUGUAGAU GUGGCAGCUCAAAAAUUGGC3′; SEQ ID NO: 132). A partial replacement of 8 DNA nucleotides at 3′ led to a complete loss of Cas12p collateral cleavage activity (Hybrid 8 DNA; 5′AGAUUUCUACUUUUGUAGAU GUGGCAGCUCAA(AAATTGGC)3′; SEQ ID NO: 133).

Cas12p was pre-incubated with their respective sgRNA or hybrid guides (1 uM complex). The reaction was initiated by diluting Cas12p complexes to a final concentration of 37.5 nM Cas12p:37.5 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 600 nM TTATTATT ssDNA FQ reporter (SEQ ID NO: 121) substrates in a 40 μl reaction. Reactions (40 μl, 384-well microplate format) were incubated in a fluorescence plate reader (SpectraMax® M2) for 40 minutes at 25° C. with fluorescence measurements taken every 1 minute (ssDNA FQ substrates=λex: 485 nm; λem: 538 nm). The result showed the quantification of maximum fluorescence signal generated after 30 minutes. Non-template negative control (NTC) fluorescence values were calculated from reactions carried out in the absence of target plasmid. Error bars represent the mean±s.d., where n=3 replicates.

FIG. 32 shows that the DNA-RNA chimeric guides used enable efficient collateral Cas12p activity.

### Example 16: Characterization of Collateral Cleavage Activity

FIG. 33 shows agarose gels showing the collateral activity for Cas12a.1 and Cas12p protein/guide complexes using the following substrates: (A) M13mp18 single-stranded DNA (Cat #N4040S, NEB); and (B) M13mp18 RF I double-stranded DNA (Cat #N4018S, NEB). Cas12a.1 and Cas12p exhibit collateral activity and cleavage ssDNA circular DNA (FIG. 33, Panel A), but not dsDNA circular DNA (FIG. 33, Panel B). The reaction was initiated by diluting Cas12p/guide or Cas12a.1/guide complexes to a final concentration of 37.5 nM Cas12p:37.5 nM sgRNA:10 nM activator or 75 nM Cas12a.1:75 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 1 uL of M13mp18 single-stranded DNA (Cat #N4040S, NEB) and M13mp18 RF I double-stranded DNA (Cat #N4018S, NEB) at 25° C. for 1 h. Control groups without the Cas enzyme, guide or activator were included and non-collateral cleavage was observed.

Cleavage efficiency Cas12p showed a similar cleavage efficiency for at least the T, A, or C homopolymeric reporter (7 nt in length), whereas Cas12a.1 demonstrated a higher efficiency in poly C cleavage but also cleaved polyA and poly T sequences. Cas12p displayed cleavage at 25° C. for T, A, or C homopolymeric reporter evidenced by increased fluorescence, whereas Cas12a.1 only demonstrated cleavage response at 37° C. with the 5′6-FAM-TTATTATT-3IABkFQ3′ reporter sequence (SEQ ID NO: 121).

The reaction was initiated by diluting Cas12p or Cas12a.1 complexes to a final concentration of 37.5 nM Cas12p:37.5 nM sgRNA:10 nM activator or 75 nM Cas12a.1:75 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 600 nM ssDNA FQ reporter substrates (5′6-FAM-TTATTATT-3IABkFQ3′ (SEQ ID NO: 121), 5′6-FAM-AAAAAAA-3IABkFQ3′, 5′6-FAM-TTTTTTT-3IABkFQ3′, 5′6-FAM-CCCCCCC-3IABkFQ3′ or 5′6-FAM-C*GGGC*GGG-3IABkFQ3′ from IDT (Integrated DNA Technologies, Inc)) in a 40 μl reaction. Reactions (40 μl, 384-well microplate format) were incubated in a fluorescence plate reader (SpectraMax® M2) at 25° C. or 37° C. with fluorescence measurements taken every 1 minute (ssDNA FQ substrates=λex: 485 nm; λem: 538 nm). Background-corrected fluorescence values were calculated by subtracting fluorescence values obtained from reactions carried out in the absence of target plasmid. Error bars represent the mean±s.d., where n=3 replicates. FIG. 34 shows the differential efficiency in cleavage of homopolymeric reporters, at 25° C. and 37° C. The results show that Cas12p cleaved poly T, poly A and poly C, whereas Cas12a.1 showed a preference for polyC cleavage.

The specificity of trans-cleavage activity (collateral activity) was tested using a customized ssRNA 5′6-FAM rArUrArUrArUrA-3IABkFQ3′ and RNaseAlert™ (a commercially available RNA reporter) from IDT (Integrated DNA Technologies, Inc) as RNA reporters. The results showed that Cas12p is able to cleave RNA reporters used but Cas12a.1 is not. Detection assays were performed at 37° C. using Cas12p or Cas12a.1 complexes to a final concentration of 37.5 nM Cas12p:37.5 nM sgRNA:10 nM activator or 75 nM Cas12a.1:75 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 600 nM of RNA FAMQ reporter substrates (ssRNA 5′6-FAM rArUrArUrArUrA-3IABkFQ3 and RNaseAlert (Cat N 11-04-03-03-IDT)) in a 40 μl reaction. Reactions were incubated in a fluorescence plate reader (SpectraMax® M2) and background-corrected fluorescence values were calculated by subtracting fluorescence values obtained from reactions carried out in the absence of target plasmid. Error bars represent the mean±s.d., where n=3 replicates. FIG. 35 shows the result of these data, and shows the collateral cleavage ability of Cas12p but not of Cas12a.1, to cleave a RNA reporter.

The kinetics of collateral cleavage (trans-cleavage) activity using DNA and RNA reporters was assessed for Cas12p. Experiments with an RNA substrate showed a cleavage rate of ssRNA only 3-fold slower than a ssDNA reporter. The cleavage rate of Cas12a.1 for the ssRNA substrate was at least 1.10⁴-fold slower than for ssDNA, confirming that ssDNA is the choice substrate for Cas12a.1 collateral cleavage. Detection assays were performed at 37° C. using Cas12p or Cas12a.1 complexes to a final concentration of 37.5 nM Cas12p:37.5 nM sgRNA:10 nM activator or 75 nM Cas12a.1:75 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 600 nM of ssDNA FAMQ reporter substrates (ssDNA 5′6-FAM TTATTATT-3IABkFQ3 (SEQ ID NO: 121)) or RNaseAlert (Cat N 11-04-03-03-IDT)) in a 40 μl reaction. Reactions were incubated in a fluorescence plate reader (SpectraMax® M2) for up to 40 minutes with fluorescence measurements taken every 1 minute (λex: 535 nm; λem: 595 nm). Background-corrected fluorescence values were calculated by subtracting fluorescence values obtained from reactions carried out in the absence of target plasmid. The resulting data were fit to a single exponential decay curve (GraphPad Software), according to the following equation: Fraction cleaved=A×(1−exp(−k×t)), where A is the amplitude of the curve, k is the first-order rate constant, and t is time. Error bars represent the mean±s.d., where n=3 replicates. FIG. 36 shows the results of these data, and shows the kinetics of collateral cleavage activity of Cas12p and Cas12a.1, using DNA and RNA as reporters.

Reporters composed of DNA and RNA nucleotides led to efficient collateral Cas12p and Cas12a.1 activity. FQ Hybrid/56-FAM/TT rArUrU ATT/3IABkFQ/ or /56-FAM/TT ATrU rArUrU/3IABkFQ/1ed to a maintained Cas12p collateral activity compared to the ssDNA or RNA reporters (ssDNA FAMQ reporter substrates (ssDNA 5′6-FAM TTATTATT-3IABkFQ3 (SEQ ID NO: 121)) or RNaseAlert (Cat N 11-04-03-03-IDT))). Whereas Cas12a.1 showed a slight decrease efficiency in trans-cleavage of chimeric reporters in comparison with the ssDNA. The reaction was initiated by diluting Cas12p or Cas12a.1 complexes to a final concentration of 37.5 nM Cas12p:37.5 nM sgRNA:10 nM activator or 75 nM Cas12a.1:75 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 600 nM of ssDNA FAMQ reporter substrates (ssDNA 5′6-FAM TTATTATT-3IABkFQ3 (SEQ ID NO: 121), DNA-RNA chimeric reporters (/56-FAM/TT rArUrU ATT/3IABkFQ/, /56-FAM/TT ATrU rArUrU/3IABkFQ/ or RNaseAlert (Cat N 11-04-03-03-IDT)) in a 40 μl reaction. Reactions were incubated in a fluorescence plate reader (SpectraMax® M2) for up to 40 minutes with fluorescence measurements taken every 1 minute (λex: 535 nm; λem: 595 nm). Background-corrected fluorescence values were calculated by subtracting fluorescence values obtained from reactions carried out in the absence of target plasmid. Error bars represent the mean±s.d., where n=3 replicates. FIG. 37 shows the results of these data.

While the inventions have been described in conjunction with the detailed description thereof, the foregoing description is intended to illustrate and not limit the scope of the invention, which is defined by the scope of the appended claims. Other aspects, advantages, and modifications are within the scope of the following.

### Example 17: Cas12a.1 and Cas12p Mature Guide Characterization and Validation

The scaffold sequences of the mature guides were deduced in silico from the corresponding CRISPR loci. FIG. 38 shows the secondary structure of the mature guide scaffold for Cas12a.1 (5′ aaauuucuacuguaguagau 3′) (SEQ ID NO: 116; Panel A) and Cas12p (5′ agauuucuacuuuuguagau3′) (SEQ ID NO: 117; Panel B). These were validated below.

The mature guide scaffolds for Cas12a.1 and Cas12p were evaluated in vitro. These mature scaffold sequences, along with a spacer targeting the N gene from SARS-CoV-2 virus were used in this example. The reactions were initiated by diluting Cas12p or Cas12a.1 complexes to a final concentration of 37.5 nM Cas12p:37.5 nM sgRNA:10 nM activator or 75 nM Cas12a.1:75 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 600 nM of ssDNA FAMQ reporter substrates (ssDNA 5′6-FAM TTATTATT-3IABkFQ3 (SEQ ID NO: 121)) (in a 40 μl reaction. Reactions were incubated in a fluorescence plate reader (SpectraMax® M2) for up to 20 minutes with fluorescence measurements taken every 1 minute (λex: 535 nm; λem: 595 nm). Background-corrected fluorescence values were calculated by subtracting fluorescence values obtained from reactions carried out in the absence of target plasmid. Error bars represent the mean±s.d., where n=3 replicates (FIG. 39). The data in this figure show that these mature scaffold sequences provide for CRISPR-mediated detection of SARS-CoV-2.

1-24. (canceled)

25. An engineered system comprising:
a. a Cas12a.1, Cas12p, or Cas12q protein, or a nucleic acid encoding the Cas12a.1, Cas12p, or Cas12q protein; and
b. a Cas12a.1, Cas12p, or Cas12q gRNA, or a nucleic acid encoding a Cas 12a.1, Cas12p, or Cas12q gRNA,
wherein the gRNA and the Cas12a.1, Cas12p, or Cas12q protein do not naturally occur together, wherein the gRNA is capable of hybridizing to a target sequence in a target DNA, and the gRNA is capable of forming a complex with the Cas12a.1, Cas12p, or Cas12q protein.

26. The system of claim 25, comprising: a. a Cas12a.1, Cas12p, or Cas12q protein; and b. a Cas 12a.1, Cas 12p, or Cas 12q gRNA.

27. The system of claim 25, comprising: a. a nucleic acid encoding the Cas12a.1, Cas12p, or Cas12q protein; and b. a nucleic acid encoding a Cas12a.1, Cas12p, or Cas12q gRNA.

28. The system of claim 25, wherein the Cas12a.1 protein comprises the amino acid sequence of SEQ ID NO: 3, or at least 70% sequence identity thereto.

29. The system of claim 25, wherein the Cas12p protein comprises the amino acid sequence of SEQ ID NO: 4, or at least 70% sequence identity thereto.

30. The system of claim 25, wherein the Cas12q protein comprises the amino acid sequence of SEQ ID NO: 222, or at least 70% sequence identity thereto.

31. The system of claim 25, wherein the Cas12q protein comprises the amino acid sequence of SEQ ID NO: 5, or at least 70% sequence identity thereto.

32. (canceled)

33. The system of claim 25, wherein the target sequence is a sequence of a human.

34. The system of claim 25, wherein the target sequence is a sequence of a non-human primate.

35. The system of claim 25, wherein the target sequence is a bacterial or viral sequence.

36. The system of claim 25, wherein the Cas12a.1, Cas12p, or Cas12q protein is a catalytically active Cas12a.1, Cas12p, or Cas12q protein.

37. The system of claim 36, wherein the Cas12a.1, Cas12p, or Cas12q protein cleaves at a site distal to the target sequence.

38. The system of claim 25, wherein the Cas12a.1, Cas12p, or Cas12q protein is a catalytically dead Cas12a.1, Cas12p, or Cas12q protein.

39. The system of claim 25, wherein the Cas12a.1, Cas12p, or Cas12q protein comprises nickase activity.

40-70. (canceled)

71. A method of detecting a target DNA in a sample, the method comprising:
a. contacting the sample with:
i. a Cas12a.1, Cas12p, or Cas12q protein;
ii. a Cas12a.1, Cas12p, or Cas12q gRNA comprising a spacer sequence that is capable of hybridizing with a target sequence in a target DNA; and
iii. a labeled detector that does not hybridize with the spacer sequence of the gRNA; and
b. measuring a detectable signal produced by cleavage of the labeled detector by the Cas12a.1, Cas12p, or Cas12q protein, thereby detecting the target DNA.

72. The method of claim 71, wherein the labeled detector comprises a labeled single stranded DNA.

73. The method of claim 71, wherein the labeled detector comprises a labeled RNA.

74. The method of claim 73, wherein the labeled RNA is a single stranded RNA.

75. The method of claim 71, wherein the labeled detector comprises a labeled single stranded DNA/RNA chimera.

76-122. (canceled)


SEQUENCE LISTING (249 sequences)

SEQ ID NO: 1 - protein, 1038 aa, Unknown
  Cas9.1
MQRIFGLDIGTTSIGFAVIDHDRDQGVGRIHRLGARIFPEARDEKGTPLNQHRRQKRLARRQLRRRRLRRKALNELLSARGMLPRFGTSAWHDAMALDPYALRARGTEEALQPVEVGRALYHLAQRRHFKPRDEAAEADEQEVGDQEAETKREKLLQALRRSGRTLGQELAARGPHERKRHEHALRSTVETEFERLLTAQARHHEILRDPEFVEELRETIFAQRPVFWRTSTLGTCPFVPGAPLCPKGAWLSRQRRMLEQVNNLAITGGNARPLDHEERRAILAVLQTQASMSWGAVRTALKPLFKARGEAGAERRLRFNLEEGGGKTLLGNPLEAKLARIFGEAWATHPHRDAIRETIHDRLFAATYNAKGAQRIVILPASQRAERMRGVIAGLQADFGLSHEQAMALAELPLTPGWEPYSSEALRALMPKLEEGVRFGALVVAPEWEDWREATFPQRERPTGEVLDLLPSPKCHDESRRQTRLRNPTVLRTQNELRKVVNNLIRAHGKPDIIRVEVAREVGLSKREREDRYNGMRRQERQRQAAIKDLQAKGFAEPSRADVEKWLLWKESKETCPYTGDKICFDALFRRGEFQVEHIWPRSRSFDDSFRNKTLCRRDVNLAKGNQTPFEFFESRPEEWEAVKRRLDGLQAKRAGGEGMARGKVKRFVASTLPDDFAQRQLNDTGWAAREAVAFLKRLWPDEGQAAPVRVQAVTGRVTAQLRHLGGLDGVLSDGARKTRDDHRHHAVDALVVACTHPGMTERLSRYWQQKEDERAERPQLDPPWPTIRADAEAAKDLIVVSHRVRKKISGPFHKETVYGATDEREVTRGLEYEKFVTRKRVEDLTKSMLADIRDDRVRQIVTAWVAERGGDPKKAFPPYPTLGSSGPEIRKVRVLIRRQPTLMARAATGFADLGANHHVAIYKTADERFAFEVVSLLEVARRVDRGEPPVKRQRGDEKLVMSLAQGDLIRFAKTPDAEAAIWRVQKIATKGQISLLHHDDASPKEPSLFEPMVGGLMARNPEKLAVDPIGRVRKAGD

SEQ ID NO: 2 - protein, 1375 aa, Unknown
  Cas9.2
MKKEKVYMGLDLGTNSVGWAVTDNDYKVLKFKRRAMWGVRLFNEANPAVERRVARSNRRRLARKKQRVAWLKEIFKNSISEIDPEFFDRLEQSALWAEDKNVAGKYSLFNEKKLTDKTFYRKFPTVFHLKKALMDGKIKKPDIRFVYLALSHYLQNRGHFLLENELNSVEDIDIRDIFNSLNERIHVLIDSGDDMVPAFDLTNLDDLKQIATDTNISGKTQEKEAFIKTLLNGAKQPALEAIIKLCTGGSANLSKIFGDMFEFESEIKSISFEKANFEDEIAPKLQDCLGDYYQIIELAQQIYSWYTLYKVCSGRPSVSHAKVEDYEKHKEQLSHLKVLVRKHFSKNVYREIFRKEDDKIHNYVSYISGKKDRDEFYKYLKKTLEKKSTFKKTSEFENISRAIEQQNYLPKQRVKDNSVVPQQLYKQEIVKILNNLSSHYPFLSQKTDGISNREKIIKIFEYRIPYYVGPLCDIHRAGDDGFSWLVRDCSKKITPWNFEQVVDIPQSAENFIKNMTRKCTYLKQYNVLPKNSLLYSEYSVLNELNNVRIKTKKLTPKLKEKMLNTLFRQKKNISITSLIHWLVSEGVYEKGEIEKSDVSGVDSNFTSSLSAAISFDRIIGEKMKNKKTQKMVEEIINWLALFSDKKILQQKIVEKYQDKVSQEQIGKILRLNLSGWGRLSSEFLQLKNSQPGEHDGKTLINIMRQTQMNLMEIIHSPQFSFNTVIETEAKKQLTGHITHSHVEALYCSPVVKKQIWQALQIALELKKTLKKDPNKIFVETTRHEGEKKRTTSRHKQLLELYQAAKSHLPDLTKSIKELNDALKDTEPEKMKRKKLFHYYKQLGRCMYTGRPISLEDLFTNKYDIDHIYPQSLTKDDSFTNTVLVERLSNAEKSDAFPLDSKTRKDRQGLWRCLRRNGLITKEKYYRLTRETPLSEEEKAAFIRRQLVETSQTTKEVIRFLATLFPKSKVVYVKSGNVSDFRRDFSPSLPENKTNGKDPKGITDYSMIKVREINDLHHAKDAYLNIVVGNVYDTKFRYRGKDLTAIVREKARQYHLSRLFLYSTDGAWIGAADENRGKQRPSIETVIAEMRRNSCQVTWEAVFKKGQLWDMNAKSKRPGLLPIKKELSDTAKYGGYQGKTASYFVVVEYENKKGEREKKLESVPIYVKALSKQKPDAVNSFLRDTLGLEKPSVMVDNIKIGSIVEINGARMVLTGNNEVLVFGRIASQLILDITMAAYLKRMFKLLADTAKIKENNVYFKNCGYLDKETNLAVYDTFIAKLKLPRYAQIITHSLYEKMESNRDVFINLSLADQCNLLAGVLPALQCNSQNADLSLLGEGKAVGNIAFSKNAILKKNQVRLVDCSITGLFENSRNMA

SEQ ID NO: 3 - protein, 1254 aa, Unknown
  Cas12a.1
MKVSTWDSFTNQYPLTKTLRFELKPVGKTLQKIQDRNLITEDEQRQKDFNKVKKIMDGYYKQFIEECLEGAKIPLKKLEENNNAYTKLKKDPYNKKLREEYAKLQKQLRKLIHDEINKKEEFKYLFKKEFIKKILPEWLEKKGKKEELKEIEKFDKWVTYFSGFFNNRKNVFSSDEISTSMIYRIVNDNLPKFLDDVSRFGEITRYKEFDANQIEENFESELNGEKLKDFFNLKNFNNCLNQEGIEKFNLIIGGKSEEGNNKIKGLNELVNELAQKQADKNEQKKVRKLKLAPLFKQILSDRKSSSFAFEKFEENTEVFDAIDEFYDKISLETLKKIEATLEKLEEKDLELVYLKNDRCLTGISQEVFGDRERVLQALREYAKTELGLKTDKKIEKWMKKGRYSIHEIESGLKKIGSTGHPICNYFSKLEEKKTNLIQEIKKARTEYEKISDKKKKLTAESQEPNVARIKALLDSIMRLYHFIKPLNINFKNKKEKDSEALETDNDFYNDFDESFAELGNIIPLYNQVRNYVTQKPFSTEKFKLNFENPKLLSGWDKNKEKDYYSVILRKEESYYLAIMTPKQKNVFDELERLPAGKNYFEKIDYKLLPTPEKNLPRILFAKKNISFYKPSKEIEAIRNHSAHTKHGNPQNGFKKRDFRLSDCHKMIDFYKKSIQKHPEWKEYDFQFKKTEDYVDISEFYKEVSDQGYKIEFKKISEKYLLDLVEEGKLYLFQIWNKDFSKYSEGRKNLHTIYWKELFSKENLSDITYKLNGEAEIFYRPKSMERKVTHPKNQKIENKDPIKGKKFSKFKYDFIKNKRYTEDRFFFHCPITLNFQARDGSKTINKRVNDHIRETKDDIFVLSIDRGERHLAYYTLLNSKGEIQEQGSFNVISDDKERKRDYHEKLDEREKERDKARKSWQKIETIKKLKDGYLSQIVHKIAKLAIEKNAIIVLEDLNLDFKRGRLKIEKQVYQKFEKKLIDKLNYLVFKERTEKEAGGSLNAYQLTGKFEGFKKLGKETGIIYYVPAAYTSKICPKTGFVNLLRPKFKNIEKAKEFFKKFNYIKYDSSEGLFEFNFDYSKFIKNGKKETKIIQDNWSVYSNGTKLVGFRNKNKNNSWDTKEVKPNEKLKILFKEYGVSFQKDENIISQIASQNKKAFFENLIKIFKTILMLRNSRKDPEEDYVLSCVKDENGEFFDSRKAKDNEPKDADANGAYHIGLKGLMLLERIKANKGKKKLDLLISRNDFINFAVERSK

SEQ ID NO: 4 - protein, 1281 aa, Unknown
  Cas12p
MKKSIFDQFVNQYALSKTLRFELKPVGETGRMLEEAKVFAKDETIKKKYEATKPFFNKLHREFVEEALNEVELAGLPEYFEIFKYWKRYKKKFEKDLQKKEKELRKSVVGFFNAQAKEWAKKYETLGVKKKDVGLLFEENVFAILKERYGNEEGSQIVDESTGKDVSIFDSWKGFTGYFIKFQETRKNFYKDDGTATALATRIIDQNLKRFCDNLLIFESIRDKIDFSEVEQTMGNSIDKVFSVIFYSSCLLQEGIDFYNCVLGGETLPNGEKRQGINELINLYRQKTSEKVPFLKLLDKQILSEKEKFMDEIENDEALLDTLKIFRKSAEEKTTLLKNIFGDFVMNQGKYDLAQIYISRESLNTISRKWTSETDIFEDSLYEVLKKSKIVSASVKKKDGGYAFPEFIALIYVKSALEQIPTEKFWKERYYKNIGDVLNKGFLNGKEGVWLQFLLIFDFEFNSLFEREIIDENGDKKVAGYNLFAKGFDDLLNNFKYDQKAKVVIKDFADEVLHIYQMGKYFAIEKKRSWLADYDIDSFYTDPEKGYLKFYENAYEEIIQVYNKLRNYLTKKPYSEDKWKLNFENPTLADGWDKNKEADNSTVILKKDGRYYLGLMARGRNKLFDDRNLPKILEGVENGKYEKVVYKYFPDQAKMFPKVCFSTKGLEFFQPSEEVITIYKNSEFKKGYTFNVRSMQRLIDFYKDCLVRYEGWQCYDFRNLRKTEDYRKNIEEFFSDVAMDGYKISFQDVSESYIKEKNQNGDLYLFEIKNKDWNEGANGKKNLHTIYFESLFSADNIAMNFPVKLNGQAEIFYRPRTEGLEKERIITKKGNVLEKGDKAFHKRRYTENKVFFHVPITLNRTKKNPFQFNAKINDFLAKNSDINVIGVDRGEKQLAYFSVISQRGKILDRGSLNVINGVNYAEKLEEKARGREQARKDWQQIEGIKDLKKGYISQVVRKLADLAIQYNAIIVFEDLNMRFKQIRGGIEKSVYQQLEKALIDKLTFLVEKEEKDVEKAGHLLKAYQLAAPFETFQKMGKQTGIVFYTQAAYTSRIDPVTGWRPHLYLKYSSAEKAKADLLKFKKIKFVDGRFEFTYDIKSFREQKEHPKATVWTVCSCVERFRWNRYLNSNKGGYDHYSDVTKFLVELFQEYGIDFERGDIVGQIEVLETKGNEKFFKNFVFFFNLICQIRNTNASELAKKDGKDDFILSPVEPFFDSRNSEKFGEDLPKNGDDNGAFNIARKGLVIMDKITKFADENGGCEKMKWGDLYVSNVEWDNFVANK

SEQ ID NO: 5 - protein, 1137 aa, Unknown
  Cas12q
MINIDELKNLYKVQKTITFELKNKWENKNDENDRVEFLKTQEWVESLFKVDEENFDEKESIPNLLDFGQKIASLFYKLSEDIANNQIDTRVLKVSKFLLEEIDRNQYHEKKNKPTKVKEMNPNTNKSYIKEYKLSDQNTLYVLLKIMEDEGRGLQKFLYDKADRLNLYNQKVRRDFALKESNEQQKFSGNANYYGNIKLLIDSLEDAVRIIGYFTFDDQAENAQINEFKSVKQEMNNNEASYQALKDFAIDNAKKEIELTTLNHRAVNKDPKKIQEQIEEVENFEEDINQLKHQISALNDKKFDVVSRLKHALIKMLPELNLLDAESEQGREVQQIYQDKKNGLELDDFKFNLLKHHQWQKTIFKYIKLEGLVLPDLYAENKQDKIKVYIENYRQSGERISKKAREELGKIDKREEFNGNDELKKAWYEYKDFCRDKRNKSVELGNKKSLYNAIKREVLRQKMCNHFAVLVSDGEDTSPYYYLILIPNENSDEMNRTFKELKASEGNWKMLDYNRLTFKALEKLALLRSSTFEIADQELQEEAKKIWEEYKEKAYKDFKNKKLLQGLSGRQREEKKQELQKESLNRVINYLIRCIQSLPDSGKYNFNFKEPHQYQSLEEFAEEIDRQGYHCAWKNVSKDKLMELEAMEKIKVFKLHNKDFRKVKLNDSKHNPNLFTLYWLDAMNLDKVNVRLLPEVDLYKRAKETQLKLFERDVKCNINNQKIKSIKEKNRLFQDKLYASFKLEFYPENEGLGFEQVNDKVNNFCGSDTAYYLGLDRGEKELVTFCLVDSDGRLVKNGDWTKFKEVNYADKLKQFYYSKGEIESTQQQLLEARDNIKQATNTEDKESMKLNYKKLELKLKQQNLLAQEFIKKAYCGYLIDSINEILREYPNTYLVLEDLDIAGKADPESGMTNKEQNLNKTMGASVYQAIENAIVNKFKYRTVKLSDIKGLQTVPNVVKVEDLREVKEVEDGEHKFGLIRSVKSKDQIGNILFVDEGETSNTCPNCGFNSDWFKRDVDFDLEIVATVNGQKNAVIEQNDKKYCFPGEIYKLEIINKEYETNKRNLAMIFKPRAKACRKFINNNLDKNDYFYCPYCAFSSKNCNNPKLQNGDFVVYSGDDVAAYNVAIRGINLLNNIK

SEQ ID NO: 6 - DNA, 36 bp, Unknown
  Cas12a.1 Direct Repeat
gtttaaggccttgacaaaatttctactgtagtagat

SEQ ID NO: 7 - DNA, 36 bp, Unknown
  Cas12p Direct Repeat
atctacaaaagtagaaatctaatagggatattcgag

SEQ ID NO: 8 - DNA, 36 bp, Unknown
  Cas12q Direct Repeat
atctacaaaagtagaaattaaataggtctatttgag

SEQ ID NO: 9 - DNA, 36 bp, Unknown
  Cas9.1 Direct Repeat
actgtagcaagacgaagggccggcgcaatccgcagc

SEQ ID NO: 10 - protein, 1031 aa, Unknown
  Cas9.3
MIFGLDVGTTSIGFALISLDEDKETGCIVHSGCRVFPEGVTEDKKESRNKARREARLRRRQLRRKKENRKRLAQFLHETSLLPVFGSTEWKNLMDNTHSNPYELRSAALKKQLQPFELGKVIYHLAKHRGFKATKLDELMAESDEKKELGVVKDGIKELDHKLGDQTLGVYLASIPPSEKKRGRYLGRYMIQEELEQILEYQKHYNPELITSTFKKHLNSLIFSQRPTFWRLNTLGTCSLEQNESVCPKHSWIGQQFIMMQKVNDLRIVEPHPRHLTMEERTQLIQGLCKQKIMSFGGIRKLLHLPKGTVFNFETYQDKEDKRGLPGNAIEAALSTIFGSEWKHLPHKDAIRSSLSNRIWSISYNRVGNKRIEIRADESYQNQRQTVKQEMMKDWNIAEDQAEQLVQLPIPPQWLRFSEKAIQKLLPDLESGVPLQTAIKEHYPETLKSSEVEHELLPSSPHLVPELRNPTVNRALNELRKVVNNIIRSYGKPDIIRIELARDLKLGKKKKLEITKKNRQREQERKEAKNQLEKEGVKPTGMNIEKFLLWQESDGLDLYTGQKISFAALFKQTEYDIEHIIPRSRSFNNTFFNKTLAHNEINRQKGNMIPKEFFGDGETWHAFVTRVNQSKLPLEKKEKLLIPHYDAIASEEMTERQLRDTAYIATEAKTYLQTLGIPVQPTNGRATASLRRVWGINSIWATEFGLEEESKKAAGEKIRDDHRHHAVDAAVVALTSPGRIKRLSTFYQYRKEMKPDDFPLPWETFRADLITSLHKIIISHRVQRKISGPLHEETAYGFTKKKSETDPTAYYFVTRKTLDKDFKPNKVKDIVDPAVRHLIGEHLQKFDNNPAVAFAPENRPHMPLRKGGWGPPIKKVRIQIARNPQFMVSRQKNPISYYDSGDNHHMAIYGTHLDDGTVDPETVSFEVVSRFEVNQRASKNEPLVKPQNENGVPLLFTLVKNNVLIWNEPGEEEQMHLVRWTTANKGRIFHKPLWMSGTPPIEISISVKNLISYGGRKVSVDPIGNIFPCND

SEQ ID NO: 11 - protein, 1308 aa, Unknown
  Cas9.4
MKKILGLDLGTDSIGWTIVQQNEEKKFKLIDKGVRIFQKGVGEEKNNEFSLAKERTTHRNTRKKYRRTKQRKVRLLRELIKHGMCPLSFDELELWSKYRKGKPYIYPLSNKGFTQWLKLNPYDLRERAIKPDEKLTPLELGRIFYHITQRRGFKSNRKDNSEDSEGVVKTSISQLREEMEGKTLGQFFNNELKKGNKVRKKYTAREDYHHEFNEICNIQKIDNKTKAALEREIFFQRALKSQRHLVGKCTLEPKKPRCPLSAIPYEEFRALQFINSIRIKDAEENLMPLTQKEREVIQSLFFRKSKPSFPFNDIKKILEKHNGQRLTFNYPEKLQIIGSPTIALLKSVFGEEWASLSVAYTKKDGTTGTINSEDVWHALFEFEHNDKLEDFLKQRLKLSDDNIQKLIKGNLKQGYASLSRKAINNILPFLKDGHIYTHAVFLAKIPEIIGRKQWLHSKDQIVNWFLKSAEELPLKNRLCKIVNNLITEFNETYANADPKYILDDSDKKSINRSLQHDFGPKTWNKFSSEKKDELQKETERLFLSQINKGNASAPYIKPYRQDEELKQYLIDNFNIKQEEAERIYHPSAIDIFDEAPYNDDGIKLLQSPRTPSARNPMAMRALHELRYLLNQLLSQRGIDEHTVIHLEMSRELNNQNKRLAIQRYQQARNEEHQEYAKEIKKIFKEQTQKEIEPTEADILKYRLWKEQEHNCLYTGRKIGIADFIGDNSNVDIEHTWPRSKSFDNSTANKTLCDSHYNRNIKKNKIPYDLPNFKESAIIEGKQYDPIKARLKDWEEKCNHLKELAAKYRYNAKRASTKEQKDKALQNAHFYQMHHEYWKDKIFRFTGKEIRNSFKNSQLVDTGIINKYARAYLQTVFNKVFTIKGTLTADFRKAWGIQNPDTSKSRQRHTHHAIDAAVVACLTRDRYDFLTQWYRAEEKGNERKKHIIQERMKPWTTFVQDIKAFENSILVSHHTRKTSAKQTRKRLRENGKIVKDPNGNPIYSKGDTFRNRLHKDTFYGAILRPQIDKEGKTVTDENGNPKLTTQYVVKKPVTDLKETDIKNIVDSKIKSLFESKKLNEIQKEGISIPPSKPEGKETPIKSVRLKQPFNPIPLREHTHLSQKPHKQYYHVQNEGNFLMAIYEETSASKKPEKTFELISNLQAADYYKASNKENREQYPIVPERKFITKRNKEIELPLKQIIYIGQMVMLYENSPEELKSKNEEELFKCLYKIVGITSMTIQAKYEYGVFILKHHAISTPYSELKPKDGDFSWEGNIEAMRKQLHSRIKVVIENLDFKITPTGKIEWLF

SEQ ID NO: 12 - DNA, 25 bp, Unknown
  Cas12q spacer
gcttggaaatatgtcttatttatca

SEQ ID NO: 13 - DNA, 3765 bp, Unknown
  Cas12a.1
atgaaggtctcgacttgggattcgtttacaaaccaataccccctaacgaaaactctacgcttcgaattaaagccagtcggcaaaacactgcagaaaattcaagatcgcaacctgattacagaagacgaacaacgccaaaaagatttcaacaaagtcaaaaaaataatggacggatactacaagcaattcatagaagaatgcttggaaggtgccaagataccgttaaaaaaattggaagaaaacaacaacgcttacacgaaactgaaaaaagacccttacaacaaaaaattaagggaagaatacgcaaaactccaaaaacaattaaggaaactaattcacgacgaaataaataaaaaagaagaattcaaatacttgttcaagaaagaattcatcaaaaaaatattgccggaatggctcgaaaaaaaagggaaaaaagaggaactcaaagaaatcgaaaaattcgataaatgggttacctactttagcggtttttttaacaaccgcaaaaacgttttttcaagcgatgaaatttcgacgtcaatgatttacaggatagtcaacgacaacctaccgaaattcctagatgacgtttcacgcttcggagaaataaccagatacaaggaatttgacgccaaccaaatagaagaaaactttgaaagcgagttgaacggagagaaattaaaagattttttcaacttgaaaaacttcaacaactgccttaaccaagaaggaatagaaaaattcaacttaatcataggaggcaaaagcgaagaaggcaacaataaaataaagggcttaaacgaattagtcaacgaactcgcccaaaaacaagcggacaaaaacgagcaaaaaaaggttagaaaattaaaactcgcgccgttattcaagcaaatcttaagtgaccgcaaatcctcctcgttcgcattcgaaaaattcgaggaaaatacggaggtattcgatgcaatagacgaattttacgataaaataagcttggaaacactcaaaaaaatagaagcgaccctcgaaaagctagaagaaaaagatttggaattagtttacttgaaaaacgatagatgcctaacaggaatttcacaagaagtattcggggatcgggaaagagtacttcaagccctaagggaatacgcgaaaaccgaactcggcctcaaaaccgacaaaaaaatagaaaaatggatgaaaaaaggcaggtattcaatccacgaaatagagagcggcctcaaaaaaatcggttcaaccggacacccgatatgtaattatttctcaaaactagaagaaaaaaagacaaacttgattcaagaaataaaaaaagcgcgcactgaatatgaaaaaataagtgacaaaaaaaagaaattaactgctgaaagccaagagcccaacgtcgcaagaataaaggcgttactggactcaataatgcggctataccacttcataaaacccctcaacatcaacttcaaaaacaagaaagaaaaggattcagaggcacttgaaaccgataacgatttctataacgatttcgacgaatcgtttgcggaactagggaatataatcccactatacaatcaagtcagaaactatgttacgcaaaaaccgttcagcaccgaaaaattcaagttaaactttgaaaatcccaaactcctaagcggctgggacaaaaacaaggaaaaagactattattctgttatattgagaaaagaggagtcatactacttagccattatgaccccaaaacaaaaaaacgtttttgacgaactggaacggcttccggctggaaaaaattattttgaaaaaatagactacaaattattgcctaccccagaaaaaaatctacctagaatattatttgcaaaaaaaaacatttcattttacaagccatcaaaagaaatcgaagcgattcgtaatcactctgcccacaccaagcatggaaacccacaaaacgggttcaaaaaaagggatttccgattaagcgattgccataaaatgattgacttttacaaaaagagcattcaaaaacaccccgaatggaaagaatacgatttccaattcaaaaaaacggaagattacgtcgacatatcagaattttataaagaagtatccgaccaaggctataaaatagaattcaaaaaaataagcgaaaaatatttgcttgacttggtcgaagaaggaaaactttacttattccaaatttggaacaaggacttttcgaagtattcggaaggccgtaaaaacctgcacacaatttactggaaagaactattctccaaagaaaacctttcagacataacttacaaattaaacggcgaagccgaaatattctaccgcccaaagtcaatggaaaggaaagtaactcacccaaaaaaccaaaaaatagaaaacaaagacccgattaaagggaaaaaattcagtaaattcaaatacgactttataaaaaacaaaaggtacaccgaagaccgtttcttcttccactgcccgataaccttgaacttccaggcgcgcgatggcagcaaaacgattaacaagcgggtcaacgaccacatacgcgaaacaaaagatgacattttcgtgttaagcattgaccgcggggaaaggcacttggcgtactacacgctattgaattcaaaaggagaaatccaagaacaaggctctttcaacgtaatctcggacgacaaagaaagaaaacgtgattaccacgaaaaactggatgaacgcgaaaaagaacgcgacaaagcaaggaaaagctggcagaaaatcgagaccataaagaaattgaaggatggctacctatcccaaatcgtacacaaaatcgctaaactcgcaatagaaaaaaacgcgataatcgtcttggaagacctgaacttagacttcaagcgcgggagattaaaaatcgagaagcaagtataccaaaagttcgagaaaaaactaatagacaaactcaattacttggttttcaaggaaagaaccgaaaaagaagccggcggatccctaaacgcataccaactaaccggaaaatttgaaggatttaagaaactcggaaaagaaacaggtataatatactacgttcccgcggcgtacacctcgaagatttgcccgaaaacaggcttcgtaaatctgttaagacctaaattcaagaacatagaaaaagctaaggaattcttcaaaaaattcaactacatcaaatacgattcgagcgaaggcttattcgaattcaacttcgactactccaaattcattaaaaacggaaaaaaagaaacaaaaataattcaagacaattggtcggtttactcgaacggaacgaaactagtcggcttcagaaacaagaataaaaacaattcatgggatacaaaggaagtcaaaccgaacgaaaaactaaaaatattgttcaaagaatacggggtttccttccaaaaagacgaaaatattataagccaaatagccagccaaaacaaaaaagctttctttgaaaacctcattaaaatctttaaaacgattttaatgttacgcaactcaagaaaagaccccgaagaagattacgtactttcctgcgtaaaagacgaaaacggcgaattcttcgactcaagaaaagctaaagacaacgagcccaaggacgccgacgcgaacggcgcttaccacatagggttgaaaggattaatgctcttggaaagaataaaggccaacaaaggaaagaaaaaactcgatttactaatcagcaggaacgacttcatcaacttcgcagttgaacggagcaagtaa

SEQ ID NO: 14 - DNA, 3846 bp, Unknown
  Cas12p
atgaaaaaatctatttttgatcagtttgtaaatcagtatgctctttctaaaacgttgcggtttgaattgaagccggtgggggagacggggaggatgcttgaggaggcgaaggtttttgctaaagatgaaacaatcaagaaaaaatatgaggcaaccaagcctttttttaataaattgcatcgtgaatttgtagaggaggctttaaatgaggtggaattagctggtttgcctgaatattttgaaatatttaaatattggaaaaggtataaaaagaagtttgaaaaggatttgcagaagaaagaaaaagaattgcggaaatcagttgtaggtttttttaatgcacaggcaaaggaatgggcgaaaaaatatgaaactttgggtgtgaagaaaaaagatgtgggacttttatttgaagaaaatgtttttgctatattgaaggaaaggtacggaaatgaggagggatcacaaattgttgatgaaagtacaggaaaagatgtttcgatatttgatagttggaagggctttacagggtattttattaaattccaggaaactcgtaagaatttttataaggatgatggcacggctactgctttggctacaaggattattgatcaaaatttgaagcgtttttgtgataatttactaatatttgaaagtattagagataaaattgatttttcagaggtagaacaaactatgggaaactctattgataaggttttttcagtaattttttatagttcctgtttacttcaggaaggaattgatttttataattgtgttttaggtggggagactctgccaaatggtgaaaagagacagggaataaatgagcttattaatctctataggcaaaaaactagtgagaaagtaccttttttaaagttgcttgataagcagattttgagtgaaaaagagaagtttatggatgaaattgaaaatgatgaggctctcttggatactcttaaaatatttagaaaatcggctgaagaaaaaaccactttgttaaaaaatatttttggtgattttgttatgaatcagggtaagtatgatttagcgcagatttatatttccagagaatctttaaatactatttcacggaaatggaccagtgaaacagatatatttgaggattcattatatgaagtgttaaagaaatcaaaaatagtttctgcctctgtaaaaaagaaagatggagggtacgctttccctgagtttattgcgcttatttatgtgaaaagtgctcttgaacaaattcctactgaaaaattttggaaggagcgatattataaaaatattggagatgttttgaataaagggtttttgaatggtaaggaaggtgtctggttacaatttttattgatttttgattttgaatttaattctctttttgaaagagaaataattgatgaaaatggagacaagaaagtggccggatataatttgtttgccaagggttttgatgatcttttgaataactttaaatatgatcaaaaagctaaggttgttattaaggattttgcagatgaggttttacatatttatcagatgggaaaatattttgctattgaaaagaaacgttcttggttggctgattatgatattgattcattttatactgatcctgaaaaaggttatttgaagttttatgaaaatgcgtatgaagagattattcaagtttataataaattgcgaaattacctaacgaagaaaccttatagtgaggataaatggaaacttaattttgagaatccaactttagctgatgggtgggacaaaaataaagaagctgataattctacagttattttgaaaaaggatggtcgctattatttagggttgatggctcgcgggcgaaataaactttttgatgatagaaatttaccaaaaattttggagggcgttgagaatgggaaatatgagaaagttgtatataagtattttccggatcaggcaaaaatgtttccaaaagtttgtttttcaactaaaggtttggagtttttccaaccttcggaggaagtcattactatttacaaaaattctgaattcaaaaaagggtatacttttaatgtaaggagtatgcagaggcttattgatttttataaagattgtcttgttagatatgaggggtggcaatgttatgattttagaaatttgagaaagacagaagattatcggaagaatattgaagagtttttcagcgatgttgctatggatgggtataaaatatcctttcaggatgtctcggaaagttatattaaagagaaaaatcagaatggggatttatatttatttgagataaaaaataaagattggaatgaaggcgcaaatggaaagaaaaatttgcacactatatattttgaatctcttttttcggctgataatattgccatgaattttcccgttaagttgaatggacaagcggaaattttttatcggccaagaacagaggggctggagaaagaaaggataatcactaaaaagggtaatgttttggaaaaaggagataaagcttttcataaaagaaggtatacggaaaacaaagttttttttcatgttccgattacacttaatcgaacaaaaaaaaatccatttcaatttaatgcaaaaattaatgattttttggctaaaaattctgatataaatgttattggggtcgatcgtggggagaagcaattagcatatttttctgttatttcacagagaggcaaaattttggataggggtagtttaaatgtgataaatggagttaattatgcagagaaattagaagaaaaagctagagggcgtgagcaggcgcgtaaggattggcagcagattgaaggtattaaagatttaaagaagggatatatttctcaggtagttagaaagctagccgatttagcaattcagtataatgcgattattgtttttgaagatttgaacatgcggtttaagcagattcgtggaggtattgaaaaaagtgtttatcagcagttggagaaggctttgattgataaattaacttttttggttgaaaaggaagaaaaagatgtagaaaaggcaggtcatttgttaaaagcttaccagcttgctgctccgtttgagacttttcagaaaatgggtaaacaaacggggattgttttttatacacaggctgcatatacttcacgaattgatcctgttacaggttggcggcctcacttgtatttgaaatattccagtgcggagaaggcaaaggcggatttattaaaatttaaaaagataaagtttgtggatggccggtttgagtttacttatgatattaagagttttcgtgaacaaaaggaacatccaaaggcgactgtctggacggtgtgttcttgcgtggagagatttcgttggaatagatatttaaatagcaataaaggtggttatgaccattacagtgatgtgacgaagttcttggtagagctttttcaagagtatgggattgattttgaaagaggggatattgtcgggcaaattgaggttttggaaacgaagggaaatgaaaaattttttaagaatttcgtttttttctttaatttgatttgtcagataagaaatactaatgcgtcggagttggcaaaaaaagatggaaaagatgattttattctttcaccggtggaaccgttttttgatagcagaaattcggagaagtttggggaggatttgccaaaaaatggggatgataatggggcatttaatattgcgaggaaagggcttgttattatggataaaattacaaaatttgcagatgagaatggtgggtgcgagaagatgaagtggggagatttgtatgtttctaatgtggagtgggataattttgtagctaataaatga

SEQ ID NO: 15 - DNA, 3414 bp, Unknown
  Cas12q
atgataaatattgacgaattaaaaaatttatataaagttcaaaaaacaattacttttgaattaaaaaataaatgggaaaataagaatgatgaaaatgatagagttgagtttttaaagactcaagaatgggtggaatctttattcaaagttgatgaggagaattttgatgaaaaggagtcaattccgaacttgttagatttcggccaaaagattgcgagtcttttttataagttgagtgaagatatcgctaataatcaaattgatacacgggttttaaaagtgagcaagtttttgttggaggagatcgatagaaatcaatatcatgagaaaaaaaataaaccaacaaaggttaaggagatgaatccaaatacaaataagagttatattaaggagtataagttatcagatcaaaatacattgtatgttctgttgaagataatggaagatgaagggcggggtttacaaaaatttttatatgataaggcagacagattaaatttatataatcagaaggtaagaagagatttcgctttaaaagaaagtaacgaacagcagaagttttcgggtaacgctaattattacggaaacataaaattgttgattgattcattggaagacgctgttcgtattattggttatttcacgtttgatgatcaagcagaaaatgctcaaataaatgaattcaagagcgttaagcaggaaatgaataacaatgaagcttcgtatcaggctttgaaagattttgctattgataacgcaaaaaaagaaattgaacttacaactctaaatcatagggctgttaacaaggatccaaaaaagatacaagaacagattgaagaagtggaaaattttgaagaagatataaatcaattgaagcaccaaatttctgcgcttaatgataaaaaatttgatgtagtgtcaagattaaagcatgcattaattaaaatgttaccggagttgaatttgttagatgctgaaagcgagcaaggtagagaggttcagcaaatatatcaagataaaaagaatggtttggaattagacgattttaagttcaatttgcttaaacatcatcaatggcagaaaaccatttttaaatacattaaattagagggtttggttttacctgatttatatgccgaaaacaaacaagataagattaaagtgtatattgaaaattatcgacaaagcggagaaaggataagtaaaaaggcacgcgaggagttgggcaagatcgataaaagagaggaatttaatggtaatgatgaactaaagaaagcgtggtacgaatacaaagatttttgcagagacaagcgtaataaatccgtggaattgggcaataagaaatcactgtacaatgccatcaagcgtgaggttttaaggcagaaaatgtgtaatcattttgccgtattggtgagtgatggggaagatacatcgccttattattatttgatattaattcccaatgaaaacagtgatgaaatgaacaggacattcaaagagcttaaagcatccgaaggaaattggaagatgctcgattataacagattaacttttaaagctttggaaaaattggcattattgcgcagctctacatttgaaattgcagaccaagaactacaagaagaagctaaaaaaatttgggaagaatataaagaaaaggcgtataaagattttaagaataaaaaattattacaagggctatccggtcgccaaagagaagaaaaaaaacaagaattgcaaaaagaaagtttaaatcgagttataaattatttaattcgttgcattcagtcgttgccggatagcggtaaatacaattttaattttaaagaaccgcatcaatatcagagcttggaagagtttgcggaagaaattgatagacagggttatcattgcgcttggaagaatgtaagcaaagacaagcttatggagctggaggcgatggaaaaaattaaagtatttaaattgcataataaggattttagaaaagttaaacttaacgattcgaaacacaatccgaatctttttactttatattggcttgacgcgatgaatttggataaagtcaatgttcgtttattgcccgaggtggatttatataaaagagccaaagaaacgcaactaaaattattcgaaagagatgtaaagtgcaatattaataatcaaaaaataaaatcaattaaagaaaaaaatagattatttcaagataaactttacgcttcattcaagctggaattttatccagaaaacgaaggtttgggttttgaacaagtcaatgataaagtgaataatttttgcggaagtgatacagcgtattatttgggtttggataggggtgagaaagaattggttacgttttgcttggttgattctgatgggcggttggttaagaacggagattggacgaagtttaaagaggttaactatgcggataaattaaagcaattttattattcaaaaggtgaaatagaatctactcaacaacaacttttggaagctcgagacaatattaaacaagctactaacacggaggataaagaatcgatgaaattaaactataaaaaattagagttgaaactaaaacaacagaatttgttagcgcaggagtttattaaaaaagcttattgcggttatttgatagattcaataaatgaaatattacgggaatatccaaatacgtatcttgtattagaggatttggatatagcaggtaaagctgaccccgaaagcggcatgaccaataaagaacaaaatttaaataaaacaatgggtgccagcgtttatcaagctattgaaaatgccatagtaaataagtttaaataccgtactgttaaattatccgatatcaaaggtttgcaaactgtaccgaatgtagtgaaggtggaagatttgcgcgaagttaaggaagtggaagatggtgagcataaatttggtttgataagatccgtgaaatcaaaggatcaaattggcaatattctgtttgtggatgaaggagaaacatctaatacttgcccgaattgcggatttaacagcgattggtttaagcgggatgttgattttgatttggagattgtggctactgtaaacggtcagaaaaatgcggttatagaacaaaacgacaaaaagtactgttttcccggtgaaatttataagttagaaataattaataaagaatacgaaacaaataaacggaatttagccatgatttttaaaccgcgcgcaaaagcttgtagaaaatttataaataataatttggataagaatgactatttttattgcccgtattgcgctttttctagcaagaactgcaataatccaaaattgcaaaacggtgattttgtggtatattcgggtgatgatgtggcggcatacaatgtagcgatcagaggtattaaccttttaaacaatataaaatag

SEQ ID NO: 16 - DNA, 3822 bp, Artificial Sequence
  Cas12a.1 codon optimized version
atggggtcaagtcatcaccaccaccaccactcaagtggactagtaccccgtggcagcatgaaagttagcacctgggatagcttcaccaaccagtacccgctgaccaagaccctgcgttttgagctgaagccggtgggtaaaaccctgcagaagatccaagaccgtaacctgattaccgaggacgaacagcgtcaaaaggatttcaacaaggttaagaaaatcatggatggttactacaagcagttcatcgaggaatgcctggaaggcgcgaagatcccgctgaagaaactggaggaaaacaacaacgcgtacaccaaactgaagaaagacccgtataacaagaaactgcgtgaggaatacgcgaagctgcagaaacaactgcgtaaactgatccacgatgagattaacaagaaagaggaattcaagtacctgtttaagaaagaattcatcaagaaaattctgccggaatggctggagaagaaaggtaagaaagaggaactgaaagagatcgaaaagttcgacaaatgggtgacctactttagcggcttctttaacaaccgtaagaacgttttcagcagcgacgagattagcaccagcatgatctatcgtattgtgaacgataacctgccgaaattcctggacgatgttagccgttttggtgaaattacccgttacaaggagttcgacgcgaaccagatcgaggaaaactttgagagcgaactgaacggtgaaaaactgaaggatttctttaacctgaaaaacttcaacaactgcctgaaccaagaaggcattgagaaatttaacctgatcattggtggcaagagcgaggaaggtaataacaaaatcaagggcctgaacgaactggtgaacgagctggcgcaaaaacaagcggacaagaacgagcagaagaaagttcgtaaactgaagctggcgccgctgttcaaacaaatcctgagcgatcgtaagagcagcagctttgcgttcgaaaaatttgaggaaaacaccgaggtgttcgacgcgatcgatgaattttatgacaagattagcctggagaccctgaagaaaatcgaagcgaccctggagaaactggaggaaaaggacctggaactggtttacctgaaaaacgatcgttgcctgaccggtatcagccaggaagtgttcggcgaccgtgagcgtgttctgcaagcgctgcgtgaatacgcgaaaaccgagctgggtctgaagaccgataagaaaatcgagaagtggatgaagaaaggtcgttatagcatccacgagattgaaagcggcctgaagaaaatcggtagcaccggccacccgatttgcaactacttcagcaaactggaggaaaagaaaaccaacctgatccaggaaattaagaaagcgcgtaccgagtatgaaaagatcagcgacaagaaaaagaaactgaccgcggaaagccaagagccgaacgtggcgcgtatcaaagcgctgctggatagcattatgcgtctgtatcacttcatcaagccgctgaacatcaacttcaagaacaagaaagagaaggacagcgaagcgctggagaccgacaacgatttttacaacgacttcgatgaaagctttgcggagctgggcaacatcattccgctgtacaaccaagtgcgtaactatgttacccaaaaaccgttcagcaccgagaaattcaagctgaactttgaaaacccgaagctgctgagcggttgggacaaaaacaaggaaaaagattactatagcgtgattctgcgtaaagaggaaagctactatctggcgatcatgaccccgaagcagaaaaacgttttcgacgagctggaacgtctgccggcgggcaaaaattacttcgagaagatcgattacaagctgctgccgaccccggaaaagaacctgccgcgtatcctgttcgcgaagaaaaacattagcttttacaagccgagcaaagagatcgaagcgattcgtaaccacagcgcgcacaccaaacacggtaacccgcagaacggcttcaagaaacgtgactttcgtctgagcgattgccacaagatgatcgacttctacaagaaaagcattcagaaacacccggaatggaaggagtatgattttcaattcaagaaaaccgaggactacgtggatatcagcgaattctataaagaggtttctgaccagggttacaagatcgaattcaagaaaattagcgagaaatacctgctggacctggtggaggaaggtaaactgtacctgttccaaatctggaacaaggatttcagcaagtacagcgaaggccgtaaaaacctgcacaccatctattggaaagaactgttcagcaaggagaacctgagcgatattacctataagctgaacggcgaggcggaaatcttttaccgtccgaaaagcatggagcgtaaggttacccacccgaagaaccagaaaatcgaaaacaaagacccgatcaagggtaagaaattcagcaagttcaagtatgacttcatcaagaacaagcgttacaccgaggatcgtttctttttccactgcccgatcaccctgaactttcaagcgcgtgacggcagcaaaaccatcaacaagcgtgtgaacgatcacattcgtgagaccaaagacgatatcttcgttctgagcattgatcgtggtgaacgtcacctggcgtactataccctgctgaacagcaagggtgaaattcaggagcaaggcagctttaacgtgatcagcgacgataaggagcgtaaacgtgactatcacgaaaaactggatgagcgtgaaaaggagcgtgacaaggcgcgtaaaagctggcagaaaatcgagaccattaagaaactgaaggatggctacctgagccaaatcgtgcacaagattgcgaaactggcgatcgagaaaaacgcgatcattgttctggaagacctgaacctggatttcaagcgtggtcgtctgaagattgagaaacaggtgtaccaaaaattcgaaaagaaactgatcgacaagctgaactatctggtttttaaagaacgtaccgaaaaagaggcgggtggtagcctgaacgcgtatcagctgaccggtaaattcgagggctttaagaaactgggcaaggaaaccggcatcatttactatgtgccggcggcgtacaccagcaaaatctgcccgaagaccggcttcgttaacctgctgcgtccgaagttcaagaacatcgaaaaggcgaaggagtttttcaagaagttcaactacatcaagtacgacagcagcgaaggtctgtttgagttcaacttcgattacagcaagttcatcaagaacggcaagaaagagaccaaaatcattcaggacaactggagcgtgtatagcaacggtaccaagctggttggcttccgtaacaagaacaaaaacaacagctgggataccaaggaagtgaaaccgaacgagaagctgaaaattctgttcaaagagtacggtgttagctttcaaaaggacgaaaacatcattagccagatcgcgagccaaaacaagaaagcgtttttcgagaacctgatcaagattttcaaaaccattctgatgctgcgtaacagccgtaaagacccggaggaagattacgtgctgagctgcgttaaggacgaaaacggcgagtttttcgacagccgtaaggcgaaagataacgagccgaaagacgcggatgcgaacggcgcgtaccacattggtctgaagggcctgatgctgctggaacgtatcaaggcgaacaaaggtaagaaaaagctggacctgctgatcagccgtaacgatttcattaactttgcggttgagcgtagcaagtaa

SEQ ID NO: 17 - DNA, 3903 bp, Artificial Sequence
  Cas12p codon optimized
atgggatcaagtcatcaccaccaccaccactcaagtggactagtacccaggggaagcatgaagaagagcattttcgatcagttcgttaaccagtacgcgctgagcaagaccctgcgtttcgagctgaaaccggtgggtgaaaccggccgtatgctggaggaagcgaaggttttcgcgaaggatgaaaccattaagaaaaagtacgaagcgaccaagccgttctttaacaaactgcaccgtgaattcgtggaggaagcgctgaacgaggttgaactggcgggcctgccggagtacttcgaaatcttcaagtactggaagcgttacaaaaagaaattcgagaaggacctgcagaagaaagagaaggaactgcgtaaaagcgtggttggtttctttaacgcgcaagcgaaggagtgggcgaagaaatatgaaaccctgggcgtgaagaaaaaggatgttggtctgctgttcgaggaaaacgtgtttgcgattctgaaagaacgttacggtaacgaggaaggcagccagattgtggacgagagcaccggcaaggatgttagcatcttcgacagctggaagggttttaccggctatttcatcaaatttcaggaaacccgtaagaacttctacaaagatgatggtaccgcgaccgcgctggcgacccgtatcattgatcaaaacctgaaacgtttctgcgacaacctgctgatctttgagagcattcgtgataagatcgacttcagcgaggttgaacagaccatgggcaacagcatcgataaggtgttcagcgttatcttttatagcagctgcctgctgcaagaaggtatcgacttttacaactgcgtgctgggtggtgaaaccctgccgaacggtgaaaagcgtcagggcattaacgaactgatcaacctgtaccgtcaaaagaccagcgagaaagttccgttcctgaagctgctggacaaacagattctgagcgagaaggaaaaatttatggatgagatcgaaaacgacgaggcgctgctggataccctgaagattttccgtaaaagcgcggaggaaaagaccaccctgctgaaaaacatcttcggcgattttgtgatgaaccagggtaaatatgacctggcgcaaatctacattagccgtgaaagcctgaacaccattagccgtaagtggaccagcgaaaccgatatcttcgaagacagcctgtacgaggtgctgaaaaagagcaaaatcgtgagcgcgagcgttaaaaagaaagacggtggctacgcgttcccggagtttatcgcgctgatttatgttaaaagcgcgctggaacagattccgaccgagaagttctggaaagaacgttactataagaacatcggcgatgtgctgaacaagggtttcctgaacggtaaagaaggcgtttggctgcaatttctgctgatctttgacttcgaatttaacagcctgttcgagcgtgaaatcattgatgagaacggcgacaagaaagtggcgggttataacctgttcgcgaagggttttgacgatctgctgaacaacttcaaatacgaccagaaggcgaaagtggttattaaggattttgcggacgaagttctgcacatttatcaaatgggcaaatacttcgcgatcgagaagaaacgtagctggctggcggactatgatattgacagcttctacaccgatccggagaagggttacctgaaattttatgaaaacgcgtacgaggaaatcattcaggtttataacaagctgcgtaactacctgaccaagaaaccgtatagcgaggacaagtggaaactgaacttcgaaaacccgaccctggcggatggttgggacaagaacaaagaggcggataacagcaccgtgattctgaagaaagacggtcgttactatctgggcctgatggcgcgtggtcgtaacaagctgttcgacgatcgtaacctgccgaaaatcctggagggtgttgaaaacggcaagtacgaaaaggtggtttacaagtacttcccggatcaggcgaagatgttcccgaaagtgtgctttagcaccaaaggcctggaattctttcaaccgagcgaggaagttatcaccatttacaagaacagcgagttcaagaaaggttatacctttaacgtgcgtagcatgcagcgtctgattgatttctataaagactgcctggttcgttacgaaggttggcaatgctatgattttcgtaacctgcgtaagaccgaggactaccgtaaaaacatcgaggaattctttagcgatgtggcgatggacggctacaagattagcttccaggacgttagcgagagctatatcaaggagaagaaccaaaacggtgatctgtacctgtttgagatcaagaacaaagactggaacgaaggtgcgaacggcaagaaaaacctgcacaccatttatttcgagagcctgtttagcgcggataacatcgcgatgaacttcccggtgaaactgaacggccaggcggagatcttttaccgtccgcgtaccgaaggtctggagaaggaacgtatcattaccaagaaaggcaacgttctggaaaagggtgacaaagcgttccacaagcgtcgttacaccgagaacaaagtgttctttcacgttccgattaccctgaaccgtaccaagaaaaacccgttccaatttaacgcgaagatcaacgacttcctggcgaaaaacagcgatatcaacgtgattggtgttgaccgtggcgagaaacagctggcgtattttagcgtgattagccaacgtggcaagatcctggaccgtggtagcctgaacgtgatcaacggcgttaactacgcggagaagctggaggaaaaagcgcgtggtcgtgaacaggcgcgtaaggattggcagcaaatcgagggcattaaagacctgaagaaaggttatattagccaggtggttcgtaaactggcggatctggcgatccaatacaacgcgatcattgtgttcgaggacctgaacatgcgttttaagcaaattcgtggtggcatcgagaaaagcgtttatcagcaactggaaaaggcgctgatcgataaactgaccttcctggtggagaaggaagaaaaggacgttgaaaaggcgggtcacctgctgaaagcgtaccagctggcggcgccgttcgaaacctttcagaagatgggtaaacaaaccggcattgtgttttatacccaagcggcgtacaccagccgtatcgatccggttaccggctggcgtccgcacctgtacctgaaatatagcagcgcggaaaaggcgaaagcggacctgctgaagttcaagaaaattaagttcgtggatggtcgtttcgagtttacctacgacatcaagagcttccgtgagcagaaggaacacccgaaagcgaccgtgtggaccgtttgcagctgcgttgagcgttttcgttggaaccgttatctgaacagcaacaaaggtggctacgatcactatagcgacgtgaccaagttcctggttgagctgtttcaggaatacggcatcgacttcgaacgtggtgatattgtgggccaaatcgaggttctggaaaccaagggtaacgagaagttctttaagaacttcgtgttctttttcaacctgatctgccagattcgtaacaccaacgcgagcgaactggcgaagaaagacggcaaggacgatttcattctgagcccggttgagccgtttttcgatagccgtaacagcgagaagttcggcgaagacctgccgaaaaacggtgacgataacggcgcgtttaacatcgcgcgtaaaggtctggttattatggataagatcaccaaattcgcggacgagaacggtggctgcgaaaagatgaaatggggtgacctgtatgtgagcaatgtggagtgggataactttgtggcgaataaataa

SEQ ID NO: 18 - DNA, 3471 bp, Artificial Sequence
  Cas12q codon optimized
atggggtcctcccatcatcaccaccaccactcttcaggcttggtaccgcgtggttccatgatcaacatagacgaattgaaaaatttatataaggtgcaaaagaccatcactttcgaacttaagaacaagtgggagaacaaaaatgatgagaacgacagagtagagttcttgaagactcaggagtgggtcgaaagccttttcaaggtcgatgaagagaactttgatgagaaagagtctatccctaacttgttagacttcggacagaagattgcgtccttgttttacaagctgagcgaggacatagcgaacaaccaaattgatacgcgggtattgaaagtctcgaaattccttttagaggaaattgatagaaatcaataccacgagaaaaaaaacaagcccacaaaggtaaaagaaatgaatcccaacacaaacaaaagttatataaaagaatataagctgtccgaccaaaacacactgtacgtgttattaaagataatggaagatgaaggtcggggattacaaaaatttttgtacgataaagcggaccggttaaacctgtacaatcaaaaagttcggagagacttcgccttaaaggaatcaaatgagcaacaaaaattctctggaaatgccaactactatgggaatataaagctgcttatagatagcttagaagatgcagtccggatcattgggtatttcactttcgacgatcaagcagaaaacgcacaaatcaatgaatttaagtccgttaaacaggaaatgaataataatgaagcgtcttaccaagcactgaaagacttcgctattgataacgcaaaaaaagagatagaattgacgacgttgaaccaccgggcggtcaacaaggatccaaaaaagattcaagaacagattgaggaagtcgaaaatttcgaagaagatattaaccagttaaagcatcagatatcagccttgaatgataagaagtttgacgtggttagcagattaaagcacgctcttataaaaatgttaccagaactgaatcttttggatgctgagtcggaacagggccgtgaagtccagcagatatatcaagacaaaaaaaacgggttggagcttgatgactttaaatttaaccttttaaaacatcatcaatggcaaaaaacgatcttcaagtatattaagcttgagggcttagttctgccagacctttacgcggaaaacaaacaagataaaatcaaggtttatattgagaattatagacagagtggtgagcgtatttctaagaaggcgagagaggaattaggaaaaatcgataaacgcgaagagttcaatggaaatgacgaacttaagaaggcatggtatgagtataaggacttctgtagagacaaacgtaataagagcgtggaacttggcaataagaagtcgctgtacaatgccataaagcgcgaagttttgcggcaaaaaatgtgcaaccatttcgctgtgctggtgtccgacggtgaagatacttccccttattattatctgatattaatcccgaacgagaactccgatgaaatgaatagaacgttcaaggaattgaaggcctccgaggggaattggaagatgttggattacaatcgtctgaccttcaaagccttggagaaattggccctgttacggtcgtctaccttcgagatagcggatcaggaactgcaagaagaggcaaaaaagatctgggaggagtacaaggaaaaggcgtacaaagacttcaaaaacaaaaagttattacagggtttatcgggaagacagcgggaggagaaaaagcaagaattgcaaaaggagagcctgaatagagtaatcaattacttgatcagatgcattcagtcattgcccgacagcggaaaatacaactttaactttaaagagcctcatcaataccaatcgcttgaagagtttgccgaggagattgatcggcaaggttatcactgtgcttggaaaaacgtttctaaagataaactgatggaattggaagcgatggaaaagattaaggttttcaaacttcataacaaagactttcgcaaggtaaaactgaacgactccaagcacaaccctaatctttttactttgtactggttagacgccatgaatttggataaggttaacgtccgcctgttaccggaagttgacctttacaagagagctaaggaaacacagctgaaattgttcgaacgtgatgtgaaatgcaatatcaataaccaaaagattaaatctatcaaggagaagaatagactgtttcaggacaagttgtatgctagttttaagttagagttttatccagaaaacgaaggattaggtttcgagcaggtaaatgacaaggtcaataacttctgcggtagcgatacggcctattatcttgggcttgatcgtggagagaaagagcttgttacattctgcctggtggactctgatggccgcctggtaaaaaacggagactggaccaagtttaaagaggtgaactatgccgacaaactgaagcaattctactactcaaaaggcgaaatagagagtacccaacaacagctgttagaagcccgggacaatattaaacaagcgaccaacacggaagataaggagtccatgaaactgaattataagaaactggaactgaagttaaaacaacagaatttgctggcgcaagaattcataaaaaaagcgtactgcggctaccttatcgatagcattaatgagattctgagagaatatccaaatacttatcttgtcttagaggatttggatatcgcgggtaaagcggatccagagtcggggatgactaataaagagcagaacttaaacaaaacgatgggggcttcagtataccaggccattgagaatgcgatcgtaaataaattcaaatatcgcaccgtgaaattgtccgatatcaagggccttcagactgtacctaatgtagtgaaggtcgaagacttacgggaagtgaaagaggttgaagatggggaacacaagttcgggttaataagatcagttaagagcaaggatcaaatcggtaacatactttttgtcgacgagggggagaccagtaacacttgtccgaattgcggttttaatagtgattggtttaaacgcgatgttgattttgacttagaaatagtcgctactgtaaacgggcaaaagaatgccgtgattgagcaaaatgacaaaaaatactgtttcccgggcgaaatatataaattggaaatcattaataaagagtacgaaacaaacaagcgtaatcttgccatgatttttaaacctcgggccaaagcgtgccgtaaatttatcaataataatttagataagaacgattatttctattgtccctactgcgccttctcgtcgaagaattgtaacaacccgaaactgcagaacggcgatttcgtggtatattcaggagacgatgttgctgcttacaatgttgctatcagaggaattaacctgctgaacaatattaaatag

SEQ ID NO: 19 - DNA, 36 bp, Unknown; identical to SEQ ID NO: 6
  Cas12a.1 repeat

SEQ ID NO: 20 - DNA, 36 bp, Unknown
  Cas12p repeat
ctcgaatatccctattagatttctacttttgtagat

SEQ ID NO: 21 - DNA, 36 bp, Unknown; identical to SEQ ID NO: 8
  Cas12q repeat

SEQ ID NO: 22 - DNA, 30 bp, Unknown
  Cas12a.1 spacer
cgtcgtattgagtgctagtactggtttgag

SEQ ID NO: 23 - DNA, 32 bp, Unknown
  Cas12p spacer
attaaattacataatgagccaacacggcgacc

SEQ ID NO: 24 - DNA, 33 bp, Unknown
  Cas12q spacer
ctagctcctctacgtctttattttcaccctcat

SEQ ID NO: 25 - DNA, 28 bp, Unknown
  Cas12a.1 - Cas12p target
gtggcagctcaaaaattggctacaaaac

SEQ ID NO: 26 - DNA, 36 bp, Unknown; identical to SEQ ID NO: 20
  Cas12p direct repeat

SEQ ID NO: 27 - DNA, 36 bp, Unknown
  Cas12q direct repeat
ctcaaatagacctatttaatttctacttttgtagat

SEQ ID NO: 28 - DNA, 36 bp, Unknown
  Cas12a.1 Direct Repeat
gtttaaggccttgacaaaatttccactgtagtggat

SEQ ID NO: 29 - DNA, 37 bp, Unknown
  Cas12a.1 Direct Repeat
ggtttaaggccttgacaaaatttctcctgtaggagat

SEQ ID NO: 30 - DNA, 36 bp, Unknown
  Cas12a.1 Direct Repeat
gtttaaggccttgacaaaatttcccctgtaggggat

SEQ ID NO: 31 - DNA, 36 bp, Unknown
  Cas12p Direct Repeat
atctacaaaagtagaagtctaatagggacattcgag

SEQ ID NO: 32 - DNA, 36 bp, Unknown
  Cas12p Direct Repeat
atctacaaaagtagaaagctaatagggctattcgag

SEQ ID NO: 33 - DNA, 36 bp, Unknown
  Cas12p Direct Repeat
atctacaaaagtagaaggctaatagggccattcgag

SEQ ID NO: 34 - DNA, 36 bp, Unknown
  Cas12p Direct Repeat
ctcgaatatccctattagatttcgacttttgtcgat

SEQ ID NO: 35 - DNA, 36 bp, Unknown
  Cas12p Direct Repeat
ctcgaatatccctattagatttctccttttggagat

SEQ ID NO: 36 - DNA, 36 bp, Unknown
  Cas12p Direct Repeat
ctcgaatatccctattagatttcggcttttgccgat

SEQ ID NO: 37 - DNA, 36 bp, Unknown
  Cas12q Direct Repeat
atctacaaaagtagaaattgaataggtctattcgag

SEQ ID NO: 38 - DNA, 36 bp, Unknown
  Cas12q Direct Repeat
atctacaaaagtagaaattaaagaggtctctttgag

SEQ ID NO: 39 - DNA, 36 bp, Unknown
  Cas12q Direct Repeat
atctacaaaagtagaaattgggtaggtctacccgag

SEQ ID NO: 40 - DNA, 36 bp, Unknown
  Cas12q Direct Repeat
ctcaaatagacctatttaatttccacttttgtggat

SEQ ID NO: 41 - DNA, 36 bp, Unknown
  Cas12q Direct Repeat
ctcaaatagacctatttaatttctccttttggagat

SEQ ID NO: 42 - DNA, 36 bp, Unknown
  Cas12q Direct Repeat
ctcaaatagacctatttaatttccccttttggggat

SEQ ID NO: 43 - DNA, 487 bp, Artificial Sequence
  KPC gB template 1 sequence
ttcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagaca

SEQ ID NO: 44 - DNA, 480 bp, Artificial Sequence
  NDM gB template 1 sequence
ccaaattaagatcatctatttactaggcctcgcatttgcggggtttttaatgctgaataaaaggaaaacttgatggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcgggg

SEQ ID NO: 45 - DNA, 125 bp, Artificial Sequence
  OXA gBlock 1 template sequence
cgaagccaatggtgactatattattcgggctaaaactggatactcgactagaatcgaacctaagattggctggtgggtcggttgggttgaacttgatgataatgtgtggttttttgcgatgaata

SEQ ID NO: 46 - DNA, 490 bp, Artificial Sequence
  MecA gBlock1 template sequence
tacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaa

SEQ ID NO: 47 - DNA, 232 bp, Artificial Sequence
  hHPRT1 template sequence
ctctgtatgttatatgtcacattttgtaattaacagcttgctggtgaaaaggaccccacgaagtgttggatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaat

SEQ ID NO: 48 - RNA, 100 nt, Artificial Sequence
  DENV ssRNA target  template sequence
ugacgaagaccaugcucacuggacagaagcaaaaaugcugcuggacaacaucaacacaccagaagggauuauaccagcucucuuugaaccagaaagggag

SEQ ID NO: 49 - RNA, 100 nt, Artificial Sequence
  ZIK ssRNA target  template sequence
ccacacuggaacaacaaagaagcacugguagaguucaaggacgcacaugccaaaaggcaaacugucgugguucuagggagucaagaaggagcaguucaca

SEQ ID NO: 50 - RNA, 100 nt, Artificial Sequence
  HANT ssRNA target  template sequence
agaggcaacuugcagauuugguggcagcucaaaaauuggcuacaaaaccaguugauccaacagggcuugagccugaugaucaucuaaaggaaaaaucauc

SEQ ID NO: 51 - DNA, 23 bp, Artificial Sequence
  Target sequence KPC 1
ttgctgaaggagttgggcggccc

SEQ ID NO: 52 - DNA, 23 bp, Artificial Sequence
  Target sequence NDM 1
gcgatctggttttccgccagctc

SEQ ID NO: 53 - DNA, 21 bp, Artificial Sequence
  Target sequence Ctrol + hHPRT1 1
ggttaaagatggttaaatgat

SEQ ID NO: 54 - DNA, 23 bp, Artificial Sequence
  Target sequence S16 cntl Ecoli 1
cagtagttatccccctccatcag

SEQ ID NO: 55 - DNA, 23 bp, Artificial Sequence
  Target sequence DENV1
cttctgtccagtgagcatggtct

SEQ ID NO: 56 - DNA, 23 bp, Artificial Sequence
  Target sequence DENV2
tggttcaaagagagctggtataa

SEQ ID NO: 57 - DNA, 23 bp, Artificial Sequence
  Target sequence ZIK1
ggcatgtgcgtccttgaactcta

SEQ ID NO: 58 - DNA, 23 bp, Artificial Sequence
  Target sequence ZIK2
ccttttggcatgtgcgtccttga

SEQ ID NO: 59 - DNA, 23 bp, Artificial Sequence
  Target sequence OXA1
agcccgaataatatagtcaccat

SEQ ID NO: 60 - DNA, 23 bp, Artificial Sequence
  Target sequence OXA 1b
agcccgaataatatagtcgccat

SEQ ID NO: 61 - DNA, 23 bp, Artificial Sequence
  Target sequence HANTAndes1
gtggcagctcaaaaattggctac

SEQ ID NO: 62 - DNA, 23 bp, Artificial Sequence
  Target sequence HANTAndes2
gatgatcatcaggctcaagccct

SEQ ID NO: 63 - DNA, 23 bp, Artificial Sequence
  Target sequence MecA1
tctttttgccaacctttaccatc

SEQ ID NO: 64 - DNA, 9165 bp, Artificial Sequence
  Cas12a.1 expression vector sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagatttacactttatgcttccggctcgtatgtttctagagaattcaaataattttgtttaactttaagaaggagatttaaatatggggtcaagtcatcaccaccaccaccactcaagtggactagtaccccgtggcagcatgaaagttagcacctgggatagcttcaccaaccagtacccgctgaccaagaccctgcgttttgagctgaagccggtgggtaaaaccctgcagaagatccaagaccgtaacctgattaccgaggacgaacagcgtcaaaaggatttcaacaaggttaagaaaatcatggatggttactacaagcagttcatcgaggaatgcctggaaggcgcgaagatcccgctgaagaaactggaggaaaacaacaacgcgtacaccaaactgaagaaagacccgtataacaagaaactgcgtgaggaatacgcgaagctgcagaaacaactgcgtaaactgatccacgatgagattaacaagaaagaggaattcaagtacctgtttaagaaagaattcatcaagaaaattctgccggaatggctggagaagaaaggtaagaaagaggaactgaaagagatcgaaaagttcgacaaatgggtgacctactttagcggcttctttaacaaccgtaagaacgttttcagcagcgacgagattagcaccagcatgatctatcgtattgtgaacgataacctgccgaaattcctggacgatgttagccgttttggtgaaattacccgttacaaggagttcgacgcgaaccagatcgaggaaaactttgagagcgaactgaacggtgaaaaactgaaggatttctttaacctgaaaaacttcaacaactgcctgaaccaagaaggcattgagaaatttaacctgatcattggtggcaagagcgaggaaggtaataacaaaatcaagggcctgaacgaactggtgaacgagctggcgcaaaaacaagcggacaagaacgagcagaagaaagttcgtaaactgaagctggcgccgctgttcaaacaaatcctgagcgatcgtaagagcagcagctttgcgttcgaaaaatttgaggaaaacaccgaggtgttcgacgcgatcgatgaattttatgacaagattagcctggagaccctgaagaaaatcgaagcgaccctggagaaactggaggaaaaggacctggaactggtttacctgaaaaacgatcgttgcctgaccggtatcagccaggaagtgttcggcgaccgtgagcgtgttctgcaagcgctgcgtgaatacgcgaaaaccgagctgggtctgaagaccgataagaaaatcgagaagtggatgaagaaaggtcgttatagcatccacgagattgaaagcggcctgaagaaaatcggtagcaccggccacccgatttgcaactacttcagcaaactggaggaaaagaaaaccaacctgatccaggaaattaagaaagcgcgtaccgagtatgaaaagatcagcgacaagaaaaagaaactgaccgcggaaagccaagagccgaacgtggcgcgtatcaaagcgctgctggatagcattatgcgtctgtatcacttcatcaagccgctgaacatcaacttcaagaacaagaaagagaaggacagcgaagcgctggagaccgacaacgatttttacaacgacttcgatgaaagctttgcggagctgggcaacatcattccgctgtacaaccaagtgcgtaactatgttacccaaaaaccgttcagcaccgagaaattcaagctgaactttgaaaacccgaagctgctgagcggttgggacaaaaacaaggaaaaagattactatagcgtgattctgcgtaaagaggaaagctactatctggcgatcatgaccccgaagcagaaaaacgttttcgacgagctggaacgtctgccggcgggcaaaaattacttcgagaagatcgattacaagctgctgccgaccccggaaaagaacctgccgcgtatcctgttcgcgaagaaaaacattagcttttacaagccgagcaaagagatcgaagcgattcgtaaccacagcgcgcacaccaaacacggtaacccgcagaacggcttcaagaaacgtgactttcgtctgagcgattgccacaagatgatcgacttctacaagaaaagcattcagaaacacccggaatggaaggagtatgattttcaattcaagaaaaccgaggactacgtggatatcagcgaattctataaagaggtttctgaccagggttacaagatcgaattcaagaaaattagcgagaaatacctgctggacctggtggaggaaggtaaactgtacctgttccaaatctggaacaaggatttcagcaagtacagcgaaggccgtaaaaacctgcacaccatctattggaaagaactgttcagcaaggagaacctgagcgatattacctataagctgaacggcgaggcggaaatcttttaccgtccgaaaagcatggagcgtaaggttacccacccgaagaaccagaaaatcgaaaacaaagacccgatcaagggtaagaaattcagcaagttcaagtatgacttcatcaagaacaagcgttacaccgaggatcgtttctttttccactgcccgatcaccctgaactttcaagcgcgtgacggcagcaaaaccatcaacaagcgtgtgaacgatcacattcgtgagaccaaagacgatatcttcgttctgagcattgatcgtggtgaacgtcacctggcgtactataccctgctgaacagcaagggtgaaattcaggagcaaggcagctttaacgtgatcagcgacgataaggagcgtaaacgtgactatcacgaaaaactggatgagcgtgaaaaggagcgtgacaaggcgcgtaaaagctggcagaaaatcgagaccattaagaaactgaaggatggctacctgagccaaatcgtgcacaagattgcgaaactggcgatcgagaaaaacgcgatcattgttctggaagacctgaacctggatttcaagcgtggtcgtctgaagattgagaaacaggtgtaccaaaaattcgaaaagaaactgatcgacaagctgaactatctggtttttaaagaacgtaccgaaaaagaggcgggtggtagcctgaacgcgtatcagctgaccggtaaattcgagggctttaagaaactgggcaaggaaaccggcatcatttactatgtgccggcggcgtacaccagcaaaatctgcccgaagaccggcttcgttaacctgctgcgtccgaagttcaagaacatcgaaaaggcgaaggagtttttcaagaagttcaactacatcaagtacgacagcagcgaaggtctgtttgagttcaacttcgattacagcaagttcatcaagaacggcaagaaagagaccaaaatcattcaggacaactggagcgtgtatagcaacggtaccaagctggttggcttccgtaacaagaacaaaaacaacagctgggataccaaggaagtgaaaccgaacgagaagctgaaaattctgttcaaagagtacggtgttagctttcaaaaggacgaaaacatcattagccagatcgcgagccaaaacaagaaagcgtttttcgagaacctgatcaagattttcaaaaccattctgatgctgcgtaacagccgtaaagacccggaggaagattacgtgctgagctgcgttaaggacgaaaacggcgagtttttcgacagccgtaaggcgaaagataacgagccgaaagacgcggatgcgaacggcgcgtaccacattggtctgaagggcctgatgctgctggaacgtatcaaggcgaacaaaggtaagaaaaagctggacctgctgatcagccgtaacgatttcattaactttgcggttgagcgtagcaagtaataaggatccctcgagttgacagctagctcagtcctaggtataatgctagcgtttaaggccttgacaaaatttctactgtagtagatgtggcagctcaaaaattggctacaaaacgtttaaggccttgacaaaatttctactgtagtagatctagcataaccccttggggcctctaaacgggtcttgaggggttttttgcatatgctgaaaggaggaactatatccggat

SEQ ID NO: 65 - DNA, 9246 bp, Artificial Sequence
  Cas12p expression vector sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagatttacactttatgcttccggctcgtatgtttctagagaattcaaataattttgtttaactttaagaaggagatttaaatatgggatcaagtcatcaccaccaccaccactcaagtggactagtacccaggggaagcatgaagaagagcattttcgatcagttcgttaaccagtacgcgctgagcaagaccctgcgtttcgagctgaaaccggtgggtgaaaccggccgtatgctggaggaagcgaaggttttcgcgaaggatgaaaccattaagaaaaagtacgaagcgaccaagccgttctttaacaaactgcaccgtgaattcgtggaggaagcgctgaacgaggttgaactggcgggcctgccggagtacttcgaaatcttcaagtactggaagcgttacaaaaagaaattcgagaaggacctgcagaagaaagagaaggaactgcgtaaaagcgtggttggtttctttaacgcgcaagcgaaggagtgggcgaagaaatatgaaaccctgggcgtgaagaaaaaggatgttggtctgctgttcgaggaaaacgtgtttgcgattctgaaagaacgttacggtaacgaggaaggcagccagattgtggacgagagcaccggcaaggatgttagcatcttcgacagctggaagggttttaccggctatttcatcaaatttcaggaaacccgtaagaacttctacaaagatgatggtaccgcgaccgcgctggcgacccgtatcattgatcaaaacctgaaacgtttctgcgacaacctgctgatctttgagagcattcgtgataagatcgacttcagcgaggttgaacagaccatgggcaacagcatcgataaggtgttcagcgttatcttttatagcagctgcctgctgcaagaaggtatcgacttttacaactgcgtgctgggtggtgaaaccctgccgaacggtgaaaagcgtcagggcattaacgaactgatcaacctgtaccgtcaaaagaccagcgagaaagttccgttcctgaagctgctggacaaacagattctgagcgagaaggaaaaatttatggatgagatcgaaaacgacgaggcgctgctggataccctgaagattttccgtaaaagcgcggaggaaaagaccaccctgctgaaaaacatcttcggcgattttgtgatgaaccagggtaaatatgacctggcgcaaatctacattagccgtgaaagcctgaacaccattagccgtaagtggaccagcgaaaccgatatcttcgaagacagcctgtacgaggtgctgaaaaagagcaaaatcgtgagcgcgagcgttaaaaagaaagacggtggctacgcgttcccggagtttatcgcgctgatttatgttaaaagcgcgctggaacagattccgaccgagaagttctggaaagaacgttactataagaacatcggcgatgtgctgaacaagggtttcctgaacggtaaagaaggcgtttggctgcaatttctgctgatctttgacttcgaatttaacagcctgttcgagcgtgaaatcattgatgagaacggcgacaagaaagtggcgggttataacctgttcgcgaagggttttgacgatctgctgaacaacttcaaatacgaccagaaggcgaaagtggttattaaggattttgcggacgaagttctgcacatttatcaaatgggcaaatacttcgcgatcgagaagaaacgtagctggctggcggactatgatattgacagcttctacaccgatccggagaagggttacctgaaattttatgaaaacgcgtacgaggaaatcattcaggtttataacaagctgcgtaactacctgaccaagaaaccgtatagcgaggacaagtggaaactgaacttcgaaaacccgaccctggcggatggttgggacaagaacaaagaggcggataacagcaccgtgattctgaagaaagacggtcgttactatctgggcctgatggcgcgtggtcgtaacaagctgttcgacgatcgtaacctgccgaaaatcctggagggtgttgaaaacggcaagtacgaaaaggtggtttacaagtacttcccggatcaggcgaagatgttcccgaaagtgtgctttagcaccaaaggcctggaattctttcaaccgagcgaggaagttatcaccatttacaagaacagcgagttcaagaaaggttatacctttaacgtgcgtagcatgcagcgtctgattgatttctataaagactgcctggttcgttacgaaggttggcaatgctatgattttcgtaacctgcgtaagaccgaggactaccgtaaaaacatcgaggaattctttagcgatgtggcgatggacggctacaagattagcttccaggacgttagcgagagctatatcaaggagaagaaccaaaacggtgatctgtacctgtttgagatcaagaacaaagactggaacgaaggtgcgaacggcaagaaaaacctgcacaccatttatttcgagagcctgtttagcgcggataacatcgcgatgaacttcccggtgaaactgaacggccaggcggagatcttttaccgtccgcgtaccgaaggtctggagaaggaacgtatcattaccaagaaaggcaacgttctggaaaagggtgacaaagcgttccacaagcgtcgttacaccgagaacaaagtgttctttcacgttccgattaccctgaaccgtaccaagaaaaacccgttccaatttaacgcgaagatcaacgacttcctggcgaaaaacagcgatatcaacgtgattggtgttgaccgtggcgagaaacagctggcgtattttagcgtgattagccaacgtggcaagatcctggaccgtggtagcctgaacgtgatcaacggcgttaactacgcggagaagctggaggaaaaagcgcgtggtcgtgaacaggcgcgtaaggattggcagcaaatcgagggcattaaagacctgaagaaaggttatattagccaggtggttcgtaaactggcggatctggcgatccaatacaacgcgatcattgtgttcgaggacctgaacatgcgttttaagcaaattcgtggtggcatcgagaaaagcgtttatcagcaactggaaaaggcgctgatcgataaactgaccttcctggtggagaaggaagaaaaggacgttgaaaaggcgggtcacctgctgaaagcgtaccagctggcggcgccgttcgaaacctttcagaagatgggtaaacaaaccggcattgtgttttatacccaagcggcgtacaccagccgtatcgatccggttaccggctggcgtccgcacctgtacctgaaatatagcagcgcggaaaaggcgaaagcggacctgctgaagttcaagaaaattaagttcgtggatggtcgtttcgagtttacctacgacatcaagagcttccgtgagcagaaggaacacccgaaagcgaccgtgtggaccgtttgcagctgcgttgagcgttttcgttggaaccgttatctgaacagcaacaaaggtggctacgatcactatagcgacgtgaccaagttcctggttgagctgtttcaggaatacggcatcgacttcgaacgtggtgatattgtgggccaaatcgaggttctggaaaccaagggtaacgagaagttctttaagaacttcgtgttctttttcaacctgatctgccagattcgtaacaccaacgcgagcgaactggcgaagaaagacggcaaggacgatttcattctgagcccggttgagccgtttttcgatagccgtaacagcgagaagttcggcgaagacctgccgaaaaacggtgacgataacggcgcgtttaacatcgcgcgtaaaggtctggttattatggataagatcaccaaattcgcggacgagaacggtggctgcgaaaagatgaaatggggtgacctgtatgtgagcaatgtggagtgggataactttgtggcgaataaataataaggatccctcgagttgacagctagctcagtcctaggtataatgctagcatctacaaaagtagaaatctaatagggatattcgaggtggcagctcaaaaattggctacaaaacatctacaaaagtagaaatctaatagggatattcgagctagcataaccccttggggcctctaaacgggtcttgaggggttttttgcatatgctgaaaggaggaactatatccggat

SEQ ID NO: 66 - DNA, 8658 bp, Artificial Sequence
  Cas12q expression vector sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagatttacactttatgcttccggctcgtatgtttctagagaattcaaataattttgtttaactttaagaaggagatttaaatatggggtcctcccatcatcaccaccaccactcttcaggcttggtaccgcgtggttccatgatcaacatagacgaattgaaaaatttatataaggtgcaaaagaccatcactttcgaacttaagaacaagtgggagaacaaaaatgatgagaacgacagagtagagttcttgaagactcaggagtgggtcgaaagccttttcaaggtcgatgaagagaactttgatgagaaagagtctatccctaacttgttagacttcggacagaagattgcgtccttgttttacaagctgagcgaggacatagcgaacaaccaaattgatacgcgggtattgaaagtctcgaaattccttttagaggaaattgatagaaatcaataccacgagaaaaaaaacaagcccacaaaggtaaaagaaatgaatcccaacacaaacaaaagttatataaaagaatataagctgtccgaccaaaacacactgtacgtgttattaaagataatggaagatgaaggtcggggattacaaaaatttttgtacgataaagcggaccggttaaacctgtacaatcaaaaagttcggagagacttcgccttaaaggaatcaaatgagcaacaaaaattctctggaaatgccaactactatgggaatataaagctgcttatagatagcttagaagatgcagtccggatcattgggtatttcactttcgacgatcaagcagaaaacgcacaaatcaatgaatttaagtccgttaaacaggaaatgaataataatgaagcgtcttaccaagcactgaaagacttcgctattgataacgcaaaaaaagagatagaattgacgacgttgaaccaccgggcggtcaacaaggatccaaaaaagattcaagaacagattgaggaagtcgaaaatttcgaagaagatattaaccagttaaagcatcagatatcagccttgaatgataagaagtttgacgtggttagcagattaaagcacgctcttataaaaatgttaccagaactgaatcttttggatgctgagtcggaacagggccgtgaagtccagcagatatatcaagacaaaaaaaacgggttggagcttgatgactttaaatttaaccttttaaaacatcatcaatggcaaaaaacgatcttcaagtatattaagcttgagggcttagttctgccagacctttacgcggaaaacaaacaagataaaatcaaggtttatattgagaattatagacagagtggtgagcgtatttctaagaaggcgagagaggaattaggaaaaatcgataaacgcgaagagttcaatggaaatgacgaacttaagaaggcatggtatgagtataaggacttctgtagagacaaacgtaataagagcgtggaacttggcaataagaagtcgctgtacaatgccataaagcgcgaagttttgcggcaaaaaatgtgcaaccatttcgctgtgctggtgtccgacggtgaagatacttccccttattattatctgatattaatcccgaacgagaactccgatgaaatgaatagaacgttcaaggaattgaaggcctccgaggggaattggaagatgttggattacaatcgtctgaccttcaaagccttggagaaattggccctgttacggtcgtctaccttcgagatagcggatcaggaactgcaagaagaggcaaaaaagatctgggaggagtacaaggaaaaggcgtacaaagacttcaaaaacaaaaagttattacagggtttatcgggaagacagcgggaggagaaaaagcaagaattgcaaaaggagagcctgaatagagtaatcaattacttgatcagatgcattcagtcattgcccgacagcggaaaatacaactttaactttaaagagcctcatcaataccaatcgcttgaagagtttgccgaggagattgatcggcaaggttatcactgtgcttggaaaaacgtttctaaagataaactgatggaattggaagcgatggaaaagattaaggttttcaaacttcataacaaagactttcgcaaggtaaaactgaacgactccaagcacaaccctaatctttttactttgtactggttagacgccatgaatttggataaggttaacgtccgcctgttaccggaagttgacctttacaagagagctaaggaaacacagctgaaattgttcgaacgtgatgtgaaatgcaatatcaataaccaaaagattaaatctatcaaggagaagaatagactgtttcaggacaagttgtatgctagttttaagttagagttttatccagaaaacgaaggattaggtttcgagcaggtaaatgacaaggtcaataacttctgcggtagcgatacggcctattatcttgggcttgatcgtggagagaaagagcttgttacattctgcctggtggactctgatggccgcctggtaaaaaacggagactggaccaagtttaaagaggtgaactatgccgacaaactgaagcaattctactactcaaaaggcgaaatagagagtacccaacaacagctgttagaagcccgggacaatattaaacaagcgaccaacacggaagataaggagtccatgaaactgaattataagaaactggaactgaagttaaaacaacagaatttgctggcgcaagaattcataaaaaaagcgtactgcggctaccttatcgatagcattaatgagattctgagagaatatccaaatacttatcttgtcttagaggatttggatatcgcgggtaaagcggatccagagtcggggatgactaataaagagcagaacttaaacaaaacgatgggggcttcagtataccaggccattgagaatgcgatcgtaaataaattcaaatatcgcaccgtgaaattgtccgatatcaagggccttcagactgtacctaatgtagtgaaggtcgaagacttacgggaagtgaaagaggttgaagatggggaacacaagttcgggttaataagatcagttaagagcaaggatcaaatcggtaacatactttttgtcgacgagggggagaccagtaacacttgtccgaattgcggttttaatagtgattggtttaaacgcgatgttgattttgacttagaaatagtcgctactgtaaacgggcaaaagaatgccgtgattgagcaaaatgacaaaaaatactgtttcccgggcgaaatatataaattggaaatcattaataaagagtacgaaacaaacaagcgtaatcttgccatgatttttaaacctcgggccaaagcgtgccgtaaatttatcaataataatttagataagaacgattatttctattgtccctactgcgccttctcgtcgaagaattgtaacaacccgaaactgcagaacggcgatttcgtggtatattcaggagacgatgttgctgcttacaatgttgctatcagaggaattaacctgctgaacaatattaaatagctagcataaccccttggggcctctaaacgggtcttgaggggttttttgctgaaaggaggaactatatccggat

SEQ ID NO: 67 - DNA, 8367 bp, Artificial Sequence
  Cas9.1 expression vector sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagatttacactttatgcttccggctcgtatgtttctagagaattcaaataattttgtttaactttaagaaggagatttaaatatgggcagcagccatcatcatcatcatcacagcagcggcctggtgccgcgcggcagcatgcagaggattttcggcctcgatatcggcaccacgtccatcggctttgcggtcatcgaccacgaccgcgaccaaggcgtcggccgcatccaccggctgggcgcgcgcatcttcccggaagcgcgcgacgagaagggaacaccgctcaaccagcatcggcggcaaaagcgtctcgcgcgccgccaattgcgccggcgccggcttcggcgcaaggcgctcaacgaactgctttcggcccgcgggatgctgccgcgcttcggcacgtccgcttggcacgacgcgatggcgctcgacccttacgcgctccgtgcacggggtacggaggaggcgttgcagccggtagaggtcggtcgggctctctatcacctcgcccagcgtcgccacttcaagccacgggacgaggctgcggaagccgacgagcaggaggtgggcgatcaggaggccgagaccaagcgtgagaagctgctgcaggcgttgcgccgcagcggtcgaacgctgggccaggaactggcggcgcgcggtccgcacgagcgcaagcggcacgagcacgctttgcgctcgaccgtcgagaccgagttcgagcggctcctcaccgcgcaagcgcggcatcacgagatccttcgcgatcccgagttcgtcgaggaactgagagagaccatcttcgcgcaacggcccgtcttttggcggacgagcacgctcggcacgtgcccgttcgttccaggcgcaccgttgtgcccgaagggtgcttggctctcccgccagcggcgcatgctggagcaggtcaacaacctcgccatcaccggcggcaacgcgcgtccgctcgaccacgaggagcgacgagcgatcctcgccgtcttacagacgcaggccagcatgagctggggcgcggtccgaaccgcgcttaagccgctcttcaaggcacgcggcgaggcgggcgccgagcgtcggctccggttcaatctcgaagagggcggcggtaagacgctgctcgggaacccgctggaagcgaagctcgcccggatcttcggcgaagcctgggccacgcaccctcaccgcgacgcgatccgtgagacgatccatgaccgccttttcgccgcgacctataacgcgaagggcgcgcagcgcatcgtcatccttccggcatcccaacgcgctgaacggatgcggggggtcatcgccggcctccaagcggatttcggcctttcccacgagcaggcgatggcgcttgcggagctgccgctgacgcccggctgggaaccctattcgagcgaagcccttcgcgcgttaatgccgaagctggaggaaggcgtgcgcttcggcgccctcgtcgtggcccctgaatgggaagattggcgcgaggccaccttcccccagcgcgagcggccgaccggcgaggtgctcgacctcttgccttcaccgaaatgccacgatgagagccgccggcagacgcggctgcggaacccgacggtgctgcgcacgcagaacgagctgcgcaaggtcgtcaacaacctgatccgggcgcacggcaagcccgacatcatccgcgtcgaggtcgcccgcgaggtggggctttccaagcgcgagcgtgaagatcgctacaacgggatgcggcgccaggagcgccagcggcaagcggcgatcaaagacctccaagccaagggcttcgccgagccgtcgcgcgccgacgtcgagaagtggcttttgtggaaggagagcaaggagacctgcccttacacgggggacaagatctgcttcgacgctctgtttcgccgcggtgagtttcaagtggagcacatctggccgcgctcgcgctcgttcgacgacagcttccgcaacaagaccctgtgtcggcgcgacgtgaacctcgccaagggtaaccaaacgcccttcgagttcttcgagagccgacccgaggagtgggaggccgtgaagcgccgcctcgatggcttgcaggccaagcgggcaggcggtgaggggatggcgcgcggcaaggtgaagcgcttcgtcgcgagcacgttgccggacgatttcgcgcagcgtcagctcaacgacacgggctgggcggcgcgcgaggcggtggccttcctcaagcggctgtggccggacgaggggcaagccgcgccggtccgcgtccaggcggtcacggggcgggtgacggcgcagcttcgccacctggggggcctcgatggcgtgctgtcggacggtgctcgaaagacgcgtgacgaccaccgccatcacgccgtcgatgcgctggtcgtcgcctgcacgcatccgggcatgaccgagcggctcagccgctactggcagcagaaggaggacgagcgcgccgaacgaccgcagctggacccaccgtggcccacgatccgagcggacgccgaggcggccaaggacttaatcgtcgtctcgcaccgggtgcgcaagaagatctcgggaccgttccacaaggaaaccgtctatggcgcgaccgacgagcgcgaggtcacgcgcgggcttgagtacgagaaattcgtcacgcggaagcgcgtcgaggacctgacgaaatccatgctcgccgacatccgcgacgacagggtgcggcaaattgtgacggcgtgggtggccgagcgcggcggcgacccgaagaaggcgtttccgccctatccgacgctggggtcgagcggacccgagatccgcaaggtgcgcgttctgatccgccggcagcccaccttgatggcacgggcagcgacgggcttcgctgatctcggagcgaaccaccatgtcgccatctacaagaccgccgacgagcgattcgccttcgaggtcgtcagcttgctggaggtcgccaggcgcgtcgaccgcggtgaaccgcccgtgaagagacagcgaggcgacgagaagctcgtgatgtctttggcgcagggcgatctgatacggttcgccaaaacgcccgatgcggaagcagcaatttggcgtgttcagaaaatcgcaactaaaggtcagatatcgctccttcaccacgatgacgcttcgccgaaggagccgagtctctttgaaccgatggttggtgggttgatggctcggaacccggagaagctggcagtcgatcccatcggccgagtgcgcaaggcaggcgactgactagcataaccccttggggcctctaaacgggtcttgaggggttttttgcatatgctgaaaggaggaactatatccggat

SEQ ID NO: 68 - DNA, 9378 bp, Artificial Sequence
  Cas 9.2 expression vector sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagatttacactttatgcttccggctcgtatgtttctagagaattcaaataattttgtttaactttaagaaggagatttaaatatgggcagcagccatcatcatcatcatcacagcagcggcctggtgccgcgcggcagcatgaagaaagaaaaagtgtacatggggctagatcttggcacgaactctgtcggctgggcggtaacggataacgactataaggtgctcaagtttaaacggcgcgctatgtggggggttcggctctttaatgaagccaatccggctgtcgagaggcgtgttgcccgttcaaatcgccgtcgcctggcccgaaaaaaacaacgcgtggcttggctgaaagaaatatttaagaattccattagcgaaattgatccggaatttttcgaccgtcttgaacaaagcgcgctttgggcagaagacaaaaatgtcgccggaaaatactccctttttaatgagaaaaaattaaccgataagacattctatcggaaatttcccaccgtttttcacctaaaaaaagcgcttatggacggcaaaataaaaaaacctgatattcgctttgtatatcttgccttgtcccactatctgcaaaacagaggccattttctcttggaaaatgagctgaacagtgttgaagatatagacattcgggatatttttaacagtcttaatgaaagaattcatgttcttattgacagcggtgatgatatggttcctgcttttgatttgacaaaccttgatgatttgaaacaaattgccacagacacaaatatatccgggaaaacgcaggaaaaagaagcctttataaaaaccctgttaaatggggccaaacagcctgccttagaggcaattattaaattatgtacaggcggctcggctaatttatcaaaaatctttggtgatatgtttgaatttgaaagtgaaatcaaatcaatatcattcgaaaaggccaacttcgaagatgaaatcgctcccaagctgcaagattgtctgggagattactatcagattattgagctggctcagcagatttacagctggtacacgctttataaggtatgcagcggtcgaccgtcggtctctcacgccaaagtggaggattacgaaaaacacaaagaacagctgtcccacctaaaagtgctggtaagaaaacacttttcgaaaaatgtctaccgggaaatattccgaaaagaagacgacaaaatccataactatgtatcctacatatccggcaaaaaggaccgcgacgaattttataaatatctcaaaaaaacgttagaaaaaaaatctacattcaagaaaacgtctgaatttgagaatatttctcgcgccattgaacagcaaaactacctgccgaaacaacgggtcaaagacaactctgtggtgcctcagcagctatacaaacaagaaatcgtaaaaatcctcaacaacctttcatcacactacccctttttatcacaaaaaacagacgggatcagcaatcgagaaaagattatcaaaatctttgaataccgcatcccatactatgtcggtcctctttgcgatatccatcgtgcgggggatgacgggttctcctggctggttcgtgactgcagtaaaaagattactccttggaacttcgagcaagtcgtcgatatcccccagtctgctgaaaatttcattaagaacatgacccgtaaatgcacctatttaaaacagtataatgtgctgccgaaaaattctctcctctatagcgagtatagcgtactaaatgaacttaacaatgtgcgcatcaaaactaaaaagctgacccctaagctaaaagaaaaaatgctcaacacattatttcgccaaaagaagaatatttcgataacgagcttgattcattggcttgtcagtgaaggagtgtatgagaaaggggagattgaaaaatcagacgtcagcggtgttgattccaattttaccagctctctttctgcagccatttcttttgatcgtatcattggtgaaaagatgaaaaacaaaaaaacccaaaaaatggtcgaggagatcataaactggctcgcccttttttcggacaaaaaaatactacaacaaaagattgtagagaaatatcaagataaagtctcgcaagaacaaatcggaaaaattctgcgcctcaacctaagcggatggggacgactttcttcggagtttctgcaactgaaaaactcccaaccgggagaacacgacggaaaaacgctcatcaatatcatgcggcagacccagatgaatctgatggagattattcactctccccagttcagtttcaataccgttattgaaacggaggccaaaaaacagctaacgggacacattacccacagtcatgttgaggcgctgtactgctctcctgtggtcaagaaacagatatggcaggccctgcaaatcgccctggagctaaagaaaaccttaaagaaagacccgaacaaaatttttgtggagacaacccggcatgaaggggagaaaaaacggaccacaagccgtcacaaacaactactcgagttataccaagccgccaagtcccatctgcccgacctgacgaaaagtataaaggaactaaacgatgcgctaaaagatacagagccggagaagatgaaacggaaaaaactgtttcactactacaaacaactgggacgttgtatgtatacaggcaggcccatcagtctagaggatctgtttaccaataaatatgacattgatcatatttatccccagagtttaaccaaagatgacagttttactaatactgtactggtggaacggctatcaaacgcggagaaatcagacgcattccctcttgacagtaaaacaagaaaagaccgtcaaggactgtggcgctgtttacgacggaacggactaattaccaaagaaaagtactaccgcttaacacgggaaacacctctaagcgaagaagaaaaagcggcctttattcgtcgtcagctggtggaaaccagccagacaaccaaggaagtaatccgatttctggcgacccttttcccaaagtcaaaagttgtgtatgtaaagagcggcaacgtcagcgactttcgccgtgacttttccccgtccctgcccgaaaacaaaactaacggcaaagaccccaaggggataaccgactacagcatgattaaagtgagggaaatcaatgatttgcaccacgcgaaagacgcgtatttaaacatcgtggtcggcaatgtctacgacaccaaatttcgctaccgaggcaaagacctcacggccatagtgcgcgaaaaagcgaggcagtaccatttatcccgtttgtttctttactctaccgacggcgcctggatcggagcggctgatgaaaacagagggaagcaacgaccgagtattgaaaccgtgatcgcggaaatgcggcgaaatagctgtcaggtaacgtgggaagccgtctttaaaaaagggcagctgtgggacatgaacgccaaaagtaagcggccgggactgttgccgatcaagaaagaactatccgatacggcaaaatatggagggtaccaggggaagaccgcgtcttattttgtggtcgttgagtatgagaataaaaaaggcgaacgtgaaaaaaaactggaatcggtcccgatttatgtgaaagcgctcagtaaacaaaagccggacgctgtcaattctttcctacgggatacactgggtctggagaaaccaagcgtcatggtcgacaacatcaaaatcggctccatcgtcgagatcaacggggcccgaatggtccttacggggaataatgaagttctagtatttgggcgtatcgcgtcccaactgatcctggatataacgatggccgcctatctaaaacgaatgtttaagctgcttgctgacacagccaagatcaaagagaacaatgtctactttaaaaactgcggctatctggataaggagacgaacctggcagtatacgatacgtttattgccaagctgaaactgccccggtatgctcagattatcacccatagcctatatgagaagatggaaagcaatcgtgatgtgtttatcaacctttcactggccgaccagtgtaatctgctggccggcgtactgcctgcgctacagtgtaacagccaaaatgccgatctgtctcttcttggtgaaggtaaagcggtcggaaatatcgcattttcaaaaaacgcgatcctgaaaaagaatcaggtccgtcttgttgattgctccattaccgggctcttcgaaaacagcagaaatatggcataactagcataaccccttggggcctctaaacgggtcttgaggggttttttgcatatgctgaaaggaggaactatatccggat

SEQ ID NO: 69 - DNA, 1871 bp, Andes orthohantavirus
tagtagtagactccttgagaagctactgctgcgaaagctggaatgagcaccctccaagaattgcaggaaaacatcacagcacacgaacaacagctcgtgactgctcggcaaaagcttaaggatgccgagaaggcagtggaggtggacccggatgacgttaacaagagcacactacaaagtagacgggcagctgtgtctacattggagaccaaactcggagaacttaagaggcaacttgcagatttggtggcagctcaaaaattggctacaaaaccagttgatccaacagggcttgagcctgatgatcatctaaaggaaaaatcatctctgagatatgggaatgtcctggatgttaattcaattgatttggaagaaccgagtggacagactgctgattggaaggctataggagcatacatcttagggtttgcaattccgatcatcctaaaggccttatacatgctgtcaacccgtgggagacaaactgtgaaagacaacaaagggaccaggataaggtttaaggatgattcttcctttgaagaagtcaatgggatacgtaaaccaaaacacctttacgtctcaatgccaactgcacagtccactatgaaggctgaagaaatcacgccaggacgatttaggacaattgcttgtggcctttttccagcacaggtcaaagcccgaaatataataagtcctgtaatgggagtaattggatttggcttctttgtaaaggattggatggatcggatagaagagtttctggctgcagagtgtccattcttacctaagccaaaggtcgcctcagaagccttcatgtctaccaataagatgtattttctgaacagacagagacaagtcaatgaatctaaggttcaagatattatcgatttgatagaccatgctgagaccgagtctgctaccttgtttacagagattgcaacaccccattcagtctgggtgtttgcatgtgcacctgaccggtgccctccaactgcattgtatgttgcaggggtaccggaacttggtgcatttttttctatccttcaggacatgcgtaataccatcatggcatctaaatctgtagggactgcagaagagaagctaaagaaaaaatctgccttctaccaatcatacctaagaaggacacaatctatgggaatccaactggaccagaagatcataatcctttacatgctatcatggggtaaagaagctgtgaatcacttccatcttggtgatgatatggaccctgaactcaggcagctagcacaatctctgatcgatactaaggtgaaggagatctccaaccaagagccacttaagttgtaggtgcttaatgaaatcatgattgaagaaagactttccgggcttgtgccacatattaatcatctcaggacctatccttaatgtgattaatagggttttattataagggcagttaatggggttggttactaactatgggtaagggttcattaccatttttgcactagggttaaagggccactacattgtatttgcactaagggaaatgggaggtgggttagtttgtatttagttgttaagttttttataatcatatgttaatgaggaattagctatatgatatcactgattgattggctatttttaggttaagtaattgtagttaaatagttgtgttaagttagtatgttaaggtttataggttaagatttactaacaatcatattatgtcattagatgtaaatttcattcctggcttgcttctgctttcgcattgctaacctacaacaagactacctcacccactacccctcccctattctacctcaacacatactacctcacatttgatttttcttgattgcttttcaaggagcatactacta

SEQ ID NO: 70 - DNA, 23 bp, Andes orthohantavirus
gtggcagctcaaaaattggctac

SEQ ID NO: 71 - DNA, 28 bp, Unknown
  Cas12a.1 spacer
cccgattgacgctatagtaagcatcgag

SEQ ID NO: 72 - DNA, 27 bp, Unknown
  Cas12a.1 spacer
gcgtcccataaaggtatgacttgtatt

SEQ ID NO: 73 - DNA, 28 bp, Unknown
  Cas12a.1 spacer
acgcacgcagtattgaatacgcgaatag

SEQ ID NO: 74 - DNA, 27 bp, Unknown
  Cas12a.1 spacer
gttcacgtaaaacttaatcgttgaact

SEQ ID NO: 75 - DNA, 27 bp, Unknown
  Cas12a.1 spacer
tggtagtgccaacacgtgcgcccacca

SEQ ID NO: 76 - DNA, 29 bp, Unknown
  Cas12a.1 spacer
tcggtggtgggcgaacattgactgttggt

SEQ ID NO: 77 - DNA, 28 bp, Unknown
  Cas12a.1 spacer
ccgattccttctcgttcgcccgtgacca

SEQ ID NO: 78 - DNA, 28 bp, Unknown
  Cas12a.1 spacer
gttgcgggagatactacttcaatataca

SEQ ID NO: 79 - DNA, 29 bp, Unknown
  Cas12a.1 spacer
ggtagccgaaatgaattcggtataacccg

SEQ ID NO: 80 - DNA, 28 bp, Unknown
  Cas12a.1 spacer
aaccagtatcctaccgtgaagttgtcgc

SEQ ID NO: 81 - DNA, 27 bp, Unknown
  Cas12a.1 spacer
gggggtttgagtgggcaacgcaaggaa

SEQ ID NO: 82 - DNA, 27 bp, Unknown
  Cas12p spacer
ttcagatgtttgctctttgacatatcg

SEQ ID NO: 83 - DNA, 30 bp, Unknown
  Cas12p spacer
atggtgatttaaaaacaaaactcggcgcga

SEQ ID NO: 84 - DNA, 29 bp, Unknown
  Cas12p spacer
ccttgtgcaaaatagacaggttagaccgt

SEQ ID NO: 85 - DNA, 30 bp, Unknown
  Cas12p spacer
cgaaaatccagctaaactcattctctgatt

SEQ ID NO: 86 - DNA, 26 bp, Unknown
  Cas12p spacer
ttgattggaggaacaagctacataaa

SEQ ID NO: 87 - DNA, 26 bp, Unknown
  Cas12p spacer
cgtatgggtgtaatttaatcggtttg

SEQ ID NO: 88 - DNA, 27 bp, Unknown
  Cas12p spacer
atgaatcgtataagatatgatctgaat

SEQ ID NO: 89 - DNA, 27 bp, Unknown
  Cas12p spacer
aatcgtagcgataaccgaagaacaaat

SEQ ID NO: 90 - DNA, 27 bp, Unknown
  Cas12p spacer
aacccatatgttttattatcctgctga

SEQ ID NO: 91 - DNA, 25 bp, Unknown
  Cas12p spacer
tacaaaattaaggcggtctaggaga

SEQ ID NO: 92 - DNA, 25 bp, Unknown
  Cas12p spacer
tccatttgatgataaccataagaat

SEQ ID NO: 93 - DNA, 25 bp, Unknown
  Cas12p spacer
aaaataatgtaatataatacaatat

SEQ ID NO: 94 - DNA, 25 bp, Unknown
  Cas12p spacer
ctcgacactgggacaacttccgtat

SEQ ID NO: 95 - DNA, 27 bp, Unknown
  Cas12p spacer
caataaatactgattagaagaagatat

SEQ ID NO: 96 - DNA, 27 bp, Unknown
  Cas12p spacer
ctgtcaaagccatagtcttgatccagc

SEQ ID NO: 97 - DNA, 26 bp, Unknown
  Cas12p spacer
gcgcaaagcatcagcgcaatggctcg

SEQ ID NO: 98 - DNA, 25 bp, Unknown
  Cas12p spacer
tggcagagttcgggccaagtatcat

SEQ ID NO: 99 - DNA, 25 bp, Unknown
  Cas12p spacer
gtagcgttctgttacgtgccagcga

SEQ ID NO: 100 - DNA, 26 bp, Unknown
  Cas12p spacer
cggaataatgtatgtcttaccgaggc

SEQ ID NO: 101 - DNA, 25 bp, Unknown
  Cas12p spacer
ctacgattaccttaacgaccctaac

SEQ ID NO: 102 - DNA, 26 bp, Unknown
  Cas12p spacer
atgattgacacaataattaactggtt

SEQ ID NO: 103 - DNA, 25 bp, Unknown
  Cas12p spacer
ataccgtctgtacctattgggggca

SEQ ID NO: 104 - DNA, 25 bp, Unknown
  Cas12p spacer
aaaagtgctaaaattcttaacggaa

SEQ ID NO: 105 - DNA, 26 bp, Unknown
  Cas12p spacer
cttgctgaattcggctcaagcatcat

SEQ ID NO: 106 - DNA, 26 bp, Unknown
  Cas12p spacer
cagcatgggatagaacgcttccgagc

SEQ ID NO: 107 - DNA, 27 bp, Unknown
  Cas12p spacer
ccactagcatctcctaggatagttgga

SEQ ID NO: 108 - DNA, 26 bp, Unknown
  Cas12p spacer
atataagacagctccaagctcccgtt

SEQ ID NO: 109 - DNA, 27 bp, Unknown
  Cas12p spacer
tacctctggagtttaatctttgataga

SEQ ID NO: 110 - DNA, 26 bp, Unknown
  Cas12p spacer
aatgaaaaaccaaaatccgcacctta

SEQ ID NO: 111 - DNA, 26 bp, Unknown
  Cas12p spacer
gacgcatattgcatagcggtttatgc

SEQ ID NO: 112 - DNA, 25 bp, Unknown
  Cas12p spacer
ataaattcacaaactaacttgtaac

SEQ ID NO: 113 - protein, 9 aa, Unknown
  Cas12p deleted sequence
KNGNPQKGY

SEQ ID NO: 114 - protein, 4 aa, Unknown
  Cas12p deleted sequence
PAKE

SEQ ID NO: 115 - protein, 7 aa, Unknown
  Cas12p deleted sequence
KNGNPQY

SEQ ID NO: 116 - RNA, 20 nt, Artificial Sequence
  sgRNA scaffold for Cas12a1
aaauuucuacuguaguagau

SEQ ID NO: 117 - RNA, 20 nt, Artificial Sequence
  sgRNA scaffold for Cas12p
agauuucuacuuuuguagau

SEQ ID NO: 118 - DNA, 33 bp, Artificial Sequence
  target sequence
gtggcagctcaaaaattggctacaaaaccagtt

SEQ ID NO: 119 - DNA, 23 bp, Artificial Sequence
  target sequence
gatcgcgccccactgcgttctcc

SEQ ID NO: 120 - RNA, 23 nt, Artificial Sequence
  target sequence
auggcaccuguguaggucaacca

SEQ ID NO: 121 - DNA, 8 bp, Artificial Sequence
  ssDNA fluorescently labeled reporter
  misc_feature (1)..(1): FAM reporter fluroscent dye moiety attached
  misc_feature (8)..(8): Iowa Black FQ quencher moiety attached
ttattatt

SEQ ID NO: 122 - protein, 1273 aa, Artificial Sequence
  Synthetic polypeptide
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYA

SEQ ID NO: 123 - RNA, 23 nt, Artificial Sequence
  target sequence
ugugcugacucuaucauuauugg

SEQ ID NO: 124 - DNA, 70 bp, Artificial Sequence
  target sequence
  misc_feature (70)..(70): FAM reporter fluroscent dye moiety attached
tcatttagaaagtagatattgattgattttagcgaaagccaatttttgagctgccactgatgtaaaagtt

SEQ ID NO: 125 - DNA, 120 bp, Artificial Sequence
  target sequence
gctatcttaatccttaatctatcctcaaacgttctattaatggccgtgtcaatcaatatctactttctaaatgaaacttttacatcagtggcagctcaaaaattggctttcgctaaaatc

SEQ ID NO: 126 - DNA, 65 bp, Artificial Sequence
  target sequence
taagcgcccttgcgctttccccagccttcgggttggttgccttttagtgcaagggcgcgattatt

SEQ ID NO: 127 - DNA, 120 bp, Artificial Sequence
  target sequence
gattttagcgaaagccaatttttgagctgccactgatgtaaaagtttcatttagaaagtagatattgattgacacggccattaatagaacgtttgaggatagattaaggattaagatagc

SEQ ID NO: 128 - DNA, 23 bp, Artificial Sequence
  reporter sequence
  misc_feature (1)..(19): joined by phosphorothioate bonds
ccccccccccccccccccttatt

SEQ ID NO: 129 - RNA, 23 nt, Artificial Sequence
  reporter sequence
  misc_feature (1)..(19): joined by phosphorothioate bonds
ccccccccccccccccccuuauu

SEQ ID NO: 130 - DNA, 27 bp, Artificial Sequence
  chimeric guide sequence
agacacgagagggcagccaaaaatggc

SEQ ID NO: 131 - DNA, 27 bp, Artificial Sequence
  chimeric guide sequence
agacacgagagggcagccaaaaatggc

SEQ ID NO: 132 - RNA, 40 nt, Artificial Sequence
  chimeric guide sequence
agauuucuacuuuuguagauguggcagcucaaaaauuggc

SEQ ID NO: 133 - DNA, 28 bp, Artificial Sequence
  chimeric guide sequence
agacacgagagggcagccaaaaattggc

SEQ ID NO: 134 - RNA, 36 nt, Unknown
  direct repeat for Cas 9.1 crRNA
acuguagcaagacgaagggccggcgcaauccgcagc

SEQ ID NO: 135 - RNA, 36 nt, Unknown
  direct repeat for Cas 9.3 crRNA
uuacauuaguuuaaaaccaaacucccuuuggcgucg

SEQ ID NO: 136 - RNA, 36 nt, Unknown
  direct repeat for Cas 9.4 crRNA
ugacgcaaaguaaaauuuaggguuaguuuaguguug

SEQ ID NO: 137 - protein, 18 aa, Campylobacter jejuni
ARILAFDIGISSIGWAFS

SEQ ID NO: 138 - protein, 22 aa, Campylobacter jejuni
LPRRLARSARKRLARRKARLNH

SEQ ID NO: 139 - protein, 13 aa, Campylobacter jejuni
VHKINIELAREVG

SEQ ID NO: 140 - protein, 16 aa, Campylobacter jejuni
LHHAIDAVIIAYANNS

SEQ ID NO: 141 - protein, 18 aa, Clostridium perfringens
NYALGLDIGITSVGWAVI

SEQ ID NO: 142 - protein, 22 aa, Clostridium perfringens
LPRRLARGRRRLLRRKAYRVER

SEQ ID NO: 143 - protein, 13 aa, Clostridium perfringens
PVRINIELARDLA

SEQ ID NO: 144 - protein, 16 aa, Clostridium perfringens
KHHALDAAVVGVTTQG

SEQ ID NO: 145 - protein, 18 aa, Akkermansia muciniphila
SLTFSFDIGYASIGWAVI

SEQ ID NO: 146 - protein, 13 aa, Akkermansia muciniphila
FKRREYRRLRRNI

SEQ ID NO: 147 - protein, 13 aa, Akkermansia muciniphila
ISRVCVEVGKELT

SEQ ID NO: 148 - protein, 16 aa, Akkermansia muciniphila
LHHALDACVLGLIPYI

SEQ ID NO: 149 - protein, 18 aa, Bifidobacterium longum
RYRIGIDVGLNSVGLAAV

SEQ ID NO: 150 - protein, 22 aa, Bifidobacterium longum
NMSGVARRTRRMRRRKRERLHK

SEQ ID NO: 151 - protein, 13 aa, Bifidobacterium longum
PVSVNIEHVRSSF

SEQ ID NO: 152 - protein, 16 aa, Bifidobacterium longum
RHHAVDASVIAMMNTA

SEQ ID NO: 153 - protein, 18 aa, Wolinella succinogenes
VSPISVDLGGKNTGFFSF

SEQ ID NO: 154 - protein, 22 aa, Wolinella succinogenes
VGRRSKRHSKRNNLRNKLVKRL

SEQ ID NO: 155 - protein, 13 aa, Wolinella succinogenes
KVPIILEQNAFEY

SEQ ID NO: 156 - protein, 16 aa, Wolinella succinogenes
SSHAIDAVMAFVARYQ

SEQ ID NO: 157 - protein, 18 aa, Legionella pneumophila
LSPIGIDLGGKFTGVCLS

SEQ ID NO: 158 - protein, 22 aa, Legionella pneumophila
AQRRATRHRVRNKKRNQFVKRV

SEQ ID NO: 159 - protein, 13 aa, Legionella pneumophila
LIPIYLEQNRFEF

SEQ ID NO: 160 - protein, 15 aa, Legionella pneumophila
PSHAIDATLTMSIGL

SEQ ID NO: 161 - protein, 18 aa, Francisella novicida
ILPIAIDLGVKNTGVFSA

SEQ ID NO: 162 - protein, 22 aa, Francisella novicida
NNRTARRHQRRGIDRKQLVKRL

SEQ ID NO: 163 - protein, 13 aa, Francisella novicida
HIPIITESNAFEF

SEQ ID NO: 164 - protein, 16 aa, Francisella novicida
YSHLIDAMLAFCIAAD

SEQ ID NO: 165 - protein, 18 aa, Streptococcus pyogenes
KYSIGLDIGTNSVGWAVI

SEQ ID NO: 166 - protein, 22 aa, Streptococcus pyogenes
EATRLKRTARRRYTRRKNRICY

SEQ ID NO: 167 - protein, 13 aa, Streptococcus pyogenes
PENIVIEMARENQ

SEQ ID NO: 168 - protein, 16 aa, Streptococcus pyogenes
YHHAHDAYLNAVVGTA

SEQ ID NO: 169 - protein, 1368 aa, Streptococcus pyogenes [sequence omitted]

SEQ ID NO: 170 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 171 - protein, 1082 aa, Neisseria lactamica
MAAFKPNPMNYILGLDIGIASVGWAMVEIDKEENPIRLIDLGVRVFERAEVPKTGDSLAMARRLARSVRRLTRRRAHRLLRARRLLKREGVLQDADFDENGLVKSLPNTPWQLRAAALDRKLTCLEWSAVLLHLIKHRGYLSQRKNEGETADKELGALLKGVADNAHALQTGDFRTPAELALNKFEKESGHIRNQRGDYSHTFSRKDLQAELNLLFEKQKEFSNPHVSDSLKEGIETLLMAQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEAPTLMEMKAYHAISRALEKEGLKDKKSPLNLSTELQDEIGTAFSLFKTDKDITGRLKDRVQPEILEALLKHISFDKFVQISLKALRRIVPLMEQGKRYDEACAEIYGDHYCKKNAEEKIYLPPIPADEIRNPVVLRALSQARKVINCVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEEGFKERNLNDTRYVNRFLCQFVADHILLTGKGKRRVFASNGQITNLLRGFWGLRKVRAENDRHHALDAVVVACSTVAMQQKITRFVRYKEMNAFDGKTIDKETGEVLHQKAHFPQPWEFFAQEVMIRVFGKPDGKPEFEEADTPEKLRTLLAEKLSSRPEAVHEYVTPLFVSRAPNRKMSGQGHMETVKSAKRLDEGISVLRVPLTQLKLKGLEKMVNREREPKLYDALKAQLETHKDNPAKAFAEPFYKYDKAGNRTQQVKAVRIEQVQKTGVWVRNHNGIADNATMVRVDVFEKGGKYYLVPIYSWQVAKGILPDRAVVAFKDEEDWTVMDDSFEFRFVLYANDLIKLTAKKNEFLGYFVSLNRATGAIDIRTHDTDSTKGKNGIFQSVGVKTALSFQKNQIDELGKEIRPCRLKKRPPVR

SEQ ID NO: 172 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 173 - protein, 1128 aa, Streptococcus thermophilus
MSDLVLGLDIGIGSVGVGILNKVTGEIIHKNSRIFPAAQAENNLVRRTNRQGRRLTRRKKHRIVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPEEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKRLRLQIKQNISNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNPNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEEQLLDIETGELISDDEYKESVFKAPYQHFVDTLKSKEFEDSILFSYQVDSKFNRKISDATIYATRQAKVGKDKKDETYVLGKIKDIYTQDGYDAFMKIYKKDKSKFLMYRHDPQTFEKVIEPILENYPNKQMNEKGKEVPCNPFLKYKEEHGYIRKYSKKGNGPEIKSLKYYDSKLLGNPIDITPENSKNKVVLQSLKPWRTDVYFNKATGKYEILGLKYADLQFEKGTGTYKISQEKYNDIKKKEGVDSDSEFKFTLYKNDLLLVKDTETKEQQLFRFLSRTLPKQKHYVELKPYDKQKFEGGEALIKVLGNVANGGQCIKGLAKSNISIYKVRTDVLGNQHIIKNEGDKPKLDF

SEQ ID NO: 174 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 175 - protein, 1372 aa, Legionella pneumophila
MESSQILSPIGIDLGGKFTGVCLSHLEAFAELPNHANTKYSVILIDHNNFQLSQAQRRATRHRVRNKKRNQFVKRVALQLFQHILSRDLNAKEETALCHYLNNRGYTYVDTDLDEYIKDETTINLLKELLPSESEHNFIDWFLQKMQSSEFRKILVSKVEEKKDDKELKNAVKNIKNFITGFEKNSVEGHRHRKVYFENIKSDITKDNQLDSIKKKIPSVCLSNLLGHLSNLQWKNLHRYLAKNPKQFDEQTFGNEFLRMLKNFRHLKGSQESLAVRNLIQQLEQSQDYISILEKTPPEITIPPYEARTNTGMEKDQSLLLNPEKLNNLYPNWRNLIPGIIDAHPFLEKDLEHTKLRDRKRIISPSKQDEKRDSYILQRYLDLNKKIDKFKIKKQLSFLGQGKQLPANLIETQKEMETHFNSSLVSVLIQIASAYNKEREDAAQGIWFDNAFSLCELSNINPPRKQKILPLLVGAILSEDFINNKDKWAKFKIFWNTHKIGRTSLKSKCKEIEEARKNSGNAFKIDYEEALNHPEHSNNKALIKIIQTIPDIIQAIQSHLGHNDSQALIYHNPFSLSQLYTILETKRDGFHKNCVAVTCENYWRSQKTEIDPEISYASRLPADSVRPFDGVLARMMQRLAYEIAMAKWEQIKHIPDNSSLLIPIYLEQNRFEFEESFKKIKGSSSDKTLEQAIEKQNIQWEEKFQRIINASMNICPYKGASIGGQGEIDHIYPRSLSKKHFGVIFNSEVNLIYCSSQGNREKKEEHYLLEHLSPLYLKHQFGTDNVSDIKNFISQNVANIKKYISFHLLTPEQQKAARHALFLDYDDEAFKTITKFLMSQQKARVNGTQKFLGKQIMEFLSTLADSKQLQLEFSIKQITAEEVHDHRELLSKQEPKLVKSRQQSFPSHAIDATLTMSIGLKEFPQFSQELDNSWFINHLMPDEVHLNPVRSKEKYNKPNISSTPLFKDSLYAERFIPVWVKGETFAIGFSEKDLFEIKPSNKEKLFTLLKTYSTKNPGESLQELQAKSKAKWLYFPINKTLALEFLHHYFHKEIVTPDDTTVCHFINSLRYYTKKESITVKILKEPMPVLSVKFESSKKNVLGSFKHTIALPATKDWERLFNHPNFLALKANPAPNPKEFNEFIRKYFLSDNNPNSDIPNNGHNIKPQKHKAVRKVFSLPVIPGNAGTMMRIRRKDNKGQPLYQLQTIDDTPSMGIQINEDRLVKQEVLMDAYKTRNLSTIDGINNSEGQAYATFDNWLTLPVSTFKPEIIKLEMKPHSKTRRYIRITQSLADFIKTIDEALMIKPSDSIDDPLNMPNEIVCKNKLFGNELKPRDGKMKIVSTGKIVTYEFESDSTPQWIQTLYVTQLKKQP

SEQ ID NO: 176 - protein, 1629 aa, Francisella tularensis
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 177 - RNA, 36 nt, Unknown
  Cas12a.1 pre-crRNA
guuuaaggccuugacaaaauuucuacuguaguagau

SEQ ID NO: 178 - RNA, 36 nt, Unknown
  Cas12p pre-crRNA
aucuacaaaaguagaaaucuaauagggauauuggag

SEQ ID NO: 179 - RNA, 36 nt, Unknown
  Cas12p pre-crRNA
cucgaauaucccuauuagauuucuacuuuuguagau

SEQ ID NO: 180 - RNA, 36 nt, Unknown
  Cas12q pre-crRNA
aucuacaaaaguagaaauuaaauaggucuauuugag

SEQ ID NO: 181 - RNA, 36 nt, Unknown
  Cas12q pre-crRNA
cucaaauagaccuauuuaauuucuacuuuuguagau

SEQ ID NO: 182 - protein, 18 aa, Candidatus Methanomethylophilus alvus
LKIIGIDRGERNLIYVTM

SEQ ID NO: 183 - protein, 22 aa, Candidatus Methanomethylophilus alvus
RKALDVREYDNKEARRNWTKVE

SEQ ID NO: 184 - protein, 13 aa, Candidatus Methanomethylophilus alvus
NAIIVMEDLNHGF

SEQ ID NO: 185 - protein, 16 aa, Candidatus Methanomethylophilus alvus
LPQDSDANGAYNIALK

SEQ ID NO: 186 - protein, 18 aa, Synergistes jonesii
VNIIGIDRGERNLVYVSL

SEQ ID NO: 187 - protein, 22 aa, Synergistes jonesii
HAKLNQKEKERDTARKSWKTIG

SEQ ID NO: 188 - protein, 13 aa, Synergistes jonesii
NAVIVMEDLNIGF

SEQ ID NO: 189 - protein, 16 aa, Synergistes jonesii
LPIDADANGAYHIALK

SEQ ID NO: 190 - protein, 18 aa, Lachnospiraceae bacterium ND2006
PYVIGIDRGERNLLYIVV

SEQ ID NO: 191 - protein, 22 aa, Lachnospiraceae bacterium ND2006
HSLLDKKEKERFEARQNWTSIE

SEQ ID NO: 192 - protein, 13 aa, Lachnospiraceae bacterium ND2006
DAVIALEDLNSGF

SEQ ID NO: 193 - protein, 16 aa, Lachnospiraceae bacterium ND2006
LPKNADANGAYNIARK

SEQ ID NO: 194 - protein, 18 aa, Francisella tularensis
VHILSIDRGERHLAYYTL

SEQ ID NO: 195 - protein, 22 aa, Francisella tularensis
HDKLAAIEKDRDSARKDWKKIN

SEQ ID NO: 196 - protein, 13 aa, Francisella tularensis
NAIVVFEDLNFGF

SEQ ID NO: 197 - protein, 16 aa, Francisella tularensis
MPQDADANGAYHIGLK

SEQ ID NO: 198 - protein, 18 aa, Moraxella caprae
VNVIGIDRGERHLLYLTV

SEQ ID NO: 199 - protein, 22 aa, Moraxella caprae
HKILDKREIERLNARVGWGEIE

SEQ ID NO: 200 - protein, 13 aa, Moraxella caprae
NAIVVLEDLNFGF

SEQ ID NO: 201 - protein, 16 aa, Moraxella caprae
QPQNADANGAYHIALK

SEQ ID NO: 202 - protein, 18 aa, Lachnospiraceae bacterium MD335
MHIIGIDRGERNLIYLCM

SEQ ID NO: 203 - protein, 22 aa, Lachnospiraceae bacterium MD335
HQLLKTREDENKSARQSWQTIH

SEQ ID NO: 204 - protein, 13 aa, Lachnospiraceae bacterium MD335
NAIVVLEDLNFGF

SEQ ID NO: 205 - protein, 16 aa, Lachnospiraceae bacterium MD335
MPLDADANGAYNIARK

SEQ ID NO: 206 - protein, 18 aa, Prevotella albensis
THIIGIDRGERHLLYLSL

SEQ ID NO: 207 - protein, 22 aa, Prevotella albensis
HNLLEKREKERTEARHSWSSIE

SEQ ID NO: 208 - protein, 13 aa, Prevotella albensis
NAIVVLEDLNGGF

SEQ ID NO: 209 - protein, 16 aa, Prevotella albensis
FPENADANGAYNIARK

SEQ ID NO: 210 - protein, 18 aa, Unknown
  Smithella sp.
INIIGIDRGERHLLYYAL

SEQ ID NO: 211 - protein, 22 aa, Unknown
  Smithella sp.
HNLLDKKEGDRATARQEWGVIE

SEQ ID NO: 212 - protein, 13 aa, Unknown
  Smithella sp.
NAIIVMEDLNFGF

SEQ ID NO: 213 - protein, 16 aa, Unknown
  Smithella sp.
MPKNADANGAYHIALK

SEQ ID NO: 214 - protein, 18 aa, Unknown
  Porphyromonas sp.
MHVIGIDRGERNLLYICV

SEQ ID NO: 215 - protein, 22 aa, Unknown
  Porphyromonas sp.
HDLLESRDKDRQQERRNWQTIE

SEQ ID NO: 216 - protein, 13 aa, Unknown
  Porphyromonas sp.
KAVVALEDLNMGF

SEQ ID NO: 217 - protein, 16 aa, Unknown
  Porphyromonas sp.
LPKDADANGAYNIALK

SEQ ID NO: 218 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSDDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQDLIAKKTEKAKYLSLETIKLALEEFNKHRDIDKQCRFEEILANFAAIPMIFDEIAQNKDNLAQISLKYQNQGKKDLLQASAEEDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYEKFEFNIEDCRKFIDFYKESISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 219 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 220 - protein, 767 aa, Unknown
  Cas12g1
MAQASSTPAVSPRPRPRYREERTLVRKLLPRPGQSKQEFRENVKKLRKAFLQFNADVSGVCQWAIQFRPRYGKPAEPTETFWKFFLEPETSLPPNDSRSPEFRRLQAFEAAAGINGAAALDDPAFTNELRDSILAVASRPKTKEAQRLFSRLKDYQPAHRMILAKVAAEWIESRYRRAHQNWERNYEEWKKEKQEWEQNHPELTPEIREAFNQIFQQLEVKEKRVRICPAARLLQNKDNCQYAGKNKHSVLCNQFNEFKKNHLQGKAIKFFYKDAEKYLRCGLQSLKPNVQGPFREDWNKYLRYMNLKEETLRGKNGGRLPHCKNLGQECEFNPHTALCKQYQQQLSSRPDLVQHDELYRKWRREYWREPRKPVFRYPSVKRHSIAKIFGENYFQADFKNSVVGLRLDSMPAGQYLEFAFAPWPRNYRPQPGETEISSVHLHFVGTRPRIGFRFRVPHKRSRFDCTQEELDELRSRTFPRKAQDQKFLEAARKRLLETFPGNAEQELRLLAVDLGTDSARAAFFIGKTFQQAFPLKIVKIEKLYEQWPNQKQAGDRRDASSKQPRPGLSRDHVGRHLQKMRAQASEIAQKRQELTGTPAPETTTDQAAKKATLQPFDLRGLTVHTARMIRDWARLNARQIIQLAEENQVDLIVLESLRGFRPPGYENLDQEKKRRVAFFAHGRIRRKVTEKAVERGMRVVTVPYLASSKVCAECRKKQKDNKQWEKNKKRGLFKCEGCGSQAQVDENAARVLGRVFWGEIELPTAIP

SEQ ID NO: 221 - protein, 1300 aa, Francisella tularensis subsp. novicida U112; identical to SEQ ID NO: 219

SEQ ID NO: 222 - protein, 1046 aa, Unknown
  Cas12p truncated
NSIDKVFSVIFYSSCLLQEGIDFYNCVLGGETLPNGEKRQGINELINLYRQKTSEKVPFLKLLDKQILSEKEKFMDEIENDEALLDTLKIFRKSAEEKTTLLKNIFGDFVMNQGKYDLAQIYISRESLNTISRKWTSETDIFEDSLYEVLKKSKIVSASVKKKDGGYAFPEFIALIYVKSALEQIPTEKFWKERYYKNIGDVLNKGFLNGKEGVWLQFLLIFDFEFNSLFEREIIDENGDKKVAGYNLFAKGFDDLLNNFKYDQKAKVVIKDFADEVLHIYQMGKYFAIEKKRSWLADYDIDSFYTDPEKGYLKFYENAYEEIIQVYNKLRNYLTKKPYSEDKWKLNFENPTLADGWDKNKEADNSTVILKKDGRYYLGLMARGRNKLFDDRNLPKILEGVENGKYEKVVYKYFPDQAKMFPKVCFSTKGLEFFQPSEEVITIYKNSEFKKGYTFNVRSMQRLIDFYKDCLVRYEGWQCYDFRNLRKTEDYRKNIEEFFSDVAMDGYKISFQDVSESYIKEKNQNGDLYLFEIKNKDWNEGANGKKNLHTIYFESLFSADNIAMNFPVKLNGQAEIFYRPRTEGLEKERIITKKGNVLEKGDKAFHKRRYTENKVFFHVPITLNRTKKNPFQFNAKINDFLAKNSDINVIGVDRGEKQLAYFSVISQRGKILDRGSLNVINGVNYAEKLEEKARGREQARKDWQQIEGIKDLKKGYISQVVRKLADLAIQYNAIIVFEDLNMRFKQIRGGIEKSVYQQLEKALIDKLTFLVEKEEKDVEKAGHLLKAYQLAAPFETFQKMGKQTGIVFYTQAAYTSRIDPVTGWRPHLYLKYSSAEKAKADLLKFKKIKFVDGRFEFTYDIKSFREQKEHPKATVWTVCSCVERFRWNRYLNSNKGGYDHYSDVTKFLVELFQEYGIDFERGDIVGQIEVLETKGNEKFFKNFVFFFNLICQIRNTNASELAKKDGKDDFILSPVEPFFDSRNSEKFGEDLPKNGDDNGAFNIARKGLVIMDKITKFADENGGCEKMKWGDLYVSNVEWDNFVANK

SEQ ID NO: 223 - protein, 1307 aa, Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 224 - protein, 1206 aa, Lachnospiraceae bacterium MA2020
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 225 - RNA, 36 nt, Unknown
  direct repeat for guide for Cas12a.1 variant A
uaggugaugucaccuuuaaaacaguuccggaauuug

SEQ ID NO: 226 - RNA, 37 nt, Unknown
  direct repeat for guide for Cas12a.1 variant B
gguuuaaggccuugacaaaauuucuccuguaggagau

SEQ ID NO: 227 - RNA, 36 nt, Unknown
  direct repeat for guide for Cas12a.1 variant C
uaggggauguccccuuuaaaacaguuccggaauuug

SEQ ID NO: 228 - RNA, 37 nt, Unknown
  direct repeat for guide for Cas12p variant A
uagguguuuucaccuuuagauuagcccuauaagcucg

SEQ ID NO: 229 - RNA, 36 nt, Unknown
  direct repeat for guide for Cas12p variant B
uagagguuuuccucuuuagauuaucccuauaagcuc

SEQ ID NO: 230 - RNA, 36 nt, Unknown
  direct repeat for guide for Cas12p variant C
uagccguuuucggcuuuagauuaucccuauaagcuc

SEQ ID NO: 231 - RNA, 36 nt, Unknown
  direct repeat for guide for Cas12p variant D
gagcuuacagggauaaucugaagaugaaaacaugua

SEQ ID NO: 232 - RNA, 36 nt, Unknown
  direct repeat for guide for Cas12p variant E
gagcuuaucgggauaaucgaaagaugaaaacaucua

SEQ ID NO: 233 - RNA, 36 nt, Unknown
  direct repeat for guide for Cas12p variant F
gagcuuaccgggauaaucggaagaugaaaacaucua

SEQ ID NO: 234 - RNA, 36 nt, Unknown
  direct repeat for guide for Cas12q variant A
uagguguuuucaccuuuaauuuauccagauaaacuc

SEQ ID NO: 235 - RNA, 36 nt, Unknown
  direct repeat for guide for Cas12q variant B
uagagguuuuccucuuuaauuuauccagauaaacuc

SEQ ID NO: 236 - RNA, 36 nt, Unknown
  direct repeat for guide for Cas12q variant C
uagggguuuuccccuuuaauuuauccagauaaacuc

SEQ ID NO: 237 - RNA, 36 nt, Unknown
  direct repeat for guide for Cas12q variant D
gagcuuaucuggauaaguuaaagaugaaaacaucua

SEQ ID NO: 238 - RNA, 36 nt, Unknown
  direct repeat for guide for Cas12q variant E
gaguuucucuggagaaauuaaagaugaaaacaucua

SEQ ID NO: 239 - RNA, 36 nt, Unknown
  direct repeat for guide for Cas12q variant F
gagcccaucuggauggguuaaagaugaaaacaucua

SEQ ID NO: 240 - protein, 1227 aa, Candidatus Methanomethylophilus alvus
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 241 - protein, 1259 aa, Synergistes jonesii
MANSLKDFTNIYQLSKTLRFELKPIGKTEEHINRKLIIMHDEKRGEDYKSVTKLIDDYHRKFIHETLDPAHFDWNPLAEALIQSGSKNNKALPAEQKEMREKIISMFTSQAVYKKLFKKELFSELLPEMIKSELVSDLEKQAQLDAVKSFDKFSTYFTGFHENRKNIYSKKDTSTSIAFRIVHQNFPKFLANVRAYTLIKERAPEVIDKAQKELSGILGGKTLDDIFSIESFNNVLTQDKIDYYNQIIGGVSGKAGDKKLRGVNEFSNLYRQQHPEVASLRIKMVPLYKQILSDRTTLSFVPEALKDDEQAINAVDGLRSELERNDIFNRIKRLFGKNNLYSLDKIWIKNSSISAFSNELFKNWSFIEDALKEFKENEFNGARSAGKKAEKWLKSKYFSFADIDAAVKSYSEQVSADISSAPSASYFAKFTNLIETAAENGRKFSYFAAESKAFRGDDGKTEIIKAYLDSLNDILHCLKPFETEDISDIDTEFYSAFAEIYDSVKDVIPVYNAVRNYTTQKPFSTEKFKLNFENPALAKGWDKNKEQNNTAIILMKDGKYYLGVIDKNNKLRADDLADDGSAYGYMKMNYKFIPTPHMELPKVFLPKRAPKRYNPSREILLIKENKTFIKDKNFNRTDCHKLIDFFKDSINKHKDWRTFGFDFSDTDSYEDISDFYMEVQDQGYKLTFTRLSAEKIDKWVEEGRLFLFQIYNKDFADGAQGSPNLHTLYWKAIFSEENLKDVVLKLNGEAELFFRRKSIDKPAVHAKGSMKVNRRDIDGNPIDEGTYVEICGYANGKRDMASLNAGARGLIESGLVRITEVKHELVKDKRYTIDKYFFHVPFTINFKAQGQGNINSDVNLFLRNNKDVNIIGIDRGERNLVYVSLIDRDGHIKLQKDFNIIGGMDYHAKLNQKEKERDTARKSWKTIGTIKELKEGYLSQVVHEIVRLAVDNNAVIVMEDLNIGFKRGRFKVEKQVYQKFEKMLIDKLNYLVFKDAGYDAPCGILKGLQLTEKFESFTKLGKQCGIIFYIPAGYTSKIDPTTGFVNLFNINDVSSKEKQKDFIGKLDSIRFDAKRDMFTFEFDYDKFRTYQTSYRKKWAVWTNGKRIVREKDKDGKFRMNDRLLTEDMKNILNKYALAYKAGEDILPDVISRDKSLASEIFYVFKNTLQMRNSKRDTGEDFIISPVLNAKGRFFDSRKTDAALPIDADANGAYHIALKGSLVLDAIDEKLKEDGRIDYKDMAVSNPKWFEFMQTRKFDF

SEQ ID NO: 242 - protein, 1228 aa, Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 243 - protein, 1300 aa, Francisella tularensis; identical to SEQ ID NO: 219

SEQ ID NO: 244 - protein, 632 aa, Moraxella bovoculi
KRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 245 - protein, 1230 aa, Lachnospiraceae bacterium MD335
MHENNGKIADNFIGIYPVSKTLRFELKPVGKTQEYIEKHGILDEDLKRAGDYKSVKKIIDAYHKYFIDEALNGIQLDGLKNYYELYEKKRDNNEEKEFQKIQMSLRKQIVKRFSEHPQYKYLFKKELIKNVLPEFTKDNAEEQTLVKSFQEFTTYFEGFHQNRKNMYSDEEKSTAIAYRVVHQNLPKYIDNMRIFSMILNTDIRSDLTELFNNLKTKMDITIVEEYFAIDGFNKVVNQKGIDVYNTILGAFSTDDNTKIKGLNEYINLYNQKNKAKLPKLKPLFKQILSDRDKISFIPEQFDSDTEVLEAVDMFYNRLLQFVIENEGQITISKLLTNFSAYDLNKIYVKNDTTISAISNDLFDDWSYISKAVRENYDSENVDKNKRAAAYEEKKEKALSKIKMYSIEELNFFVKKYSCNECHIEGYFERRILEILDKMRYAYESCKILHDKGLINNISLCQDRQAISELKDFLDSIKEVQWLLKPLMIGQEQADKEEAFYTELLRIWEELEPITLLYNKVRNYVTKKPYTLEKVKLNFYKSTLLDGWDKNKEKDNLGIILLKDGQYYLGIMNRRNNKIADDAPLAKTDNVYRKMEYKLLTKVSANLPRIFLKDKYNPSEEMLEKYEKGTHLKGENFCIDDCRELIDFFKKGIKQYEDWGQFDFKFSDTESYDDISAFYKEVEHQGYKITFRDIDETYIDSLVNEGKLYLFQIYNKDFSPYSKGTKNLHTLYWEMLFSQQNLQNIVYKLNGNAEIFYRKASINQKDVVVHKADLPIKNKDPQNSKKESMFDYDIIKDKRFTCDKYQFHVPITMNFKALGENHFNRKVNRLIHDAENMHIIGIDRGERNLIYLCMIDMKGNIVKQISLNEIISYDKNKLEHKRNYHQLLKTREDENKSARQSWQTIHTIKELKEGYLSQVIHVITDLMVEYNAIVVLEDLNFGFKQGRQKFERQVYQKFEKMLIDKLNYLVDKSKGMDEDGGLLHAYQLTDEFKSFKQLGKQSGFLYYIPAWNTSKLDPTTGFVNLFYTKYESVEKSKEFINNFTSILYNQEREYFEFLFDYSAFTSKAEGSRLKWTVCSKGERVETYRNPKKNNEWDTQKIDLTFELKKLFNDYSISLLDGDLREQMGKIDKADFYKKFMKLFALIVQMRNSDEREDKLISPVLNKYGAFFETGKNERMPLDADANGAYNIARKGLWIIEKIKNTDVEQLDKVKLTISNKEWLQYAQEHIL

SEQ ID NO: 246 - protein, 1253 aa, Prevotella albensis
MNIKNFTGLYPLSKTLRFELKPIGKTKENIEKNGILTKDEQRAKDYLIVKGFIDEYHKQFIKDRLWDFKLPLESEGEKNSLEEYQELYELTKRNDAQEADFTEIKDNLRSSITEQLTKSGSAYDRIFKKEFIREDLVNFLEDEKDKNIVKQFEDFTTYFTGFYENRKNMYSSEEKSTAIAYRLIHQNLPKFMDNMRSFAKIANSSVSEHFSDIYESWKEYLNVNSIEEIFQLDYFSETLTQPHIEVYNYIIGKKVLEDGTEIKGINEYVNLYNQQQKDKSKRLPFLVPLYKQILSDREKLSWIAEEFDSDKKMLSAITESYNHLHNVLMGNENESLRNLLLNIKDYNLEKINITNDLSLTEISQNLFGRYDVFTNGIKNKLRVLTPRKKKETDENFEDRINKIFKTQKSFSIAFLNKLPQPEMEDGKPRNIEDYFITQGAINTKSIQKEDIFAQIENAYEDAQVFLQIKDTDNKLSQNKTAVEKIKTLLDALKELQHFIKPLLGSGEENEKDELFYGSFLAIWDELDTITPLYNKVRNWLTRKPYSTEKIKLNFDNAQLLGGWDVNKEHDCAGILLRKNDSYYLGIINKKTNHIFDTDITPSDGECYDKIDYKLLPGANKMLPKVFFSKSRIKEFEPSEAIINCYKKGTHKKGKNFNLTDCHRLINFFKTSIEKHEDWSKFGFKFSDTETYEDISGFYREVEQQGYRLTSHPVSASYIHSLVKEGKLYLFQIWNKDFSQFSKGTPNLHTLYWKMLFDKRNLSDVVYKLNGQAEVFYRKSSIEHQNRIIHPAQHPITNKNELNKKHTSTFKYDIIKDRRYTVDKFQFHVPITINFKATGQNNINPIVQEVIRQNGITHIIGIDRGERHLLYLSLIDLKGNIIKQMTLNEIINEYKGVTYKTNYHNLLEKREKERTEARHSWSSIESIKELKDGYMSQVIHKITDMMVKYNAIVVLEDLNGGFMRGRQKVEKQVYQKFEKKLIDKLNYLVDKKLDANEVGGVLNAYQLTNKFESFKKIGKQSGFLFYIPAWNTSKIDPITGFVNLFNTRYESIKETKVFWSKFDIIRYNKEKNWFEFVFDYNTFTTKAEGTRTKWTLCTHGTRIQTFRNPEKNAQWDNKEINLTESFKALFEKYKIDITSNLKESIMQETEKKFFQELHNLLHLTLQMRNSVTGTDIDYLISPVADEDGNFYDSRINGKNFPENADANGAYNIARKGLMLIRQIKQADPQKKFKFETITNKDWLKFAQDKPYLKD

SEQ ID NO: 247 - protein, 1250 aa, Smithella sp. SC K08D17
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYAWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 248 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 249 - DNA, 43 bp, Unknown
  gRNA
aaatttctactgtagtagatgtggcagctcaaaaattggctac